(19) United States
(12) Patent Application Publication
(10) Pub. No.: US 2020/0352931 A1
(43) Pub. Date: Nov. 12, 2020

(54) OXALAMIDES AS MODULATORS OF INDOLEAMINE 2,3-DIOXYGENASE

(71) Applicant: PHENEX DISCOVERY VERWALTUNGS-GMBH, Heidelberg (DE)

(72) Inventors: Christoph Steeneck, Heidelberg (DE); Olaf Kinzel, Heidelberg (DE); Simon Anderhub, Basel (CH); Martin Hornberger, Ladenburg (DE); Marta Czekanska, Heidelberg (DE); Thomas Hoffmann, Speyer (DE)

(73) Assignee: PHENEX DISCOVERY VERWALTUNGS-GMBH, Heidelberg (DE)

(21) Appl. No.: 16/771,631

(22) PCT Filed: Dec. 12, 2018
(86) PCT No.: PCT/EP2018/084483

§ 371 Date: Jun. 10, 2020

(30) Foreign Application Priority Data
Dec. 12, 2017 (EP) 17206659.9

(51) Int. Cl. A61K 31/4709; A61K 31/451; A61K 31/519; C07D 487/04; C07D 401/04; C07D 211/14; C07D 207/06; C07D 401/14; A61K 31/55; A61K 31/40; C07D 491/107; A61K 31/438

(52) U.S. Cl. CPC A61K 31/4709; A61K 31/451; A61K 31/519; C07D 487/04; C07D 401/04; A61K 31/438; C07D 207/06; C07D 401/14; A61K 31/55; A61K 31/40; C07D 491/107; C07D 211/14

(57) ABSTRACT

The present invention relates to novel compounds which act as modulators of indoleamine 2,3-dioxygenase (IDO1) and to the use of said compounds in the prophylaxis and/or treatment of diseases or conditions mediated by indoleamine 2,3-dioxygenase. The invention further relates to pharmaceutical compositions comprising the novel compounds.

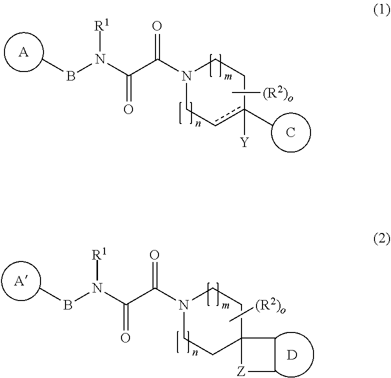

The present invention relates to novel compounds which act as modulators of indoleamine 2,3-dioxygenase (IDO1) and to the use of said compounds in the prophylaxis and/or treatment of diseases or conditions mediated by indoleamine 2,3-dioxygenase. The invention further relates to pharmaceutical compositions comprising the novel compounds.

Tryptophan is an essential amino acid and naturally serves as a building block for proteins. The majority of adult Tryptophan intake is not utilized for protein synthesis though, but channeled into two conversion pathways. The first pathway leading to the production of Serotonine degrades approximately 1% of ingested Tryptophan, whereas the majority of ˜90% of Tryptophan fuels the so called Kynurenine pathway (Le Floc'h et al.; Amino Acids. 2011; 41(5):1195-205).

The Kynurenine pathway of Tryptophan degradation is initialized by a specific set of enzymes, including Indoleamine 2,3-dioxygenase 1 (IDO1) and Tryptophan 2,3-dioxygenase (TDO2). The product of this reaction, N-Formylkynurenine is subsequently converted to Kynurenine, which can be further metabolized to such diverse products as Xanthurenic acid, Anthranilic acid or Nicotinamide to name a few (Stone, Darlington; Nat Rev Drug Discov. 2002; 1(8):609-20).

Under physiological conditions the expression of TDO is restricted to the liver (Bertazzo et al.; Biochim Biophys Acta. 2001; 15; 1527(3):167-75) and the brain (Miller et al.; Neurobiol Dis. 2004; 15(3):618-29). IDO1 in contrast is found in a variety of tissues such as lung, digestive tract, uterus and secondary lymphoid organs (Théate et al.; Cancer Immunol Res. 2015; 3(2):161-72) and is readily (further) induced by pro inflammatory cytokines (Taylor, Feng; FASEB J. 1991; 5(11):2516-22 1991).

Initially, IDO1 has been implicated in a protective role in fetal rejection. Mice, treated with the IDO1 inhibitor 1-Methyl-Tryptophan lost their allogeneic concepti in a T cell dependent manner (Munn et al.; Science. 1998; 281(5380):1191-3).

It was then conceived that IDO1 creates an immunosuppressive environment by catabolizing Tryptophan, thereby locally depleting this amino acid and creating immune privilege sites. Tryptophan depletion is most likely sensed through the General Control Nonderepressable Kinase 2 (GCN2) and leads to activation of the integrated stress response of cells (Munn et al.; Immunity. 2005; 22(5):633-42) with consecutive inhibition of T cell proliferation (Munn et al.; J Exp Med. 1999; 189(9):1363-72). Additionally, a low Tryptophan environment also sensitizes activated T cells to apoptosis via Fas (Lee et al.; Immunology. 2002; 107(4):452-60). More recently, the mechanism of how IDO1 can lead to immune suppression has been expanded, focusing on the catabolites of Tryptophan enzymatic conversion by IDO1, collectively called Kynurenines. It has been demonstrated that Kynurenine, 3-Hydroxykynurenine, 3-Hydroxyanthranilic acid and Quinolinic acid led to dose dependent inhibition of T cell proliferation (Terness et al.; J Exp Med. 2002; 196(4):447-57). In part, this may be due to cell type specific apoptosis of Thymocytes in response to incubation with the aforementioned Tryptophan metabolites (Fallarino et al.; Cell Death Differ. 2002; 9(10):1069-77).

The immune suppression observed concurrently with IDO1 expression is also associated with an increase in T cells displaying a regulatory phenotype (Treg). Treg cells are important to maintain immune homeostasis and induce immune tolerance to avoid inappropriate immune response as is the case in autoimmune disease (Sakaguchi et al.; Eur J Immunol. 2007; 37 Suppl 1:S116-23). In mice, expression of the transcription factor FOXP3 is an important marker for regulatory T cells (Fontenot et al.; Nat Immunol. 2003; 4(4):330-6) and co-cultivation of murine naïve CD4+ T cells with IDO positive Dendritic cells led to a remarkable increase in FOXP3 expression of the CD4+ population. This polarization could be mimicked by incubation of naïve CD4+ T cells in low Tryptophan medium supplemented with Kynurenines and was shown to be dependent on GCN2 (Fallarino et al.; Transpl Immunol. 2006; 17(1):58-60). In humans, AML patients with elevated levels of IDO1 also displayed an increase in circulating Treg cells. Analogous to the situation in mice, human CD3+ cells were polarized towards a regulatory phenotype in an IDO1 dependent manner when co-cultivated with IDO1 positive cells derived from AML patients (Curti et al.; Blood. 2007; 109(7):2871-7).

Several Kynurenines such as Kynurenine itself, 3-Hydroxykynurenine and Kynurenic acid also serve as ligands for the Aryl Hydrocarbon Receptor (AHR) albeit with differentially reported efficacies (DiNatale et al.; Toxicol Sci. 2010; 115(1):89-97, Mezrich et al.; J Immunol 2010; 185(6):3190-8). This is of particular interest because firstly, the AHR has been implicated in the transcriptional regulation of IDO1 via a self-sustaining autocrine feed-forward loop with the AHR acting either directly on IDO1 transcription (Li et al.; J Immunol® 2016; 197(3):962-70) or with IL-6 as mediator (Litzenburger et al.; Oncotarget. 2014; 5(4):1038-51). Secondly, because the polarization of naïve CD4+ T cells towards Treg cells by Kynurenines is dependent on the AHR (Kimura et al.; Proc Natl Acad Sci USA. 2008 Jul. 15; 105(28):9721-6, Mezrich et al.; J Immunol. 2010; 185(6):3190-8).

Whether the depletion of Tryptophan or the generation of Kynurenines or the combined action of both is the key in creating an immune suppressive environment needs to be further investigated. The net result though, is a key factor not only for immune homeostasis in healthy individuals but also for how tumors can escape immune surveillance.

The importance of IDO1 for cancer development is supported by several lines of evidence. IDO1 has been detected in most human tumors, such as prostate, pancreas, lung, ovarian, colorectal cancer, melanoma and leukemia (Uyttenhove et al.; Nat Med. 2003; 9(10):1269-74; Hanagiri et al.; J Clin Cell Immunol 2014, 5:5, Okamoto et al.; Clin Cancer Res. 2005; 11(16):6030-9; Ferdinande et al.; Br J Cancer. 2012; 106(1):141-7, Brody et al.; Cell Cycle. 2009; 8(12):1930-4, Chamuleau et al.; Haematologica. 2008; 93(12):1894-8, Theáte et al.; Cancer Immunol Res. 2015; 3(2):161-72). Interestingly, IDO1 positive cells were also often found in immune cells in the tumor stroma and adjoining tumor draining lymph nodes (Astigiano et al.; Neoplasia. 2005; 7(4):390-6, Chen et al.; Breast Cancer Res. 2014; 16(4):410, Polak et al.; Br J Cancer. 2007; 96(12):1879-87, Theáte et al.; Cancer Immunol Res. 2015; 3(2):161-72). A negative correlation of IDO1 expression either in tumor or in stromal cells with markers of disease progression has been observed in most of these cases.

Apart from these correlative analysis, elegant studies using mouse models underpinned the importance of IDO1 in tumor immune escape. When immunogenic mouse tumor cells lacking IDO1 were injected into immune competent mice, no tumor growth was observed. In contrast, if the cells constitutively expressed IDO1, tumors grew as expected. Pharmacologic inhibition of IDO1 in turn, resulted in a marked reduction of tumor outgrowth. As indicated above, this effect was dependent on the hosts' immune system, as immune compromised mice injected with the IDO1 positive and negative cell lines developed tumors to the same extent. Also, lower numbers of CD8+ T cells were found in mice injected with IDO1 positive cells in comparison to mice injected with IDO1 negative cells (Uyttenhove et al.; Nat Med. 2003; 9(10):1269-74).

Although tumor derived IDO1 is a decisive factor for immune escape, research also investigated the role of IDO1 in immune cells. Munn et al. found a subset of plasmacytoid Dendritic cells in Tumor draining lymph nodes expressing IDO1. Although these cells comprised less than 1% of all lymph node cells they acted as potent and dominant suppressors of T cell proliferation (Munn et al.; J Clin Invest. 2004; 114(2): 280-290). The relative contribution of IDO1 from immune cells versus tumor derived IDO1 is still under debate. Koblish et al. observed that pharmacologic inhibition of IDO1 reduced tumor size, when IDO1 positive tumor cells were transplanted into immune competent IDO1 −/− mice (Koblish et al.; Mol Cancer Ther. 2010; 9(2):489-98). In contrast, Banerjee et al. reported no effect on tumor size when using a syngeneic mouse tumor model in IDO1 negative mice and administration of an IDO1 inhibitor (Banerjee et al.; Oncogene. 2008; 27(20):2851-7). Both studies though, were able to demonstrate the efficacy of IDO inhibitors in preclinical mouse models as single agents. Moreover, synergistic or additive effects were observed when IDO1 inhibitors where used in combination with chemotherapeutics, irradiation, tumor vaccines or immune checkpoint inhibitors (Muller et al.; Nat Med. 2005; 11(3):312-9, Hou et al.; Cancer Res. 2007 Jan. 15; 67(2):792-801, Sharma et al.; Blood. 2009 Jun. 11; 113(24):6102-11, Spranger et al.; J Immunother Cancer. 2014; 2:3)

The studies referenced herein did not report any potent toxicity of IDO1 inhibition and it is of interest to note that IDO knockout mice are viable and exhibit no major abnormal phenotype apart from defects in acquired tolerance (Mellor et al.; J Immunol. 2003; 171(4):1652-5). Therefore it seems unlikely that IDO1 inhibition in humans will encounter profound dose limiting toxicities.

Apart from its relevance for tumor immune evasion, IDO1 is implicated in a plethora of other medical conditions.

Throughout HIV disease progression, an altered Th17/Treg balance has been observed, favoring the latter in later stages. Favre et al. were able to demonstrate a crucial role for the Kynurenine 3-Hydroxykynurenine in this process and it is therefore hypothesized that patients with HIV may benefit from IDO1 inhibition together with antiretroviral therapy (Favre et al.; Sci Transl Med. 2010 May 19; 2(32):32ra36.).

IDO has also been implicated in the onset and progression of other viral and bacterial infections such as tuberculosis (TB), hanta virus infection, and leprae (Kim et al. 2017; Immunology. 151(2):177-190; Adu-Gyamfi et al.; Clin. Infect Dis. 2017 Oct. 15; 65(8):1356-1358; Koivula et al.; Pathog Dis. 2017 February; 75(1); de Mattos Barbosa et al.; Microbes Infect. 2017 Jul. 3.) The description of the involvement of IDO in cardiovascular disease, endometriosis, type 2 diabetic nephropathy, chronic obstructive pulmonary disease (COPD), and osteoporosis, (H. Mangge et al.; Curr Med Chem. 2014 June; 21(17): 1931-1937; Mei et al.; Mol Med Rep. 2017 April; 15(4):2255-2260; Wei et al.; Cell Death Dis. 2016 Dec. 1; 7(12); Zhang et al.; J Diabetes Complications. 2017 January; 31(1):223-227; Gulcev et al.; Int J Chron Obstruct Pulmon Dis. 2016 Sep. 29; 11:2435-2446; Refaey et al.; J Bone Miner Res. 2017 Jul. 20; Meier et al.; Lung. 2017 June; 195(3):303-311) suggest that such disease states could be treated with positive outcome with IDO inhibitors

As expected from the mode of action in the immune system, IDO is also involved in autoimmune diseases like rheumatoid arthritis, multiple sclerosis (MS), inflammatory bowel disease (IBD), psoriasis, and systemic lupus erythematosus, (Merlo et al.; Clin Immunol. 2017 June; 179:8-16; Lovelace et al.; Front Immunol. 2016 Aug. 4; 7:246; Negrotto et al. J Immunol. 2017 Mar. 1; 198(5):1900-1909; Sznurkowska et al. J Biol Regul Homeost Agents. 2017 January-March; 31(1):125-131; Jamie et al.; J Allergy Clin Immunol. 2016 June; 137(6): 1830-1840; Wang et al.; Arthritis Rheumatol. 2014 August; 66(8): 2234-2245) again here the inhibition of the enzyme could lead to beneficial effects in treating these diseases.

IDO1 also seems to be involved in disorders of the central nervous system because its downstream products 3-Hydroxykynurenine and quinolinic acid act as neurotoxins (Okuda et al.; J Neurochem. 1998; 70(1):299-307, Schwarcz et al.; Science. 1983; 219(4582):316-8). Thereby, IDO1 is also implicated in the disease development of Huntington's disease, Amyotrophic lateral sclerosis, Alzheimer's disease, Parkinson's disease, Schizophrenia and epilepsy (Thevandavakkam et al.; CNS Neurol Disord Drug Targets. 2010; 9(6):791-800; Chen et al.; Neurotox Res. 2010; 18(2):132-42; Guillemin et al.; Neuropathol Appl Neurobiol. 2005; 31(4):395-404; Lim et al.; Prog Neurobiol. 2016; pii: S0301-0082(15)30055-1, Kegel et al.; Int J Tryptophan Res. 2014; 7: 15-22; Singh et al.; Neurochem Int. 2017 March; 104:27-33; Kaur et al.; Epilepsy Behav. 2017 July; 72:8-16.).

IDO1 inhibitors may therefore be of high potential value for the treatment of HIV and CNS disorders and the reported preclinical data on efficacy against tumors either alone or in combination with other drugs validate the use of IDO1 inhibitors as a treatment option for antineoplastic therapies.

Compounds acting as IDO1 inhibitors are known in the art. WO 2006/122150 discloses compounds with a N-hydroxyamidino motif as potential modulators of IDO1. The efficacy of compounds having said motif is demonstrated e.g. in WO 2008/036642, WO 2008/036643, WO 2008/036652, WO 2008/036653 and WO 2008/05178.

Compounds having an oxalamide motif are described as inhibitors for other receptors than IDO1. As an example, the compounds disclosed in WO 2018/049089 can be mentioned.

The compounds detailed herein and compositions thereof as well as the methods described will serve to meet the future need for potent IDO1 inhibitors.

It is the object of the present invention to provide novel compounds which are suitable as potent IDO1 inhibitors.

Said object is solved by the compounds of Formulae (1) and (2)

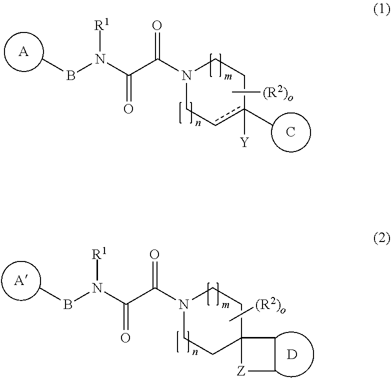

wherein A, A′, B, C, D, Y, Z, R¹, R², m, n and o are defined as in the appended claims.

It is further an object of the present invention to provide compounds according to Formulae (1) and (2) for use in the prophylaxis and/or treatment of diseases and conditions mediated by indoleamine 2,3-dioxygenase.

The present invention further relates to the use of the compounds according to Formulae (1) or (2) for the preparation of a medicament for the treatment and/or prophylaxis of a disease or condition mediated by indoleamine 2,3-dioxygenase.

Moreover, the present invention also relates to a method for treating or preventing a disease or condition mediated by indoleamine 2,3-dioxygenase, the method comprising administering an effective amount of a compound according to Formulae (1) and (2) to a patient in need thereof.

Accordingly, the present invention provides a compound represented by Formulae (1) or (2)

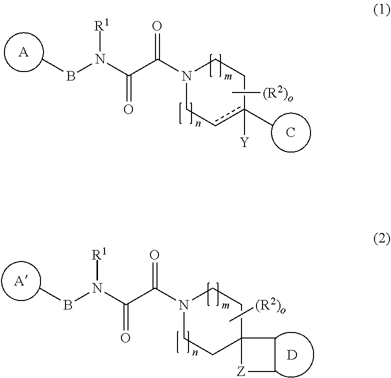

an enantiomer, diastereomer, tautomer or pharmaceutically acceptable salt thereof wherein

A and A′ represent C₃₋₁₀ cycloalkyl, which may be optionally fused with a phenyl ring being unsubstituted or substituted with 1 to 3 R^{a}, 3- to 10-membered heterocycloalkyl containing 1 to 4 heteroatoms independently selected from O, N and S. 6- to 10-membered mono or bicyclic aryl or 5- to 10-membered mono or bicyclic heteroaryl containing 1 to 4 heteroatoms independently selected from O, N and S,

wherein cycloalkyl, heterocycloalkyl, aryl and heteroaryl are unsubstituted or substituted with 1 to 5 substituents independently selected from the group consisting of halogen, OH, R^{x}, O—R^{x}, OC(O)—R^{x}, S—R^{x}, S(O)₂—R^{x}, S(O)₂N(R¹)₂, N(R¹)₂, NR¹C(O)R^{x}, C(O)N(R¹)₂, C(O)O—R^{x}, C(O)—R^{x}, CN, COOH, 5- or 6-membered heteroaryl containing 1 to 4 heteroatoms independently selected from O, N and S and 6-membered aryl,

wherein heteroaryl and aryl are unsubstituted or substituted with 1 to 5 substituents independently selected from the group consisting of halogen, CN, OR¹, C₁₋₆-alkyl, C₃₋₆-cycloalkyl and halo-C₁₋₆-alkyl, or

two substituents on the same carbon atom or on two different carbon atoms form together with the carbon atom to which they are attached a C₃₋₁₀ cycloalkyl group, wherein optionally one carbon atom in the cycloalkyl ring may be replaced by a heteroatom selected from O, N and S and wherein the (hetero)cyclic ring may be unsubstituted or substituted by 1 to 3 substituents independently selected from the group consisting of halogen, C₁₋₆-alkyl, halo-C₁₋₆-alkyl or oxo;

R^{a} represents halogen, CN, C₁₋₆-alkyl, halo-C₁₋₆-alkyl, hydroxy-C₁₋₆-alkyl, C₃₋₆-cycloalkyl or halo-C₃₋₆-cycloalkyl;

R^{x} represents C₁₋₆-alkyl, C₃₋₆-cycloalkyl or 3- to 6 membered heterocyclyl containing 1 to 2 heteroatoms independently selected from O, N and S,

wherein alkyl, cycloalkyl and heterocycloalkyl are unsubstituted or substituted with 1 or 6 substituents independently selected from the group consisting of halogen, C₁₋₆-alkyl, halo-C₁₋₆-alkyl, OR¹ and CN;

B represents a bond or C₁₋₂-alkylene wherein alkylene is unsubstituted or substituted with one or two C₁₋₄-alkyl;

C represents 6- to 10-membered mono- or bicyclic aryl or 5- to 14-membered mono-, bi- or tricyclic heteroaryl containing 1 to 4 heteroatoms independently selected from O, N and S,

wherein aryl and heteroaryl are unsubstituted or substituted with 1 to 5 substituents independently selected from the group consisting of halogen, OH, R^{y}, O—R^{y}, OC(O)—R^{y}, S—R^{y}, S(O)₂—R^{y}, S(O)₂N(R¹)₂, N(R¹)₂, NR¹C(O)R^{y}, C(O)N(R¹)₂, C(O)O—R^{y}, C(O)—R^{y}, CN, COOH, 5- or 6-membered heteroaryl containing 1 to 4 heteroatoms independently selected from O, N and S and 6-membered aryl,

wherein aryl and heteroaryl are unsubstituted or substituted with 1 to 5 substituents independently selected from the group consisting of halogen, CN, OR¹, C₁₋₆-alkyl, C₃₋₆-cycloalkyl and halo-C₁₋₆-alkyl, or

two substituents on the aryl or heteroaryl ring systems together with the carbon atoms to which they are attached form a 5- or 6-membered heterocyclic ring containing 1 or 2 heteroatoms independently selected from O, N and S,

wherein the heterocylic ring is unsubstituted or substituted with 1 or 2 substituents independently selected from the group consisting of halogen, C₁₋₆-alkyl, halo-C₁₋₆-alkyl, CN and oxo;

R^{y} represents C₁₋₆-alkyl, C₃₋₆-cycloalkyl or 3- to 6 membered heterocyclyl containing 1 to 2 heteroatoms independently selected from O, N and S,

wherein alkyl, cycloalkyl and heterocycloalkyl are unsubstituted or substituted with 1 to 6 substituents independently selected from the group consisting of halogen, C₁₋₆-alkyl, halo-C₁₋₆-alkyl, OR¹, CN and phenyl;

D represents 6- to 10-membered mono- or bicyclic aryl or 5- to 10-membered mono- or bicyclic heteroaryl containing 1 to 4 heteroatoms independently selected from O, N and S,

wherein aryl and heteroaryl are unsubstituted or substituted with 1 to 5 substituents independently selected from the group consisting of halogen, OH, R^{z}, O—R^{z}, OC(O)—R^{z}, S—R^{z}, S(O)₂—R^{z}, S(O)₂N(R¹)₂, N(R¹)₂, NR¹C(O)R^{z}, C(O)N(R¹)₂, C(O)O—R^{z}, C(O)—R^{z}, CN, COOH, 5- or 6-membered heteroaryl containing 1 to 4 heteroatoms independently selected from O, N and S and 6-membered aryl,

wherein aryl and heteroaryl are unsubstituted or substituted with 1 to 5 substituents independently selected from the group consisting of halogen, CN, OR¹, C₁₋₆-alkyl, C₃₋₆-cycloalkyl and halo C₁₋₆-alkyl, or

two substituents on the aryl or heteroaryl ring systems together with the carbon atom to which they are attached form a 5- or 6-membered heterocyclic ring containing 1 or 2 heteroatoms independently selected from O, N and S,

wherein the heterocylic ring is unsubstituted or substituted with 1 or 2 substituents independently selected from the group consisting of halogen, C₁₋₆-alkyl, halo-C₁₋₆-alkyl, CN and oxo;

R^{z} represents C₁₋₆-alkyl, C₃₋₆-cycloalkyl or 3- to 6 membered heterocyclyl containing 1 to 2 heteroatoms independently selected from O, N and S,

wherein alkyl, cycloalkyl and heterocycloalkyl are unsubstituted or substituted with 1 to 6 substituents independently selected from the group consisting of halogen, C₁₋₆-alkyl, halo-C₁₋₆-alkyl, OR¹ and CN;

Y is absent or represents hydrogen, OR⁴, halogen, C₁₋₆ alkyl or halo-C₁₋₆ alkyl;

Z represents —C₂₋₃-alkylene-, —O—C₁₋₂-alkylene-, —C₁₋₂-alkylene-O—, —NR³C(O)—C₀₋₁-alkylene-, —C(O)NR³—C₀₋₁-alkylene-, —C₀₋₁-alkylene-NR³C(O)—, —C₀₋₁-alkylene C(O)NR³—, —S(O)ₜ—C₁₋₂-alkylene-, —C₁₋₂-alkylene-S(O)ₜ—, —NR⁹—C₁₋₂-alkylene- or —C₁₋₂-alkylene-NR⁹—,

wherein alkylene is unsubstituted or substituted with 1 or 2 substituents independently selected from the group consisting of OR⁴, C₁₋₆-alkyl, halogen and halo-C₁₋₆-alkyl;

R⁹ is hydrogen, C₁₋₆-alkyl, halo-C₁₋₆-alkyl, C(O)—C₁₋₆-alkyl, or C(O)-halo-C₁₋₆-alkyl;

R¹ is hydrogen or C₁₋₆-alkyl;

R² is halogen, C₁₋₆ alkyl, C₃₋₆-cycloalkyl, halo-C₁₋₆-alkyl, OR⁴, S(O)₂N(R¹)₂, S(O)₂—C₁₋₆-alkyl, S(O)₂—C₃₋₆-cycloalkyl, S(O)₂-halo-C₁₋₆-alkyl, S(O)₂N(R¹)₂, C(O)N(R¹)₂, CN or oxo or

two R² on the same carbon atom form together with the carbon atom to which they are attached a C₃₋₁₀-cycloalkyl group, or two R² at different carbon atoms form together a —CH₂—, —CH(CH₃)—, —C(CH₃)₂—, CH₂—CH(CH₃)—, —CH₂—CH₂— or —CH₂—CH₂—CH₂— group;

R³ is hydrogen or C₁₋₆-alkyl;

R⁴ is hydrogen or C₁₋₆-alkyl;

m is 0-2;

n is 0-2;

o is 0-4; and

t is 0, 1 or 2;

with the proviso that C is not

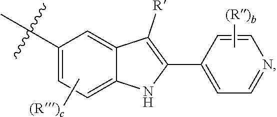

wherein R′ is H, Cl, CN, C₁₋₄-alkyl, C₁₋₃-fluoroalkyl, C₁₋₃-hydroxy-fluoroalkyl, C₃₋₆-cycloalkyl, —C(O)O(C₁₋₃-alkyl) or tetrahydropyranyl, R″ is halogen, CN, OH, C₁₋₃-alkyl, C₁₋₂-fluoroalkyl, O—C₁₋₃-alkyl or C₃₋₆-cycloalkyl, R′″ is F, Cl, CN, C₁₋₂-alkyl, C₁₋₂-fluoro-alkyl or OCH₃, b is 0 to 4 and c is 0, 1 or 2.

In a preferred embodiment in combination with any of the above and below embodiments, the compound is represented by Formula (1).

In a preferred embodiment of the compounds according to Formula (1) in combination with any of the above and below embodiments, A represents C₃₋₁₀ cycloalkyl, which may be optionally fused with a phenyl ring being unsubstituted or substituted with 1 to 3 R^{a}, 3- to 10-membered heterocycloalkyl containing 1 to 4 heteroatoms independently selected from O, N and S, 6- to 10-membered mono or bicyclic aryl or 5- to 10-membered mono or bicyclic heteroaryl containing 1 to 4 heteroatoms independently selected from O, N and S,

wherein cycloalkyl, heterocycloalkyl, aryl and heteroaryl are unsubstituted or substituted with 1 to 5 substituents independently selected from the group consisting of halogen, OH, R^{x}, O—R^{x}, OC(O)—R^{x}, S—R^{x}, S(O)₂—R^{x}, S(O)₂N(R¹)₂, N(R¹)₂, NR¹C(O)R, C(O)N(R¹)₂, C(O)O—R^{x}, C(O)—R^{x}, CN, COOH, 5- or 6-membered heteroaryl containing 1 to 4 heteroatoms independently selected from O, N and S and 6-membered aryl, wherein heteroaryl and aryl are unsubstituted or substituted with 1 to 5 substituents independently selected from the group consisting of halogen, CN, OR¹, C₁₋₆-alkyl, C₃₋₆-cycloalkyl and halo-C₁₋₆-alkyl, or

two substituents on the same carbon atom or on two different carbon atoms form together with the carbon atom to which they are attached a C₃₋₁₀ cycloalkyl group, wherein optionally one carbon atom in the cycloalkyl ring may be replaced by a heteroatom selected from O, N and S and wherein the (hetero)cyclic ring may be unsubstituted or substituted by 1 to 3 substituents independently selected from the group consisting of halogen, C₁₋₆-alkyl, halo-C₁₋₆-alkyl or oxo;

R^{a} represents halogen, CN, C₁₋₆-alkyl, halo-C₁₋₆-alkyl, hydroxy-C₁₋₆-alkyl, C₃₋₆-cycloalkyl or halo-C₃₋₆-cycloalkyl; and

R^{x} represents C₁₋₆-alkyl, C₃₋₆-cycloalkyl or 3- to 6 membered heterocyclyl containing 1 to 2 heteroatoms independently selected from O, N and S,

wherein alkyl, cycloalkyl and heterocycloalkyl are unsubstituted or substituted with 1 or 6 substituents independently selected from the group consisting of halogen, C₁₋₆-alkyl, halo C₁₋₆-alkyl, OR¹ and CN.

In a preferred embodiment of the compounds according to Formula (1) in combination with any of the above and below embodiments, A represents C₃₋₆-cycloalkyl, 3- to 7-membered heterocycloalkyl containing 1 to 4 heteroatoms independently selected from O, N and S. 6- to 10-membered mono or bicyclic aryl or 5- to 10-membered mono or bicyclic heteroaryl containing 1 to 4 heteroatoms independently selected from O, N and S,

wherein cycloalkyl, heterocycloalkyl, aryl and heteroaryl are unsubstituted or substituted with 1 to 5 substituents independently selected from the group consisting of halogen, OH, R^{x}, O—R^{x}, OC(O)—R^{x}, S—R^{x}, S(O)₂—R^{x}, S(O)₂N(R¹)₂, N(R¹)₂, NR¹C(O)R^{x}, C(O)N(R¹)₂, C(O)O—R^{x}, C(O)—R^{x}, CN, COOH, 5- or 6-membered heteroaryl containing 1 to 4 heteroatoms independently selected from O, N and S and 6-membered aryl,

wherein heteroaryl and aryl are unsubstituted or substituted with 1 to 5 substituents independently selected from the group consisting of halogen, CN, OR¹, C₁₋₆-alkyl, C₃₋₆-cycloalkyl and halo-C₁₋₆-alkyl, or

two substituents on the same carbon atom or on two different carbon atoms form together with the carbon atom to which they are attached a C₃₋₁₀ cycloalkyl group, wherein optionally one carbon atom in the cycloalkyl ring may be replaced by a heteroatom selected from O, N and S and wherein the (hetero)cyclic ring may be unsubstituted or substituted by 1 to 3 substituents independently selected from the group consisting of halogen, C₁₋₆-alkyl, halo-C₁₋₆-alkyl or oxo; and

R^{x} represents C₁₋₆-alkyl, C₃₋₆-cycloalkyl or 3- to 6 membered heterocyclyl containing 1 to 2 heteroatoms independently selected from O, N and S,

wherein alkyl, cycloalkyl and heterocycloalkyl are unsubstituted or substituted with 1 or 6 substituents independently selected from the group consisting of halogen, C₁₋₆-alkyl, halo C₁₋₆-alkyl, OR¹ and CN.

In a more preferred embodiment of the compounds according to Formula (1) in combination with any of the above and below embodiments, A represents 6- to 10-membered mono or bicyclic aryl or 5- to 10-membered mono or bicyclic heteroaryl containing 1 to 4 heteroatoms independently selected from O, N and S, wherein aryl and heteroaryl are unsubstituted or substituted with 1 to 5 substituents independently selected from the group consisting of halogen, OH, R, O—R^{x}, OC(O)—R^{x}, S—R^{x}, N(R¹)₂, NR¹C(O)R^{x}, C(O)N(R¹)₂, C(O)O—R^{x}, C(O)—R^{x}, CN, COOH, 5- or 6-membered heteroaryl containing 1 to 4 heteroatoms independently selected from O, N and S and 6-membered aryl,

wherein heteroaryl and aryl are unsubstituted or substituted with 1 to 5 substituents independently selected from the group consisting of halogen, CN, OR¹, C₁₋₆-alkyl, C₃₋₆-cycloalkyl and halo-C₁₋₆-alkyl, or two substituents on the same carbon atom or on two different carbon atoms form together with the carbon atom to which they are attached a C₃₋₁₀ cycloalkyl group, wherein optionally one carbon atom in the cycloalkyl ring may be replaced by a heteroatom selected from O, N and S and wherein the (hetero)cyclic ring may be unsubstituted or substituted by 1 to 3 substituents independently selected from the group consisting of halogen, C₁₋₆-alkyl, halo-C₁₋₆-alkyl or oxo; and

R^{x} represents C₁₋₆-alkyl or C₃₋₆-cycloalkyl,

wherein alkyl and cycloalkyl are unsubstituted or substituted with 1 or 6 substituents independently selected from the group consisting of halogen, C₁₋₆-alkyl, halo C₁₋₆-alkyl, OR¹ and CN.

In a further preferred embodiment of the compounds according to Formula (1) in combination with any of the above and below embodiments, A represents phenyl or 5- or 6-membered heteroaryl containing 1 to 4 heteroatoms independently selected from O, N and S, wherein phenyl and heteroaryl are substituted with 1 to 4 substituents independently selected from the group consisting of halogen, OH, R, O—R^{x}, CN, COOH and 5- or 6-membered heteroaryl containing 1 to 4 heteroatoms independently selected from O, N and S,

wherein heteroaryl and aryl are unsubstituted or substituted with 1 to 5 substituents independently selected from the group consisting of halogen, CN, OR¹, C₁₋₆-alkyl, C₃₋₆-cycloalkyl and halo-C₁₋₆-alkyl; and

R^{x} represents C₁₋₆-alkyl or C₃₋₆-cycloalkyl,

wherein alkyl and cycloalkyl are unsubstituted or substituted with 1 or 6 substituents independently selected from the group consisting of halogen, C₁₋₆-alkyl, halo-C₁₋₆-alkyl, OR¹ and CN.

In a more preferred embodiment of the compounds according to Formula (1) in combination with any of the above and below embodiments, A is

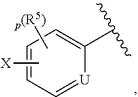

wherein

X represents hydrogen, halogen, C₁₋₆-alkyl, C₃₋₆-cycloalkyl, O—C₁₋₆-alkyl, S—C₁₋₆-alkyl, CN or 5- or 6-membered heteroaryl containing 1 to 4 heteroatoms independently selected from O, N and S,

wherein alkyl and cycloalkyl are unsubstituted or substituted with halogen, C₁₋₆-alkyl, halo-C₁₋₆-alkyl, OR¹ or CN;

R⁵ is independently selected from hydrogen, halogen and C₁₋₆-alkyl;

U is N or CR⁵; and

p is 0, 1, 2, or 3.

In a more preferred embodiment of the compounds according to Formula (1) in combination with any of the above and below embodiments, A represents phenyl which is unsubstituted or substituted with 1 to 4 substituents independently selected from the group consisting of halogen, OH, R, O—R^{x}, OC(O)—R^{x}, S—R^{x}, S(O)₂—R^{x}, S(O)₂N(R)₂, N(R)₂, NR¹C(O)R, C(O)N(R)₂, C(O)O—R^{x}, C(O)—R^{x}, CN, COOH, 5- or 6-membered heteroaryl containing 1 to 4 heteroatoms independently selected from O, N and S and 6-membered aryl,

wherein heteroaryl and aryl are unsubstituted or substituted with 1 to 5 substituents independently selected from the group consisting of halogen, CN, OR¹, C₁₋₆-alkyl, C₃₋₆-cycloalkyl and halo-C₁₋₆-alkyl, and

R^{x} represents C₁₋₆-alkyl, C₃₋₆-cycloalkyl or 3- to 6 membered heterocyclyl containing 1 to 2 heteroatoms independently selected from O, N and S,

wherein alkyl, cycloalkyl and heterocycloalkyl are unsubstituted or substituted with 1 or 6 substituents independently selected from the group consisting of halogen, C₁₋₆-alkyl, halo-C₁₋₆-alkyl, OR¹ and CN.

In a preferred embodiment of the compounds according to Formula (1) in combination with any of the above and below embodiments, R represents C₁₋₆-alkyl or C₃₋₆-cycloalkyl,

wherein alkyl and cycloalkyl are unsubstituted or substituted with 1 or 4 substituents independently selected from the group consisting of halogen, C₁₋₆-alkyl, halo C₁₋₆-alkyl, OR¹ and CN.

In an even more preferred embodiment of the compounds according to Formula (1) in combination with any of the above and below embodiments, A represents

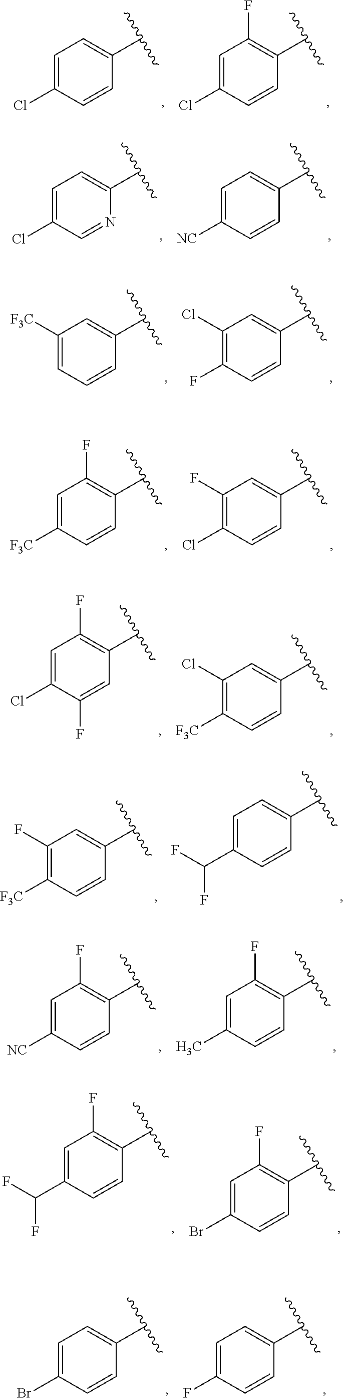

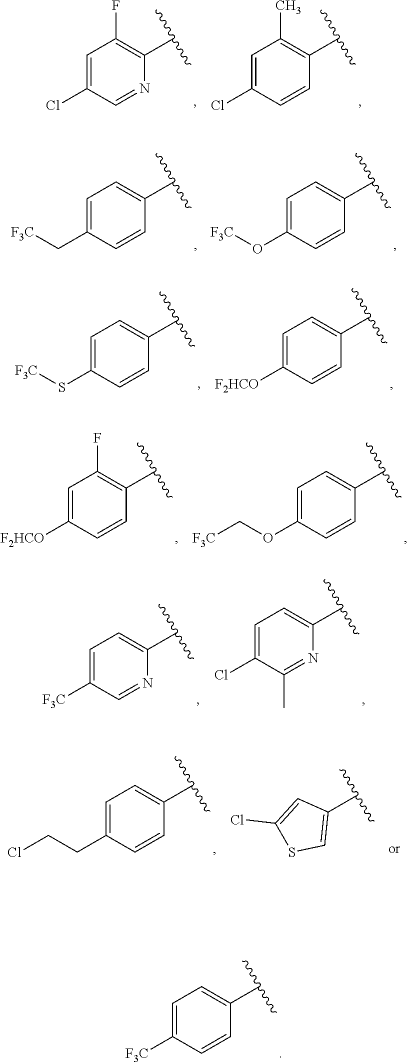

In a more preferred embodiment of the compounds according to Formula (1) in combination with any of the above and below embodiments, A represents

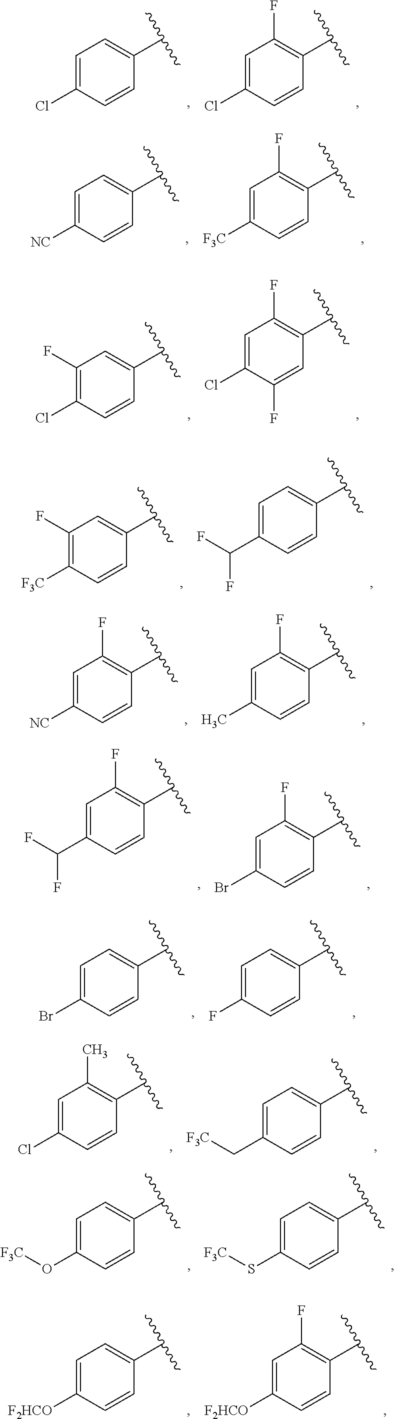

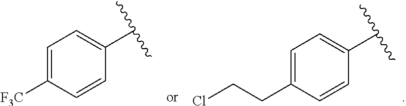 [SMILES: *c1ccc(C(F)(F)F)cc1 ; *c1ccc(CCCl)cc1]

In an equally more preferred embodiment of the compounds according to Formula (1) in combination with any of the above and below embodiments, A represents

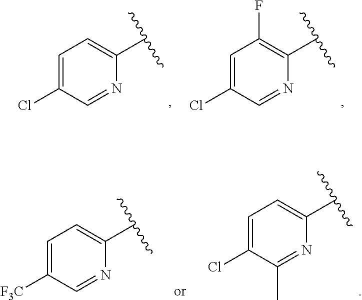

In a further preferred embodiment of the compounds according to Formula (1) in combination with any of the above and below embodiments, B represents a bond.

In a further preferred embodiment of the compounds according to Formula (1) in combination with any of the above and below embodiments, R¹ is hydrogen.

In an equally preferred embodiment of the compounds according to Formula (1) in combination with any of the above and below embodiments, R¹ is C₁₋₆ alkyl.

In a preferred embodiment of the compounds according to Formula (1) in combination with any of the above and below embodiments, R² is halogen, C₁₋₆ alkyl, C₃₋₆ cycloalkyl, halo-C₁₋₆-alkyl, OR⁴, CN, oxo or two R² on the same carbon atom form together with the carbon atom to which they are attached a C₃₋₆ cycloalkyl group, or two R² at different carbon atoms form together a —CH₂—, —CH(CH₃)—, —C(CH₃)₂—, CH₂—CH(CH₃)—, —CH₂—CH₂— or —CH₂—CH₂—CH₂— and o is 0, 1 or 2.

In a more preferred embodiment of the compounds according to Formula (1) in combination with any of the above and below embodiments, R² is halogen, C₁₋₆-alkyl, C₃₋₆-cycloalkyl, halo-C₁₋₆-alkyl, OR⁴ or oxo or two R² at different carbon atoms form together a —CH₂—, —CH(CH₃)—, —C(CH₃)₂—, CH₂—CH(CH₃)—, —CH₂—CH₂— or —CH₂—CH₂—CH₂— and o is 0, 1 or 2.

In a more preferred embodiment of the compounds according to Formula (1) in combination with any of the above and below embodiments, o is 0.

In a preferred embodiment of the compounds according to Formula (1) in combination with any of the above and below embodiments,

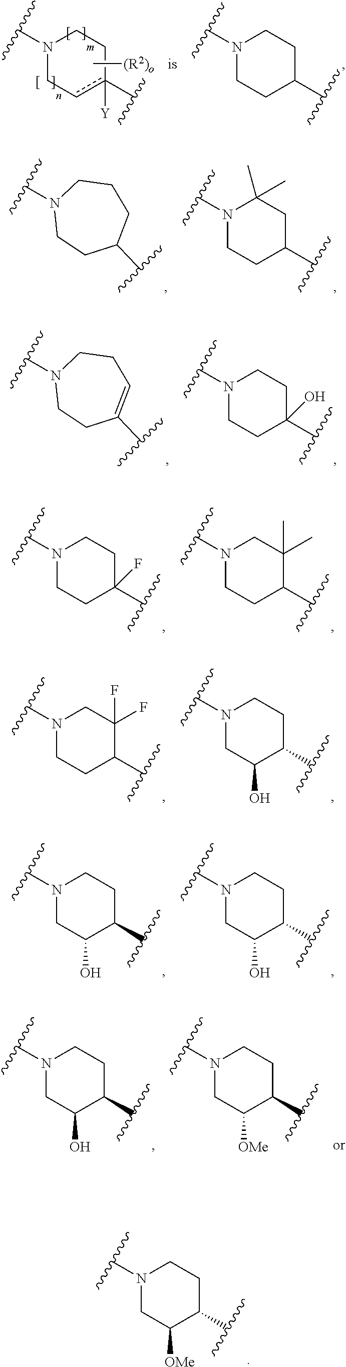

In a more preferred embodiment of the compounds according to Formula (1) in combination with any of the above and below embodiments,

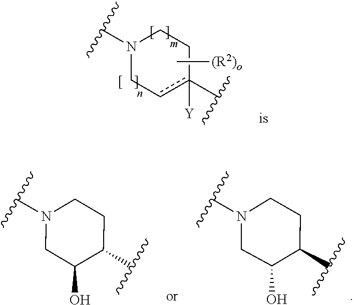

In a further more preferred embodiment of the compounds according to Formula (1) in combination with any of the above and below embodiments,

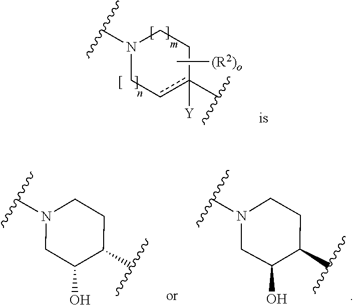

In an equally more preferred embodiment of the compounds according to Formula (1) in combination with any of the above and below embodiments,

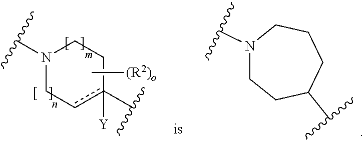

And in an additionally more preferred embodiment of the compounds according to Formula (1) in combination with any of the above and below embodiments,

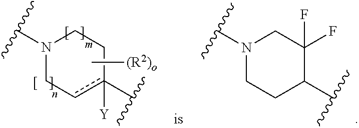

In a preferred embodiment of the compounds according to Formula (1) in combination with any of the above and below embodiments, C represents a 6- to 10-membered mono- or bicyclic aryl or 5- to 10-membered mono- or bicyclic heteroaryl containing 1 to 3 heteroatoms independently selected from O, N and S,

wherein aryl and heteroaryl are unsubstituted or substituted with 1 to 5 substituents independently selected from the group consisting of halogen, OH, R^{y}, O—R^{y}, OC(O)—R^{y}, S—R^{y}, N(R¹)₂, NR¹C(O)R^{y}, C(O)N(R¹)₂, C(O)O—R, C(O)—R^{y}, CN, COOH, 5- or 6-membered heteroaryl containing 1 to 4 heteroatoms independently selected from O, N and S and 6-membered aryl,

wherein aryl and heteroaryl are unsubstituted or substituted with 1 to 5 substituents independently selected from the group consisting of halogen, CN, OR¹, C₁₋₆-alkyl, C₃₋₆-cycloalkyl, and halo-C₁₋₆-alkyl; and

R^{y} represents C₁₋₆-alkyl or C₃₋₆-cycloalkyl,

wherein alkyl or cycloalkyl and are unsubstituted or substituted with 1 to 6 substituents independently selected from the group consisting of halogen, C₁₋₆-alkyl, halo-C₁₋₆-alkyl, OR¹, CN and phenyl.

In a preferred embodiment of the compounds according to Formula (1) in combination with any of the above and below embodiments, R^{y} represents C₁₋₆-alkyl or C₃₋₆-cycloalkyl,

wherein alkyl and cycloalkyl are unsubstituted or substituted with 1 or 4 substituents independently selected from the group consisting of halogen, C₁₋₆-alkyl, halo C₁₋₆-alkyl, OR¹ and CN.

In a more preferred embodiment of the compounds according to Formula (1) in combination with any of the above and below embodiments, C represents phenyl or 6- or 10-membered mono-bicyclic heteroaryl containing 1 to 3 heteroatoms independently selected from O, N and S,

wherein phenyl and heteroaryl are unsubstituted or substituted with 1 to 3 substituents independently selected from halogen, C₁₋₆-alkyl, halo-C₁₋₆-alkyl, O—C₁₋₆-alkyl, O-halo-C₁₋₆-alkyl, OH, CN, COOR⁴, 5- or 6-membered heteroaryl containing 1 to 4 heteroatoms independently selected from O, N and S and phenyl.

In an even more preferred embodiment of the compounds according to Formula (1) in combination with any of the above and below embodiments, C represents a 10-membered bicyclic heteroaryl containing 1 to 3 heteroatoms independently selected from O, N and S,

wherein heteroaryl is unsubstituted or substituted with 1 to 3 substituents independently selected from halogen, C₁₋₆-alkyl, halo-C₁₋₆-alkyl, O—C₁₋₆-alkyl, O-halo-C₁₋₆-alkyl, OH, CN, COOR⁴, 5- or 6-membered heteroaryl containing 1 to 4 heteroatoms independently selected from O, N and S and phenyl.

In an even more preferred embodiment of the compounds according to Formula (1) in combination with any of the above and below embodiments, C is

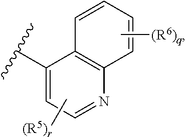

wherein

R⁶ is independently selected from the group consisting of halogen, OH, R, O—R^{x}, OC(O)—R^{x}, S—R^{x}, S(O)₂—R^{x}, S(O)₂N(R¹)₂, N(R¹)₂, NR¹C(O)R^{x}, C(O)N(R¹)₂, C(O)O—R^{x}, C(O)—R^{x}, CN, COOH, 5- or 6-membered heteroaryl containing 1 to 4 heteroatoms independently

selected from O, N and S and 6-membered aryl, wherein heteroaryl and aryl are unsubstituted or substituted with 1 to 5 substituents independently selected from the group consisting of halogen, CN, OR¹, C₁₋₆-alkyl, C₃₋₆-cycloalkyl and halo-C₁₋₆-alkyl;

q is 0, 1, 2, 3 or 4; and

r is 0, 1 or 2.

In most preferred embodiment of the compounds according to Formula (1) in combination with any of the above and below embodiments, C represents

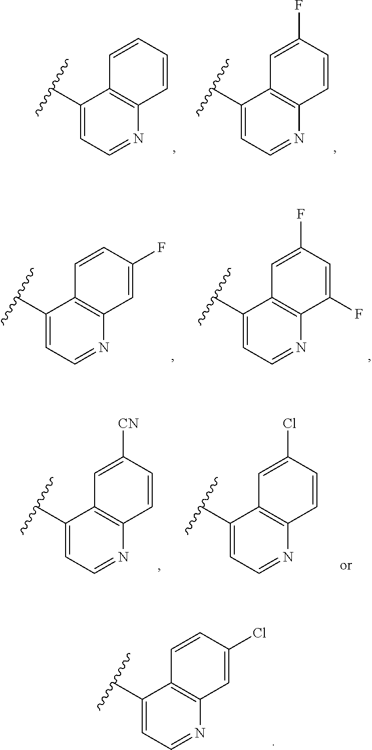

In a preferred embodiment of the compounds according to Formula (1) in combination with any of the above and below embodiments, m is 0, 1 or 2 and n is 0 or 1.

In a more preferred embodiment of the compounds according to Formula (1) in combination with any of the above and below embodiments, m is 1 or 2 and n is 1.

In a most preferred embodiment of the compounds according to Formula (1) in combination with any of the above and below embodiments, m and n are both 1.

In a preferred embodiment of the compounds according to Formula (1) in combination with any of the above and below embodiments, the compound is represented by the following formula (1-1)

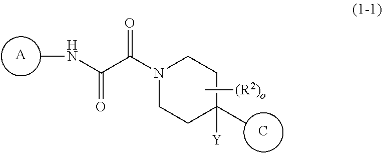

wherein A represents C₃₋₁₀ cycloalkyl, which may be optionally fused with a phenyl ring being unsubstituted or substituted with 1 to 3 R^{a}, 3- to 10-membered heterocycloalkyl containing 1 to 4 heteroatoms independently selected from O, N and S, 6- to 10-membered mono or bicyclic aryl or 5- to 10-membered mono or bicyclic heteroaryl containing 1 to 4 heteroatoms independently selected from O, N and S,

wherein cycloalkyl, heterocycloalkyl, aryl and heteroaryl are unsubstituted or substituted with 1 to 5 substituents independently selected from the group consisting of halogen, OH, R^{x}, O—R^{x}, OC(O)—R^{x}, S—R^{x}, S(O)₂—R^{x}, S(O)₂N(R¹)₂, N(R¹)₂, NR¹C(O)R^{x}, C(O)N(R¹)₂, C(O)O—R^{x}, C(O)—R^{x}, CN, COOH, 5- or 6-membered heteroaryl containing 1 to 4 heteroatoms independently selected from O, N and S and 6-membered aryl,

wherein heteroaryl and aryl are unsubstituted or substituted with 1 to 5 substituents independently selected from the group consisting of halogen, CN, OR¹, C₁₋₆-alkyl, C₃₋₆-cycloalkyl and halo-C₁₋₆-alkyl, or

two substituents on the same carbon atom or on two different carbon atoms form together with the carbon atom to which they are attached a C₃₋₁₀ cycloalkyl group, wherein optionally one carbon atom in the cycloalkyl ring may be replaced by a heteroatom selected from O, N and S;

R^{a} represents halogen, CN, C₁₋₆-alkyl, halo-C₁₋₆-alkyl, hydroxy-C₁₋₆-alkyl, C₃₋₆-cycloalkyl or halo-C₃₋₆-cycloalkyl;

R^{x} represents C₁₋₆-alkyl, C₃₋₆-cycloalkyl or 3- to 6 membered heterocyclyl containing 1 to 2 heteroatoms independently selected from O, N and S,

wherein alkyl, cycloalkyl and heterocycloalkyl are unsubstituted or substituted with 1 or 2 substituents independently selected from the group consisting of halogen, C₁₋₆-alkyl, halo-C₁₋₆-alkyl, OR¹ and CN;

C represents 6- to 10-membered mono- or bicyclic aryl or 5- to 14-membered mono-, bi- or tricyclic heteroaryl containing 1 to 4 heteroatoms independently selected from O, N and S,

wherein aryl and heteroaryl are unsubstituted or substituted with 1 to 5 substituents independently selected from the group consisting of halogen, OH, R^{y}, O—R^{y}, OC(O)—R^{y}, S—R^{y}, S(O)₂—R^{y}, S(O)₂N(R¹)₂, N(R¹)₂, NR¹C(O)R^{y}, C(O)N(R¹)₂, C(O)O—R^{y}, C(O)—R^{y}, CN, COOH, 5- or 6-membered heteroaryl containing 1 to 4 heteroatoms independently selected from O, N and S and 6-membered aryl,

wherein aryl and heteroaryl are unsubstituted or substituted with 1 to 5 substituents independently selected from the group consisting of halogen, CN, OR¹, C₁₋₆-alkyl; C₁₋₆-cycloalkyl, and halo C₁₋₆-alkyl, or

two substituents on the aryl or heteroaryl ring systems together with the carbon atoms to which they are attached form a 5- or 6-membered heterocyclic ring containing 1 or 2 heteroatoms independently selected from O, N and S,

wherein the heterocylic ring is unsubstituted or substituted with 1 or 2 substituents independently selected from the group consisting of halogen, C₁₋₆-alkyl, halo-C₁₋₆-alkyl, CN and oxo;

R^{y} represents C₁₋₆-alkyl, C₃₋₆-cycloalkyl or 3- to 6 membered heterocyclyl containing containing 1 to 2 heteroatoms independently selected from O, N and S,

wherein alkyl, cycloalkyl and heterocycloalkyl are unsubstituted or substituted with 1 or 2 substituents independently selected from the group consisting of halogen, C₁₋₆-alkyl, halo C₁₋₆-alkyl, OR¹ and CN;

Y is hydrogen, OR⁴, halogen, C₁₋₆-alkyl or halo-C₁₋₆-alkyl;

R² is halogen, C₁₋₆ alkyl, C₃₋₆-cycloalkyl, halo-C₁₋₆

-alkyl, OR⁴, S(O)₂N(R¹)₂, S(O)₂—C₁₋₆-alkyl, S(O)₂—C₃₋₆-cycloalkyl, S(O)₂-halo-C₁₋₆-alkyl, S(O)₂N(R¹)₂, C(O)N(R¹)₂, CN, C(O)OR⁴ or oxo, or

two R² on the same carbon atom form together with the carbon atom to which they are attached a C₃₋₁₀ cycloalkyl group, or

two R² at different carbon atoms form together a —CH₂—, —CH(CH₃)—, —C(CH₃)₂—, CH₂—CH(CH₃)—, —CH₂—CH₂— or —CH₂—CH₂—CH₂— group; and

o is 0-4.

In an even more preferred embodiment of the compounds according to Formula (1) in combination with any of the above and below embodiments, C represents a 10-membered bicyclic heteroaryl containing 1 to 3 heteroatoms independently selected from O, N and S,

wherein heteroaryl is unsubstituted or substituted with 1 to 3 substituents independently selected from halogen, C₁₋₆-alkyl, halo-C₁₋₆-alkyl, O—C₁₋₆-alkyl, O-halo-C₁₋₆-alkyl, OH, CN, COOR⁴, 5- or 6-membered heteroaryl containing 1 to 4 heteroatoms independently selected from O, N and S and phenyl.

In a more preferred embodiment of the compounds according to Formula (1) in combination with any of the above and below embodiments, the compound is represented by the following formula (1-2)

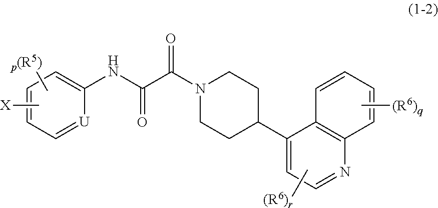

wherein

R⁵ is independently selected from hydrogen, halogen and C₁₋₆-alkyl;

R⁶ is independently selected from the group consisting of halogen, OH, R, O—R^{x}, OC(O)—R^{x}, S—R^{x}, S(O)₂—R^{x}, S(O)₂N(R¹)₂, N(R¹)₂, NR¹C(O)R^{x}, C(O)N(R¹)₂, C(O)O—R^{x}, C(O)—R^{x}, CN, COOH, 5- or 6-membered heteroaryl containing 1 to 4 heteroatoms independently selected from O, N and S and 6-membered aryl,

wherein heteroaryl and aryl are unsubstituted or substituted with 1 to 5 substituents independently selected from the group consisting of halogen, CN, OR¹, C₁₋₆-alkyl, C₃₋₆-cycloalkyl and halo-C₁₋₆-alkyl;

R^{x} represents C₁₋₆-alkyl, C₃₋₆-cycloalkyl or 3- to 6 membered heterocyclyl containing 1 to 2 heteroatoms independently selected from O, N and S,

wherein alkyl, cycloalkyl and heterocycloalkyl are unsubstituted or substituted with 1 or 6 substituents independently selected from the group consisting of halogen, C₁₋₆-alkyl, halo C₁₋₆-alkyl, OR¹ and CN;

X is hydrogen, halogen, C₁₋₆-alkyl, O—C₁₋₆-alkyl, S—C₁₋₆-alkyl, CN or 5- or 6-membered heteroaryl containing 1 to 4 heteroatoms independently selected from O, N and S,

wherein alkyl and cycloalkyl are unsubstituted or substituted with halogen, C₁₋₆-alkyl, halo C₁₋₆-alkyl, OR¹ or CN;

U is N or CR⁵;

p is 0, 1, 2 or 3;

q is 0, 1, 2, 3 or 4; and

r is 0, 1 or 2.

In an even more preferred embodiment of the compounds according to Formula (1) in combination with any of the above and below embodiments, the compound is represented by the following formula (1-2)

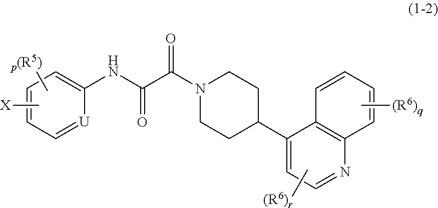

wherein

R⁵ is independently selected from hydrogen, halogen and C₁₋₆-alkyl;

R⁶ is independently selected from the group consisting of halogen, R, O—R^{x}, OC(O)—R^{x}, S—R^{x}, S(O)₂—R^{x}, S(O)₂N(R¹)₂, N(R¹)₂, NR¹C(O)R, C(O)N(R¹)₂, C(O)O—R^{x}, C(O)—R^{x}, CN, COOH, 5- or 6-membered heteroaryl containing 1 to 4 heteroatoms independently selected from O, N and S and 6-membered aryl,

wherein heteroaryl and aryl are unsubstituted or substituted with 1 to 5 substituents independently selected from the group consisting of halogen, CN, OR¹, C₁₋₆-alkyl, C₃₋₆-cycloalkyl and halo-C₁₋₆-alkyl;

R^{x} represents C₁₋₆-alkyl, C₃₋₆-cycloalkyl or 3- to 6 membered heterocyclyl containing 1 to 2 heteroatoms independently selected from O, N and S,

wherein alkyl, cycloalkyl and heterocycloalkyl are unsubstituted or substituted with 1 or 6 substituents independently selected from the group consisting of halogen, C₁₋₆-alkyl, halo C₁₋₆-alkyl, OR¹ and CN;

X is hydrogen, halogen, C₁₋₆-alkyl, O—C₁₋₆-alkyl, S—C₁₋₆-alkyl, CN or 5- or 6-membered heteroaryl containing 1 to 4 heteroatoms independently selected from O, N and S,

wherein alkyl and cycloalkyl are unsubstituted or substituted with halogen, C₁₋₆-alkyl, halo C₁₋₆-alkyl, OR¹ or CN;

U is N or CR⁵;

p is 0, 1, 2 or 3;

q is 0, 1, 2, 3 or 4; and

r is 0, 1 or 2.

In a further more preferred embodiment of the compounds according to Formula (1) in combination with any of the above and below embodiments, U is CR⁵, in particular CH.

In a most preferred embodiment of the compounds according to Formula (1), the compound is selected from

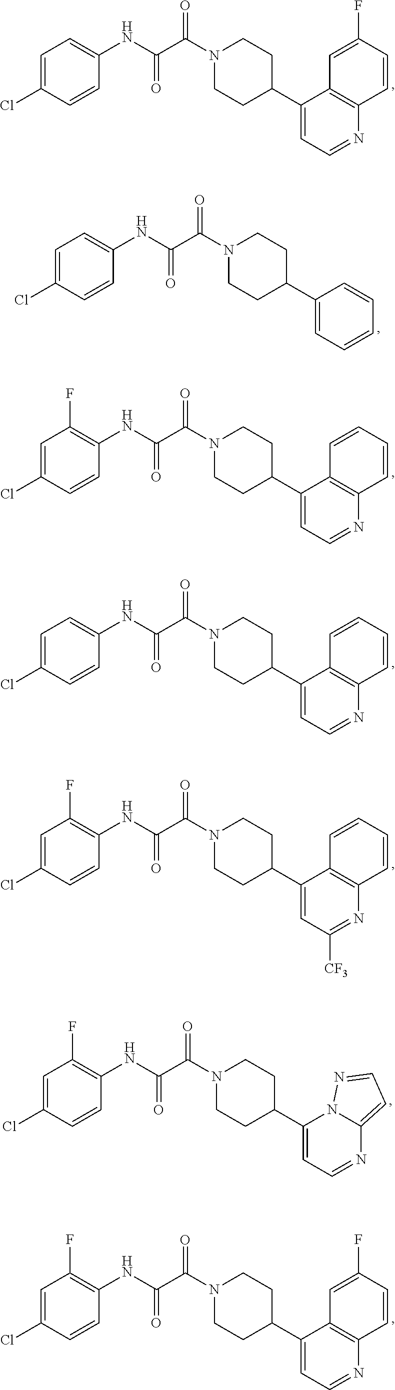

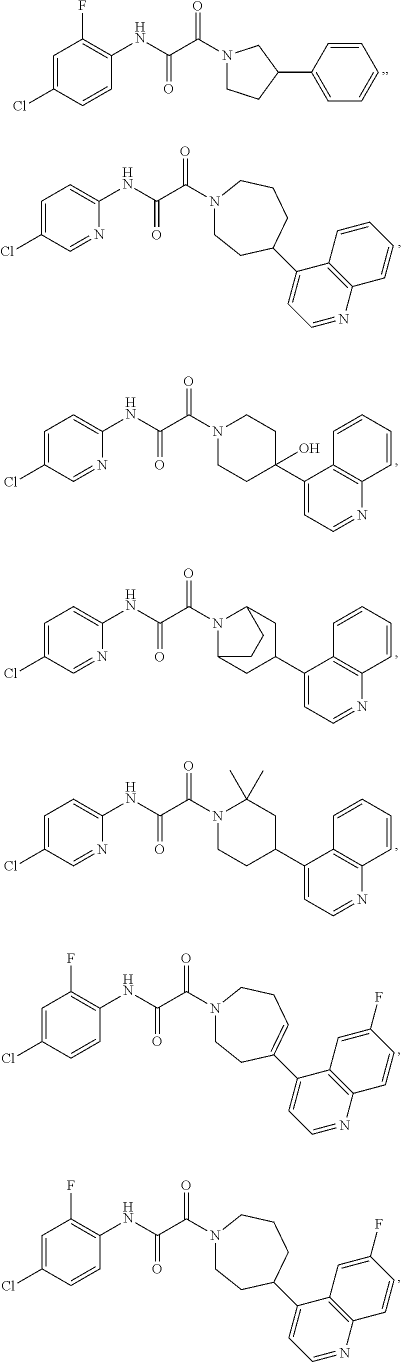

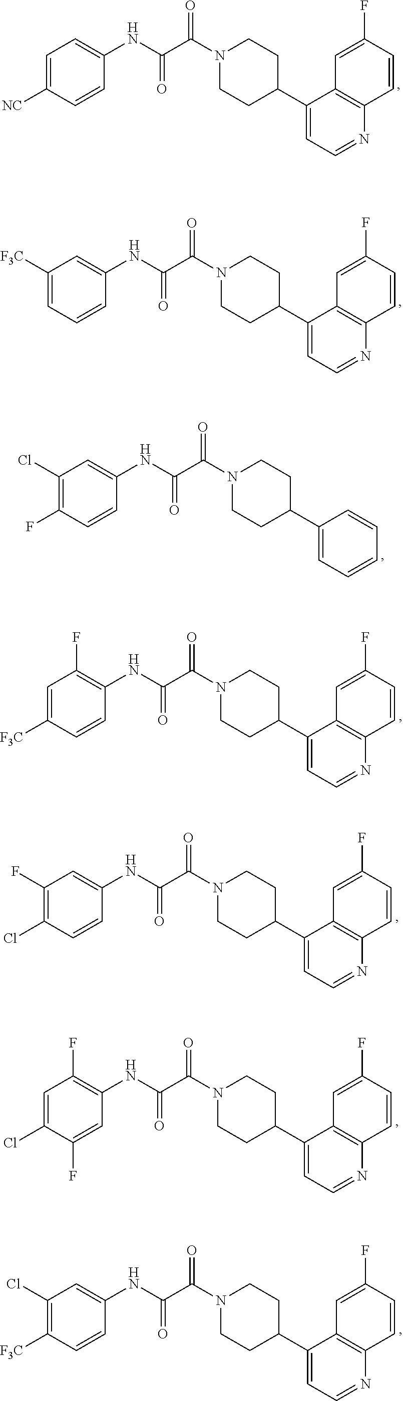

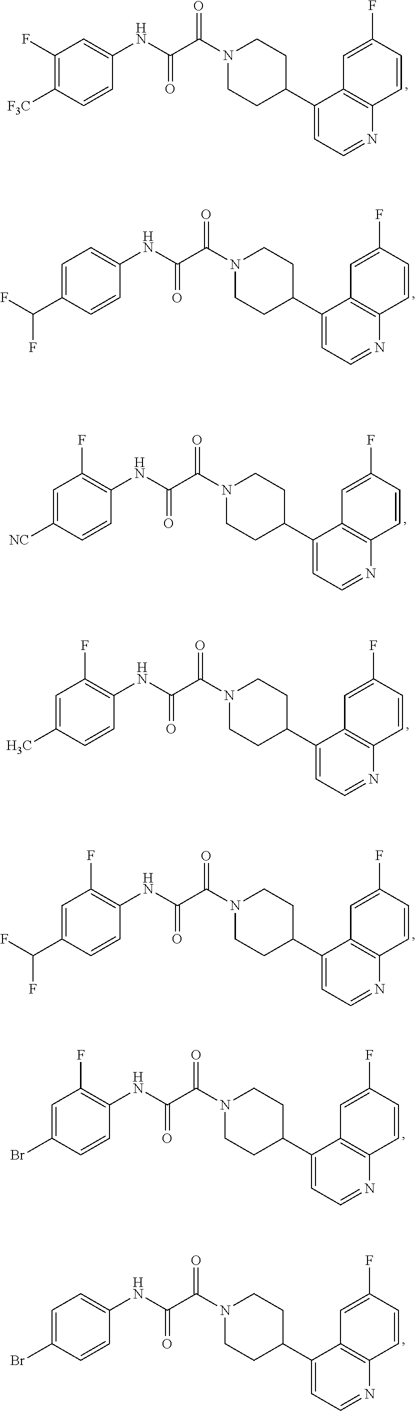

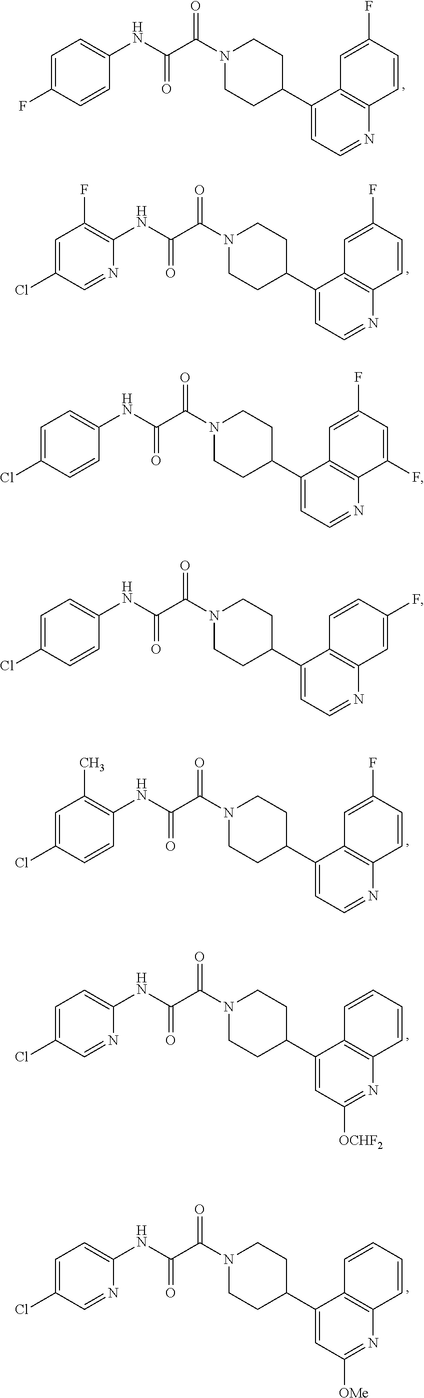

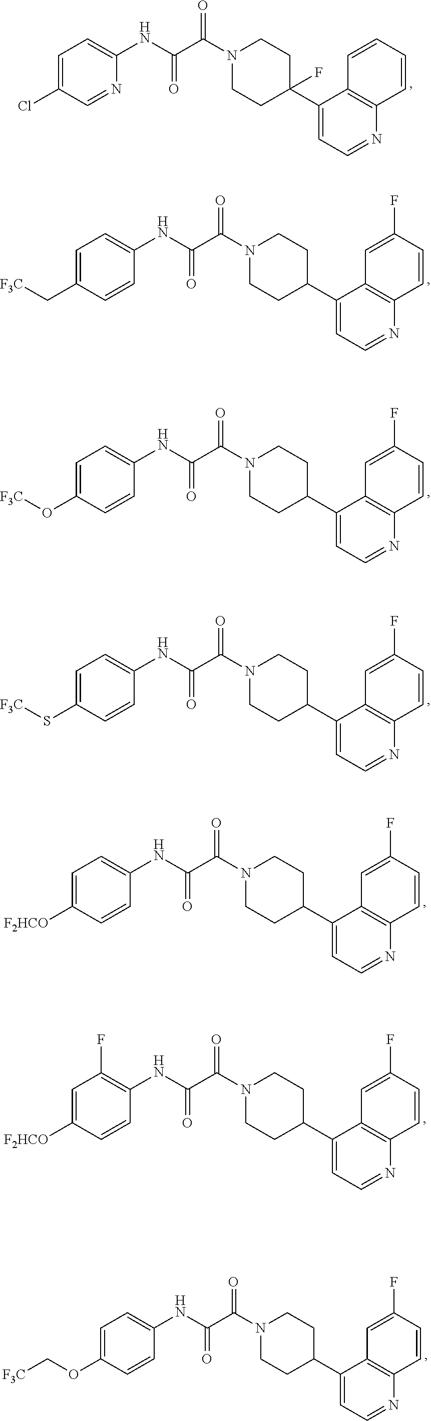

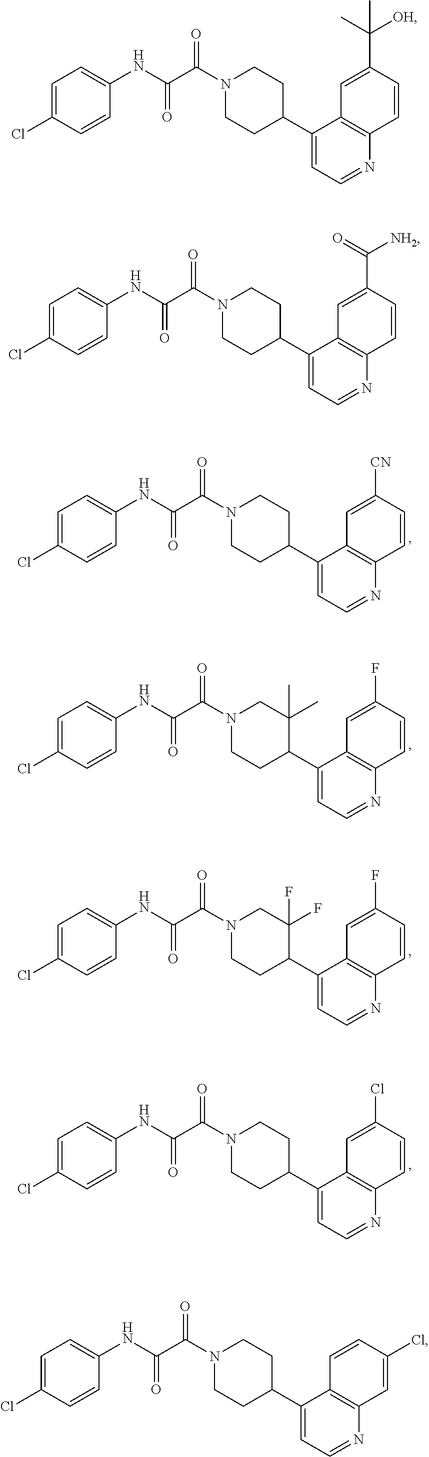

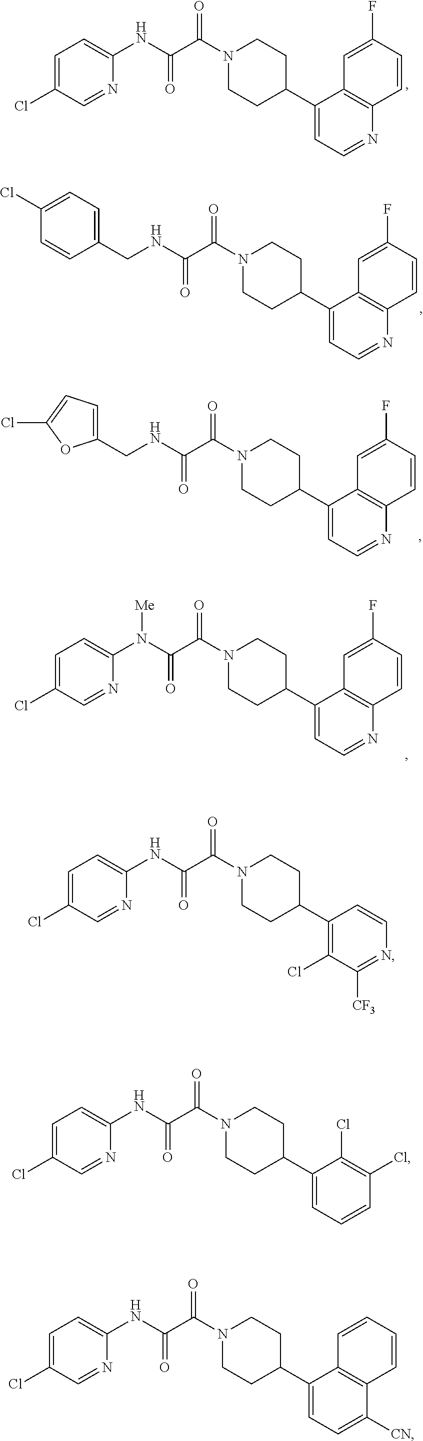

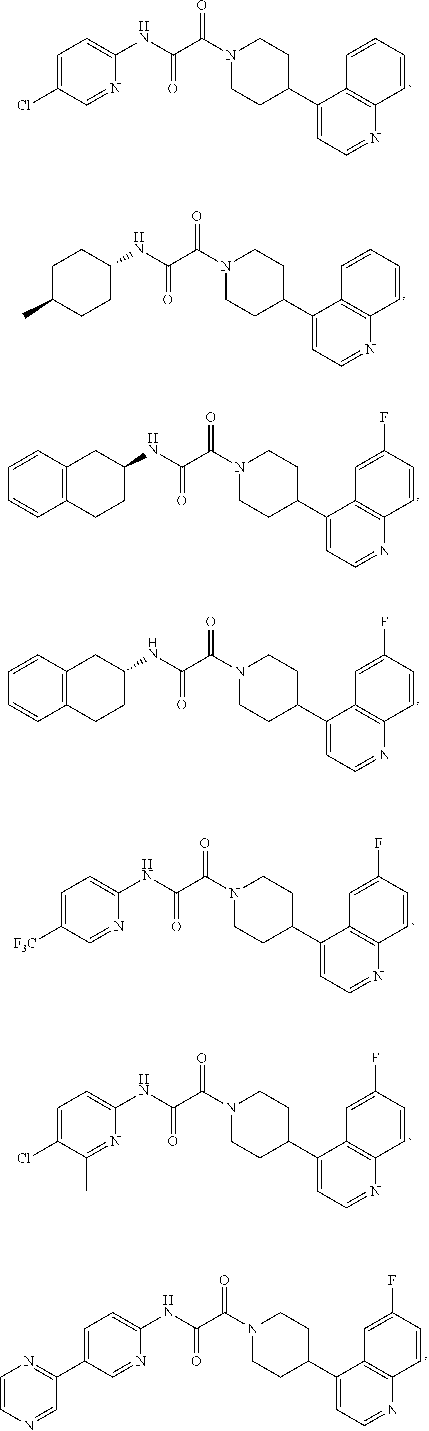

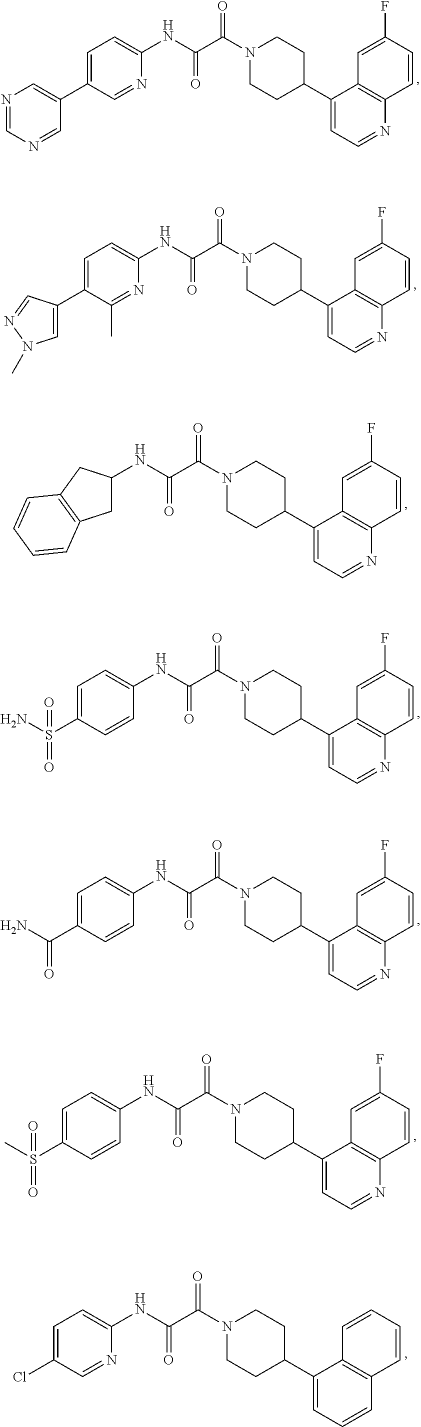

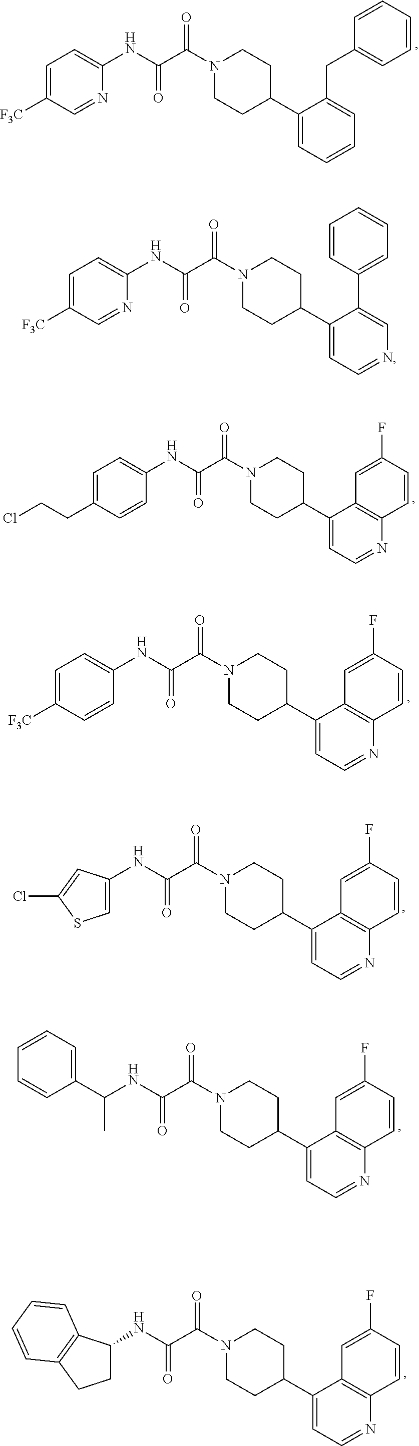

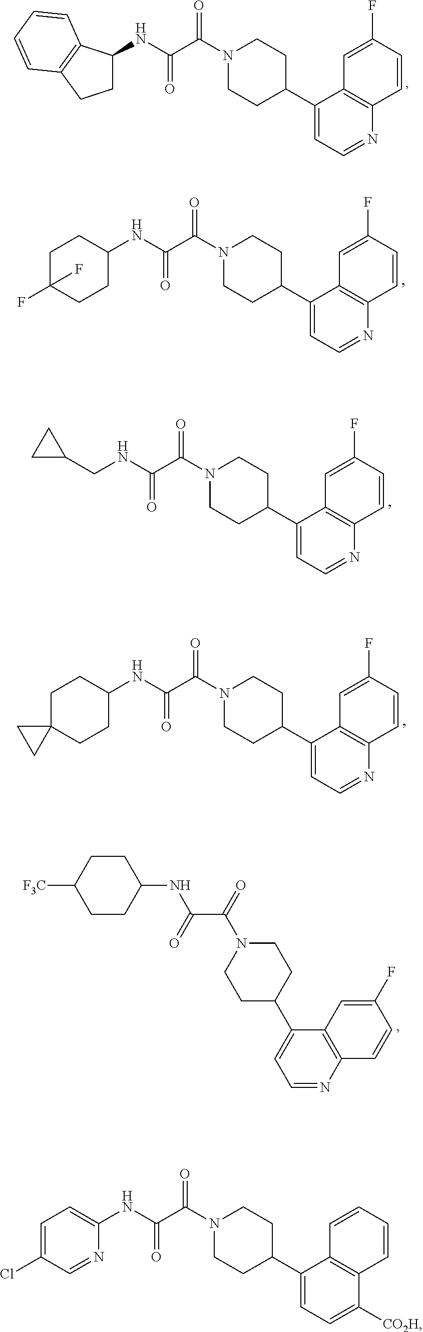

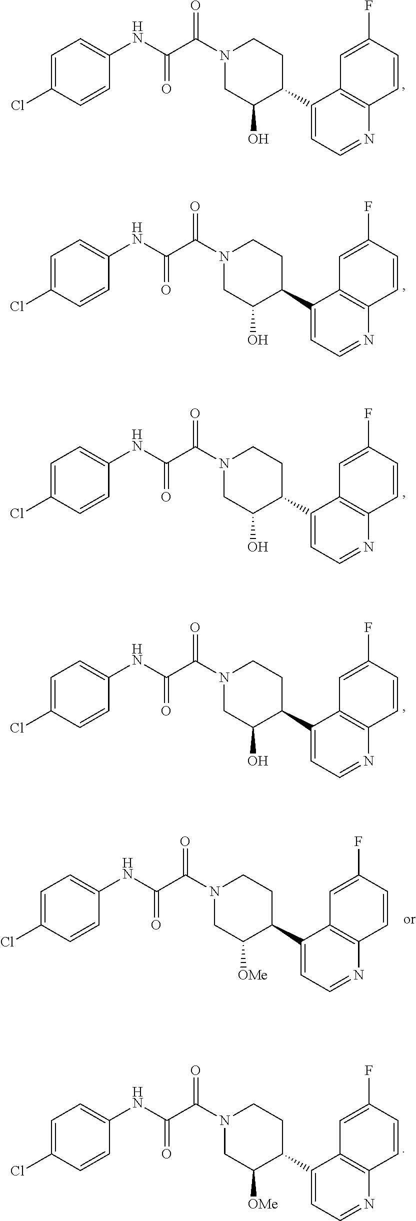 [SMILES: O=C(Nc1ccc(Cl)cc1)C(=O)N1CC[C@H](c2ccnc3ccc(F)cc23)[C@@H](O)C1 ; O=C(Nc1ccc(Cl)cc1)C(=O)N1CC[C@@H](c2ccnc3ccc(F)cc23)[C@H](O)C1 ; O=C(Nc1ccc(Cl)cc1)C(=O)N1CC[C@H](c2ccnc3ccc(F)cc23)[C@H](O)C1 ; O=C(Nc1ccc(Cl)cc1)C(=O)N1CC[C@@H](c2ccnc3ccc(F)cc23)[C@@H](O)C1 ; CO[C@@H]1CN(C(=O)C(=O)Nc2ccc(Cl)cc2)CC[C@H]1c1ccnc2ccc(F)cc12 ; CO[C@H]1CN(C(=O)C(=O)Nc2ccc(Cl)cc2)CC[C@@H]1c1ccnc2ccc(F)cc12]

In an uppermost preferred embodiment of the compounds according to Formula (1) in combination any of the above and below embodiments, the compound is selected from

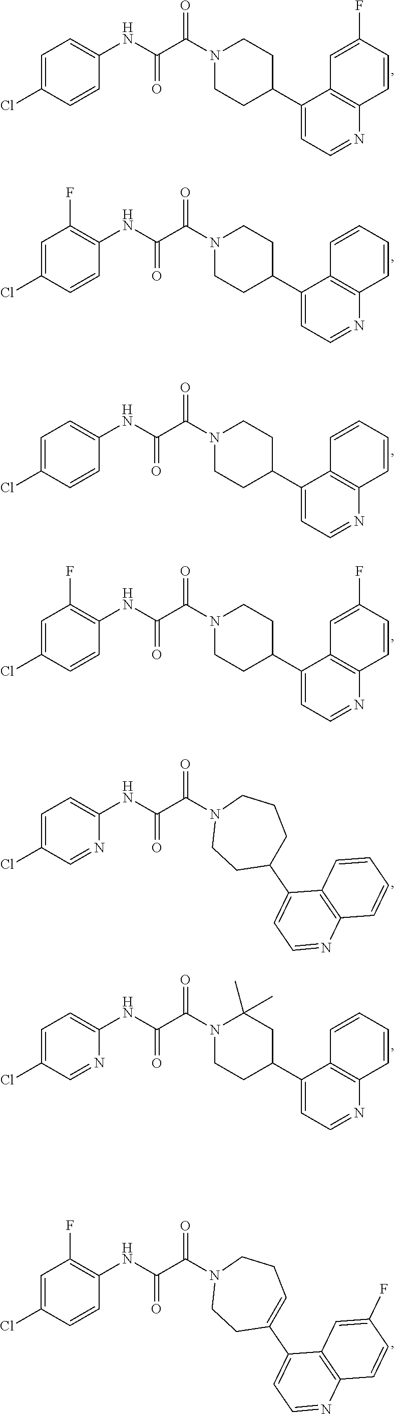

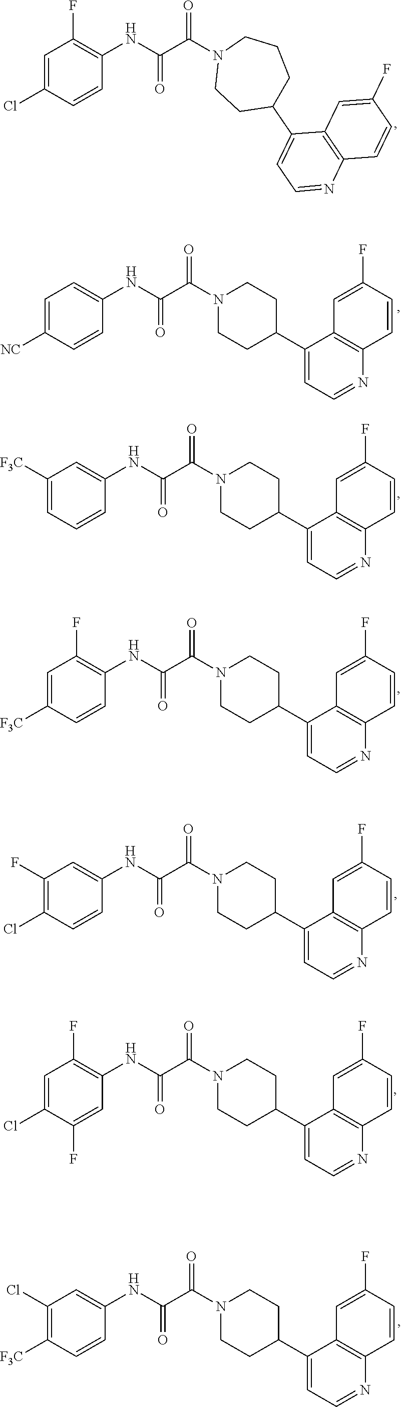

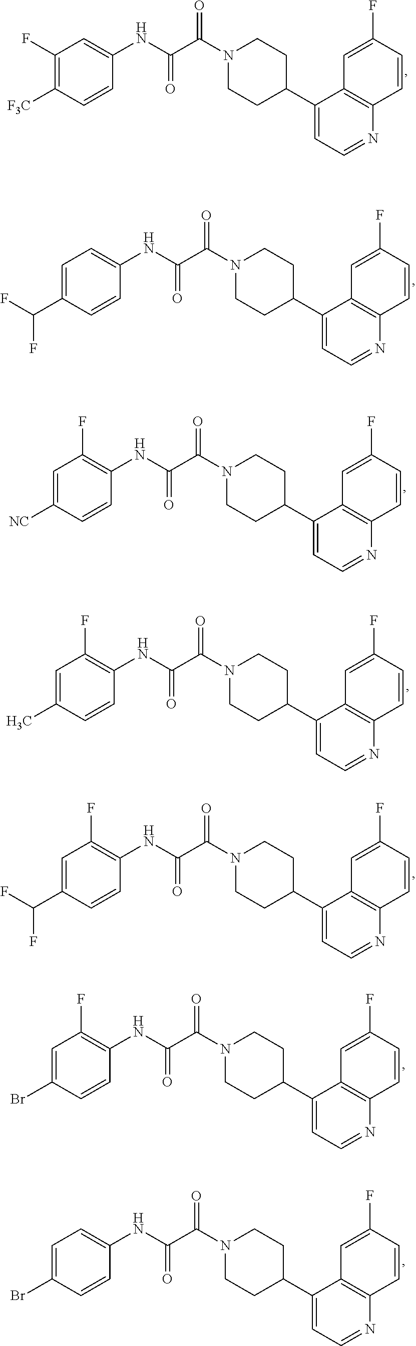

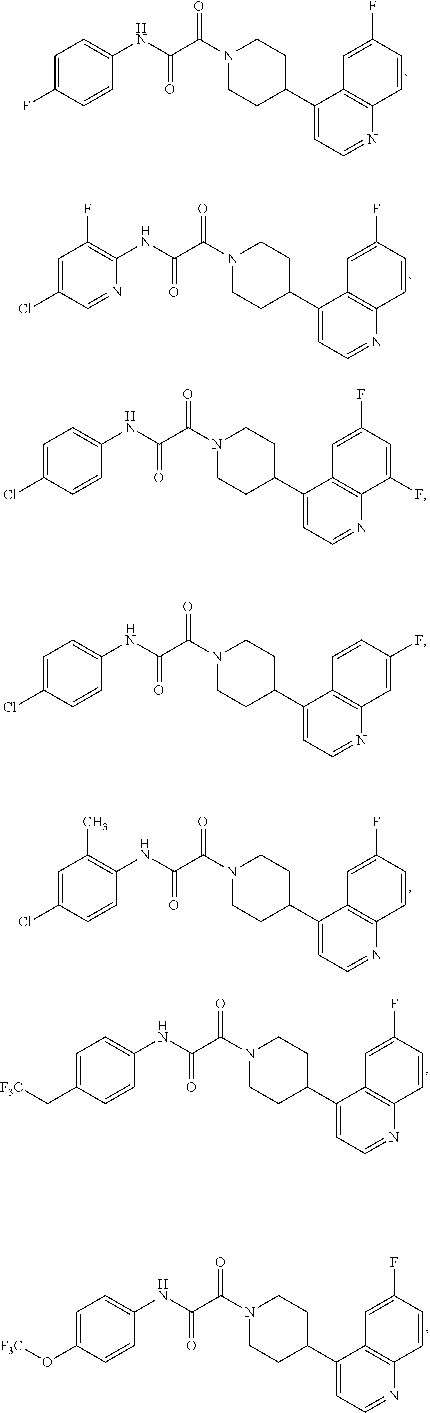

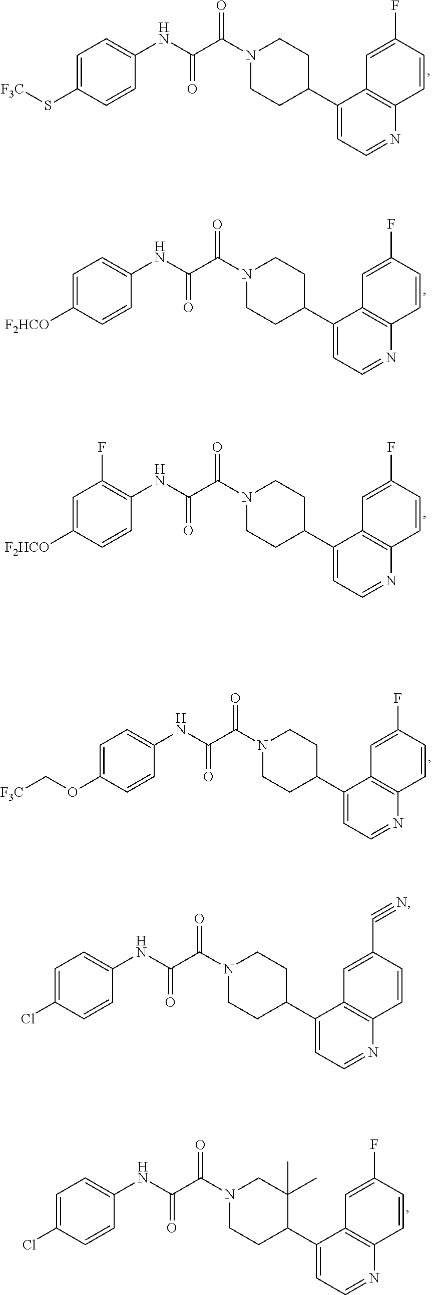 [SMILES: O=C(Nc1ccc(SC(F)(F)F)cc1)C(=O)N1CCC(c2ccnc3ccc(F)cc23)CC1 ; O=C(Nc1ccc(OC(F)F)cc1)C(=O)N1CCC(c2ccnc3ccc(F)cc23)CC1 ; O=C(Nc1ccc(OC(F)F)cc1F)C(=O)N1CCC(c2ccnc3ccc(F)cc23)CC1 ; O=C(Nc1ccc(OCC(F)(F)F)cc1)C(=O)N1CCC(c2ccnc3ccc(F)cc23)CC1 ; N#Cc1ccc2nccc(C3CCN(C(=O)C(=O)Nc4ccc(Cl)cc4)CC3)c2c1 ; CC1(C)CN(C(=O)C(=O)Nc2ccc(Cl)cc2)CCC1c1ccnc2ccc(F)cc12]

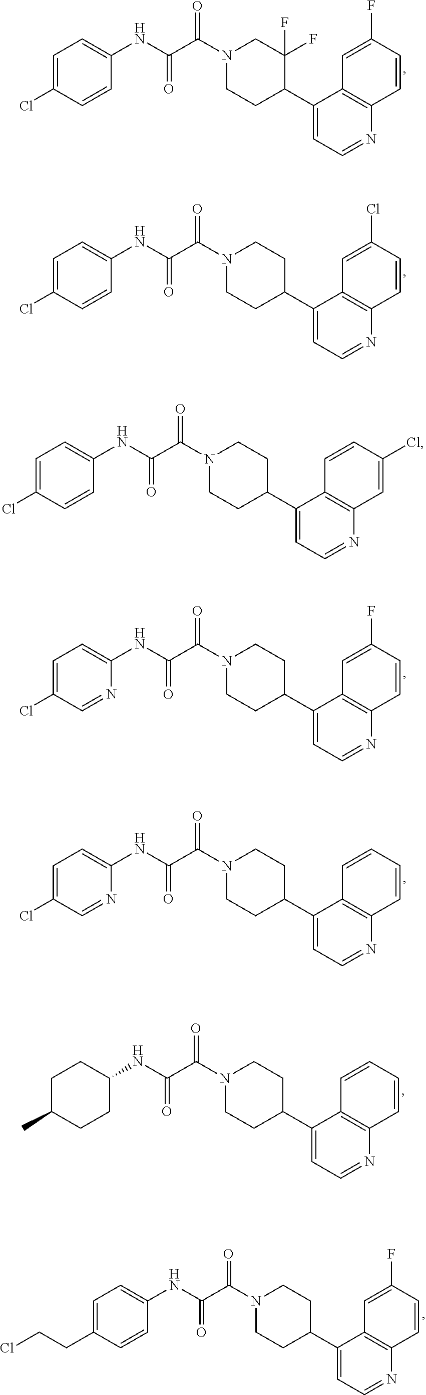

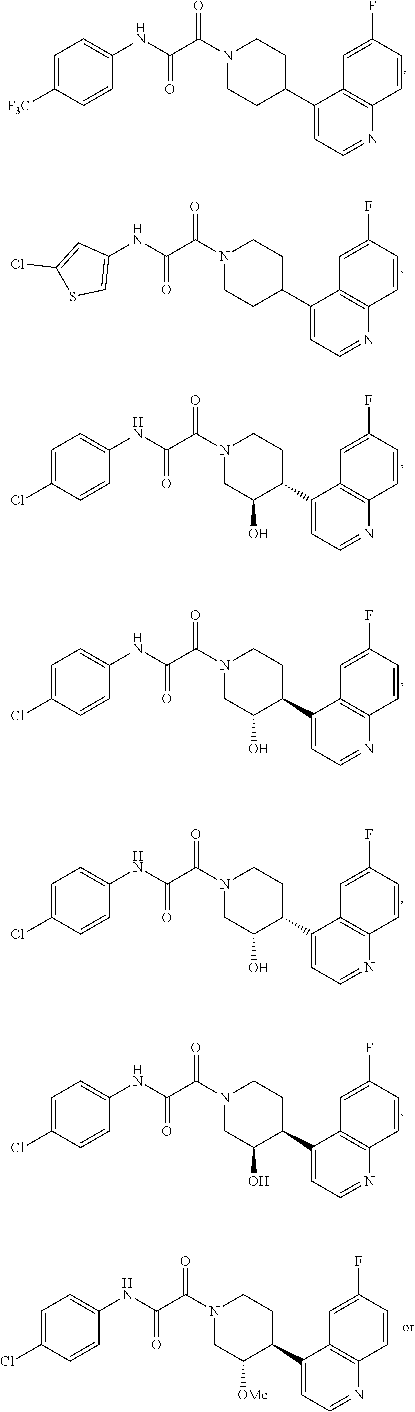

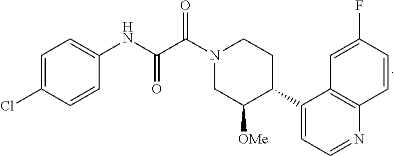 [SMILES: CO[C@H]1CN(C(=O)C(=O)Nc2ccc(Cl)cc2)CC[C@@H]1c1ccnc2ccc(F)cc12]

In a further uppermost preferred embodiment of the compounds according to Formula (1) in combination any of the above and below embodiments, the compound is selected from

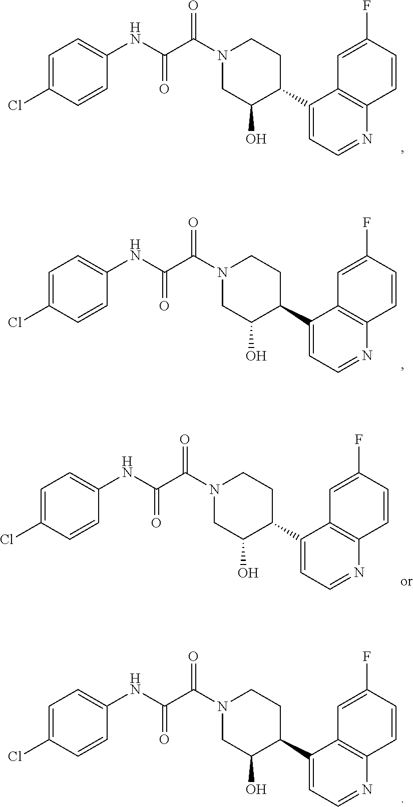 [SMILES: O=C(Nc1ccc(Cl)cc1)C(=O)N1CC[C@H](c2ccnc3ccc(F)cc23)[C@@H](O)C1 ; O=C(Nc1ccc(Cl)cc1)C(=O)N1CC[C@@H](c2ccnc3ccc(F)cc23)[C@H](O)C1 ; O=C(Nc1ccc(Cl)cc1)C(=O)N1CC[C@H](c2ccnc3ccc(F)cc23)[C@H](O)C1 ; O=C(Nc1ccc(Cl)cc1)C(=O)N1CC[C@@H](c2ccnc3ccc(F)cc23)[C@@H](O)C1]

In a preferred embodiment in combination with any of the above and below embodiments, the compound is represented by Formula (2).

In a preferred embodiment of the compounds according to Formula (2) in combination with any of the above and below embodiments, A′ represents C₃₋₁₀ cycloalkyl, which may be optionally fused with a phenyl ring being unsubstituted or substituted with 1 to 3 R^{a}, 3- to 10-membered heterocycloalkyl containing 1 to 4 heteroatoms independently selected from O, N and S, 6- to 10-membered mono or bicyclic aryl or 5- to 10-membered mono or bicyclic heteroaryl containing 1 to 4 heteroatoms independently selected from O, N and S,

wherein cycloalkyl, heterocycloalkyl, aryl and heteroaryl are unsubstituted or substituted with 1 to 5 substituents independently selected from the group consisting of halogen, OH, R^{x}, O—R^{x}, OC(O)—R^{x}, S—R^{x}, S(O)₂—R^{x}, S(O)₂N(R¹)₂, N(R¹)₂, NR¹C(O)R^{x}, C(O)N(R)₂, C(O)O—R^{x}, C(O)—R^{x}, CN, COOH, 5- or 6-membered heteroaryl containing 1 to 4 heteroatoms independently selected from O, N and S and 6-membered aryl,

wherein heteroaryl and aryl are unsubstituted or substituted with 1 to 5 substituents independently selected from the group consisting of halogen, CN, OR¹, C₁₋₆-alkyl, C₃₋₆-cycloalkyl and halo-C₁₋₆-alkyl, or

two substituents on the same carbon atom or on two different carbon atoms form together with the carbon atom to which they are attached a C₃₋₁₀ cycloalkyl group, wherein optionally one carbon atom in the cycloalkyl ring may be replaced by a heteroatom selected from O, N and S and wherein the (hetero)cyclic ring may be unsubstituted or substituted by 1 to 3 substituents independently selected from the group consisting of halogen, C₁₋₆-alkyl, halo-C₁₋₆-alkyl or oxo;

R^{a} represents halogen, CN, C₁₋₆-alkyl, halo-C₁₋₆-alkyl, hydroxy-C₁₋₆-alkyl, C₃₋₆-cycloalkyl or halo-C₃₋₆-cycloalkyl;

R^{x} represents C₁₋₆-alkyl, C₃₋₆-cycloalkyl or 3- to 6 membered heterocyclyl containing 1 to 2 heteroatoms independently selected from O, N and S,

wherein alkyl, cycloalkyl and heterocycloalkyl are unsubstituted or substituted with 1 or 6 substituents independently selected from the group consisting of halogen, C₁₋₆-alkyl, halo C₁₋₆-alkyl, OR¹ and CN.

In a preferred embodiment of the compounds according to Formula (2) in combination with any of the above and below embodiments, A′ represents C₃₋₁₀ cycloalkyl, which may be optionally fused with a phenyl ring being unsubstituted or substituted with 1 to 3 R^{a}, 3- to 10-membered heterocycloalkyl containing 1 to 4 heteroatoms independently selected from O, N and S, 6- to 10-membered mono or bicyclic aryl or 5- to 10-membered mono or bicyclic heteroaryl containing 1 to 4 heteroatoms independently selected from O, N and S,

wherein cycloalkyl, heterocycloalkyl, aryl and heteroaryl are unsubstituted or substituted with 1 to 5 substituents independently selected from the group consisting of halogen, OH, R^{x}, O—R^{x}, OC(O)—R^{x}, S—R^{x}, S(O)₂—R^{x}, S(O)₂N(R¹)₂, N(R¹)₂, NR¹C(O)R, C(O)N(R¹)₂, C(O)O—R^{x}, C(O)—R^{x}, CN, COOH, 5- or 6-membered heteroaryl containing 1 to 4 heteroatoms independently selected from O, N and S and 6-membered aryl,

wherein heteroaryl and aryl are unsubstituted or substituted with 1 to 5 substituents independently selected from the group consisting of halogen, CN, OR¹, C₁₋₆-alkyl, C₃₋₆-cycloalkyl and halo-C₁₋₆-alkyl, or

two substituents on the same carbon atom or on two different carbon atoms form together with the carbon atom to which they are attached a C₃₋₁₀ cycloalkyl group, wherein optionally one carbon atom in the cycloalkyl ring may be replaced by a heteroatom selected from O, N and S and wherein the (hetero)cyclic ring may be unsubstituted or substituted by 1 to 3 substituents independently selected from the group consisting of halogen, C₁₋₆-alkyl, halo-C₁₋₆-alkyl or oxo;

R^{a} represents halogen, CN, C₁₋₆-alkyl, halo-C₁₋₆-alkyl, hydroxy-C₁₋₆-alkyl, C₃₋₆-cycloalkyl or halo-C₃₋₆-cycloalkyl;

R^{x} represents C₁₋₆-alkyl, C₃₋₆-cycloalkyl or 3- to 6 membered heterocyclyl containing 1 to 2 heteroatoms independently selected from O, N and S,

wherein alkyl, cycloalkyl and heterocycloalkyl are unsubstituted or substituted with 1 or 6 substituents independently selected from the group consisting of halogen, C₁₋₆-alkyl, halo C₁₋₆-alkyl, OR¹ and CN.

In a further preferred embodiment of the compounds according to Formula (2) in combination with any of the above and below embodiments, A′ represents C₃₋₆ cycloalkyl, 3- to 7-membered heterocycloalkyl containing 1 to 4 heteroatoms independently selected from O, N and S, 6- to 10-membered mono or bicyclic aryl or 5- to 10-membered monocyclic heteroaryl containing 1 to 4 heteroatoms independently selected from O, N and S,

wherein cycloalkyl, heterocycloalkyl, aryl and heteroaryl are unsubstituted or substituted with 1 to 5 substituents independently selected from the group consisting of halogen, OH, R^{x}, O—R^{x}, OC(O)—R^{x}, S—R^{x}, N(R¹)₂, NR¹C(O)R^{x}, C(O)N(R¹)₂, C(O)O—R^{x}, C(O)—R^{x}, CN, COOH, 5- or 6-membered heteroaryl containing 1 to 4 heteroatoms independently selected from O, N and S and 6-membered aryl,

wherein heteroaryl and aryl are unsubstituted or substituted with 1 to 5 substituents independently selected from the group consisting of halogen, CN, OR¹, C₁₋₆-alkyl, C₃₋₆-cycloalkyl and halo-C₁₋₆-alkyl, or

two substituents on the same carbon atom or on two different carbon atoms form together with the carbon atom to which they are attached a C₃₋₁₀ cycloalkyl group, wherein optionally one carbon atom in the cycloalkyl ring may be replaced by a heteroatom selected from O, N and S and wherein the (hetero)cyclic ring may be unsubstituted or substituted by 1 to 3 substituents independently selected from the group consisting of halogen, C₁₋₆-alkyl, halo-C₁₋₆-alkyl or oxo; and

R^{x} represents C₁₋₆-alkyl or C₃₋₆-cycloalkyl,

wherein alkyl and cycloalkyl are unsubstituted or substituted with 1 or 6 substituents independently selected from the group consisting of halogen, C₁₋₆-alkyl, halo C₁₋₆-alkyl, OR¹ and CN.

In a preferred embodiment of the compounds according to Formula (2) in combination with any of the above and below embodiments, R represents C₁₋₆-alkyl or C₃₋₆-cycloalkyl,

wherein alkyl and cycloalkyl are unsubstituted or substituted with 1 or 4 substituents independently selected from the group consisting of halogen, C₁₋₆-alkyl, halo C₁₋₆-alkyl, OR¹ and CN.

In a more preferred embodiment of the compounds according to Formula (2) in combination with any of the above and below embodiments, A′ represents 6- to 10-membered mono or bicyclic aryl or 5- to 10-membered cyclic heteroaryl containing 1 to 4 heteroatoms independently selected from O, N and S, wherein aryl and heteroaryl are substituted with 1 to 4 substituents independently selected from the group consisting of halogen, C₁₋₆-alkyl, halo-C₁₋₆-alkyl, OH, CN and COOH.

In a more preferred embodiment of the compounds according to Formula (1) in combination with any of the above and below embodiments, A′ is

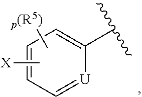

wherein

X is hydrogen, halogen, C₁₋₆-alkyl, C₃₋₆-cycloalkyl, O—C₁₋₆-alkyl, S—C₁₋₆-alkyl, CN or 5- or 6-membered heteroaryl containing 1 to 4 heteroatoms independently selected from O, N and S,

wherein alkyl and cycloalkyl are unsubstituted or substituted with halogen, C₁₋₆-alkyl, halo-C₁₋₆-alkyl, OR¹ or CN;

R⁵ is independently selected from hydrogen, halogen and C₁₋₆-alkyl;

U is N or CR⁵; and

p is 0, 1, 2, or 3.

In an even more preferred embodiment of the compounds according to Formula (2) in combination with any of the above and below embodiments, A′ represents

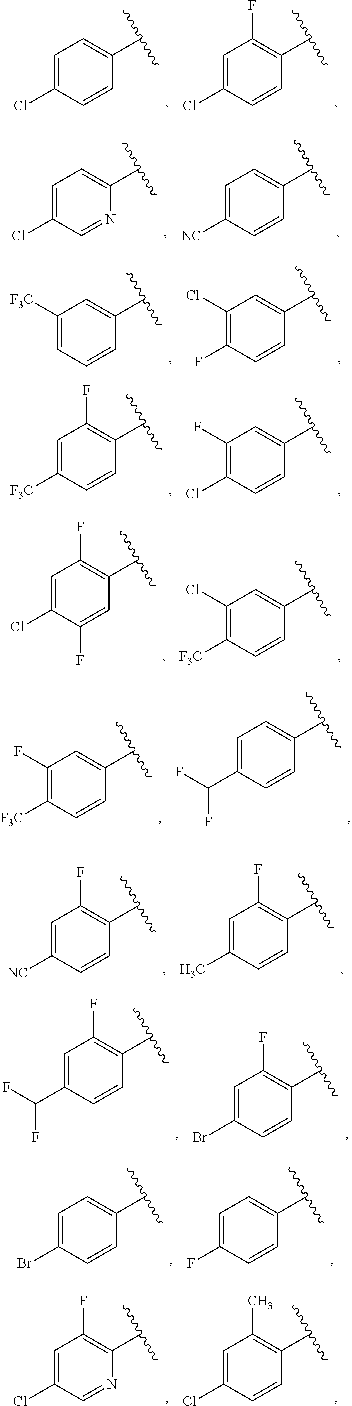

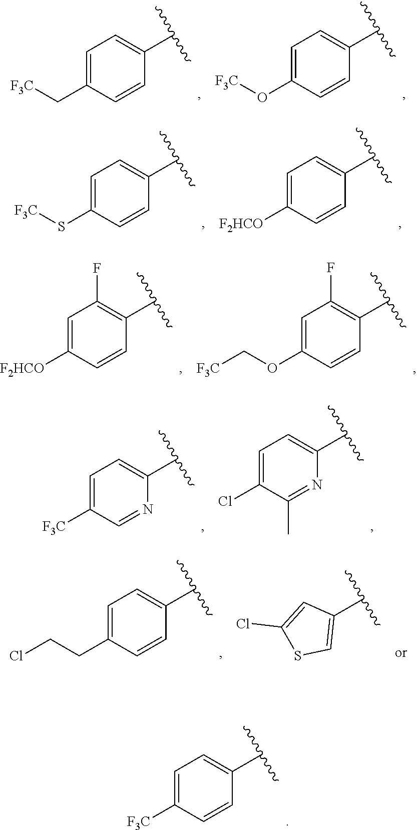

In a further more preferred embodiment of the compounds according to Formula (2) in combination with any of the above and below embodiments, A′ represents phenyl which is unsubstituted or substituted with 1 to 4 substituents independently selected from the group consisting of halogen, OH, R, O—R^{x}, OC(O)—R^{x}, S—R^{x}, S(O)₂—R^{x}, S(O)₂N(R)₂, N(R¹)₂, NR¹C(O)R, C(O)N(R)₂, C(O)O—R^{x}, C(O)—R^{x}, CN, COOH, 5- or 6-membered heteroaryl containing 1 to 4 heteroatoms independently selected from O, N and S and 6-membered aryl,

wherein heteroaryl and aryl are unsubstituted or substituted with 1 to 5 substituents independently selected from the group consisting of halogen, CN, OR¹, C₁₋₆-alkyl, C₃₋₆-cycloalkyl and halo-C₁₋₆-alkyl, and

R^{x} represents C₁₋₆-alkyl, C₃₋₆-cycloalkyl or 3- to 6 membered heterocyclyl containing 1 to 2 heteroatoms independently selected from O, N and S,

wherein alkyl, cycloalkyl and heterocycloalkyl are unsubstituted or substituted with 1 or 6 substituents independently selected from the group consisting of halogen, C₁₋₆-alkyl, halo-C₁₋₆-alkyl, OR¹ and CN.

In a most preferred embodiment of the compounds according to Formula (2) in combination with any of the above and below embodiments, A′ represents

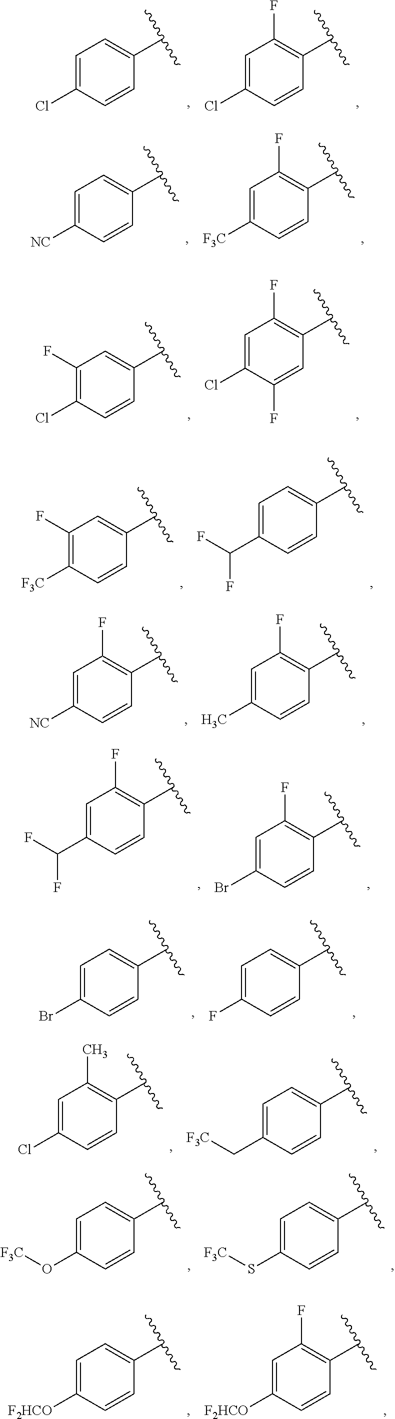

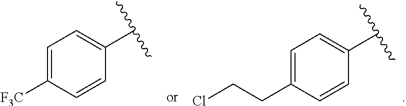 [SMILES: *c1ccc(C(F)(F)F)cc1 ; *c1ccc(CCCl)cc1]

In a further more preferred embodiment of the compounds according to Formula (2) in combination with any of the above and below embodiments, A′ represents

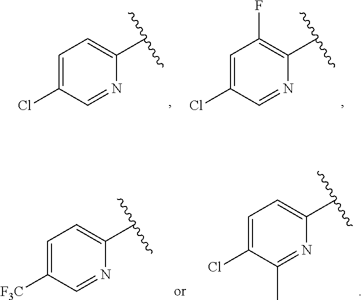

In a further preferred embodiment of the compounds according to Formula (2) in combination with any of the above and below embodiments, B represents a bond.

In a further preferred embodiment of the compounds according to Formula (2) in combination with any of the above and below embodiments, R¹ is hydrogen.

In an equally preferred embodiment of the compounds according to Formula (2) in combination with any of the above and below embodiments, R¹ is C₁₋₆ alkyl.

In a preferred embodiment of the compounds according to Formula (2) in combination with any of the above and below embodiments, R² is halogen, C₁₋₆ alkyl, C₃₋₆-cycloalkyl, halo-C₁₋₆-alkyl, OR⁴, CN, oxo or two R² on the same carbon atom form together with the carbon atom to which they are attached a C₃₋₁₀-cycloalkyl group, or two R² at different carbon atoms form together a —CH₂—, —CH(CH₃)—, —C(CH₃)₂—, CH₂—CH(CH₃)—, —CH₂—CH₂— or —CH₂—CH₂—CH₂— and o is 0, 1 or 2.

In a more preferred embodiment of the compounds according to Formula (2) in combination with any of the above and below embodiments, o is 0.

In a preferred embodiment of the compounds according to Formula (2) in combination with any of the above and below embodiments, Z represents —C₂₋₃-alkylene-, —O—C₁₋₂-alkylene-, —C₁₋₂-alkylene-O—, —NR³C(O)—C₀₋₁-alkylene-, —C(O)NR³—C₀₋₁-alkylene-, —C₀₋₁-alkylene-NR³C(O)—, —C₀₋₁-alkylene C(O)NR³, —NR⁹—C₁₋₂-alkylene- or —C₁₋₂-alkylene-NR⁹—,

wherein alkylene is unsubstituted or substituted with 1 or 2 substituents independently selected from the group consisting of OR⁴, C₁₋₆-alkyl, halogen and halo-C₁₋₆-alkyl; and

R⁹ is hydrogen, C₁₋₆-alkyl, halo-C₁₋₆-alkyl, C(O)—C₁₋₆-alkyl, or C(O)-halo-C₁₋₆-alkyl.

In a more preferred embodiment of the compounds according to Formula (2) in combination with any of the above and below embodiments, Z represents —C₂₋₃-alkylene-, —O—C₁₋₂-alkylene-, —C₁₋₂-alkylene-O—, —NR³C(O)—C₀₋₁-alkylene-, —C(O)NR³—C₁-alkylene-, —C₀₋₁-alkylene-NR³C(O)—, —C₁-alkylene-C(O)NR³, —NR⁹—C₁₋₂-alkylene- or —C₁₋₂-alkylene-NR⁹—,

wherein alkylene is unsubstituted or substituted with 1 or 2 substituents independently selected from the group consisting of OR⁴, C₁₋₆-alkyl, halogen and halo-C₁₋₆-alkyl; and

R⁹ is hydrogen, C₁₋₆-alkyl, halo-C₁₋₆-alkyl, C(O)—C₁₋₆-alkyl, or C(O)-halo-C₁₋₆-alkyl.

In a more preferred embodiment of the compounds according to Formula (2) in combination with any of the above and below embodiments, Z represents —C₂₋₃-alkylene-, —O—C₁₋₂-alkylene- or —C₁₋₂-alkylene-O—,

wherein alkylene is unsubstituted or substituted with 1 or 2 substituents independently selected from the group consisting of C₁₋₆-alkyl, halogen and halo-C₁₋₆-alkyl.

In a most preferred embodiment of the compounds according to Formula (2) in combination with any of the above and below embodiments, Z represents —CH₂—CH₂—, —O—CH₂— or —CH₂—O—.

In an uppermost preferred embodiment of the compounds according to Formula (2) in combination with any of the above and below embodiments, Z represents —O—CH₂—.

In a further uppermost preferred embodiment of the compounds according to Formula (2) in combination with any of the above and below embodiments, Z represents —CH₂—O—.

In a preferred embodiment of the compounds according to Formula (2) in combination with any of the above and below embodiments, D represents 6- to 10-membered mono or bicyclic aryl or 5- to 10-membered mono or bicyclic heteroaryl containing 1 to 3 heteroatoms independently selected from O, N and S,

wherein aryl and heteroaryl are unsubstituted or substituted with 1 to 5 substituents independently selected from the group consisting of halogen, OH, R^{z}, O—R^{z}, OC(O)—R^{z}, S—R^{z}, N(R¹)₂, NR¹C(O)R^{z}, C(O)N(R¹)₂, C(O)O—R^{z}, C(O)—R^{z}, CN, COOH, 5- or 6-membered heteroaryl containing 1 to 4 heteroatoms independently selected from O, N and S and 6-membered aryl,

wherein aryl and heteroaryl are unsubstituted or substituted with 1 to 5 substituents independently selected from the group consisting of halogen, CN, OR¹, C₁₋₆-alkyl, C₃₋₆-cycloalkyl and halo C₁₋₆-alkyl, or

two substituents on the aryl or heteroaryl ring systems together with the carbon atom to which they are attached form a 5- or 6-membered heterocyclic ring containing 1 or 2 heteroatoms independently selected from O, N and S,

wherein the heterocylic ring is unsubstituted or substituted with 1 or 2 substituents independently selected from the group consisting of halogen, C₁₋₆-alkyl, halo-C₁₋₆-alkyl, CN and oxo; and

R^{z} represents C₁₋₆-alkyl or C₃₋₆-cycloalkyl,

wherein alkyl and cycloalkyl are unsubstituted or substituted with 1 to 6 substituents independently selected from the group consisting of halogen, C₁₋₆-alkyl, halo-C₁₋₆-alkyl, OR¹ and CN.

In a preferred embodiment of the compounds according to Formula (2) in combination with any of the above and below embodiments, R^{z} represents C₁₋₆-alkyl or C₃₋₆-cycloalkyl,

wherein alkyl and cycloalkyl are unsubstituted or substituted with 1 or 4 substituents independently selected from the group consisting of halogen, C₁₋₆-alkyl, halo-C₁₋₆-alkyl, OR¹ and CN.

In a more preferred embodiment of the compounds according to Formula (2) in combination with any of the above and below embodiments, D represents phenyl or 5- or 6-membered mono- or bicyclic heteroaryl containing 1 to 3 heteroatoms independently selected from O, N and S,

wherein phenyl and heteroaryl are unsubstituted or substituted with 1 to 3 substituents independently selected from halogen, halo-C₁₋₆-alkyl, O—C₁₋₆-alkyl, OH, and N(R¹)₂.

In a preferred embodiment of the compounds according to Formula (2) in combination with any of the above and below embodiments, m is 0, 1 or 2 and n is 0 or 1.

In a more preferred embodiment of the compounds according to Formula (2) in combination with any of the above and below embodiments, m is 1 or 2 and n is 1.

In a most preferred embodiment of the compounds according to Formula (2) in combination with any of the above and below embodiments, m and n are both 1.

In a further preferred embodiment in combination with any of the above and below embodiments, the compound is represented by Formula (2-1) or (2-2)

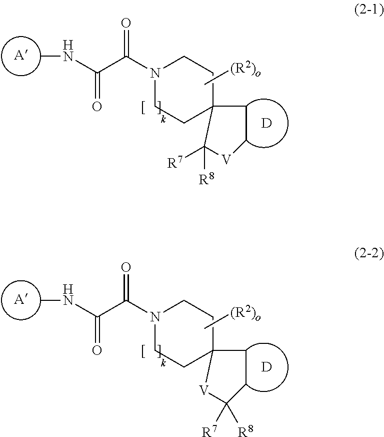

wherein A′ represents C₃₋₁₀ cycloalkyl, which may be optionally fused with a phenyl ring, 3- to 10-membered heterocycloalkyl containing 1 to 4 heteroatoms independently selected from O, N and S, 6- to 10-membered mono or bicyclic aryl or 5- to 10-membered mono or bicyclic heteroaryl containing 1 to 4 heteroatoms independently selected from O, N and S,

wherein cycloalkyl, heterocycloalkyl, aryl and heteroaryl are unsubstituted or substituted with 1 to 5 substituents independently selected from the group consisting of halogen, OH, R^{x}, O—R^{x}, OC(O)—R^{x}, S—R^{x}, S(O)₂—R^{x}, S(O)₂N(R¹)₂, N(R¹)₂, NR¹C(O)R, C(O)N(R¹)₂, C(O)O—R^{x}, C(O)—R^{x}, CN, COOH, 5- or 6-membered heteroaryl containing 1 to 4 heteroatoms independently selected from O, N and S and 6-membered aryl,

wherein heteroaryl and aryl are unsubstituted or substituted with 1 to 5 substituents independently selected from the group consisting of halogen, CN, OR¹, C₁₋₆-alkyl, C₃₋₆-cycloalkyl and halo-C₁₋₆-alkyl, or

two substituents on the same carbon atom or on two different carbon atoms form together with the carbon atom to which they are attached a C₃₋₁₀ cycloalkyl group, wherein optionally one carbon atom in the cycloalkyl ring may be replaced by a heteroatom selected from O, N and S;

R^{x} represents C₁₋₆-alkyl, C₃₋₆-cycloalkyl or 3- to 6 membered heterocyclyl containing 1 to 2 heteroatoms independently selected from O, N and S,

wherein alkyl, cycloalkyl and heterocycloalkyl are unsubstituted or substituted with 1 to 6 substituents independently selected from the group consisting of halogen, C₁₋₆-alkyl, halo-C₁₋₆-alkyl, OR¹ and CN;

D represents 6- to 10-membered mono- or bicyclic aryl or 5- to 10-membered mono- or bicyclic heteroaryl containing 1 to 4 heteroatoms independently selected from O, N and S,

wherein aryl and heteroaryl are unsubstituted or substituted with 1 to 5 substituents independently selected from halogen, OH, R^{z}, O—R^{z}, OC(O)—R^{z}, S—R^{z}, S(O)₂—R^{z}, S(O)₂N(R¹)₂, N(R¹)₂, NR¹C(O)R^{z}, C(O)N(R¹)₂, C(O)O—R^{z}, C(O)—R^{z}, CN, COOH, 5- or 6-membered heteroaryl containing 1 to 4 heteroatoms independently selected from O, N and S and 6-membered aryl,

wherein aryl and heteroaryl are unsubstituted or substituted with 1 to 5 substituents independently selected from the group consisting of halogen, CN, OR¹, C₁₋₆-alkyl; C₃₋₆-cycloalkyl, and halo-C₁₋₆-alkyl, or

two substituents on the aryl or heteroaryl ring systems together with the carbon atom to which they are attached form a 5- or 6-membered heterocyclic ring containing 1 or 2 heteroatoms independently selected from O, N and S,

wherein the heterocylic ring is unsubstituted or substituted with 1 or 2 substituents independently selected from the group consisting of halogen, C₁₋₆-alkyl, halo-C₁₋₆-alkyl, CN and oxo;

R^{z} represents C₁₋₆-alkyl, C₃₋₆-cycloalkyl or 3- to 6 membered heterocyclyl containing 1 to 2 heteroatoms independently selected from O, N and S,

wherein alkyl, cycloalkyl and heterocycloalkyl are unsubstituted or substituted with 1 to 6 substituents independently selected from the group consisting of halogen, C₁₋₆-alkyl, halo-C₁₋₆-alkyl, OR¹ and CN;

V is O or CR⁷R⁸;

R² is halogen, C₁₋₆ alkyl, C₃₋₆ cycloalkyl, halo-C₁₋₆-alkyl, OR⁴, S(O)₂N(R¹)₂, S(O)₂—C₁₋₆-alkyl, S(O)₂—C₃₋₆-cycloalkyl, S(O)₂-halo-C₁₋₆-alkyl, S(O)₂N(R¹)₂, C(O)N(R¹)₂, CN, C(O)OR⁴ or oxo, or

two R² on the same carbon atom form together with the carbon atom to which they are attached a C₃₋₁₀ cycloalkyl group, or

two R² at different carbon atoms form together a —CH₂—, —CH(CH₃)—, —C(CH₃)₂—, CH₂—CH(CH₃)—, —CH₂—CH₂— or —CH₂—CH₂—CH₂— group;

R⁷ and R⁸ are independently selected from the group consisting of hydrogen, halogen, C₁₋₆ alkyl, halo-C₁₋₆-alkyl and OR⁴;

o is 0-4; and

k is 1 or 2.

In a more preferred embodiment in combination with any of the above and below embodiments, the compound is represented by the following Formulae (2-3) and (2-4)

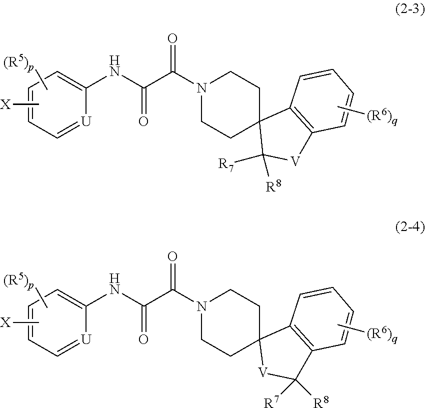

wherein

X is hydrogen, halogen, C₁₋₆-alkyl, C₃₋₆-cycloalkyl, O—C₁₋₆-alkyl, S—C₁₋₆-alkyl, CN or 5- or 6-membered heteroaryl containing 1 to 4 heteroatoms independently selected from O, N and S,

wherein alkyl and cycloalkyl are unsubstituted or substituted with halogen, C₁₋₆-alkyl, halo-C₁₋₆-alkyl, OR¹ or CN;

R⁵ is independently selected from hydrogen, halogen and C₁₋₆-alkyl;

R⁶ is independently selected from halogen, OH, R^{z}, O—R^{z}, OC(O)—R^{z}, S—R^{z}, S(O)₂—R^{z}, S(O)₂N(R)₂, N(R)₂, NR¹C(O)R^{z}, C(O)N(R)₂, C(O)O—R^{z}, C(O)—R^{z}, CN, COOH, 5- or 6-membered heteroaryl containing 1 to 4 heteroatoms independently selected from O, N and S and 6-membered aryl,

wherein aryl and heteroaryl are unsubstituted or substituted with 1 to 5 substituents independently selected from the group consisting of halogen, CN, OR¹, C₁₋₆-alkyl; C₃₋₆-cycloalkyl, and halo-C₁₋₆-alkyl,

R^{z} represents C₁₋₆-alkyl, C₃₋₆-cycloalkyl or 3- to 6 membered heterocyclyl containing 1 to 2 heteroatoms independently selected from O, N and S,

wherein alkyl, cycloalkyl and heterocycloalkyl are unsubstituted or substituted with 1 to 6 substituents independently selected from the group consisting of halogen, C₁₋₆-alkyl, halo-C₁₋₆-alkyl, OR¹ and CN;

U is N or CR⁵;

V is O or CR⁷R⁸;

p is 0, 1, 2 or 3; and

q is 0, 1, 2, 3 or 4.

In a further more preferred embodiment of the compounds according to Formulae (2-3) and (2-4) in combination with any of the above and below embodiments, U is CR⁵, in particular CH.

In a further more preferred embodiment of the compounds according to Formulae (2-3) and (2-4) in combination with any of the above and below embodiments, V is —O— and R⁷ and R⁸ both represent hydrogen.

In a most preferred embodiment of the compounds according to Formula (2) in combination with any of the above and below embodiments, the compounds according to Formula (2) are selected from

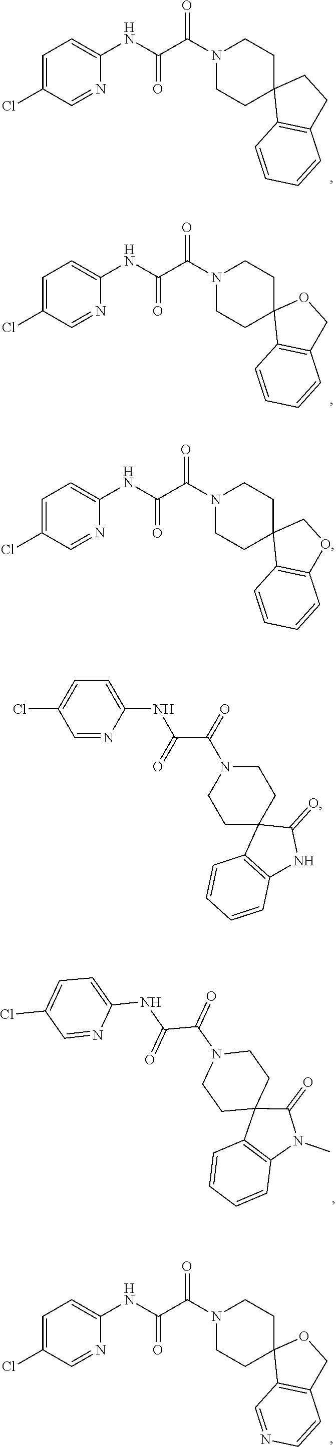 [SMILES: O=C(Nc1ccc(Cl)cn1)C(=O)N1CCC2(CCc3ccccc32)CC1 ; O=C(Nc1ccc(Cl)cn1)C(=O)N1CCC2(CC1)OCc1ccccc12 ; O=C(Nc1ccc(Cl)cn1)C(=O)N1CCC2(CC1)COc1ccccc12 ; O=C(Nc1ccc(Cl)cn1)C(=O)N1CCC2(CC1)C(=O)Nc1ccccc12 ; CN1C(=O)C2(CCN(C(=O)C(=O)Nc3ccc(Cl)cn3)CC2)c2ccccc21 ; O=C(Nc1ccc(Cl)cn1)C(=O)N1CCC2(CC1)OCc1ccncc12]

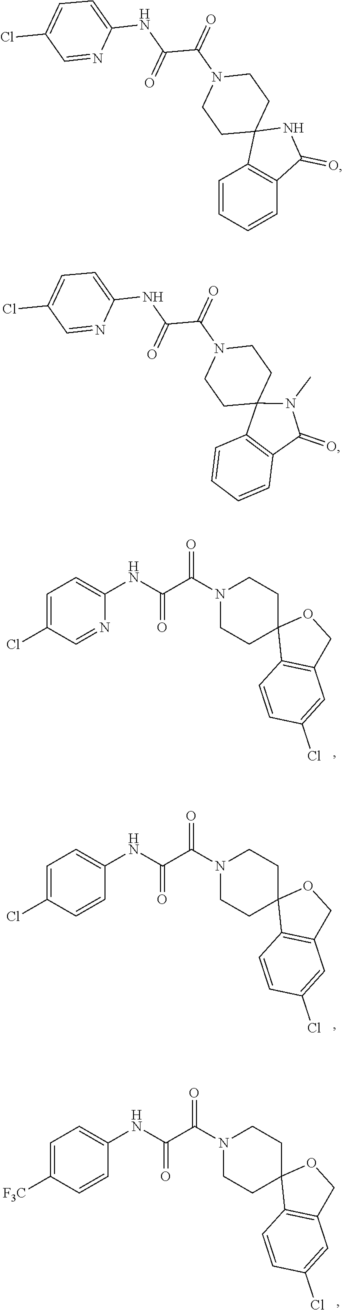 [SMILES: O=C(Nc1ccc(Cl)cn1)C(=O)N1CCC2(CC1)NC(=O)c1ccccc12 ; CN1C(=O)c2ccccc2C12CCN(C(=O)C(=O)Nc1ccc(Cl)cn1)CC2 ; O=C(Nc1ccc(Cl)cn1)C(=O)N1CCC2(CC1)OCc1cc(Cl)ccc12 ; O=C(Nc1ccc(Cl)cc1)C(=O)N1CCC2(CC1)OCc1cc(Cl)ccc12 ; O=C(Nc1ccc(C(F)(F)F)cc1)C(=O)N1CCC2(CC1)OCc1cc(Cl)ccc12]

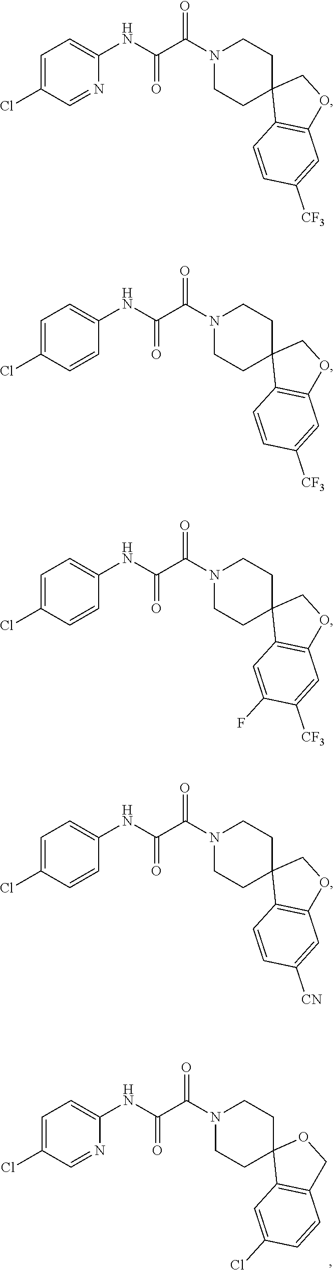 [SMILES: O=C(Nc1ccc(Cl)cn1)C(=O)N1CCC2(CC1)COc1cc(C(F)(F)F)ccc12 ; O=C(Nc1ccc(Cl)cc1)C(=O)N1CCC2(CC1)COc1cc(C(F)(F)F)ccc12 ; O=C(Nc1ccc(Cl)cc1)C(=O)N1CCC2(CC1)COc1cc(C(F)(F)F)c(F)cc12 ; N#Cc1ccc2c(c1)OCC21CCN(C(=O)C(=O)Nc2ccc(Cl)cc2)CC1 ; O=C(Nc1ccc(Cl)cn1)C(=O)N1CCC2(CC1)OCc1ccc(Cl)cc12]

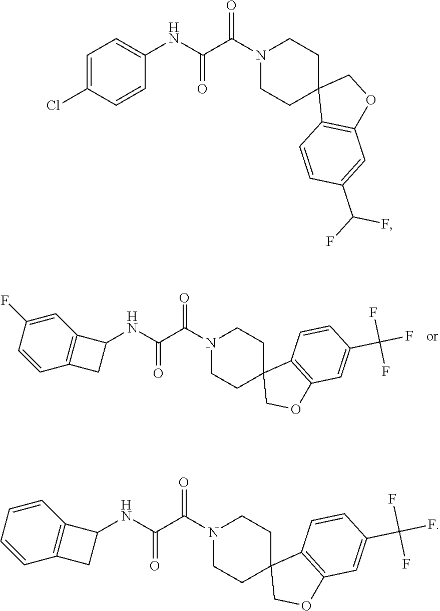 [SMILES: O=C(Nc1ccc(Cl)cc1)C(=O)N1CCC2(CC1)COc1cc(C(F)F)ccc12 ; O=C(NC1Cc2ccc(F)cc21)C(=O)N1CCC2(CC1)COc1cc(C(F)(F)F)ccc12 ; O=C(NC1Cc2ccccc21)C(=O)N1CCC2(CC1)COc1cc(C(F)(F)F)ccc12]

In an uppermost preferred embodiment of the compounds according to Formula (2) in combination with any of the above and below embodiments, the compound according to Formula (2) is selected from

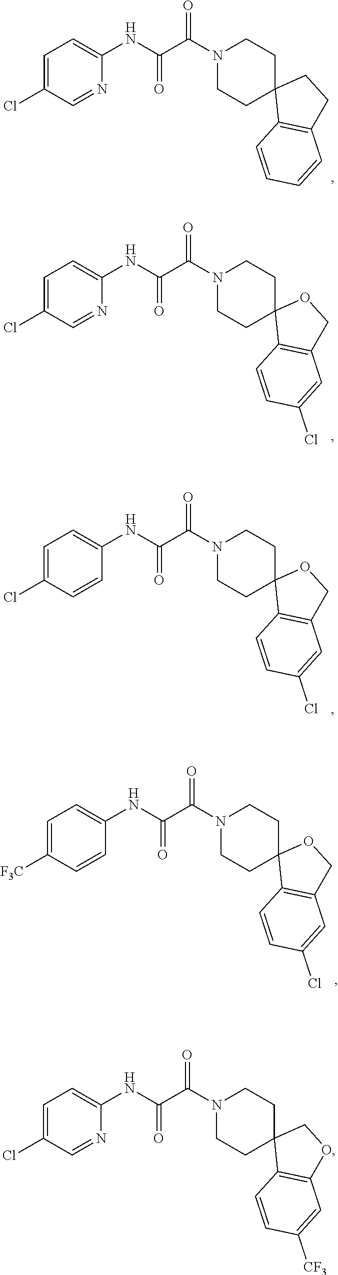 [SMILES: O=C(Nc1ccc(Cl)cn1)C(=O)N1CCC2(CCc3ccccc32)CC1 ; O=C(Nc1ccc(Cl)cn1)C(=O)N1CCC2(CC1)OCc1cc(Cl)ccc12 ; O=C(Nc1ccc(Cl)cc1)C(=O)N1CCC2(CC1)OCc1cc(Cl)ccc12 ; O=C(Nc1ccc(C(F)(F)F)cc1)C(=O)N1CCC2(CC1)OCc1cc(Cl)ccc12 ; O=C(Nc1ccc(Cl)cn1)C(=O)N1CCC2(CC1)COc1cc(C(F)(F)F)ccc12]

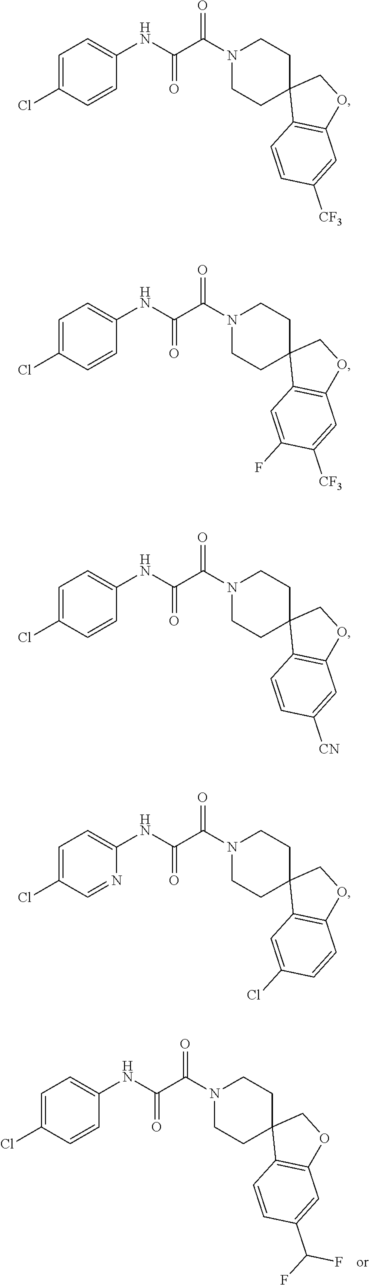 [SMILES: O=C(Nc1ccc(Cl)cc1)C(=O)N1CCC2(CC1)COc1cc(C(F)(F)F)ccc12 ; O=C(Nc1ccc(Cl)cc1)C(=O)N1CCC2(CC1)COc1cc(C(F)(F)F)c(F)cc12 ; N#Cc1ccc2c(c1)OCC21CCN(C(=O)C(=O)Nc2ccc(Cl)cc2)CC1 ; O=C(Nc1ccc(Cl)cn1)C(=O)N1CCC2(CC1)COc1ccc(Cl)cc12 ; O=C(Nc1ccc(Cl)cc1)C(=O)N1CCC2(CC1)COc1cc(C(F)F)ccc12]

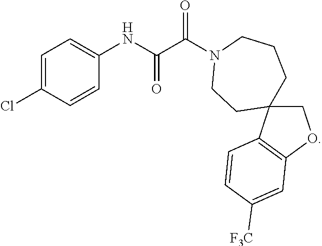 [SMILES: O=C(Nc1ccc(Cl)cc1)C(=O)N1CCCC2(CC1)COc1cc(C(F)(F)F)ccc12]

In the context of the present invention “C₁₋₆-alkyl” means a saturated alkyl chain having 1, 2, 3, 4, 5, or 6 carbon atoms which may be straight chained or branched. Examples thereof include methyl, ethyl, propyl, isopropyl, n-butyl, isobutyl, tert-butyl, n-pentyl, isopentyl, neopentyl and hexyl.

The term “halo-C₁₋₆-alkyl” means that one or more hydrogen atoms in the alkyl chain are replaced by a halogen atom which may be the same or different. Preferred example thereof include CHF₂, CF₃, CH₂CH₂Cl, and CH₂CF₃.

A “C_{x-y}-alkylene” means that the respective group is divalent and connects the attached residue with the remaining part of the molecule. X is an integer selected from 0, 1, and 2 and y is an integer selected from 0, 1, 2, and 3. Moreover, in the context of the present invention, “C₀-alkylene” is meant to represent a bond. An alkylene group may be straight chained or branched.

A C₃₋₁₀-cycloalkyl group or C₃₋₁₀-carbocycle means a saturated or partially unsaturated mono-, bi-, spiro-, or multicyclic ring system comprising 3, 4, 5, 6, 7, 8, 9, or 10 carbon atoms. Examples include cyclopropyl, cyclobutyl, cyclopentyl, cyclohexyl, cyclohexenyl, bicyclo[2.2.2]octyl, bicyclo[2.2.1]heptyl, adamantyl, spiro[3.3]heptane and pentacyclo[4.2.0.0^{2,5}.0^{3,8}.0^{4,7}]octyl. As an example, a C₃₋₆-cycloalkyl group means a cycloalkyl ring having 3, 4, 5 or 6 carbon atoms. The C₃₋₁₀-cycloalkyl group can be connected to the remainder of the molecule via a bond or the cycloalkyl group may share a carbon at the attachment point with the remainder of the molecule. Illustrative examples of the attachment possibilities are shown below:

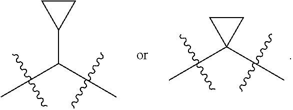 [SMILES: *C(*)C1CC1 ; *C1(*)CC1]

A 3- to 10-membered heterocycloalkyl group means a saturated or partially unsaturated mono-, bi-, tri-, spiro or multicyclic ring system having 3, 4, 5, 6, 7, 8, 9 or 10 ring members. Similarly, a 3- to 6-membered heterocycloalkyl group means a saturated or partially unsaturated mono-, bi-, spiro or multicyclic ring system having 3, 4, 5 or 6 ring members. The heterocycloalkyl comprises up to 5 heteroatoms, such as 1, 2, 3, 4 or 5 heteroatoms, preferably 1, 2 or 3 heteroatoms, more preferably 1 or 2 heteroatoms and most preferably 1 heteroatom, wherein the heteroatoms are independently selected from N, O, S, S(O) and S(O)₂, preferably N, O and S. Examples thereof include epoxidyl, azetidinyl, oxetanyl, pyrrolidinyl, tetrahydrofuranyl, piperidinyl, piperazinyl tetrahydropyranyl, 1,4-dioxanyl, morpholinyl, 4-quinuclidinyl, 1,4-dihydropyridinyl, 2-azaspiro[3.3]heptane and 3,6-dihydro-2H-thiopyranyl. The heterocycloalkyl group can be connected to the remainder of the molecule via a carbon atom or nitrogen atom.

A 5-14-membered mono-, bi- or tricyclic heteroaromatic ring system (within the application also referred to as heteroaryl) containing up to 4 heteroatoms means a monocyclic heteroaromatic ring such as pyrrolyl, imidazolyl, furanyl, thiophenyl, pyridinyl, pyrimidinyl, pyrazinyl, pyrazolyl, oxazolyl, isoxazolyl, triazolyl, oxadiazolyl and thiadiazolyl. It further means a bicyclic ring system wherein the heteroatom(s) may be present in one or both rings including the bridgehead atoms. Examples thereof include quinolinyl, isoquinolinyl, quinoxalinyl, benzimidazolyl, benzisoxazolyl, benzodioxanyl, benzofuranyl, benzoxazolyl, indolyl, indolizinyl, pyrazolo[1,5-a]pyrimidinyl and dibenzo[b,d]furanyl. The nitrogen or sulphur atom of the heteroaryl system may also be optionally oxidized to the corresponding N-oxide, S-oxide or S,S-dioxide. If not stated otherwise, the heteroaryl system can be connected via a carbon or nitrogen atom. Examples for N-linked heterocycles are

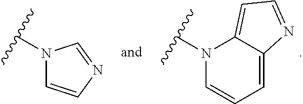 [SMILES: *n1ccnc1 ; *n1cccc2nccc1-2]

A 6-10-membered mono- or bicyclic aromatic ring system (within the application also referred to as aryl) means an aromatic carbon cycle such as phenyl or naphthyl.

Halogen is selected from fluorine, chlorine, bromine and iodine.

The compounds of the present invention are further intended to include all possible geometric isomers. Cis and trans geometric isomers of the compounds of the present invention are described and may be isolated as a mixture of isomers or as separated forms. A bond in a structure diagram represented by a wavy line “ [symbol] ” is intended to indicate that the structure represents the cis or the trans isomer, or a mixture of the cis and trans isomers in any ratio.

Compounds of the present invention also include tautomeric forms. Tautomeric forms result from the swapping of a single bond with an adjacent double bond together with the concomitant migration of a proton.

The compounds of the present invention can be in the form of a pharmaceutically acceptable salt or a solvate. The term “pharmaceutically acceptable salts” refers to salts prepared from pharmaceutically acceptable non-toxic bases or acids, including inorganic bases or acids and organic bases or acids. In case the compounds of the present invention contain one or more acidic or basic groups, the invention also comprises their corresponding pharmaceutically or toxicologically acceptable salts, in particular their pharmaceutically utilizable salts. Thus, the compounds of the present invention which contain acidic groups can be used according to the invention, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine or amino acids.

The compounds of the present invention which contain one or more basic groups, i.e. groups which can be protonated can be used according to the invention in the form of their addition salts with inorganic or organic acids. Examples of suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. If the compounds of the present invention simultaneously contain acidic and basic groups in the molecule, the invention also includes, in addition to the salt forms mentioned, inner salts or betaines (zwitterions). The respective salts can be obtained by customary methods which are known to the person skilled in the art like, for example, by contacting these with an organic or inorganic acid or base in a solvent or dispersant, or by anion exchange or cation exchange with other salts. The present invention also includes all salts of the compounds of the present invention which, owing to low physiological compatibility, are not directly suitable for use in pharmaceuticals but which can be used, for example, as intermediates for chemical reactions or for the preparation of pharmaceutically acceptable salts.

Further the compounds of the present invention may be present in the form of solvates, such as those which include as solvate water, or pharmaceutically acceptable solvates, such as alcohols, in particular ethanol.

Any formula or structure given herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, fluorine and chlorine, such as, but not limited to ²H (deuterium, D), ³H (tritium), ¹¹C, ¹³C, ¹⁴C, ¹⁵N, ¹⁸F, ³⁵S, ³⁶Cl and ¹²⁵I. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

The disclosure also includes “deuterated analogs” of compounds of Formula (I) in which from 1 to n hydrogens attached to a carbon atom is/are replaced by deuterium, in which n is the number of hydrogens in the molecule. Such compounds may exhibit increased resistance to metabolism and thus be useful for increasing the half-life of any compound of Formula (I) when administered to a mammal, e.g. a human. See, for example, Foster in Trends Pharmacol. Sci. 1984:5; 524. Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

The compounds of the present invention are useful as inhibitors of IDO1. Hence, they are potential therapeutic agents for the prophylaxis and/or treatment of IDO1-mediated diseases or conditions such as cancer, viral and bacterial infections such as HIV infection, hanta virus infection, tuberculosis, leprae, depression, epilepsy, schizophrenia, neurodegenerative diseases such as Alzheimer's disease and Huntington's disease, trauma, age-related cataracts, organ transplantation, cardiovascular disease, endometriosis, type 2 diabetic nephropathy, chronic obstructive pulmonary disease (COPD), osteoporosis, asthma, rheumatoid arthritis, multiple sclerosis, inflammatory bowel disease, psoriasis, and systemic lupus erythematosus.

In a preferred embodiment, the compounds are used in the prophylaxis and/or treatment of cancer.

Examples of cancer types that may be treated using the compounds and compositions described herein include but are not limited to carcinomas, sarcomas, lymphomas and leukemias, germ cell tumors and blastomas, cancer of adrenal gland, bladder, brain, breast, bone, cervix, colorectum, colon, connective tissue, endometrium, esophagus, head, liver, lung, mesothelial lining, muscle, neck, ovary, pancreas, prostate, skin, stomach, testis, thyroid, white blood cell, or glioblastoma, mesothelioma, melanoma, renal cell carcinoma, gastric carcinoma, choriocarcinoma, cutaneous basocellular carcinoma, testicular seminoma and ovarian dysgerminoma. In a recent review by Hornyák et al. examples of such cancer types are given (Hornyák et al. Front Immunol. 2018 Jan. 31; 9:151).

Furthermore, the present invention provides pharmaceutical compositions comprising at least one compound of the present invention, or a pharmaceutically acceptable salt or solvate thereof as active ingredient together with a pharmaceutically acceptable carrier.

“Pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing at least one compound of the present invention and a pharmaceutically acceptable excipient.

The pharmaceutical composition of the present invention may additionally comprise one or more other compounds as active ingredients like a prodrug compound or other therapeutic agents.

Additional therapeutic agents are preferably selected from known cancer therapeutics. Examples thereof include PD-1 agent, PD-L1 agent, CTLA-4 agent as well as chemotherapeutic agents, anticancer vaccines, oncolytic viruses, cytokine therapy, TLR agonists, STING agonists, as well as other immuno oncology therapeutics. The compounds of the present invention may also be administered to a patient while the patient undergoes irradiation therapy.

Examples of PD-1 agents include, but are not limited to, Pembrolizumab, Cemiplimab and Nivolumab.

Examples of PD-L1 agents include, but are not limited to, Atezolizumab, Avelumab and Durvalumab.

Examples of CTLA-4 agents include, but are not limited to, Ipilimumab.

Examples of chemotherapeutic agents include, but are not limited to, Cyclophosphamide, Busulfan, Carmustin, Temozolimide, Procarbazin, Trabectedin, Cisplatin, Carboplatin, Methotrexat, Pemetrexed, 6-Mercatopurine, 6-Thioguanine, Cladibine, Clofarabine, Nelarabine, Pentostatine, 5-Fluorouracil, Cytarabine, Gemcitabine, Azacitidine, Vincristine, Vinblastine, Vindesine, Paclitaxel, Docetaxel, Cabazitaxel, Ixabepilone, Eribulin, Estramustine phosphate, Topotecan, Irinotecan, Etoposide, Teniposide, Dactinomycin, Bleomycin, Doxorubicin, Daunorubicin, Epirubicin, Idarubicin, Mitoxantron, all-trans retinoic acid, Bexarotene, As₂O₃, Imatinib, Nilotinib, Dasatinib, Bosutinib, Ponatinib, Erlotinib, Gefitinib, Afatinib, Osimertinib, Lapatinib, Crizotinib, Ceritinib, Axitinib, Cabozantinib, Lanvatinib, Nintedanib, Pazopanib, Regorafenib, Sorafenib, Sunitinib, Ruxolitinib, Dovitinib, Ibrutinib, Idelalisib, Vemurafenib, Dabrafenib, Trametinib, Cobimetinib, Palbociclib, Temsirolismus, Everolimus, Bortezomib, Carfilzomib, Vismodegib, Panobinostat, Olaparib, Venetoclax, Rituximab, Trastuzumab, Pertuzumab, Cetuximab, Panitumumab, Necitumumab, Bevacizumab, Ramucirumab, Olaratumab, Mifamurtide, Elotuzumab, Catumaxomab, Blinatumomab, Rituximab, Daratumumab, Alemtuzumab, Prednisone, Buserelin, Goserelin, Leuprorelin, Histrelin, Triptorelin, Degarelix, Abarelix, Flutamide, Bicalutmide, Enzalutamide, Arbiraterone, Tamoxifen, Toremifen, Exemestane, Letrozole, Anastrozole, Fulvestrant, Thalidomide, Lenalidomide, Pomalidomide,

Examples of anticancer vaccines include, but are not limited to, Hepa-VAC-101 and Sipuleucel-T.

Examples of oncolytic viruses include, but are not limited to, H101, Talimogene laherparepvec.

Examples of Toll like receptor agonists include, but are not limited to, Imiquimod, Resiquimod, monophosphoryl lipid A, BCG, CpG ODNs, Motolimod, GSK1795091 and Telratolimod.

Examples of STING agonists include, but are not limited to, ADU-S100 and MK-1454.

Examples of cytokine therapy include, but are not limited to, IL-2, GM-CSF, IL-12 and IL-10.

Examples of other Immune-Oncology therapeutics that can be used in combination with the compounds of the present invention include, but are not limited to Chimeric antigen receptor, or CAR T-cell therapy, such as Tisagenlecleucel, Axicabtagen Ciloleucel, agents targeting T cell co-stimulatory (e.g. OX40) or co-inhibitory (e.g. LAG3) molecules and immune response modifying enzymes such as Asparaginase or Kynureninase.

The compositions are suitable for oral, rectal, topical, parenteral (including subcutaneous, intramuscular, and intravenous), ocular (ophthalmic), pulmonary (nasal or buccal inhalation) or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of the present invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, emulsions and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or non-aqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin.

When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

The compounds of the present invention may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of the present invention are administered orally.

The effective dosage of active ingredient employed may vary depending on the particular compound employed, the mode of administration, the condition being treated and the severity of the condition being treated. Such dosage may be ascertained readily by a person skilled in the art.

When treating or preventing IDO mediated conditions for which compounds of the present invention are indicated, generally satisfactory results are obtained when the compounds of the present invention are administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams, preferably from about 1 milligram to about 50 milligrams. In the case of a 70 kg adult human, the total daily dose will generally be from about 7 milligrams to about 350 milligrams. This dosage regimen may be adjusted to provide the optimal therapeutic response.

### ABBREVIATIONS

Herein and throughout the application, the following abbreviations may be used.
Ac acetyl
AIBN 2,2′-(diazene-1,2-diyl)bis(2-methylpropanenitrile)
br broad
CDI 1,1′-carbonyldiimidazole
d doublet
DAST diethylaminosulfur trifluoride
DCE 1,2-dichloroethane
DCM dichloromethane
DEAD diethyl diazene-1,2-dicarboxylate
DIBAL-H diisobutylaluminum hydride
DIPEA N,N-diisopropylethylamine
DMF N,N-dimethylformamide
DMSO dimethyl sulfoxide
DPPA Diphenylphosphoryl azide
EDC 1-ethyl-3-(3-dimethylaminopropyl)carbodiimide
Et₂O diethyl ether
EtOAc ethyl acetate
HATU O-(7-azabenzotriazol-1-yl)-N,N,N′,N′-tetramethyluronium hexafluorophosphate
HOBt 1-hydroxybenzotriazole
HPLC high performance liquid chromatography
i-PrOH isopropyl alcohol
LDA lithium diisopropylamide
m multiplet
mCPBA 3-chloroperoxybenzoic acid
Ms methanesulfonyl
NCS N-chlorosuccinimide
PE petroleum ether
prep preparative
rt room temperature
SFC supercritical fluid chromatography
t triplet
TEA triethylamine
TFA trifluoroacetic acid
TFAA trifluoroacetic acid anhydride
THE tetrahydrofurane

### GENERAL SCHEMES

The compounds of the present invention can be prepared by a combination of methods known in the art including the procedures described in schemes 1-4 below. The following reaction schemes are only meant to represent examples of the invention and are in no way meant to be a limit of the invention.

Scheme 1 shows the synthesis of intermediates of structure A-4. A Suzuki coupling of boronic acid or boronic acid ester A-1 with halogen A-2 or alternatively enol triflate A-1 with boronic acid or boronic acid ester A-2 affords the cyclic olefin A-3. A sequence of hydrogenation and deprotection gives intermediates of structure A-4 where Y is a hydrogen. Alternatively aryl or heteroaryl halides A-5 can be metallated with e.g. n-BuLi followed by reaction with ketones of structure A-6 to give cyclic hydroxy compounds A-7. Deprotection or a sequence of transformation of the hydroxy group with e.g. DAST followed by deprotection gives intermediates of structure A-4.

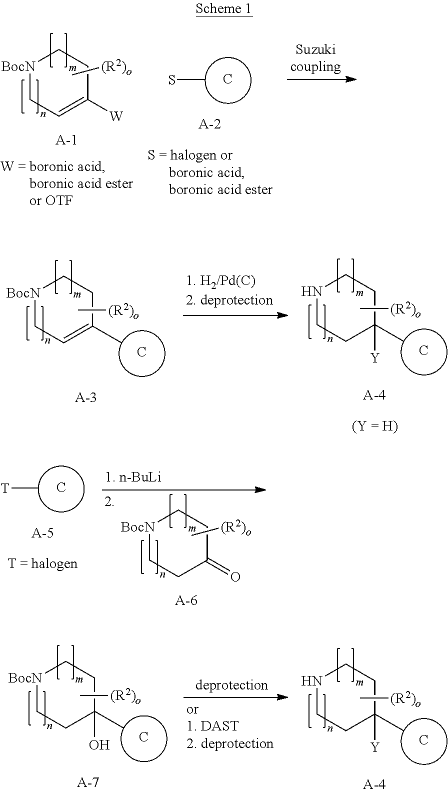

Scheme 2 shows the synthesis of compounds of structure B-3 and B-5 of the present invention. Amine B-1 undergoes amide formation with methyl 2-chloro-2-oxoacetate to give oxalamide ester intermediates of structure B-2. Direct ester aminolysis with cyclic amines A-4 or B-4 gives compounds of structure B-3 or B-5. Alternatively B-2 is saponified to the corresponding carboxylic acid which can be converted to B-3 or B-5 by amide coupling reaction using e.g. HATU.

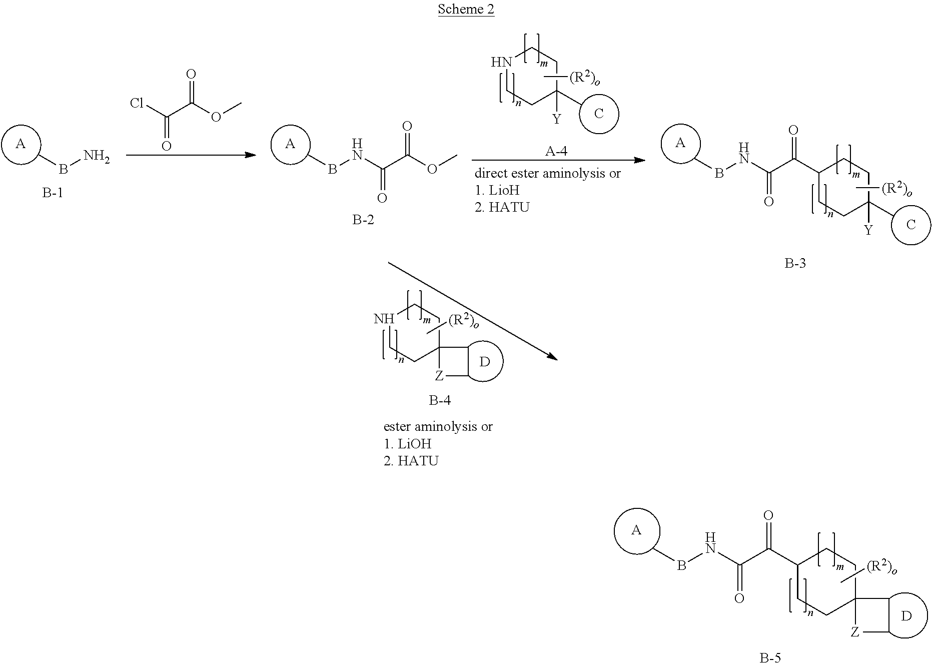

Scheme 3 shows an alternative synthesis of compounds of structure B-3. Cyclic amine A-4 undergoes amide formation with methyl 2-chloro-2-oxoacetate to give oxalamide ester intermediates of structure C-1. Direct ester aminolysis with B-1 or alternatively ester saponification followed by amide coupling reaction affords compounds of structure B-3.

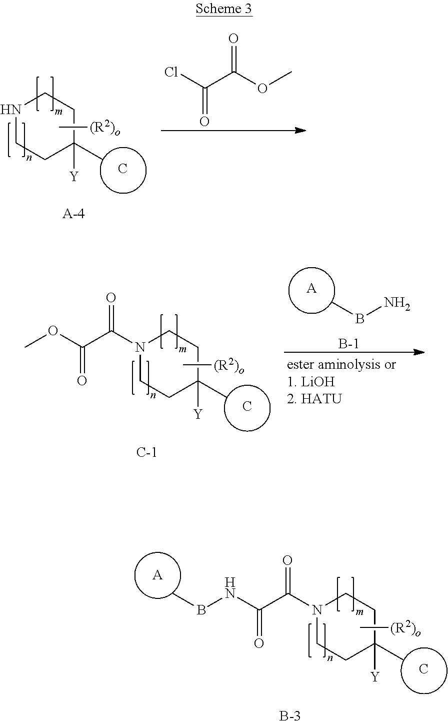

Scheme 4 shows the synthesis of intermediates of structure B-4. Pyridine C-1 can be N-alkylated with (chloromethyl)benzene at elevated temperatures. The pyridinium intermediate C-2 can be partially reduced with NaBH₄ to give the tetrahydropyridine C-3, which can be transformed into ether intermediate C-4 via Mitsunobu reaction. Radical cyclization of C-4 using n-Bu₃SnH and AIBN affords the corresponding spirocyclic intermediate C-5. Deprotection of C-5 with 1-chloroethyl carbonylchloridate leads to intermediates of structure B-4.

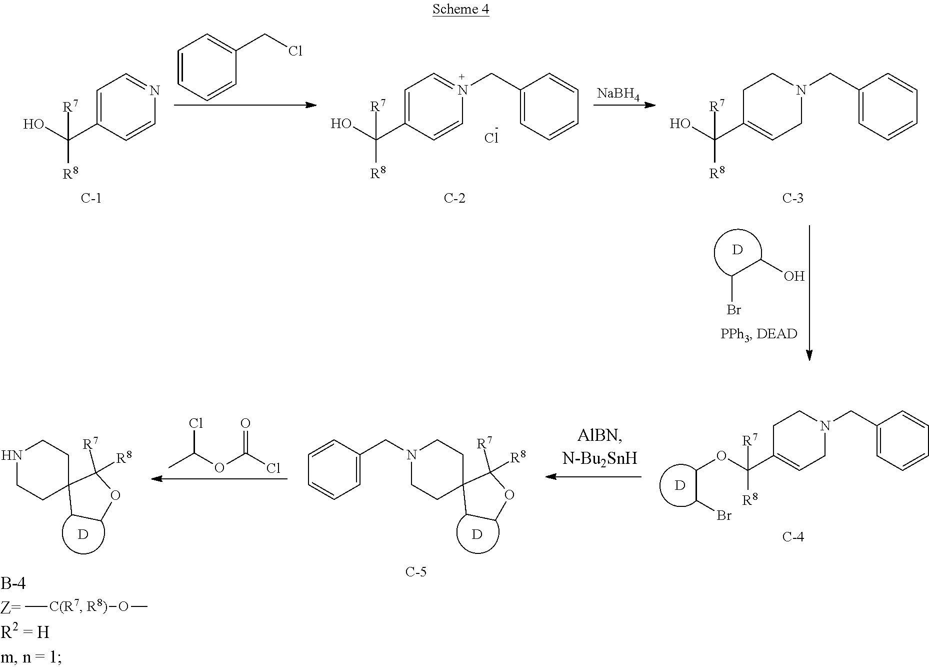

### Intermediate 1: 4-(Difluoromethyl)aniline (Int 1)

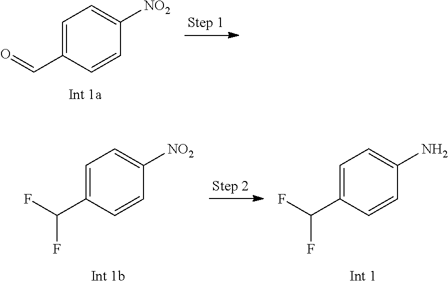 [SMILES: O=Cc1ccc([N+](=O)[O-])cc1 ; O=[N+]([O-])c1ccc(C(F)F)cc1 ; Nc1ccc(C(F)F)cc1]

### Step1: 1-(Difluoromethyl)-4-nitrobenzene (Int 1b)

To a mixture of 4-nitrobenzaldehyde (5.00 g, 33.10 mmol) in DCM (75 mL) diethylaminosulfur trifluoride (6.40 g, 39.70 mmol) was added dropwise at −70° C. The mixture was stirred at the same temperature for 10 min. The cooling bath was removed and the mixture was stirred overnight. Concentrated aqueous NaHCO₃ solution was added slowly to the mixture and the mixture was extracted with DCM (3×50 mL). The combined organic layers were dried over anhydrous Na₂SO₄, filtered and concentrated to dryness. The residue was purified by column chromatography (PE) to provide the title compound.

### Step 2: 4-(Difluoromethyl)aniline (Int 1)

To a mixture of 1-(difluoromethyl)-4-nitrobenzene (Int 1b) (3.50 g, 20.20 mmol) in THE (50 mL) was added NH₄Cl (10.8 g, 202 mmol) and iron powder (11.3 g, 202 mmol) and the mixture was stirred at rt overnight. The mixture was filtered and water was added. The mixture was extracted with EtOAc (3×50 mL) and the combined organic layers were dried over anhydrous Na₂SO₄, filtered and concentrated to dryness to give the title compound as a yellow oil.

### Intermediate 2: 4-Bromo-2-(difluoromethoxy)quinoline (Int 2)

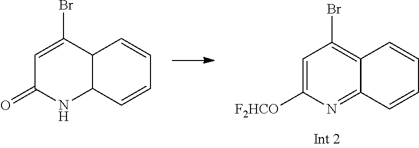 [SMILES: O=C1C=C(Br)C2C=CC=CC2N1 ; FC(F)Oc1cc(Br)c2ccccc2n1]

To a solution of 4-bromo-4a,8a-dihydroquinolin-2(1H)-one (672 mg, 3.00 mmol) in DMF (20 mL) was added sodium 2-chloro-2,2-difluoroacetate (684 mg, 4.50 mmol), and Na₂CO₃ (636 mg, 6.00 mmol) and the mixture was stirred at 80° C. for 2 h under N₂. It was cooled to rt and diluted with H₂O (20 mL). It was extracted with EtOAc (3×20 mL).

The combined organic layers were washed with brine (40 mL), dried over Na₂SO₄, filtered and concentrated. The residue was purified by silica gel column chromatography, eluting with PE/EtOAc (4:1) to give the title compound as a yellow solid.

### Intermediate 3: 4-Fluorobicyclo[4.2.0]octa-1(6),2,4-trien-7-amine hydrochloride (Int 3)

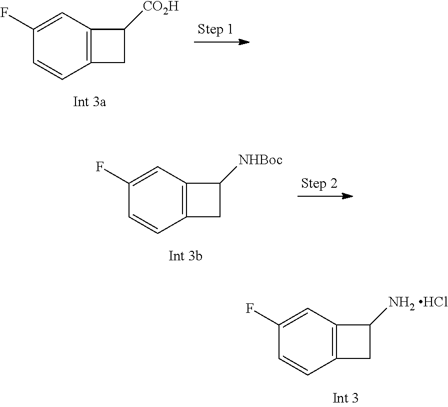

### Step 1: tert-Butyl (4-fluorobicyclo[4.2.0]octa-1(6),2,4-trien-7-yl)carbamate (Int 3b)

To a solution of 4-fluorobicyclo[4.2.0]octa-1(6),2,4-triene-7-carboxylic acid (Int 3a) (0.488 g, 3.0 mol) and TEA (0.613 mL, 4.4 mmol) was added DPPA (970 mg, 3.5 mmol) and the mixture was stirred at 85° C. for 4 h. After cooling to rt the mixture was concentrated to dryness and the residue was purified by silica gel column chromatography (EtOAc/Cyclohexane) to afford the title compound as a white solid.

### Step 2: 4-Fluorobicyclo[4.2.0]octa-1(6),2,4-trien-7-amine Hydrochloride (Int 3)

To a solution of tert-butyl (4-fluorobicyclo[4.2.0]octa-1(6),2,4-trien-7-yl)carbamate (Int 3b) (400 mg, 1.7 mmol) in EtOAc (4 mL) was added HCl in 1,4-dioxane (2.0 mL, 4 M, 8 mmol) and the mixture was stirred at rt for 2 h. The mixture was concentrated to dryness to afford the title compound as a white solid.

### Intermediate 10: Methyl 2-((3-chloro-4-fluorophenyl)amino)-2-oxoacetate (Int 10)

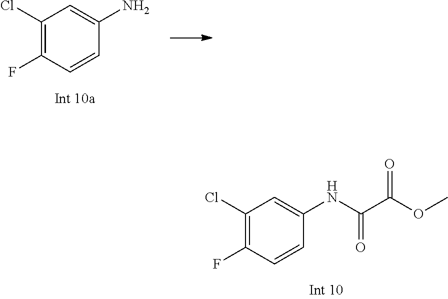

To a mixture of 3-chloro-4-fluoroaniline (1.00 g, 6.87 mmol) and TEA (1.43 ml, 10.31 mmol) in DCM (25 mL) was added methyl 2-chloro-2-oxoacetate (890 mg, 7.20 mmol). The mixture was stirred at rt overnight. The mixture was filtered and the residue was extracted with EtOAc (20 ml). The combined organic layers were concentrated to dryness to give the title compound as a white solid.

### Intermediates 10/1 to 10/29

The following Intermediates were prepared similar as described for Intermediate 10 using the appropriate amine building blocks.

| Int. # | building block | Structure |
|---|---|---|
| Int 10/1 | 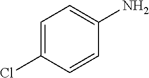 | 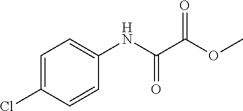 |
| Int 10/2 | 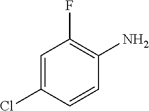 | 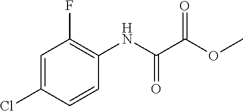 |
| Int 10/3 | 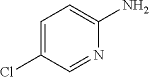 | 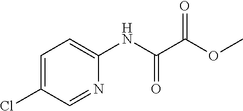 |
| Int 10/4 | 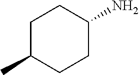 | 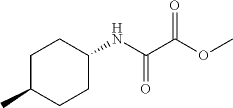 |
| Int 10/5 | 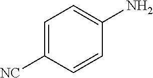 | 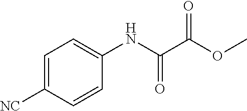 |
| Int 10/6 | 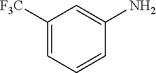 [SMILES: Nc1cccc(C(F)(F)F)c1] | 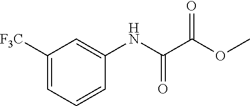 [SMILES: COC(=O)C(=O)Nc1cccc(C(F)(F)F)c1] |
| Int 10/7 | 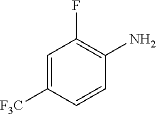 [SMILES: Nc1ccc(C(F)(F)F)cc1F] | 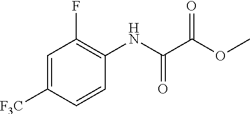 [SMILES: COC(=O)C(=O)Nc1ccc(C(F)(F)F)cc1F] |
| Int 10/8 | 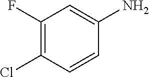 | 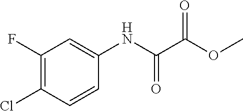 |
| Int 10/9 | 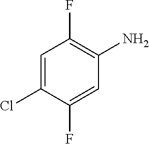 | 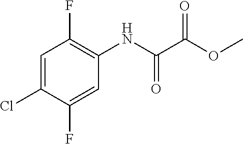 [SMILES: COC(=O)C(=O)Nc1cc(F)c(Cl)cc1F] |
| Int 10/10 | 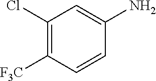 [SMILES: Nc1ccc(C(F)(F)F)c(Cl)c1] | 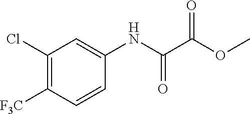 [SMILES: COC(=O)C(=O)Nc1ccc(C(F)(F)F)c(Cl)c1] |
| Int 10/11 | 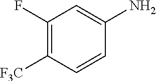 [SMILES: Nc1ccc(C(F)(F)F)c(F)c1] | 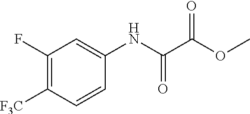 [SMILES: COC(=O)C(=O)Nc1ccc(C(F)(F)F)c(F)c1] |
| Int 10/12 | 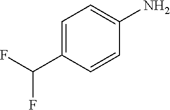 | 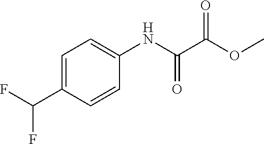 [SMILES: COC(=O)C(=O)Nc1ccc(C(F)F)cc1] |
| | Int 1 | |
| Int 10/13 | 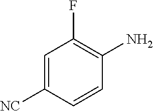 | 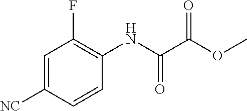 [SMILES: COC(=O)C(=O)Nc1ccc(C#N)cc1F] |
| Int 10/14 | 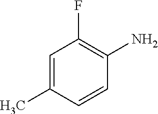 | 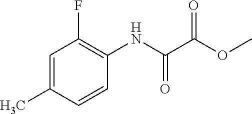 |
| Int 10/15 | 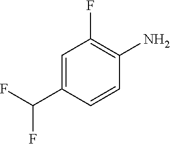 [SMILES: Nc1ccc(C(F)F)cc1F] | 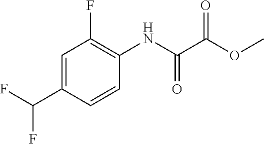 [SMILES: COC(=O)C(=O)Nc1ccc(C(F)F)cc1F] |
| Int 10/16 | 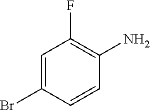 | 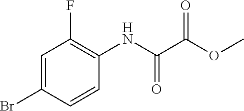 |
| Int 10/17 | 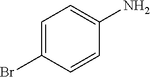 | 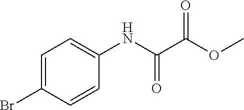 |
| Int 10/18 | 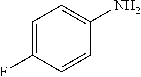 | 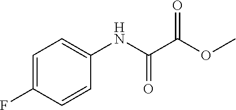 |
| Int 10/19 | 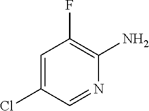 | 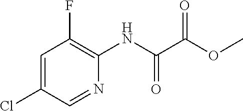 |
| Int 10/20 | 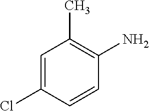 | 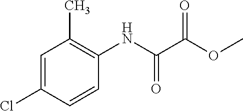 |
| Int 10/21 | 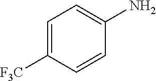 [SMILES: Nc1ccc(C(F)(F)F)cc1] | 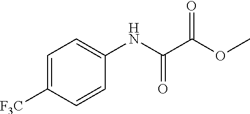 [SMILES: COC(=O)C(=O)Nc1ccc(C(F)(F)F)cc1] |
| Int 10/22 | 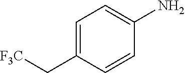 [SMILES: Nc1ccc(CC(F)(F)F)cc1] | 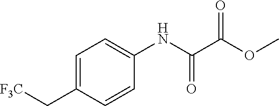 [SMILES: COC(=O)C(=O)Nc1ccc(CC(F)(F)F)cc1] |
| Int 10/23 | 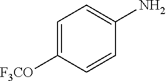 [SMILES: Nc1ccc(OC(F)(F)F)cc1] | 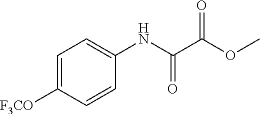 [SMILES: COC(=O)C(=O)Nc1ccc(OC(F)(F)F)cc1] |
| Int 10/24 | 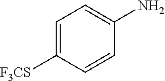 | 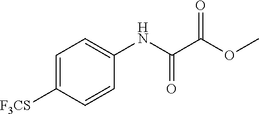 |
| Int 10/25 | 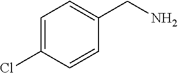 | 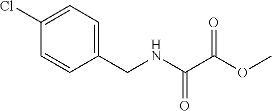 |
| Int 10/26 | 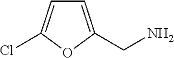 | 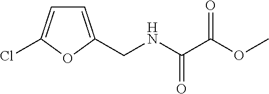 |
| Int 10/27 | 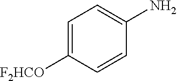 [SMILES: Nc1ccc(OC(F)F)cc1] | 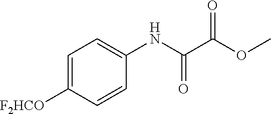 [SMILES: COC(=O)C(=O)Nc1ccc(OC(F)F)cc1] |
| Int 10/28 | 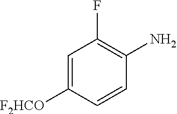 [SMILES: Nc1ccc(OC(F)F)cc1F] | 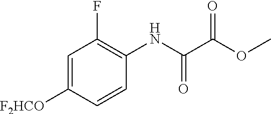 [SMILES: COC(=O)C(=O)Nc1ccc(OC(F)F)cc1F] |
| Int 10/29 | 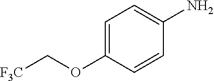 [SMILES: Nc1ccc(OCC(F)(F)F)cc1] | 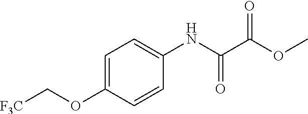 [SMILES: COC(=O)C(=O)Nc1ccc(OCC(F)(F)F)cc1] |

### Intermediate 11: Lithium 2-((5-chloropyridin-2-yl)amino)-2-oxoacetate (Int 11)

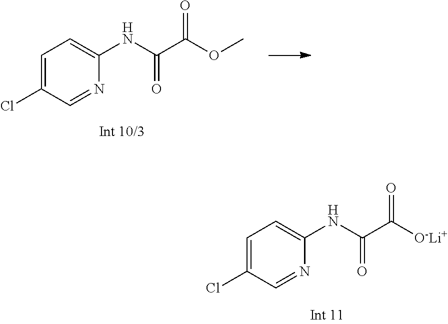

A mixture of methyl 2-((5-chloropyridin-2-yl)amino)-2-oxoacetate (Int 10/3) (1.0 g, 4.7 mmol, 1.0 eq.) and LiOH.H₂O (216 mg, 5.1 mmol, 1.1 eq.) in THF/H₂O (12 mL:3 mL) was stirred at rt for 3 h. The mixture was concentrated in vacuo to afford a white solid. Hexane (20 mL) was added to the white solid and stirred at rt for 2 h. The mixture was filtered. The solid residue was collected and dried. Acetonitrile (10 mL) was added to the residue and stirred at rt for 1 h. The residue was collected by filtration, washed with EtOAc (5 mL) and then dried to afford the title compound as a white solid.

### Intermediates 11/1 to 11/2

The following Intermediates were prepared similar as described for Intermediate 11 using the appropriate building blocks.

| Int. # | building block | Structure |
|---|---|---|
| Int 11/1 | Int 10/25 | 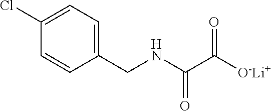 |
| Int 11/2 | Int 10/26 | 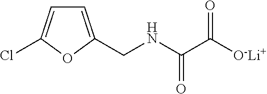 |

### Intermediate 12: Lithium 2-((5-chloropyridin-2-yl)(methyl)amino)-2-oxoacetate (Int 12)

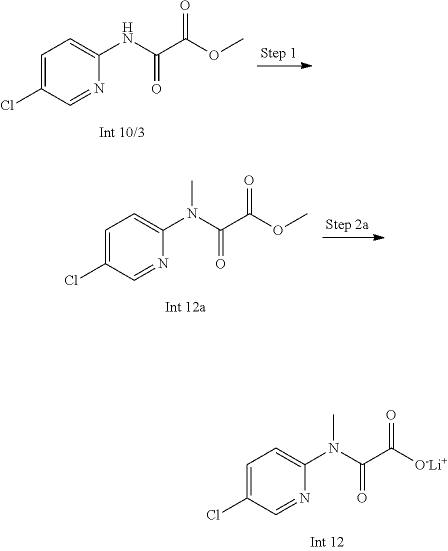

### Step 1: Methyl 2-((5-chloropyridin-2-yl)(methyl)amino)-2-oxoacetate (Int 12a)

To a solution of methyl 2-((5-chloropyridin-2-yl)amino)-2-oxoacetate (Int 11b) (214 mg, 1.0 mmol) in DMF (4 ml) was added NaH (48 mg, 1.2 mmol, 1.2 eq.) at 0′C. The mixture was stirred at 0° C. for 0.5 h. Methyliodide (156 mg, 1.1 mmol) was added and the mixture was stirred at rt for 2 h. Water was added and the mixture was extracted with EtOAc. The organic layer was washed with brine, dried over anhydrous Na₂SO₄, filtered and concentrated to dryness. The residue was purified by column chromatography on silica gel (PE/EtOAc=4:1) to give the title compound as a yellow oil.

### Step 2: Lithium 2-((5-chloropyridin-2-yl)(methyl)amino)-2-oxoacetate (Int 12)

To a solution of methyl 2-((5-chloropyridin-2-yl)(methyl)amino)-2-oxoacetate (Int 12a) (180 mg, 0.79 mmol) in THE (2 mL) and H₂O (0.5 mL) was added LiOH.H₂O (33 mg, 0.79 mmol). The mixture was stirred at rt for 3 h. The mixture was concentrated to dryness. Hexane (10 mL) was added and the suspension was stirred at rt for 1 h. The mixture was filtered and the residue was collected and dried. Acetonitrile (10 mL) was added to the residue and the suspension was stirred at rt for 1 h. The residue was collected by filtration, extracted with EtOAc (5 mL) and dried to give the title compound as a white solid.

### Intermediate 13: 2-((5-Chloropyridin-2-yl)amino)-2-oxoacetic Acid (Int 13)

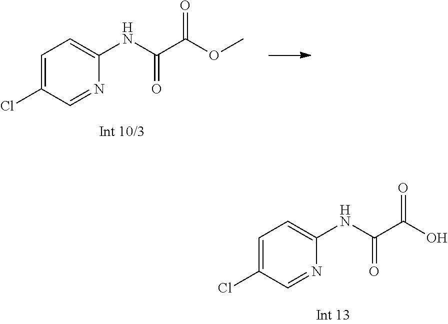

To a solution of methyl 2-((5-chloropyridin-2-yl)amino)-2-oxoacetate (Int 10/3) (0.50 g, 2.30 mmol) in THE (8 mL), MeOH (5 mL) and H₂O (5 mL) was added LiOH.H₂O (289 mg, 6.90 mmol). The mixture was stirred at rt for 3 h. It was concentrated and the residue was diluted with H₂O (10 mL). It was acidified with 1 N HCl solution to pH=6-7 and extracted with EtOAc (3×30 mL). The combined organic layers were washed with brine (30 mL), dried over Na₂SO₄, filtered and concentrated to give the title compound as a yellow solid.

### Intermediate 13/1: 2-((4-Chlorophenyl)amino)-2-oxoacetic Acid (Int 13/1)

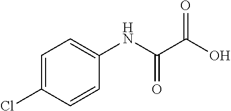 [SMILES: O=C(O)C(=O)Nc1ccc(Cl)cc1]

The title compound was prepared similar as described for intermediate 13 using methyl 2-((4-chlorophenyl)amino)-2-oxoacetate (Int 10/1) in place of methyl 2-((5-chloropyridin-2-yl)amino)-2-oxoacetate (Int 10/3).

### Intermediate 20: 6-Fluoro-4-(piperidin-4-yl)quinoline (Int 20)

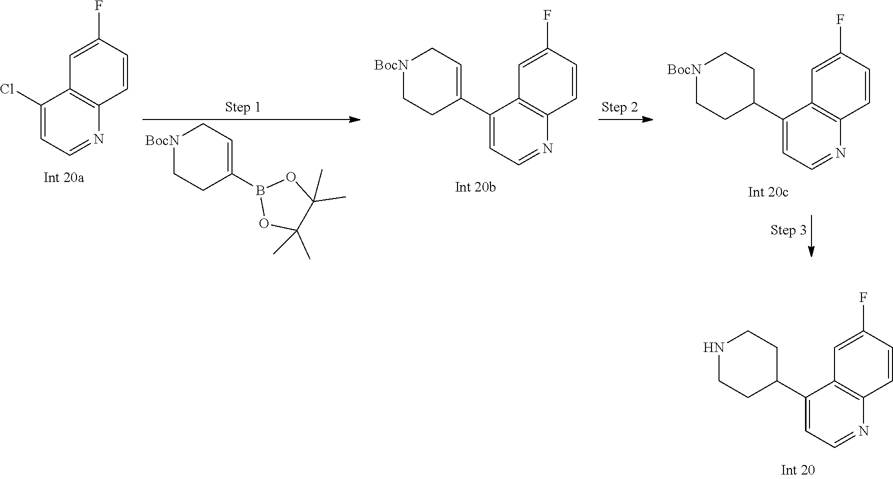 [SMILES: Fc1ccc2nccc(Cl)c2c1 ; CC(C)(C)OC(=O)N1CC=C(B2OC(C)(C)C(C)(C)O2)CC1 ; CC(C)(C)OC(=O)N1CC=C(c2ccnc3ccc(F)cc23)CC1 ; CC(C)(C)OC(=O)N1CCC(c2ccnc3ccc(F)cc23)CC1 ; Fc1ccc2nccc(C3CCNCC3)c2c1]

### Step 1: tert-Butyl 4-(6-fluoroquinolin-4-yl)-5,6-dihydropyridine-1(2H)-carboxylate (Int 20b)

To a solution of 4-chloro-6-fluoroquinoline (Int 20a) (2.00 g, 11.0 mmol) in dioxane (40 mL) and H₂O (5 mL) was added tert-butyl 4-(4,4,5,5-tetramethyl-1,3,2-dioxaborolan-2-yl)-5,6-dihydropyridine-1(2H)-carboxylate (4.43 g, 14.0 mmol), Pd(dppf)₂Cl₂ (450 mg, 0.55 mmol) and Cs₂CO₃ (7.18 g, 22.00 mmol). The mixture was stirred at 90° C. for 16 h. The mixture was concentrated to dryness and the residue was purified by silica gel column chromatography (PE:EtOAc=4:1) to give the title compound as a pale yellow solid.

### Step 2: tert-Butyl 4-(6-fluoroquinolin-4-yl)piperidine-1-carboxylate (Int 20c)

To a solution of tert-butyl 4-(6-fluoroquinolin-4-yl)-5,6-dihydropyridine-1(2H)-carboxylate (Int 20b) (3.40 g, 10.30 mmol) in EtOH (35 mL) was added Pd/C (0.68 g). The mixture was stirred under H₂ atmosphere at rt for 16 h. The mixture was filtered and the residue was washed with EtOH (15 mL). The combined organic layers were concentrated to dryness to give the title compound as a brown oil.

### Step 3: 6-Fluoro-4-(piperidin-4-yl)quinoline (Int 20)

To a solution of tert-butyl 4-(6-fluoroquinolin-4-yl)piperidine-1-carboxylate (Int 20c) (3.20 g, 9.69 mmol) in DCM (20 mL) was added HCl/dioxane (4M, 15 mL). The mixture was stirred at rt for 3 h. The mixture was concentrated and the residue was diluted with H₂O (15 mL). The pH was adjusted with saturated aqueous NaHCO₃ to pH=8-9. The mixture was extracted with DCM/i-PrOH (3/1, 3×30 mL). The combined organic layers were washed with brine (30 mL), dried over anhydrous Na₂SO₄, filtered and concentrated to dryness to give the title compound as a yellow solid.

### Intermediates 20/1 to 20/17

The following Intermediates were prepared similar as described for Intermediate 20 using the appropriate building blocks.

| Int. # | building blocks | Structure |
|---|---|---|
| Int 20/1 | 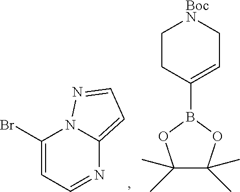 | 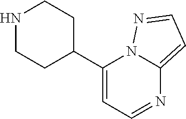 [SMILES: c1cc(C2CCNCC2)n2nccc2n1] |
| Int 20/2 | 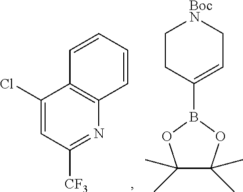 | 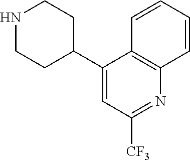 [SMILES: FC(F)(F)c1cc(C2CCNCC2)c2ccccc2n1] |
| Int 20/3 | 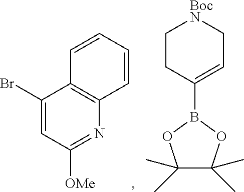 | 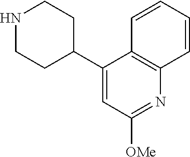 [SMILES: COc1cc(C2CCNCC2)c2ccccc2n1] |
| Int 20/4 | 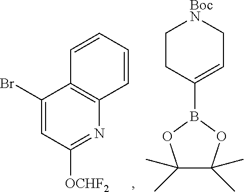 | 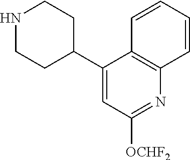 [SMILES: FC(F)Oc1cc(C2CCNCC2)c2ccccc2n1] |
| | Int 2 | |
| Int 20/5 | 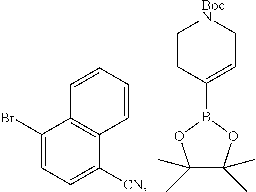 | 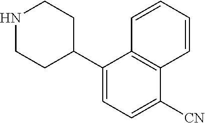 [SMILES: N#Cc1ccc(C2CCNCC2)c2ccccc12] |
| Int 20/6 | 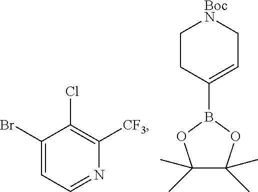 | 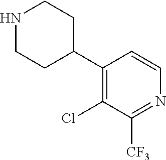 [SMILES: FC(F)(F)c1nccc(C2CCNCC2)c1Cl] |
| Int 20/7 | 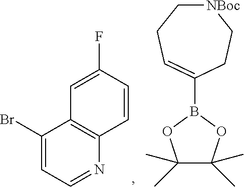 | 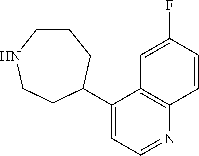 [SMILES: Fc1ccc2nccc(C3CCCNCC3)c2c1] |
| Int 20/8 | 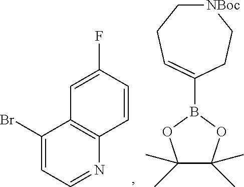 | 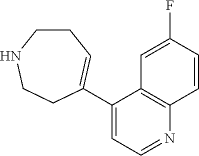 [SMILES: Fc1ccc2nccc(C3=CCCNCC3)c2c1] |
| | no hydrogenation | |
| | (Step 2) | |
| Int 20/9 | 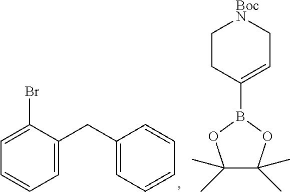 | 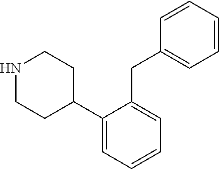 [SMILES: c1ccc(Cc2ccccc2C2CCNCC2)cc1] |
| Int 20/10 | 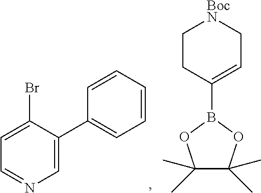 | 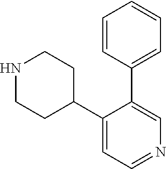 [SMILES: c1ccc(-c2cnccc2C2CCNCC2)cc1] |
| Int 20/11 | 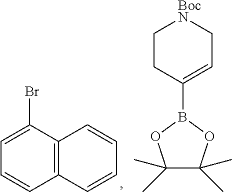 | 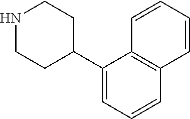 [SMILES: c1ccc2c(C3CCNCC3)cccc2c1] |
| Int 20/12 | 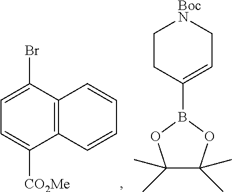 | 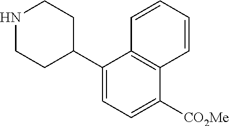 [SMILES: COC(=O)c1ccc(C2CCNCC2)c2ccccc12] |
| Int 20/13 | 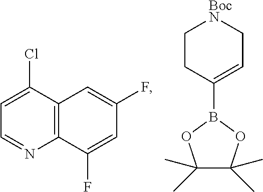 | 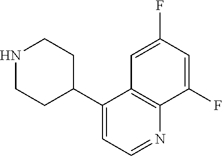 [SMILES: Fc1cc(F)c2nccc(C3CCNCC3)c2c1] |
| Int 20/14 | 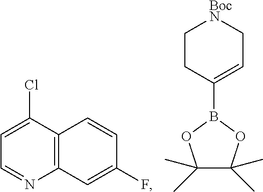 | 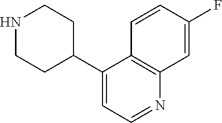 [SMILES: Fc1ccc2c(C3CCNCC3)ccnc2c1] |
| Int 20/15 | 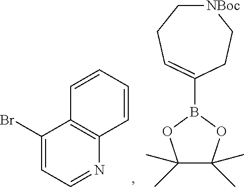 | 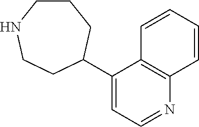 [SMILES: c1ccc2c(C3CCCNCC3)ccnc2c1] |
| Int 20/16 | 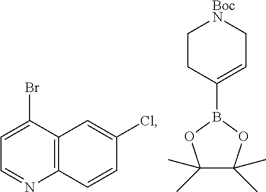 | 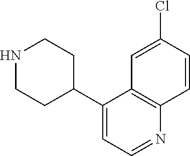 [SMILES: Clc1ccc2nccc(C3CCNCC3)c2c1] |
| Int 20/17 | 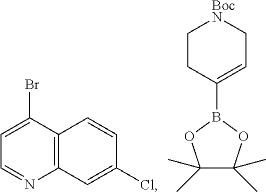 | 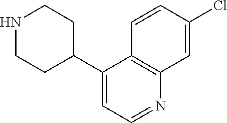 [SMILES: Clc1ccc2c(C3CCNCC3)ccnc2c1] |

### Intermediate 21: 4-(Quinolin-4-yl)piperidin-4-ol Hydrochloride (Int 21)

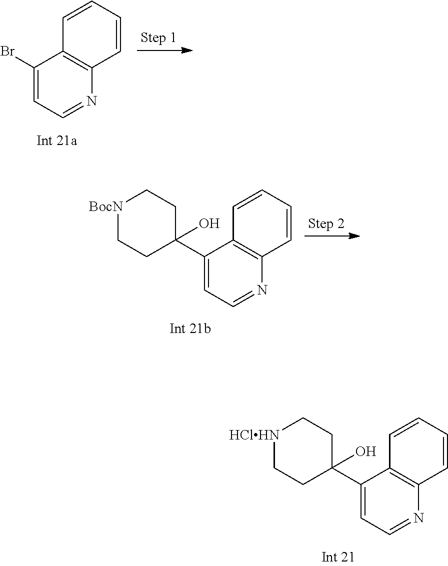

### Step 1: tert-Butyl 4-hydroxy-4-(quinolin-4-yl)piperidine-1-carboxylate (Int 21b)

To a solution of 4-bromoquinoline (500 mg, 2.42 mmol) in THE (10 mL) was added n-BuLi (1.5 mL, 2.5N in THF) at −78° C. and the mixture was stirred at this temperature for 1 h. Then tert-butyl 4-oxopiperidine-1-carboxylate (578 mg, 2.90 mmol) dissolved in THE (10 mL) was added and the mixture was stirred at rt for 3 h. Aqueous NH₄Cl (30 mL) was added and the mixture was extracted with EtOAc (2×30 mL). The combined organic layers were washed with brine (30 mL), dried over anhydrous Na₂SO₄, filtered and concentrated to dryness. The residue was purified by column chromatography (EtOAc/PE=1:4) to give the title compound as a yellow solid.

### Step 2: 4-(Quinolin-4-yl)piperidin-4-ol Hydrochloride (Int 21)

To a solution of tert-butyl 4-hydroxy-4-(quinolin-4-yl)piperidine-1-carboxylate (Int 21b) (174 mg, 0.53 mmol) in 1,4-dioxane (2 mL) was added HCl/1,4-dioxane (2 mL, 4M) at rt. The mixture was stirred for 1 h and concentrated to dryness to give the title compound as a white solid.

### Intermediate 22: 4-(4-Fluoropiperidin-4-yl)quinoline Hydrochloride (Int 22)

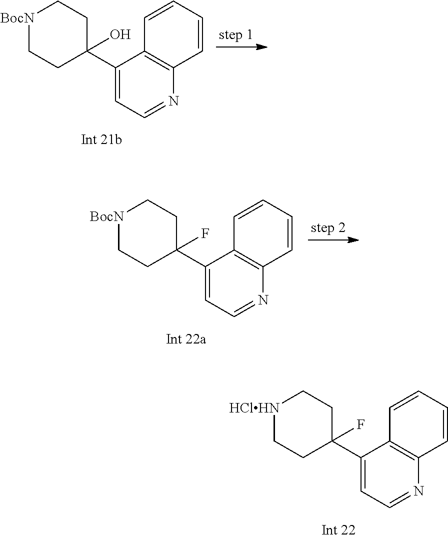

### Step 1: tert-Butyl 4-fluoro-4-(quinolin-4-yl)piperidine-1-carboxylate (Int 22a)

To a solution of tert-butyl 4-hydroxy-4-(quinolin-4-yl)piperidine-1-carboxylate (Int 21b) (781 mg, 2.38 mmol) in DCM (20 mL) was added DAST (575 mg, 3.57 mmol) at 0° C. The mixture was stirred at rt overnight. Then the mixture was diluted with water (30 mL) and extracted with DCM (3×30 mL). The combined organic layers were washed with brine (30 mL), dried over anhydrous Na₂SO₄, filtered and concentrated to dryness. The residue was purified by prep-TLC (PE/EtOAc, 4:1, v/v) to give the title compound as a brown solid.

### Step 2: 4-(4-Fluoropiperidin-4-yl)quinoline Hydrochloride (Int 22)

A mixture of tert-butyl 4-fluoro-4-(quinolin-4-yl)piperidine-1-carboxylate (Int 22a) (163 mg, 0.49 mmol) in 1,4-dioxane (3 mL) and HCl/1,4-dioxane (2 mL) was stirred at rt for 3 h. It was then concentrated to give the title compound as a yellow solid.

### Intermediate 23: 4-(8-Azabicyclo[3.2.1]octan-3-yl)quinoline Hydrochloride (Int 23)

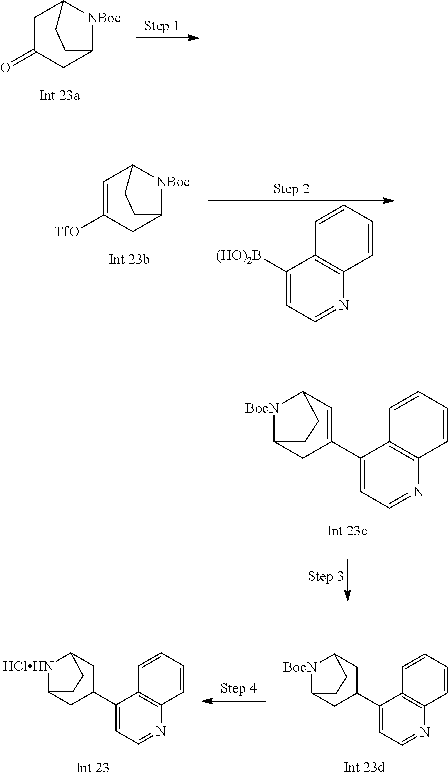

### Step 1: tert-Butyl 3-(((trifluoromethyl)sulfonyl)oxy)-8-azabicyclo[3.2.1]oct-3-ene-8-carboxylate (Int 23b)

To a solution of tert-butyl 3-oxo-8-azabicyclo[3.2.1]octane-8-carboxylate (Int 23a) (1.00 g, 4.33 mmol) in THE (10 mL) was added LDA (2 N, 3.3 mL) at −78° C. and the mixture was stirred for 10 min at the same temperature. A solution of N-phenylbis(trifluoromethanesulfonimide) (1.75 g, 4.88 mmol) in THE (8 mL) was added. The mixture was stirred at −78° C. for 30 min. The cooling bath was removed and the mixture was stirred for 1.5 h. Saturated aqueous NH₄Cl (30 mL) was added and stirring was continued for 5 min. The mixture was extracted with EtOAc (3×30 mL). The combined organic layers were washed with brine (30 mL), dried over anhydrous Na₂SO₄, filtered and concentrated to dryness. The residue was purified by column chromatography on silica gel (EtOAc/PE=1:2) to give the title compound as a yellow oil.

### Step 2: tert-Butyl 3-(quinolin-4-yl)-8-azabicyclo[3.2.1]oct-3-ene-8-carboxylate (Int 23c)

To a mixture of tert-butyl 3-(((trifluoromethyl)sulfonyl)oxy)-8-azabicyclo[3.2.1]oct-3-ene-8-carboxylate (Int 23b) (1.10 g, 3.08 mmol), quinolin-4-ylboronic acid (533 mg, 3.08 mmol) and Cs₂CO₃ (2.00 g, 6.16 mmol) in 1,4-dioxane (15 mL) was added dppfPdCl₂ (120 mg) and the mixture was stirred at 100° C. overnight. Water (30 mL) was added and the mixture was extracted with EtOAc (1×30 mL). The organic layer was washed with brine (1×30 mL), dried over Na₂SO₄, filtered and concentrated to dryness. The residue was purified by column chromatography (EtOAc/PE=1:10) to give the title compound as a white solid.

### Step 3: tert-Butyl 3-(quinolin-4-yl)-8-azabicyclo[3.2.1]octane-8-carboxylate (Int 23d)

To a solution of tert-butyl 3-(quinolin-4-yl)-8-azabicyclo[3.2.1]oct-3-ene-8-carboxylate (Int 23c) (419 mg, 1.25 mmol) in MeOH (5 mL) was added Pd/C (150 mg) and the mixture was stirred at rt under hydrogen atmosphere for 4 h. The mixture was filtered and the filtrate was concentrated to dryness. The residue was purified by column chromatography (EA:PE=1:8) to give the title compound as a white solid.

### Step 4: 4-(8-Azabicyclo[3.2.1]octan-3-yl)quinoline Hydrochloride (Int 23)

To a solution of tert-butyl 3-(quinolin-4-yl)-8-azabicyclo[3.2.1]octane-8-carboxylate (Int 23d) (316 mg, 0.93 mmol) in 1,4-dioxane (2 mL) was added HCl/1,4-dioxane (2 mL, 4M) at rt. The mixture was stirred at rt for 1 h. The mixture was concentrated to dryness to give the title compound as a white solid.

### Intermediate 23/1: 4-(2,2-Dimethylpiperidin-4-yl)quinoline Hydrochloride (Int 23/1)

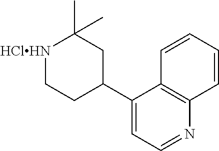

The title compound was prepared similar as described for intermediate 23 using in step 1 tert-butyl 2,2-dimethyl-4-oxopiperidine-1-carboxylate in place of tert-butyl 3-oxo-8-azabicyclo[3.2.1]octane-8-carboxylate (Int 23a).

### Intermediate 23/2: 4-(3,3-Dimethylpiperidin-4-yl)-6-fluoroquinoline Trifluoroacetic Acid Salt (Int 23/2)

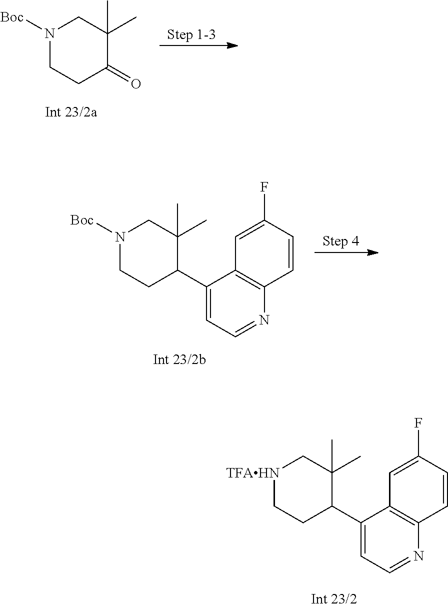

### Steps 1-3: tert-Butyl 4-(6-fluoroquinoline-4-yl)-3,3-dimethylpiperidine-1-carboxylate (Int 23/2b)

The title compound was prepared similar as described for intermediate 23 using in step 1 tert-butyl 3,3-dimethyl-4-oxopiperidine-1-carboxylate (Int 23/2a) in place of tert-butyl 3-oxo-8-azabicyclo[3.2.1]octane-8-carboxylate (Int 23a).

### Step 4: 4-(3,3-Dimethylpiperidin-4-yl)-6-fluoroquinoline Trifluoroacetic Acid Salt (Int 23/2)

To a solution of tert-butyl 4-(6-fluoroquinolin-4-yl)-3,3-dimethylpiperidine-1-carboxylate (Int 23/2b) (79 mg, 0.22 mmol) in DCM (5 mL) was added TFA (1 mL) and the mixture was stirred at rt for 2 h. The mixture was concentrated to dryness to give the title compound as a yellow solid.

### Intermediate 24: 2-(4-(Piperidin-4-yl)quinoline-6-yl)propan-2-ol Trifluoroacetic Acid Salt (Int 24)

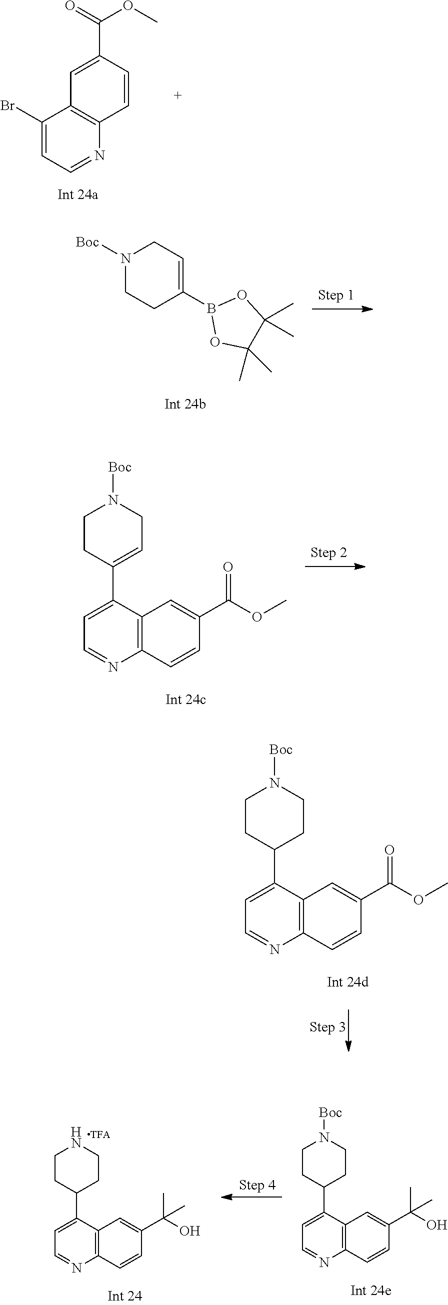 [SMILES: COC(=O)c1ccc2nccc(Br)c2c1 ; CC(C)(C)OC(=O)N1CC=C(B2OC(C)(C)C(C)(C)O2)CC1 ; COC(=O)c1ccc2nccc(C3=CCN(C(=O)OC(C)(C)C)CC3)c2c1 ; COC(=O)c1ccc2nccc(C3CCN(C(=O)OC(C)(C)C)CC3)c2c1 ; CC(C)(O)c1ccc2nccc(C3CCNCC3)c2c1 ; CC(C)(C)OC(=O)N1CCC(c2ccnc3ccc(C(C)(C)O)cc23)CC1]

### Step 1: Methyl 4-(1-(tert-butoxycarbonyl)-1,2,3,6-tetrahydropyridin-4-yl)quinoline-6-carboxylate (Int 24c)

To a solution of methyl 4-bromoquinoline-6-carboxylate (Int 24a) (2.00 g, 7.50 mmol) in dioxane (20 mL) and H₂O (2 mL) was added tert-butyl 4-(4,4,5,5-tetramethyl-1,3,2-dioxaborolan-2-yl)-3,6-dihydropyridine-1(2H)-carboxylate (Int 24b) (2.78 g, 9.00 mmol), Pd(dppf)₂Cl₂ (200 mg, 0.26 mmol) and K₂CO₃ (2.07 g, 15.00 mmol). The mixture was stirred at 90° C. for 16 h. The mixture was concentrated to dryness and the residue was purified by silica gel column chromatography (PE/EtOAc=4:1) to give the title compound as a pale yellow solid.

### Step 2: Methyl 4-(1-(tert-butoxycarbonyl)piperidin-4-yl)quinoline-6-carboxylate (Int 24d)

To a solution of methyl 4-(1-(tert-butoxycarbonyl)-1,2,3,6-tetrahydropyridin-4-yl)quinoline-6-carboxylate (Int 24c) (2.60 g, 7.06 mmol) in EtOH (25 mL) was added Pd/C (0.30 g). The mixture was stirred at rt for 16 h under H₂. The mixture was filtered and washed with EtOH (10 mL). The filtrate was concentrated to dryness to give the title compound as a brown oil.

### Step 3: tert-Butyl 4-(6-(2-hydroxypropan-2-yl)quinoline-4-yl)piperidine-1-carboxylate (Int 24e)

To a solution of methyl 4-(1-(tert-butoxycarbonyl)piperidin-4-yl)quinoline-6-carboxylate (Int 24d) (330 mg, 0.89 mmol) in THE (4 mL) was added 2M MeMgBr/THF (1.2 mL, 3.56 mmol) dropwise at 0° C. The mixture was stirred at rt for 14 h. The mixture was diluted with H₂O (30 mL), extracted with EtOAc (3×20 mL) and washed with brine (20 mL). The organic layer was dried over Na₂SO₄, filtered and concentrated to dryness. The residue was purified by preparative TLC (PE/EtOAc=3:1) to give the title compound as a white solid.

### Step 4: 2-(4-(Piperidin-4-yl)quinoline-6-yl)propan-2-ol Trifluoroacetic Acid Salt (Int 24)

To a solution of tert-butyl 4-(6-(2-hydroxypropan-2-yl)quinoline-4-yl)piperidine-1-carboxylate (Int 24e) (180 mg, 0.48 mmol) in DCM (5 mL) was added TFA (5 mL). The mixture was stirred at rt for 3 h. The mixture was concentrated to dryness to give the title compound as a yellow solid.

### Int 25: 4-(Piperidin-4-yl)quinoline-6-carboxamide Hydrochloride (Int 25)

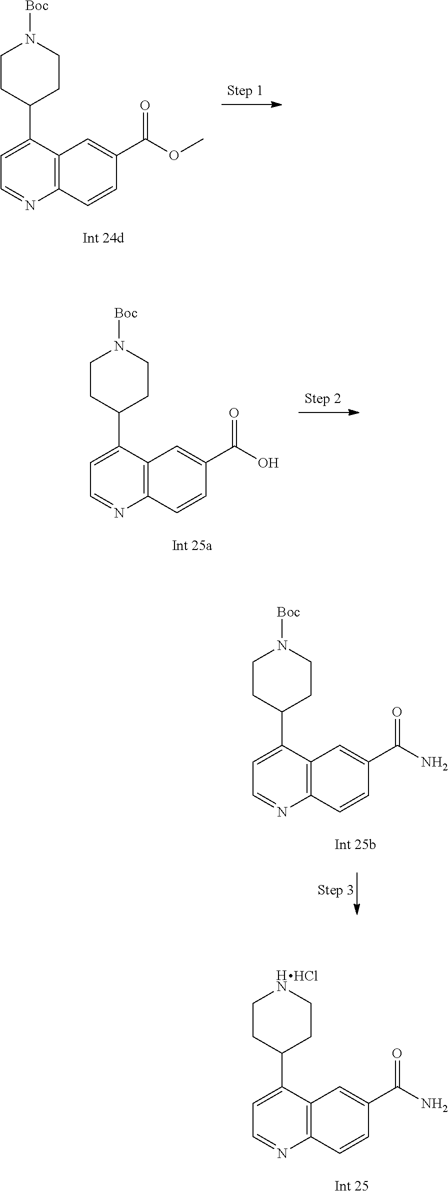 [SMILES: COC(=O)c1ccc2nccc(C3CCN(C(=O)OC(C)(C)C)CC3)c2c1 ; CC(C)(C)OC(=O)N1CCC(c2ccnc3ccc(C(=O)O)cc23)CC1 ; CC(C)(C)OC(=O)N1CCC(c2ccnc3ccc(C(N)=O)cc23)CC1 ; Cl ; NC(=O)c1ccc2nccc(C3CCNCC3)c2c1]

### Step 1: 4-(1-(tert-Butoxycarbonyl)piperidin-4-yl)quinoline-6-carboxylic Acid (Int 25a)

To a solution of methyl 4-(1-(tert-butoxycarbonyl)piperidin-4-yl)quinoline-6-carboxylate (Int 24d) (444 mg, 1.20 mmol) in EtOH (5 mL)/THF (5 mL) was added aqueous LiOH (2M, 5 mL) and the mixture was stirred at rt for 2 h. The mixture was diluted with H₂O (30 mL) and acidified to pH=4-5 using 2M HCl. The mixture was extracted with EtOAc (3×20 mL). The combined organic layers were washed with brine (30 mL), dried over Na₂SO₄, filtered and concentrated to dryness to give the title compound as a white solid.

### Step 2: tert-Butyl 4-(6-carbamoylquinolin-4-yl)piperidine-1-carboxylate (Int 25b)

To a solution of 4-(1-(tert-butoxycarbonyl)piperidin-4-yl)quinoline-6-carboxylic acid (Int 25a) (360 mg, 1.01 mmol), HATU (768 mg, 2.02 mmol) and TEA (306 mg, 3.03 mmol) in DMF (6 mL) was added NH₄Cl (66 mg) and the mixture was stirred at rt overnight. The mixture was diluted with H₂O (20 mL) and extracted with EtOAc (3×20 mL). The combined organic layers were washed with brine (20 mL), dried over Na₂SO₄, filtered and concentrated to dryness. The residue was purified by preparative TLC to give the title compound.

### Step 3: 4-(Piperidin-4-yl)quinoline-6-carboxamide Hydrochloride (Int 25)

To a solution of tert-butyl 4-(6-carbamoylquinolin-4-yl)piperidine-1-carboxylate (Int 25b) (260 mg, 0.73 mmol) in MeOH (50 mL) was added HCl/dioxane (4M, 5 mL). The mixture was stirred at rt for 3 h. The mixture was concentrated to dryness to give the title compound as a yellow solid.

### Intermediate 26: 4-(Piperidin-4-yl)quinoline-6-carbonitrile Hydrochloride (Int 26)

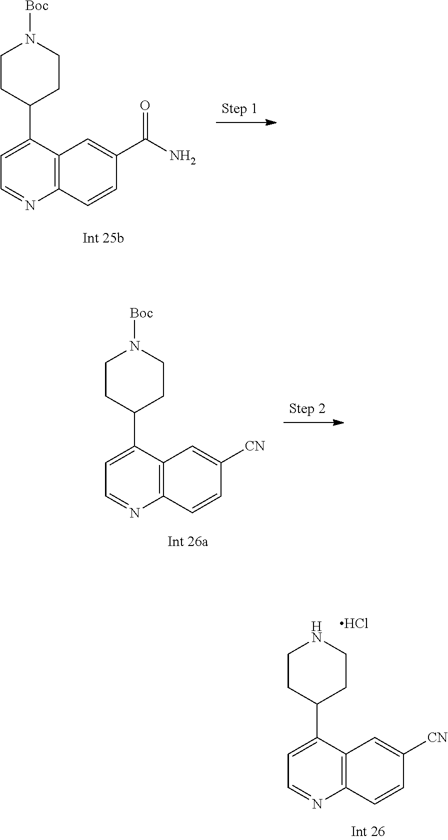 [SMILES: CC(C)(C)OC(=O)N1CCC(c2ccnc3ccc(C(N)=O)cc23)CC1 ; CC(C)(C)OC(=O)N1CCC(c2ccnc3ccc(C#N)cc23)CC1 ; Cl ; N#Cc1ccc2nccc(C3CCNCC3)c2c1]

### Step 1: tert-Butyl 4-(6-cyanoquinolin-4-yl)piperidine-1-carboxylate (Int 26a)

To a solution of tert-butyl 4-(6-carbamoylquinolin-4-yl)piperidine-1-carboxylate (Int 25b) (355 mg, 1.00 mmol) and triethylamine (3.03 mg, 3.00 mmol) in DCM (5 mL) was added TFAA (1.2 mL, 8.00 mmol) dropwise at 0° C. The mixture was stirred at rt for 2 h. The mixture was washed with 1M HCl solution (5 mL), saturated NaHCO₃ (5 mL) and brine (5 mL). The combined organic layers were dried over Na₂SO₄, filtered and concentrated to dryness. The residue was purified by preparative TLC (PE/EtOAc=2:1) to give the tilte compound as a yellow solid.

### Step 2: 4-(Piperidin-4-yl)quinoline-6-carbonitrile Hydrochloride (Int 26)

To a solution of tert-butyl 4-(6-cyanoquinolin-4-yl)piperidine-1-carboxylate (Int 26a) (270 mg, 0.80 mmol) in MeOH (5 mL) was added HCl/dioxane (4M, 5 mL). The mixture was stirred at rt for 3 h. The mixture was concentrated to dryness to give the title compound as a yellow solid.

### Intermediate 27: trans-4-(6-Fluoroquinolin-4-yl)piperidin-3-ol Trifluoroacetic Acid Salt (Int 27)

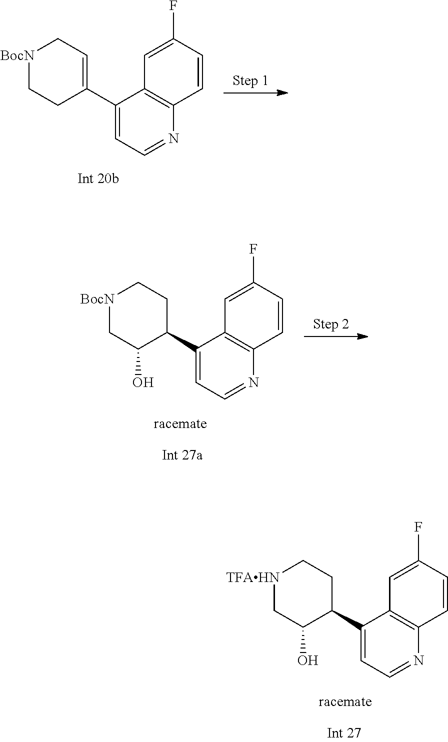

### Step 1: trans-tert-Butyl 4-(6-fluoroquinolin-4-yl)-3-hydroxypiperidine-1-carboxylate (Int 27a)

To a solution of tert-butyl 4-(6-fluoroquinolin-4-yl)-3,6-dihydropyridine-1(2H)-carboxylate (Int 20b) (2.66 g, 8.11 mmol) in THE (30 mL) was added BH₃ (1N in THF, 8 mL) at 0° C. under N₂ and the mixture was stirred at 40° C. overnight. The mixture was cooled to rt, NaOH (2N, 1.6 mL) was added. The mixture was stirred for 12 min, H₂O₂ (30%, 1.3 mL) was added at 0° C., and the mixture was stirred at rt for 3 h. The mixture was diluted with H₂O (50 mL) and extracted with EA (3×50 mL). The combined organic layers were washed with brine (50 mL), dried over Na₂SO₄, filtered and concentrated to dryness (EtOAc/PE=1:4) to give the title compound as a yellow solid.

### Step 2: trans-4-(6-Fluoroquinolin-4-yl)piperidin-3-ol Trifluoroacetic Acid Salt (Int 27)

To a solution of tert-butyl 4-(6-fluoroquinolin-4-yl)-3-hydroxypiperidine-1-carboxylate (Int 27a) (168 mg, 0.49 mmol) in DCM (5 mL) TFA (1 mL) was added and the mixture was stirred at rt for 2 h. The mixture was concentrated to dryness to give the title compound as a yellow solid.

### Intermediate 28: 4-(3,3-Difluoropiperidin-4-yl)-6-fluoroquinoline Trifluoroacetic Acid Salt (Int 28)

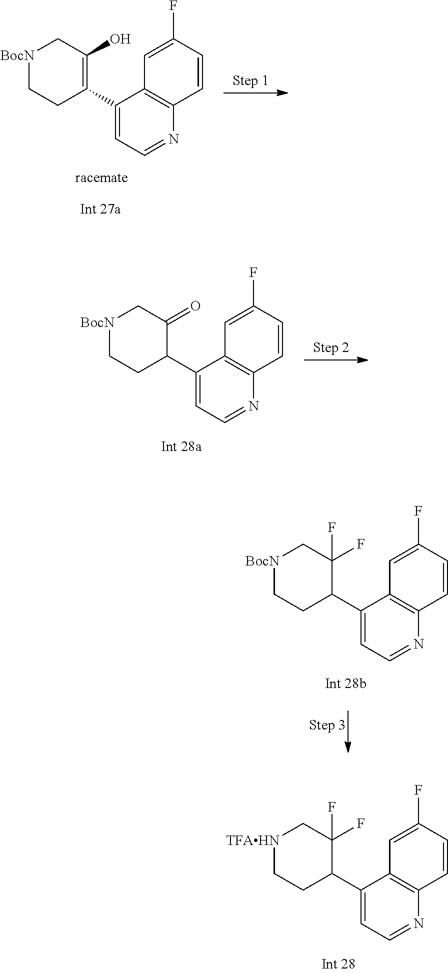

### Step 1: tert-Butyl 4-(6-fluoroquinolin-4-yl)-3-oxopiperidine-1-carboxylate (Int 28a)

To a solution of trans-tert-butyl 4-(6-fluoroquinolin-4-yl)-3-hydroxypiperidine-1-carboxylate (Int 27a) (332 mg, 0.96 mmol) in DCM (5 mL) was added Dess-Martin periodinane (1.03 g, 2.40 mmol) and the mixture was stirred at rt overnight. Aqueous NaHCO₃ (50 mL) was added and the mixture was extracted with DCM (3×50 mL). The combined organic layers were washed with brine (50 mL), dried over Na₂SO₄, filtered and concentrated to dryness to give the title compound as a white solid.

### Step 2: tert-Butyl 3,3-difluoro-4-(6-fluoroquinolin-4-yl)piperidine-1-carboxylate (Int 28b)

To a solution of tert-butyl 4-(6-fluoroquinolin-4-yl)-3-oxopiperidine-1-carboxylate (Int 28a) (299 mg, 0.87 mmol) in DCM (5 mL) was added DAST (210 mg, 1.31 mmol) at 0° C. and the mixture was stirred at rt for 1 h. Aqueous NaHCO₃ (50 mL) was added and the mixture was extracted with DCM (3×50 mL). The combined organic layers were washed with brine (50 mL), dried over Na₂SO₄, filtered and concentrated to dryness to give the title compound as a brown solid.

### Step 3: 4-(3,3-Difluoropiperidin-4-yl)-6-fluoroquinoline Trifluoroacetic Acid Salt (Int 28)

To a solution of tert-butyl 3,3-difluoro-4-(6-fluoroquinolin-4-yl)piperidine-1-carboxylate (Int 28b) (104 mg, 0.28 mmol) in DCM (5 mL) was added TFA (1 mL) and the mixture was stirred at rt for 2 h. The mixture was concentrated to dryness to give the title compound as a brown solid.

### Intermediate 29: cis-4-(6-Fluoroquinolin-4-yl)piperidin-3-ol (Int 29)

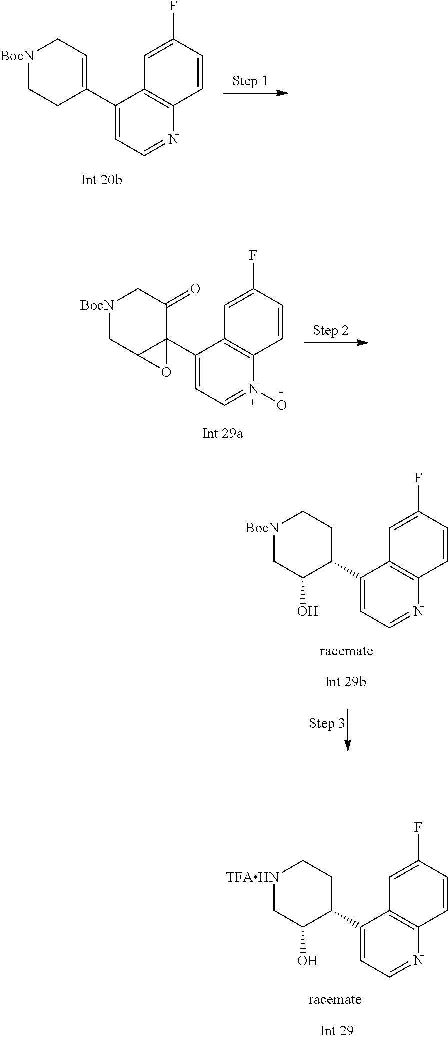

### Step 1: 4-(3-(tert-Butoxycarbonyl)-7-oxa-3-azabicyclo[4.1.0]heptan-6-yl)-6-fluoroquinoline 1-oxide (Int 29a)

To a solution of tert-butyl 4-(6-fluoroquinolin-4-yl)-5,6-dihydropyridine-1(2H)-carboxylate (Int 20b) (1.00 g, 3.05 mmol) in DCM (20 mL) was added mCPBA (1.85 g, 9.15 mmol, 85%) and the mixture was stirred at rt overnight. Aqueous Na₂SO₃ (50 mL) was added and the mixture was extracted with DCM (3×50 mL). The combined organic layers were washed with aqueous NaHCO₃ (50 mL) and brine (50 mL), dried over Na₂SO₄, filtered and concentrated to dryness to give the title compound as a yellow solid, which was used in the next step without further purification.

### Step 2: cis-tert-Butyl 4-(6-fluoroquinolin-4-yl)-3-hydroxypiperidine-1-carboxylate (Int 29b)

To a solution of 4-(3-(tert-Butoxycarbonyl)-7-oxa-3-azabicyclo[4.1.0]heptan-6-yl)-6-fluoroquinoline 1-oxide (Int 29) (1.01 g, 2.81 mmol) in MeOH (5 mL) was added Pd/C (1.00 g) and the mixture was stirred under H₂ atmosphere at rt overnight. The mixture was filtered and the filtrate was concentrated to dryness. The residue was purified by silica gel column chromatography (EtOAc/PE=1:1) to give the title compound as a yellow solid.

### Step 3: cis-4-(6-Fluoroquinolin-4-yl)piperidin-3-ol Trifluoroacetic Acid Salt (Int 29)

To a solution of cis-tert-butyl 4-(6-fluoroquinolin-4-yl)-3-hydroxypiperidine-1-carboxylate (Int 29b) (242 mg, 0.70 mmol) in DCM (5 mL) was added TFA (1 mL) and the mixture was stirred at rt for 2 h. The mixture was concentrated to dryness to give the title compound as a yellow solid.

### Intermediate 29/1: trans 6-Fluoro-4-(3-methoxypiperidin-4-yl)quinoline Trifluoroacetic Acid Salt (Int 29/1)

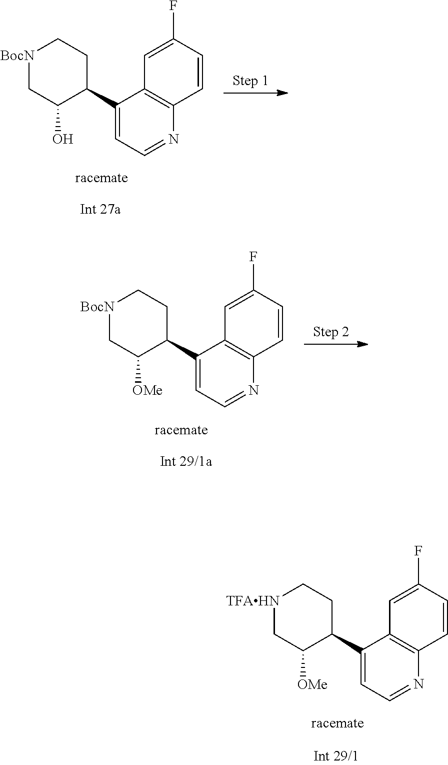

### Step 1: trans-tert-Butyl 4-(6-fluoroquinolin-4-yl)-3-methoxypiperidine-1-carboxylate (Int 29/1a)

To a solution of trans-tert-butyl 4-(6-fluoroquinolin-4-yl)-3-hydroxypiperidine-1-carboxylate (Int 27a) (314 mg, 0.91 mmol) in dry DMF (5 mL) was added NaH (91 mg, 2.28 mmol, 60%) at 0° C. and the mixture was stirred at 0° C. for 1 h. MeI was added (194 mg, 1.37 mmol) and the mixture was stirred at rt for 3 h. Aqueous NH₄Cl was added and the mixture was extracted with EtOAc (3×30 mL). The combined organic layers were washed with brine (30 mL) and concentrated to dryness to give the title compound as a yellow solid.

### Step 2: trans 6-Fluoro-4-(3-methoxypiperidin-4-yl)quinoline Trifluoroacetic Acid Salt (Int 29/1)

To a solution of trans-tert-butyl 4-(6-fluoroquinolin-4-yl)-3-methoxypiperidine-1-carboxylate (Int 29/1a) (265 mg, 0.74 mmol) in DCM (5 mL) was added TFA (1 mL) and the mixture was stirred at rt for 2 h. The mixture was concentrated to dryness to give the title compound as a yellow solid.

### Intermediate 30: Methyl 2-oxo-2-(4-(quinolin-4-yl)piperidin-1-yl)acetate (30)

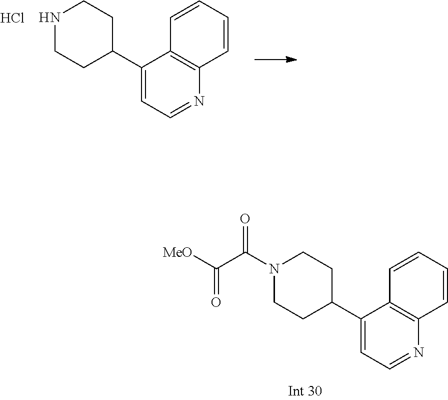 [SMILES: Cl ; c1ccc2c(C3CCNCC3)ccnc2c1 ; COC(=O)C(=O)N1CCC(c2ccnc3ccccc23)CC1]

To a solution of 4-(piperidin-4-yl)quinoline hydrochloride (150 mg, 0.53 mmol, 1.0 eq.) and TEA (107 mg, 1.06 mmol, 2.0 eq.) in DCM (3 mL) was added methyl 2-chloro-2-oxoacetate (77 mg, 0.63 mmol, 1.2 eq.) dropwise. The mixture was stirred at rt for 40 min and filtered. The filtrate was concentrated and purified by flash chromatography on silica gel (PE/EtOAc=4:1) to give the title compound as a brown solid.

### Intermediates 30/1 to 30/4

The following Intermediates were prepared similar as described for Intermediate 30 using the appropriate building blocks.

| Int. # | building blocks | Structure |
|---|---|---|
| Int 30/1 | 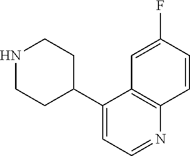 [SMILES: Fc1ccc2nccc(C3CCNCC3)c2c1] | 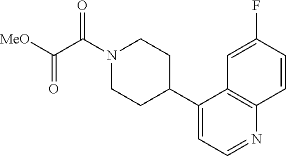 [SMILES: COC(=O)C(=O)N1CCC(c2ccnc3ccc(F)cc23)CC1] |
| | Int 20 | |
| Int 30/2 | 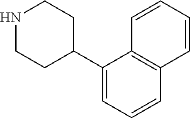 [SMILES: c1ccc2c(C3CCNCC3)cccc2c1] Int 20/11 | 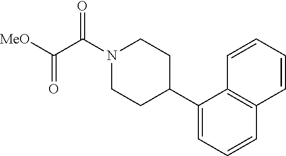 [SMILES: COC(=O)C(=O)N1CCC(c2cccc3ccccc23)CC1] |
| Int 30/3 | 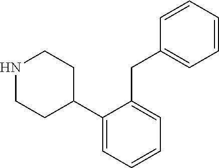 [SMILES: c1ccc(Cc2ccccc2C2CCNCC2)cc1] | 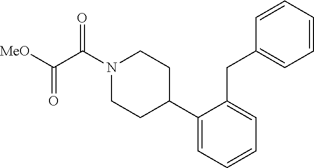 [SMILES: COC(=O)C(=O)N1CCC(c2ccccc2Cc2ccccc2)CC1] |
| | Int 20/9 | |
| Int 30/4 | 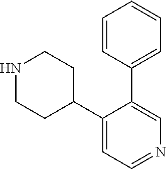 [SMILES: c1ccc(-c2cnccc2C2CCNCC2)cc1] Int 20/10 | 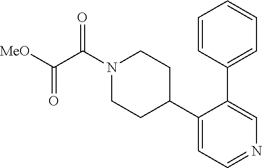 [SMILES: COC(=O)C(=O)N1CCC(c2ccncc2-c2ccccc2)CC1] |

### Intermediate 31: Lithium 2-(4-(6-fluoroquinolin-4-yl)piperidin-1-yl)-2-oxoacetate (Int 31)

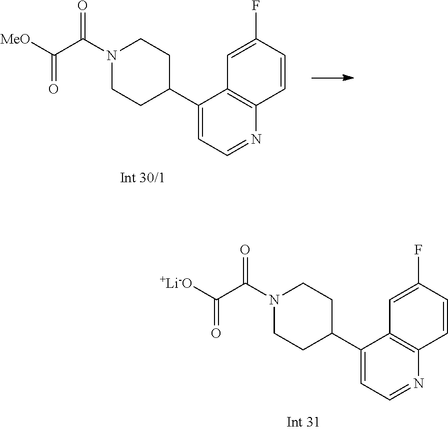

A mixture of methyl 2-(4-(6-fluoroquinolin-4-yl)piperidin-1-yl)-2-oxoacetate (Int 30/1) (1.10 g, 3.48 mmol, 1.0 eq.) and LiOH.H₂O (160.9 mg, 3.83 mmol, 1.1 eq) in THF/H₂O (20 mL:5 mL) was stirred at rt for 1 h. The reaction mixture was concentrated in vacuo, washed with hexane and EtOAc to afford the title compound as a white solid.

### Intermediate 32: 2-(4-(6-Fluoroquinolin-4-yl)piperidin-1-yl)-2-oxoacetic Acid (Int 32)

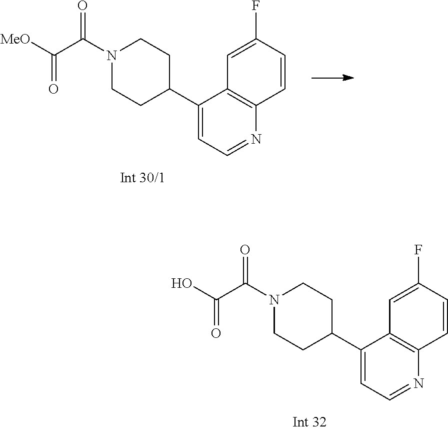 [SMILES: COC(=O)C(=O)N1CCC(c2ccnc3ccc(F)cc23)CC1 ; O=C(O)C(=O)N1CCC(c2ccnc3ccc(F)cc23)CC1]

To a mixture of methyl 2-(4-(6-fluoroquinolin-4-yl)piperidin-1-yl)-2-oxoacetate (Int 30/1) (500 mg, 1.58 mmol) in THE (15 mL) was added NaOH (64 mg, 1.6 mmol dissolved in 15 mL H₂O) and the mixture was stirred at rt for 0.5 h. The pH was adjusted to pH=2 with aqueous HCl. The mixture was concentrated to dryness and the residue was purified by column chromatography to give the title compound as a white solid.

### Intermediate 33: Sodium 2-oxo-2-(6-(trifluoromethyl)-2H-spiro[benzofuran-3,4′-piperidin]-1′-yl)acetate (Int 33)

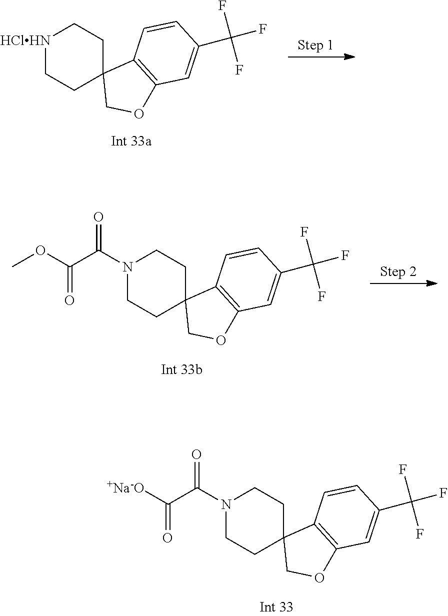

### Step 1: Methyl 2-oxo-2-(6-(trifluoromethyl)-2H-spiro[benzofuran-3,4′-piperidin]-1′-yl)acetate (Int 33b)

To a mixture of 6-(trifluoromethyl)-2H-spiro[benzofuran-3,4′-piperidine] hydrochloride (Int 33a) (200 mg, 0.681 mol) in DCM (4 mL) TEA (0.19 mL, 1.362 mmol) was added at rt. Methyl 2-chloro-2-oxoacetate (0.072 mL, 0.749 mmol) was added dropwise and the mixture was stirred at rt for 4 h. Water was added (4 mL) and the mixture was extracted with DCM (3×10 mL). The combined organic layers were washed with brine, dried over anhydrous Mg₂SO₄, filtered and concentrated to dryness. The residue was purified by silica gel column chromatography (gradient 12-100% EtOAc in cyclohexane) to afford the title compound as a white solid.

### Step 2: Sodium 2-oxo-2-(6-(trifluoromethyl)-2H-spiro[benzofuran-3,4′-piperidin]-1′-yl)acetate (Int 33)

To a mixture of methyl 2-oxo-2-(6-(trifluoromethyl)-2H-spiro[benzofuran-3,4′-piperidin]-1′-yl)acetate (Int 33b) (70.0 mg, 0.204 mmol) in THE (1.5 mL) aqueous NaOH (0.112 mL, 2M, 0.224 mmol) was added. The mixture was stirred at rt for 30 min. The mixture was concentrated to dryness to afford the title compound which was used in the next step without further purification.

### Intermediate 40: 5-Fluoro-6-(trifluoromethyl)-2H-spiro[benzofuran-3,4′-piperidine] (Int 40)

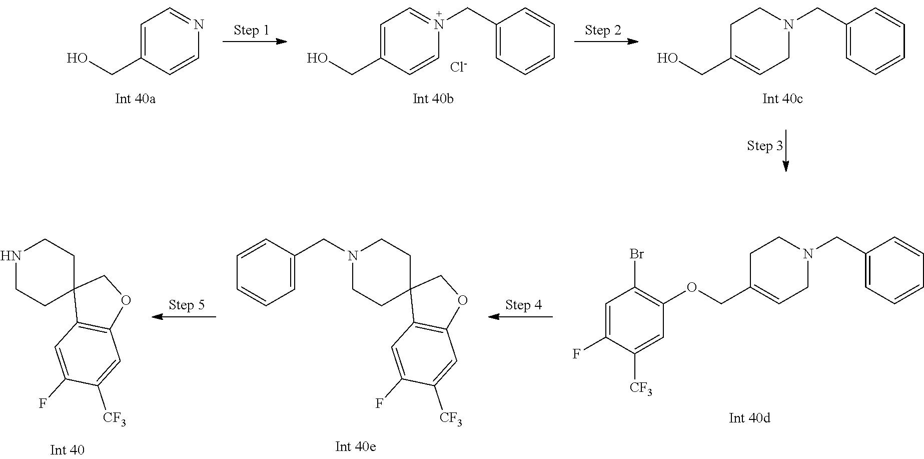

### Step 1: 1-Benzyl-4-(hydroxymethyl)pyridin-1-ium Chloride (Int 40b)

A solution of pyridin-4-ylmethanol (Int 40a) (2.18 g, 20.00 mmol) and (chloromethyl)benzene (2.52 g, 20.00 mmol) in CH₃CN (30 mL) was refluxed overnight. The mixture was concentrated to dryness to give the title compound as a white solid.

### Step 2: (1-Benzyl-1,2,3,6-tetrahydropyridin-4-yl)methanol (Int 40c)

To a solution of 1-benzyl-4-(hydroxymethyl)pyridin-1-ium chloride (Int 40b) (4.70 g, 20.00 mmol) in MeOH (50 mL) was added NaBH₄ (1.52 g, 40 mmol) at 0° C. and the mixture was stirred for 3 h. The mixture was poured into water (100 mL) and extracted with EtOAc (2×100 mL). The combined organic layers were dried over anhydrous Na₂SO₄, filtered and concentrated to dryness. The residue was purified by column chromatography on silica gel (PE/EtOAc=1:1) to give the title compound as a brown solid.

### Step 3: 1-Benzyl-4-((2-bromo-4-fluoro-5-(trifluoromethyl)phenoxy)methyl)-1,2,3,6-tetrahydropyridine (Int 40d)

To a mixture of (1-benzyl-1,2,3,6-tetrahydropyridin-4-yl)methanol (Int 40c) (557 mg, 2.75 mmol), 2-bromo-4-fluoro-5-(trifluoromethyl)phenol (711 mg, 2.75 mmol) and PPh₃ (721 mg, 2.75 mmol) in THE (20 mL) was added DEAD (479 mg, 2.75 mmol) and the mixture was stirred overnight. The mixture was concentrated to dryness and the residue was purified by column chromatography on silica gel (PE/EtOAc=5:1) to give the title compound as a brown solid.

### Step 4: 1′-Benzyl-5-fluoro-6-(trifluoromethyl)-2H-spiro[benzofuran-3,4′-piperidine] (Int 40e)

A mixture of 1-benzyl-4-((2-bromo-4-fluoro-5-(trifluoromethyl)phenoxy)methyl)-1,2,3,6-tetrahydropyridine (Int 40d) (1.47 g, 3.33 mmol), AIBN (109 mg, 0.66 mmol) and n-Bu₃SnH (1.94 g, 6.66 mmol) in toluene (20 mL) was refluxed overnight. The mixture was concentrated to dryness and the residue was purified by column chromatography on silica gel (PE/EtOAc=5:1) to give the title compound as a brown solid.

### Step 5: 5-Fluoro-6-(trifluoromethyl)-2H-spiro[benzofuran-3,4′-piperidine] (Int 40)

To a solution of 1′-benzyl-5-fluoro-6-(trifluoromethyl)-2H-spiro[benzofuran-3,4′-piperidine] (Int 40e) (730 mg, 2.00 mmol) in DCE (10 mL) was added 1-chloroethyl carbonochloridate (429 mg, 3.00 mmol) at 0° C. and the mixture was refluxed for 2 h. After cooling to rt the mixture was concentrated to dryness. The residue was dissolved in MeOH (10 mL) and the mixture was refluxed for 1 h. After cooling to rt, the mixture was partitioned between NaOH (20 mL, 1.0 M) and EtOAc (20 mL) and the aqueous layer was extracted with EtOAc (2×20 mL). The combined organic layers were concentrated to dryness and the residue was purified by column chromatography on silica gel (PE/EtOAc=2:1) to give the title compound as a brown solid.

### Intermediate 40/1: 2H-Spiro[benzofuran-3,4′-piperidine]-6-carbonitrile (Int 40/1)

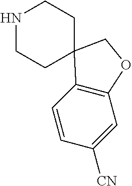 [SMILES: N#Cc1ccc2c(c1)OCC21CCNCC1]

The title compound was prepared similar as described for intermediate 40 using in step 3 4-bromo-3-hydroxybenzonitrile in place of 2-bromo-4-fluoro-5-(trifluoromethyl)phenol.

### Intermediate 41: 6-(Difluoromethyl)-2H-spiro[benzofuran-3,4′-piperidine] (Int 41)

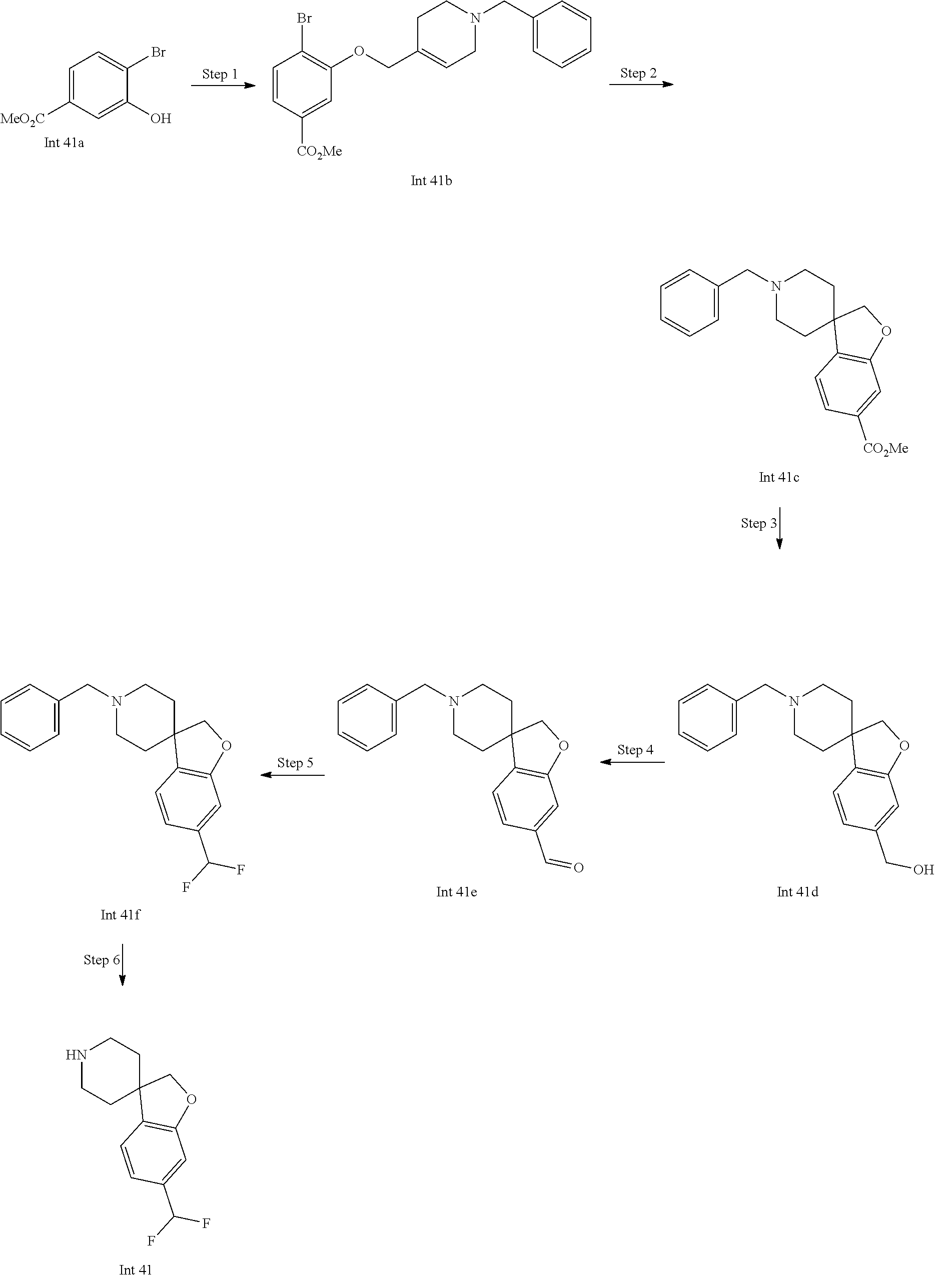

### Step 1: Methyl 3-((1-benzyl-1,2,3,6-tetrahydropyridin-4-yl)methoxy)-4-bromobenzoate (Int 41a)

To a solution of methyl 4-bromo-3-hydroxybenzoate (1.14 g, 4.93 mmol), (1-benzyl-1,2,3,6-tetrahydropyridin-4-yl)methanol (1.00 mg, 4.93 mmol) and PPh₃ (1.29 g, 4.93 mmol) in THE (30 mL) was added DEAD (858 mg, 4.93 mmol) at rt and the mixture was stirred overnight. The mixture was concentrated to dryness and the residue was purified by column chromatography on silica gel (PE/EtOAc=5:1) to give the title compound as a light yellow solid.

### Step 2: Methyl 1′-benzyl-2H-spiro[benzofuran-3,4′-piperidine]-6-carboxylate (Int 41c)

A mixture of methyl 3-((1-benzyl-1,2,3,6-tetrahydropyridin-4-yl)methoxy)-4-bromobenzoate (Int 41a) (1.23 g, 2.95 mmol), AIBN (97 mg, 0.59 mmol) and n-Bu₃SnH (1.72 g, 5.90 mmol) in toluene (20 mL) was refluxed overnight. The mixture was concentrated to dryness and the residue was purified by column chromatography on silica gel (PE/EtOAc=5:1) to give the title compound as a white solid.

### Step 3: (1′-Benzyl-2H-spiro[benzofuran-3,4′-piperidin]-6-yl)methanol (Int 41d)

To a solution of methyl 1′-benzyl-2H-spiro[benzofuran-3,4′-piperidine]-6-carboxylate (Int 41c) (696 mg, 2.07 mmol) in THE (20 mL) was added LiAlH₄ (79 mg, 2.08 mmol) at 0° C. The mixture was stirred for 3 h and then water (1 mL) was added. The mixture was filtered and the organic layer was concentrated to dryness to give the title compound as white solid.

### Step 4: 1′-Benzyl-2H-spiro[benzofuran-3,4′-piperidine]-6-carbaldehyde (Int 41e)

The mixture of (1′-benzyl-2H-spiro[benzofuran-3,4′-piperidin]-6-yl)methanol (Int 41d) (447 mg, 1.44 mmol) and MnO₂ (2.51 g, 28.8 mmol) in DCM (30 mL) was refluxed for 3 h. The mixture was filtered and concentrated to dryness to give the title compound as a brown solid which was used in the next step without further purification.

### Step 5: 1′-Benzyl-6-(difluoromethyl)-2H-spiro[benzofuran-3,4′-piperidine] (Int 41f)

To a solution of 1′-benzyl-2H-spiro[benzofuran-3,4′-piperidine]-6-carbaldehyde (Int 41e) (355 mg, 1.16 mmol) in DCM (20 mL) was added DAST (934 mg, 5.80 mmol). The mixture was refluxed for 3 h. After cooling to rt, water (20 mL) was added and the mixture was extracted with EtOAc (3×10 mL). The combined organic layers were washed with brine (20 mL), dried over Na₂SO₄, filtered and concentrated. The residue was purified by column chromatography on silica gel (PE/EtOAc=2:1) to give the title compound as a white solid.

### Step 6: 6-(Difluoromethyl)-2H-spiro[benzofuran-3,4′-piperidine] (Int 41)

To a solution of 1′-benzyl-6-(difluoromethyl)-2H-spiro[benzofuran-3,4′-piperidine] (Int 41f) (190 mg, 0.58 mmol) in DCE (10 mL) was added 1-chloroethyl carbonochloridate (249 mg, 1.74 mmol) at 0° C. and the mixture was refluxed for 2 h. After cooling to rt the mixture was concentrated. The residue was dissolved in MeOH (10 mL) and the mixture was refluxed for 1 h. After cooling to rt, the mixture was partitioned between NaOH (20 mL, 1.0 M) and EtOAc (20 mL). The aqueous layer was extracted with EtOAc (2×20 mL). The combined organic layers were concentrated to dryness and the residue was purified by column chromatography on silica gel (PE/EtOAc=2:1) to give the title compound as a brown solid.

### Intermediate 42: 6′-(Trifluoromethyl)-2′H-spiro[azepane-4,3′-benzofuran] hydrochloride (Int 42)

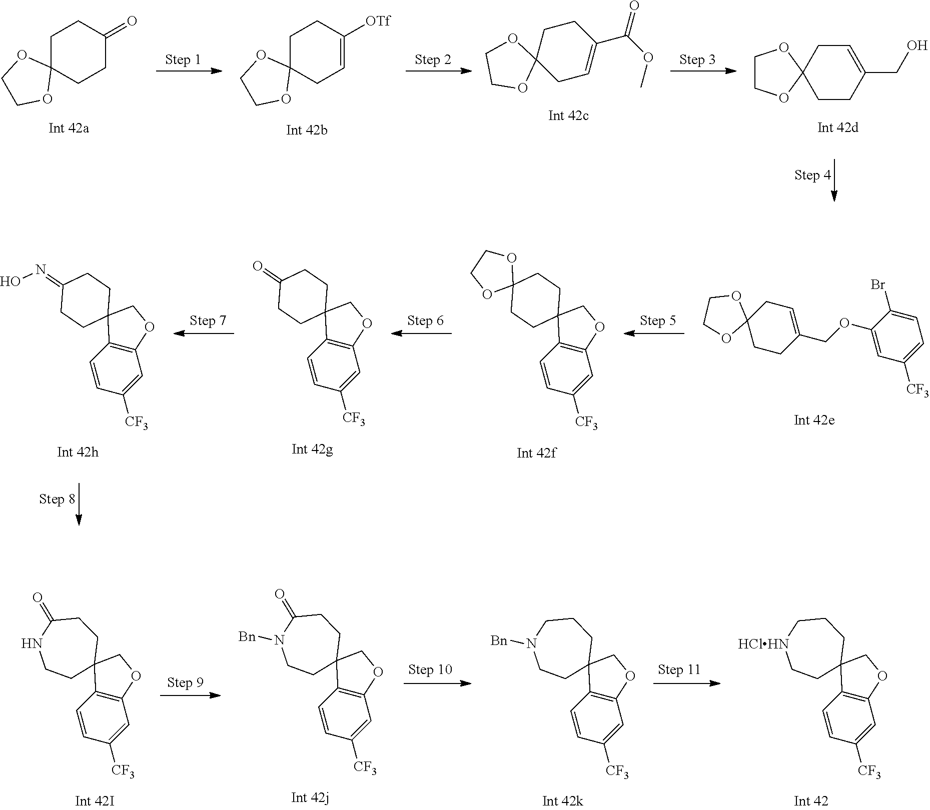

### Step 1: 1,4-Dioxaspiro[4.5]dec-7-en-8-yl Trifluoromethanesulfonate (Int 42b)

To a solution of 1,4-dioxaspiro[4.5]decan-8-one (Int 42a) (1.20 g, 7.69 mmol) and 1,1,1-trifluoro-N-phenyl-N-((trifluoromethyl)sulfonyl)methanesulfonamide (3.57 g, 10.00 mmol) in THE (50 mL) under N₂ at −78° C. was added lithium bis(trimethylsilyl)amide in THE (1 M, 10.0 mL, 10.00 mmol). The mixture was stirred at rt overnight, quenched with H₂O (50 mL), and extracted with EtOAc (3×50 mL). The combined organic layers were dried over MgSO₄, filtered and concentrated to give the title compound as a brown oil, which was directly used in the next step without further purification.

### Step 2: Methyl 1,4-dioxaspiro[4.5]dec-7-ene-8-carboxylate (Int 42c)

A mixture of 1,4-dioxaspiro[4.5]dec-7-en-8-yl trifluoromethanesulfonate (Int 42b) (7.69 mmol, crude), Pd(PPh₃)₂Cl₂ (291 mg, 1.11 mmol), and TEA (1.14 g, 11.04 mmol) in MeOH (20 mL) was stirred at rt overnight under a carbon monoxide atmosphere at 5 bar pressure. The reaction mixture was quenched with H₂O (100 mL) and extracted with EtOAc (2×100 mL). The combined organic layers were concentrated and the residue was purified by column chromatography on silica gel (PE/EtOAc=10:1) to give the title compound as a yellow oil.

### Step 3: (1,4-Dioxaspiro[4.5]dec-7-en-8-yl)methanol (Int 42d)

To a solution of methyl 1,4-dioxaspiro[4.5]dec-7-ene-8-carboxylate (Int 42c) (1.20 g, 6.06 mmol) in dry THE (50 mL) was added LiAlH₄ (230 mg, 6.06 mmol) at 0° C., and the mixture was stirred at rt for 3 h. Then 40% aqueous NaOH solution (1 mL) was added and the mixture was stirred at rt for 2 h. The mixture was filtered and the filtrate was concentrated. The residue was purified by column chromatography on silica gel (PE/EtOAc=2:1) to give the title compound as a light yellow oil.

### Step 4: 8-((2-Bromo-5-(trifluoromethyl)phenoxy)methyl)-1,4-dioxaspiro[4.5]dec-7-ene (Int 42e)

To a solution of (1,4-dioxaspiro[4.5]dec-7-en-8-yl)methanol (Int 42d) (340 mg, 2.00 mmol), 2-bromo-5-(trifluoromethyl)phenol (482 mg, 2.00 mmol) and PPh₃ (786 mg, 3.00 mmol) in THE (10 mL) was added DEAD (576 mg, 3.00 mmol) under Ar, and the mixture was stirred at rt overnight. The solvent was removed under vacuum and the residue was purified by column chromatography on silica gel (PE/EtOAc=10:1) to give the title compound as a yellow oil.

### Step 5: 6-(Trifluoromethyl)-2H-dispiro[benzofuran-3,1′-cyclohexane-4′,2″-[1,3]dioxolane](Int 42f)

A solution of 8-((2-bromo-5-(trifluoromethyl)phenoxy)methyl)-1,4-dioxaspiro[4.5]dec-7-ene (Int 42e) (393 mg, 1.00 mmol), n-Bu₃SnH (582 mg, 2.00 mmol) and AIBN (33 mg, 0.20 mmol) in toluene (10 mL) was heated to reflux for 3 h. The solvent was removed under vacuum and the residue was purified by column chromatography on silica gel (PE/EtOAc=10:1) to afford the title compound as a white solid.

### Step 6: 6-(Trifluoromethyl)-2H-spiro[benzofuran-3,1′-cyclohexan]-4′-one (Int 42g)

To a solution of 6-(trifluoromethyl)-2H-dispiro[benzofuran-3,1′-cyclohexane-4′,2″-[1,3]dioxolane] (Int 42f) (314 mg, 1.00 mmol) in THE (20 mL) was added concentrated aqueous HCl (1 mL, 12 M), and the mixture was stirred at rt overnight. The mixture was extracted with EtOAc (3×30 mL). The combined organic layers were dried over MgSO₄, filtered and concentrated in to dryness. The residue was purified by column chromatography on silica gel (PE/EtOAc=10:1) to give the title compound as a yellow solid.

### Step 7: 2H-6-(Trifluoromethyl)-2H-spiro[benzofuran-3,1′-cyclohexan]-4′-one Oxime (Int 42 h)

A solution of 6-(trifluoromethyl)-2H-spiro[benzofuran-3,1′-cyclohexan]-4′-one (Int 42g) (2.70 g, 10.00 mmol), NH₂OH.HCl (1.38 g, 20.00 mmol) and AcONa (2.46 g, 30.00 mmol) in MeOH (50 mL) was stirred at rt overnight. The mixture was concentrated. The residue was dissolved in water (50 mL) and extracted with EtOAc (3×50 mL). The combined organic layers were concentrated to give the title compound as a white solid.

### Step 8: 6′-(Trifluoromethyl)-2′H-spiro[azepane-4,3′-benzofuran]-7-one (Int 42i)

To a solution of 2H-6-(trifluoromethyl)-2H-spiro[benzofuran-3,1′-cyclohexan]-4′-one oxime (Int 42 h) (2.85 g, 10.00 mmol) in THE (30 mL) was added SOCl₂ (2.38 g, 20.00 mmol) at 0° C. and the mixture was stirred at rt overnight. The mixture was concentrated to dryness. The residue was purified by column chromatography on silica gel (PE/EtOAc=2:1) to give the title compound as a brown solid.

### Step 9: 1-Benzyl-6′-(trifluoromethyl)-2′H-spiro[azepane-4,3′-benzofuran]-7-one (Int 42j)

To a solution of 6′-(trifluoromethyl)-2′H-spiro[azepane-4,3′-benzofuran]-7-one (Int 42i) (1.14 g, 4.00 mmol) in THE (20 mL) was added NaH (320 mg, 8.00 mmol, 60% in mineral oil) at 0° C. and the mixture was stirred at rt overnight. The mixture was poured into water (20 mL) and extracted with EtOAc (3×20 mL). The combined organic layers were concentrated to dryness. The residue was purified by column chromatography on silica gel (PE/EtOAc=2:1) to give the title compound as a brown solid.

### Step 10: 1-Benzyl-6′-(trifluoromethyl)-2′H-spiro[azepane-4,3′-benzofuran] (Int 42k)

To a solution of 1-benzyl-6′-(trifluoromethyl)-2′H-spiro[azepane-4,3′-benzofuran]-7-one (Int 42j) (1.20 g, 2.90 mmol) in THE (20 mL) was added BH₃.THF (29 mL, 29.00 mmol, 1.0 M) at 0° C. and the mixture was refluxed overnight. After cooling to rt, MeOH (1.0 mL) was added and the mixture was stirred for 2 h. Aqueous HCl (1.0 mL, 1.0 M.) was added and the mixture was refluxed for 3 h. The mixture was concentrated and the residue was purified by column chromatography on silica gel (PE/EtOAc=3:1) to give the title compound as a white solid.

### Step 11: 6′-(Trifluoromethyl)-2′H-spiro[azepane-4,3′-benzofuran] hydrochloride (Int 42)

To a solution of 1-benzyl-6′-(trifluoromethyl)-2′H-spiro[azepane-4,3′-benzofuran] (Int 42k) (550 mg, 1.52 mmol) in DCE (50 mL) was added 1-chloroethyl carbonochloridate (5 mL) at 0° C., and the mixture was refluxed overnight. After cooling to rt the mixture was concentrated. The residue was dissolved in MeOH (50 mL) and the mixture was refluxed for 1 h. After cooling to rt the mixture was concentrated to dryness. The residue was washed with hexane and dried to give the title compound as a gray solid.

### Example 1: N-(4-Chloro-2-fluorophenyl)-2-(4-(6-fluoroquinolin-4-yl)piperidin-1-yl)-2-oxoacetamide (1)

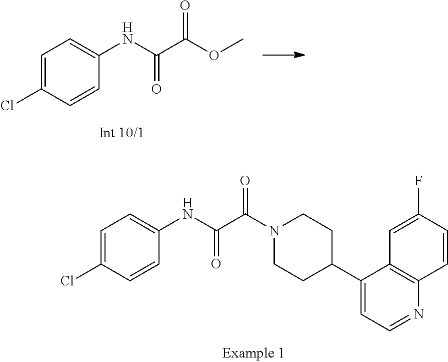 [SMILES: COC(=O)C(=O)Nc1ccc(Cl)cc1 ; O=C(Nc1ccc(Cl)cc1)C(=O)N1CCC(c2ccnc3ccc(F)cc23)CC1]

A solution of methyl 2-((4-chlorophenyl)amino)-2-oxoacetate (Int 10/1) (166 mg, 0.78 mmol), TEA (157 mg, 1.56 mmol) and 6-fluoro-4-(piperidin-4-yl)quinoline (Int 20) (160 mg, 0.52 mmol) in MeOH (6 mL) was stirred at 70° C. for 16 h. The mixture was concentrated and the residue was diluted with H₂O (20 mL). The mixture was extracted with EtOAc (3×30 mL). The combined organic layers were washed with brine (30 mL), dried over Na₂SO₄, filtered and concentrated to dryness. The residue was purified by preparative HPLC to give the title compound as a white solid. ¹H NMR (500 MHz, DMSO-d₆): δ ppm 10.96 (s, 1H), 8.84 (d, J=4.5 Hz, 1H), 8.15-8.10 (m, 2H), 7.72-7.68 (m, 3H), 7.47-7.42 (m, 3H), 4.54-4.51 (m, 1H), 3.95-3.92 (m, 1H), 3.78-3.73 (m, 1H), 3.50-3.45 (m, 1H), 3.11-3.05 (m, 1H), 2.00-1.93 (m, 2H), 1.79-1.67 (m, 2H). MS (ESI): m/z 412.2 [M+H]⁺.

### Examples 1/1 to 1/48

The following Examples were prepared similar as described for Example 1 using the appropriate building blocks.

| | Building | | |
|---|---|---|---|
| # | blocks | Structure | Analytical data |
| 1/1 | Int 10/1, 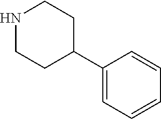 [SMILES: c1ccc(C2CCNCC2)cc1] | 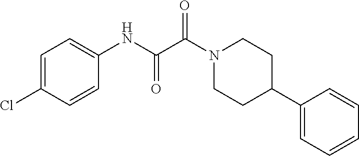 [SMILES: O=C(Nc1ccc(Cl)cc1)C(=O)N1CCC(c2ccccc2)CC1] | ¹H NMR (500 MHz, DMSO- d₆): δ ppm 10.94 (s, 1H), 7.68-7.71 (m, 2H), 7.43-7.41 (m, 2H), 7.34-7.19 (m, 5H), 4.48-4.45 (m, 1H), 3.89-3.85 (m, 1H), 3.29-3.22 (m, 1H), 2.88-2.81 (m, 2H), 1.90-1.82 (m, 2H), 1.67-1.56 (m, 2H). MS (ESI): m/z 343.0 [M + H]⁺. |
| 1/2 | Int 10/2, 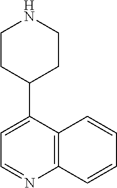 [SMILES: c1ccc2c(C3CCNCC3)ccnc2c1] | 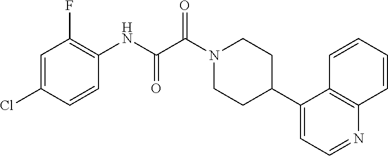 [SMILES: O=C(Nc1ccc(Cl)cc1F)C(=O)N1CCC(c2ccnc3ccccc23)CC1] | ¹H NMR (500 MHz, CD₃OD): δ ppm 9.10 (s, 1H), 8.69 (d, J = 9.0 Hz, 1H), 8.26 (d, J = 8.5 Hz, 1H), 8.17-8.14 (m, 1H), 8.03-7.97 (m, 3H), 7.35 (d, J = 9.0 Hz, 1H), 7.27 (d, J = 9.0 Hz, 1H), 4.79-4.76 (m, 1H), 4.40-4.37 (m, 1H), 4.17-4.12 (m, 1H), 3.62-3.56 (m, 1H), 3.21-3.16 (m, 1H), 2.20-1.97 (m, 4H). MS (ESI): m/z 412.1 [M + H]⁺. |
| 1/3 | Int 10/1, 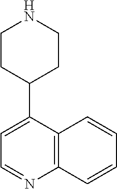 [SMILES: c1ccc2c(C3CCNCC3)ccnc2c1] | 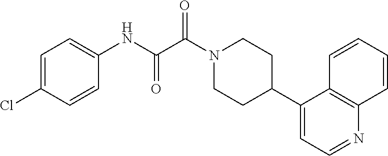 [SMILES: O=C(Nc1ccc(Cl)cc1)C(=O)N1CCC(c2ccnc3ccccc23)CC1] | ¹H NMR (500 MHz, DMSO- d₆): δ ppm 10.96 (s, 1H), 8.85 (d, J = 5.5 Hz, 1H), 8.35 (d, J = 10.5 Hz 1H), 8.05 (d, J = 10.0 Hz, 1H), 7.79-7.64 (m, 4H), 7.43-7.41 (m, 3H), 4.55-4.52 (m, 1H), 3.97-3.80 (m, 2H), 3.50-3.44 (m, 1H), 3.11-3.05 (m, 1H), 2.03-1.94 (m, 2H), 1.82-1.69 (m, 2H). MS (ESI): m/z 394.1 [M + H]⁺. |
| 1/4 | Int 10/2 Int 20/2 | 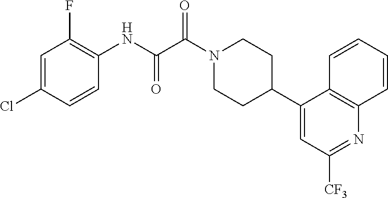 [SMILES: O=C(Nc1ccc(Cl)cc1F)C(=O)N1CCC(c2cc(C(F)(F)F)nc3ccccc23)CC1] | ¹H NMR (500 MHz, CDCl₃): δ ppm 9.39 (s, 1H), 8.31-.27 (m, 2H), 8.17 (d, J = 8.5 Hz, 1H), 7.86-7.83 (m, 1H), 7.75-7.72 (m, 1H), 7.60 (s, 1H), 7.20-7.16 (m, 2H), 5.47-5.45 (m, 1H), 4.90-4.87 (m, 1H), 3.79-3.74 (m, 1H), 3.44-3.38 (m, 1H), 3.08-3.02 (m, 1H), 2.20-2.15 (m, 2H), 2.05-1.91 (m, 2H). MS (ESI): m/z 480.0 [M + H]⁺. |
| 1/5 | Int 10/2 Int 20/1 | 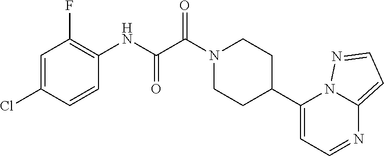 [SMILES: O=C(Nc1ccc(Cl)cc1F)C(=O)N1CCC(c2ccnc3ccnn23)CC1] | ¹H NMR (500 MHz, CDCl₃): δ ppm 9.36 (s, 1H), 8.46 (d, J = 5.0 Hz, 1H), 8.30-8.27 (m, 1H), 8.15 (s, 1H), 7.19- 7.15 (m, 2 H), 6.75 (s, 1H), 6.65 (d, J = 5.0 Hz, 1H), 5.41-5.39 (m, 1H), 4.85-4.82 (m, 1H), 3.95-3.93 (m, 1H), 3.41-3.36 (m, 1H), 3.07-3.01 (m, 1H), 2.41-2.34 (m, 2H), |
| | | | 1.92-1.78 (m, 2H). MS (ESI): |
| | | | m/z 402.0 [M + H]⁺. |
| 1/6 | Int 10/2 Int 20 | 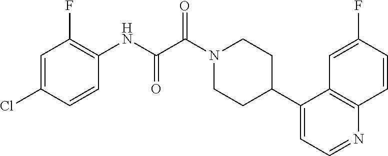 [SMILES: O=C(Nc1ccc(Cl)cc1F)C(=O)N1CCC(c2ccnc3ccc(F)cc23)CC1] | ¹H NMR (500 MHz, CDCl₃): δ ppm 9.38 (s, 1H), 8.84 (d, J = 4.5 Hz, 1H), 8.31-8.27 (m, 1H), 8.17-8.14 (m, 1H), 7.70-7.67 (m, 1H), 7.53-7.49 (m, 1H), 7.28 (d, J = 5.0 Hz, 1H), 7.19-7.15 (m, 2H), 5.44-5.41 (m, 1H), 4.88-4.84 (m, 1H), 3.57-3.49 (m, 1H), 3.40-3.36 (m, 1H), 3.04-2.98 |
| | | | (m, 1H), 2.15-2.11 (m, 2H), |
| | | | 1.98-1.91 (m, 1 H), 1.88- |
| | | | 1.81 (m, 1H). MS (ESI): m/z |
| | | | 429.9 [M + H]⁺. |
| 1/7 | Int 10/2 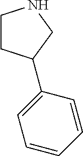 [SMILES: c1ccc(C2CCNC2)cc1] | 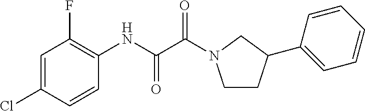 [SMILES: O=C(Nc1ccc(Cl)cc1F)C(=O)N1CCC(c2ccccc2)C1] | ¹H NMR (500 MHz, DMSO- d₆): δ ppm 10.40 (s, 1H), 7.78-7.72 (m, 1H), 7.57-7.53 (m, 1H), 7.35-7.25 (m, 6H), 4.25-3.72 (m, 3H), 3.63-3.35 (m, 2H), 3.33-3.26 (m, 1H), 2.04-1.99 (m, 1H). MS (ESI): m/z 347.0 [M + H]⁺. |
| 1/8 | Int 10/3 Int 20/15 | 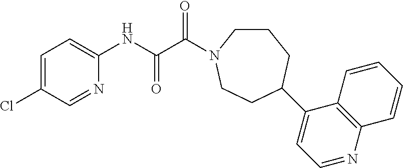 [SMILES: O=C(Nc1ccc(Cl)cn1)C(=O)N1CCCC(c2ccnc3ccccc23)CC1] | ¹H NMR (500 MHz, DMSO- d₆): δ ppm 11.50 (s, 1H), 9.08-9.05 (m, 1H), 8.48-8.44 (m, 2H), 8.17-8.09 (m, 2H), 8.00-7.94 (m, 2H), 7.86-7.70 (m, 2H), 3.89-3.52 (m, 5H), 2.10-1.93 (m, 6H). MS (ESI): m/z 409.1 [M + H]⁺. |
| 1/9 | Int 10/3 Int 21 | 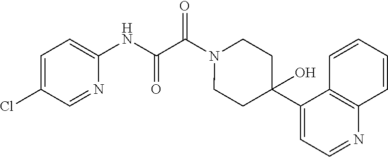 [SMILES: O=C(Nc1ccc(Cl)cn1)C(=O)N1CCC(O)(c2ccnc3ccccc23)CC1] | ¹H NMR (500 MHz, DMSO- d₆): δ ppm 11.41 (s, 1H), 9.05-9.02 (m, 2H), 8.43 (s, 1H), 8.15-8.10 (m, 2H), 7.98-7.96 (m, 1H), 7.89-7.86 (m, 1H), 7.74-7.71 (m, 2H), 6.04 (br s, 1H), 4.35-4.33 (m, 1H), 3.72-3.69 (m, 2H), 3.34-3.28 (m, 1H), 2.26-2.12 (m, 4H). MS (ESI): m/z |
| | | | 411.0 [M + H]⁺. |
| 1/10 | Int 10/3 Int 23 | 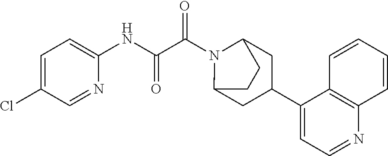 [SMILES: O=C(Nc1ccc(Cl)cn1)C(=O)N1C2CCC1CC(c1ccnc3ccccc13)C2] | ¹H NMR (500 MHz, DMSO- d₆): δ ppm 11.19 (s, 1H), 8.86-8.84 (m, 1H), 8.45-7.97 (m, 5H), 7.79-7.75 (m, 1H), 7.68-7.65 (m, 1H), 7.57-7.51 (m, 1H), 4.71-4.70 (m, 1H), 4.58-4.52 (m, 1H), 4.11-3.61 (m, 1H), 2.59-2.54 (m, 1H), 2.18-1.63 (m, 7H). MS (ESI): m/z 421.1 [M + H]⁺. |
| 1/11 | Int 10/3 Int 23/1 | 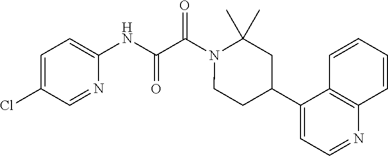 [SMILES: CC1(C)CC(c2ccnc3ccccc23)CCN1C(=O)C(=O)Nc1ccc(Cl)cn1] | ¹H NMR (500 MHz, DMSO- d₆): δ ppm 11.44 (s, 1H), 9.08 (s, 1H), 8.47-8.31 (m, 2H), 8.18-7.01 (m, 6H), 4.04-4.00 (s, 1H), 3.65-3.51 (m, 2H), 2.13-2.06 (m, 2H), 1.78-1.75 (m, 2H), 1.64 (s, 6H). MS (ESI): m/z 423.1 [M + H]⁺. |
| 1/12 | Int 10/2 Int 20/8 | 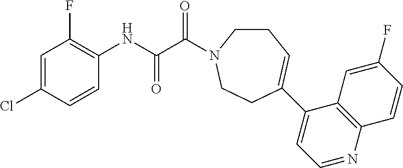 [SMILES: O=C(Nc1ccc(Cl)cc1F)C(=O)N1CCC=C(c2ccnc3ccc(F)cc23)CC1] | ¹H NMR (500 MHz, DMSO- d₆): δ ppm 10.42 (br s, 1H), 8.85-8.82 (m, 1H), 8.13-8.10 (m, 1H), 7.83-7.53 (m, 4H), 7.38-7.30 (m, 2H), 5.98-5.87 (m, 1H), 4.31-3.76 (m, 4H), 2.79-2.07 (m, 4H). MS (ESI): m/z 442.1 [M + H]⁺. |
| 1/13 | Int 10/2 Int 20/7 | 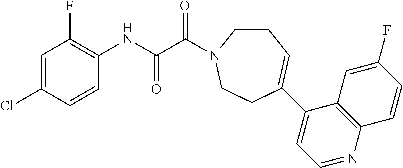 [SMILES: O=C(Nc1ccc(Cl)cc1F)C(=O)N1CCC=C(c2ccnc3ccc(F)cc23)CC1] | ¹H NMR (500 MHz, DMSO- d₆): δ ppm 10.76 (br s, 1H), 8.84-8.81 (m, 1H), 8.11-8.02 (m, 2H), 7.82-7.80 (m, 1H), 7.69-7.68 (m, 1H), 7.56-7.44 (m, 2H), 7.33-7.29 (m, 1H), 3.91-3.54 (m, 5H), 2.04-1.87 (m, 6H). MS (ESI): m/z 444.1 [M + H]⁺. |
| 1/14 | Int 10/5 Int 20 | 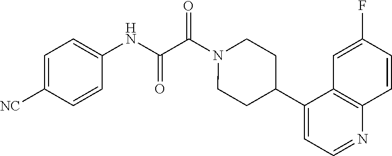 [SMILES: N#Cc1ccc(NC(=O)C(=O)N2CCC(c3ccnc4ccc(F)cc34)CC2)cc1] | ¹H NMR (500 MHz, DMSO- d₆): δ ppm 11.26 (br s, 1H), 8.85 (d, J = 4.5 Hz, 1H), 8.15-8.10 (m, 2H), 7.88-7.83 (m, 4H), 7.72-7.68 (m, 1H), 7.47 (d, J = 4.5 Hz, 1H), 4.54-4.52 (m, 1H), 3.96-3.93 (m, 1H), 3.78-3.73 (m, 1H), 3.52-3.46 (m, 1H), 3.13-3.07 (m, 1H), 2.01-1.93 (m, 2H), |
| | | | 1.79-1.68 (m, 1H). MS (ESI): |
| | | | m/z 403.2 [M + H]⁺. |
| 1/15 | Int 10/6 Int 20 | 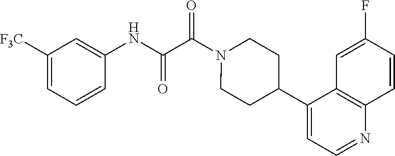 [SMILES: O=C(Nc1cccc(C(F)(F)F)c1)C(=O)N1CCC(c2ccnc3ccc(F)cc23)CC1] | ¹H NMR (500 MHz, DMSO- d₆): δ ppm 11.17 (s, 1H), 8.85 (d, J = 4 .5 Hz, 1H), 8.16-8.10 (m, 3H), 7.88 (d, J = 8.2 Hz, 1H), 7.72-7.68 (m, 1H), 7.63-7.60 (m, 1H), 7.51-7.47 (m, 2H) 4.54-4.52 (m, 1H), 4.00-3.97 (m, 1H), 3.78-3.74 (m, 1H), 3.51-3.46 (m, 1H), 3.12-3.07 (m, 1H), |
| | | | 2.01-1.93 (m, 2H), 1.81-1.67 |
| | | | (m, 2H). MS (ESI): m/z |
| | | | 446.2 [M + H]⁺. |
| 1/16 | Int 10 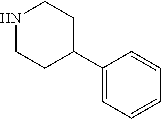 [SMILES: c1ccc(C2CCNCC2)cc1] | 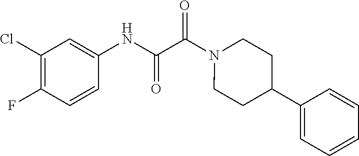 [SMILES: O=C(Nc1ccc(F)c(Cl)c1)C(=O)N1CCC(c2ccccc2)CC1] | ¹H NMR (500 MHz, DMSO- d₆): δ ppm 11.03 (s, 1H), 7.98-7.96 (s, 1H), 7.60-7.57 (m, 1H), 7.45-7.41 (s, 1H), 7.33-7.20 (m, 5H), 4.48-4.46 (m, 1H), 3.92-3.91 (m, 1H), 3.28-3.22 (m, 1H), 2.89-2.83 (m, 2H), 1.90-1.82 (m, 2H), 1.66-1.56 (m, 2H). MS (ESI): m/z 360.9 [M + H]⁺. |
| 1/17 | Int 10/7 Int 20 | 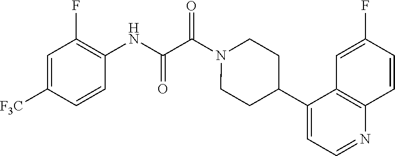 [SMILES: O=C(Nc1ccc(C(F)(F)F)cc1F)C(=O)N1CCC(c2ccnc3ccc(F)cc23)CC1] | ¹H NMR (500 MHz, DMSO- d₆): δ ppm 11.05 (s, 1H), 8.85 (d, J = 4.5 Hz, 1H), 8.19-8.10 (m, 3H), 7.81-7.79 (m, 1H), 7.72-7.62 (m, 2H), 4.53-4.50 (m, 1H), 3.93-3.91 (m, 1H), 3.78-3.73 (m, 1H), 3.52-3.47 (m, 1H), 3.10-3.05 (m, 1H), 2.00-1.72 (m, 4H) MS (ESI): m/z 464.3 [M + H]⁺. |
| 1/18 | Int 10/8 Int 20 | 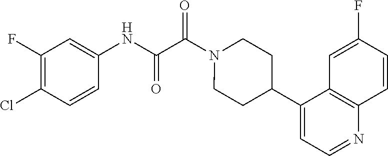 [SMILES: O=C(Nc1ccc(Cl)c(F)c1)C(=O)N1CCC(c2ccnc3ccc(F)cc23)CC1] | ¹H NMR (500 MHz, DMSO- d₆): δ ppm 11.16 (s, 1H), 8.84 (d, J = 4.5 Hz, 1H), 8.15-8.10 (m, 2H), 7.83-7.80 (m, 1H), 7.72-7.68 (m, 1H), 7.60-7.56 (m, 1H), 7.48-7.46 (m, 2H), 4.53-4.51 (m, 1H), 3.98-3.95 (m, 1H), 3.78-3.73 (m, 1H), 3.50-3.45 (m, 1H), 3.12-3.06 (m, 1H), 2.01-1.93 |
| | | | (m, 2H), 1.79-1.67 (m, 2H). |
| | | | MS (ESI): m/z 430.1 [M + H]⁺. |
| 1/19 | Int 10/9 Int 20 | 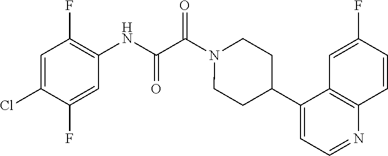 [SMILES: O=C(Nc1cc(F)c(Cl)cc1F)C(=O)N1CCC(c2ccnc3ccc(F)cc23)CC1] | ¹H NMR (500 MHz, DMSO- d₆): δ ppm 11.00 (s, 1H), 8.84 (d, J = 4.5 Hz, 1H), 8.18-7.98 (m, 3H), 7.81-7.65 (m, 2H), 7.46 (d, J = 4.4 Hz, 1H), 4.51-4.49 (m, 1H), 3.93-3.90 (m, 1H), 3.77-3.72 (m, 1H), 3.50-3.44 (m, 1H), 3.09-3.04 (m, 1H), 1.98-1.91 (m, 2H), 1.85-1.63 (m, 2H). |
| | | | MS (ESI): m/z 448.1 [M + H]⁺. |
| 1/20 | Int 10/10 Int 20 | 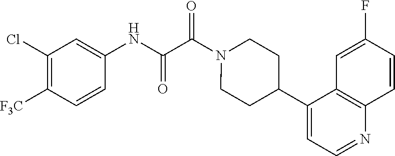 [SMILES: O=C(Nc1ccc(C(F)(F)F)c(Cl)c1)C(=O)N1CCC(c2ccnc3ccc(F)cc23)CC1] | ¹H NMR (500 MHz, DMSO- d₆): δ ppm 11.37 (s, 1H), 8.84 (d, J = 4.5 Hz, 1H), 8.19-8.01 (m, 3H), 7.89-7.63 (m, 3H), 7.47 (d, J = 4.5 Hz, 1H), 4.54-4.51 (m, 1H), 3.98-3.95 (m, 1H), 3.78-3.74 (m, 1H), 3.51-3.46 (m, 1H), 3.13-3.07 (m, 1H), 2.01-1.92 (m, 2H), 1.79-1.66 (m, 2H). |
| | | | MS (ESI): m/z 480.2 [M + H]⁺. |
| 1/21 | Int 10/11 Int 20 | 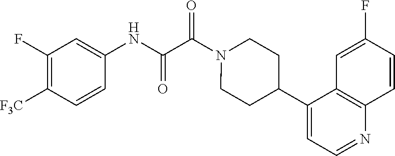 [SMILES: O=C(Nc1ccc(C(F)(F)F)c(F)c1)C(=O)N1CCC(c2ccnc3ccc(F)cc23)CC1] | ¹H NMR (500 MHz, DMSO- d₆): δ ppm 11.43 (s, 1H), 8.84 (d, J = 4.5 Hz, 1H), 8.14-8.10 (m, 2H), 7.86-7.68 (m, 4H), 7.60-7.59 (m, 1H), 7.47-7.46 (m, 1H), 4.54-4.51 (m, 1H), 3.97-3.95 (m, 1H), 3.78-3.73 (m, 1H), 3.50-3.45 (m, 1H), 3.12-3.06 (m, 1H), 2.01-1.92 (m, 2H), 1.81-1.65 |
| | | | (m, 2H). MS (ESI): m/z |
| | | | 464.2 [M + H]⁺. |
| 1/22 | Int 10/12 Int 20 | 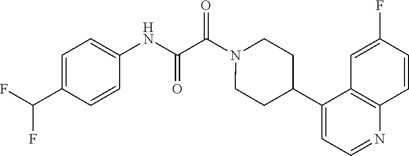 [SMILES: O=C(Nc1ccc(C(F)F)cc1)C(=O)N1CCC(c2ccnc3ccc(F)cc23)CC1] | ¹H NMR (500 MHz, DMSO- d₆): δ ppm 11.05 (br s, 1H), 8.85 (d, J = 4.5 Hz, 1H), 8.15-8.10 (m, 2H), 7.81-7.79 (m, 2H), 7.72-7.68 (m, 1H), 7.58-7.56 (m, 2H), 7.47-7.46 (m, 1H), 7.00 (t, J = 56.0 Hz, 1H), 4.54-4.52 (m, 1H), 3.95-3.92 (m, 1H), 3.78-3.73 (m, 1H), 3.50-3.48 (m, 1H), |
| | | | 3.10-3.08 (m, 1H), 2.00-1.92 |
| | | | (m, 1H), 1.80-1.68 (m, 1H). |
| | | | MS (ESI): m/z 428.2 [M + H]⁺. |
| 1/23 | Int 10/13 Int 20 | 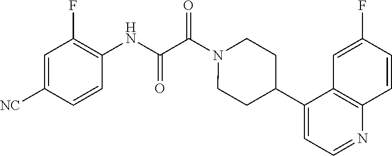 [SMILES: N#Cc1ccc(NC(=O)C(=O)N2CCC(c3ccnc4ccc(F)cc34)CC2)c(F)c1] | ¹H NMR (500 MHz, DMSO- d₆): δ ppm 11.12 (s, 1H), 8.84 (d, J = 4.5 Hz, 1H), 8.22-8.10 (m, 3H), 7.99-7.96 (m, 1H), 7.74-7.68 (m, 2H), 7.47-7.46 (m, 1H), 4.51-4.49 (m, 1H), 3.90-3.88 (m, 1H), 3.77-3.72 (m, 1H), 3.51-3.46 (m, 1H), 3.09-3.05 (m 1H), 1.99-1.91 (m, 2H), 1.83-1.71 |
| | | | (m, 2H). MS (ESI): m/z |
| | | | 421.2 [M + H]⁺. |
| 1/24 | Int 10/14 Int 20 | 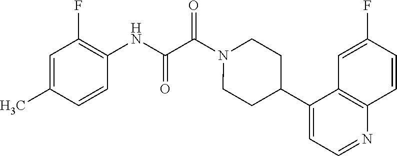 [SMILES: Cc1ccc(NC(=O)C(=O)N2CCC(c3ccnc4ccc(F)cc34)CC2)c(F)c1] | ¹H NMR (500 MHz, DMSO- d₆): δ ppm 10.55 (s, 1H), 8.84 (d, J = 4.5 Hz, 1H), 8.16-8.10 (m, 2H), 7.72-7.68 (m, 1H), 7.63-7.60 (m, 1H), 7.47-7.46 (m, 1H), 7.03-7.02 (m, 1H), 4.53-4.50 (m, 1H), 3.96-3.94 (m, 1H), 3.77-3.72 (m, 1H), 3.51-3.46 (m, 1H), 3.08-3.03 (m, 1H), 2.31 (s, |
| | | | 3H), 1.98-1.93 (m, 2H), |
| | | | 1.84-1.70 (m, 2H). MS (ESI): |
| | | | m/z 410.2 [M + H]⁺. |
| 1/25 | Int 10/15 Int 20 | 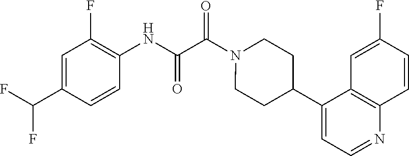 [SMILES: O=C(Nc1ccc(C(F)F)cc1F)C(=O)N1CCC(c2ccnc3ccc(F)cc23)CC1] | ¹H NMR (500 MHz, DMSO- d₆): δ ppm 10.90 (s, 1H), 8.85 (d, J = 4.5 Hz, 1H), 8.16-8.10 (m, 2H), 8.05-8.02 (m, 1H), 7.72-7.68 (m, 1H), 7.57-7.55 (m, 1H), 7.47-7.45 (m, 2H), 7.04 (t, J = 55.7 Hz, 1H), 4.53-4.50 (m, 1H), 3.94-3.92 (m, 1H), 3.78-3.73 (m, 1H), 3.52-3.47 (m, 1H), |
| | | | 3.10-3.05 (m, 1H), 1.99- |
| | | | 1.93 (m, 2H), 1.84-1.71 (m, |
| | | | 2H). MS (ESI): m/z 446.2 |
| | | | [M + H]⁺. |
| 1/26 | Int 10/16 Int 20 | 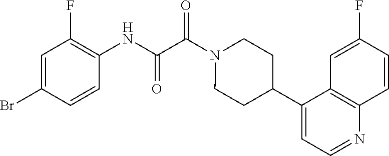 [SMILES: O=C(Nc1ccc(Br)cc1F)C(=O)N1CCC(c2ccnc3ccc(F)cc23)CC1] | ¹H NMR (500 MHz, DMSO- d₆): δ ppm 10.79 (s, 1H), 8.84 (d, J = 4.5 Hz, 1H), 8.15-8.10 (m, 2H), 7.82- 7.78 (m, 1H), 7.72-7.65 (m, 2H), 7.47-7.43 (m, 2H), 4.52-4.49 (m, 1H), 3.94-3.91 (m, 1H), 3.77-3.72 (m, 1H), 3.51-3.46 (m, 1H), 3.09-3.04 (m, 1H), 1.98-1.92 (m, 1H), |
| | | | 1.85-1.70 (m, 1H). MS (ESI): |
| | | | m/z 474.1 [M + H]⁺. |
| 1/27 | Int 10/17 Int 20 | 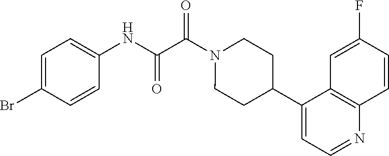 [SMILES: O=C(Nc1ccc(Br)cc1)C(=O)N1CCC(c2ccnc3ccc(F)cc23)CC1] | ¹H NMR (500 MHz, DMSO- d₆): δ ppm 10.96 (s, 1H), 8.84 (d, J = 4.5 Hz, 1H), 8.14-8.10 (m, 1H), 7.72-7.64 (m, 3H), 7.57-7.54 (m, 2H), 7.47-7.46 (m, 1H), 4.54-4.51 (m, 1H), 3.95-3.92 (m, 1H), 3.78-3.73 (m, 1H), 3.50-3.45 (m, 1H), 3.11-3.05 (m, 1H), 2.00-1.92 (m, 2H), 1.79-1.67 |
| | | | (m, 2H). MS (ESI): m/z |
| | | | 456.1 [M + H]⁺. |
| 1/28 | Int 10/18 Int 20 | 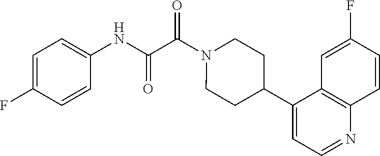 [SMILES: O=C(Nc1ccc(F)cc1)C(=O)N1CCC(c2ccnc3ccc(F)cc23)CC1] | ¹H NMR (500 MHz, DMSO- d₆): δ ppm 10.88 (s, 1H), 8.85 (d, J = 4.5 Hz, 1H), 8.15-8.10 (m, 2H), 7.72-7.68 (m, 3H), 7.47-7.46 (m, 1H), 7.23-7.19 (m, 2H), 4.55-4.52 (m, 1H), 3.97-3.95 (m, 1H), 3.78-3.73 (m, 1H), 3.51-3.45 (m, 1H), 3.11-3.05 (m, 1H), 2.01-1.93 (m, 1H), 1.80-1.68 |
| | | | (m, 1H). MS (ESI): m/z |
| | | | 396.3 [M + H]⁺. |
| 1/29 | Int 10/19 Int 20 | 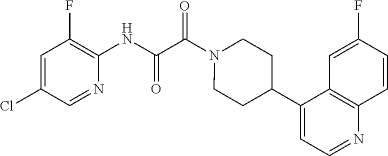 [SMILES: O=C(Nc1ncc(Cl)cc1F)C(=O)N1CCC(c2ccnc3ccc(F)cc23)CC1] | ¹H NMR (500 MHz, DMSO- d₆): δ ppm 11.28 (s, 1H), 8.84 (d, J = 4.5 Hz, 1H), 8.39 (br s, 1H), 8.21-8.20 (m, 1H), 8.15-8.10 (m, 3H), 7.72-7.68 (m, 1H), 7.48-7.47 (m, 1H), 4.49-4.47 (m, 1H), 3.91-3.88 (m, 1H), 3.77-3.72 (m, 1H), 3.54-3.49 (m, 1H), 3.09-3.04 (m, 1H), 1.98-1.96 |
| | | | (m, 2H), 1.83-1.69 (m, 2H). |
| | | | MS (ESI): m/z 431.2 [M + H]⁺. |
| 1/30 | Int 10/1 Int 20/13 | 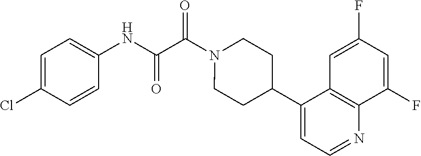 [SMILES: O=C(Nc1ccc(Cl)cc1)C(=O)N1CCC(c2ccnc3c(F)cc(F)cc23)CC1] | ¹H NMR (500 MHz, DMSO- d₆): δ ppm 10.96 (s, 1H), 8.88 (d, J = 4.5 Hz, 1H), 8.04-8-02 (m, 1H), 7.78-7.69 (m, 3H), 7.58-7.57 (m, 1H), 7.44-7.41 (m, 2H), 4.54-4.51 (m, 1H), 3.95-3.93 (m, 1H), 3.77-3.72 (m, 1H), 3.50-3.44 (m, 1H), 3.10-3.05 (m, 1H), 2.00-1.92 (m, 2H), 1.81-1.67 |
| | | | (m, 2H). MS (ESI): m/z |
| | | | 430.2 [M + H]⁺. |
| 1/31 | Int 10/1 Int 20/14 | 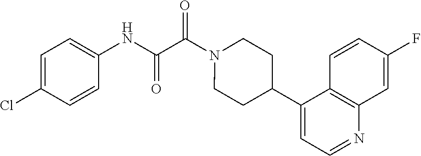 [SMILES: O=C(Nc1ccc(Cl)cc1)C(=O)N1CCC(c2ccnc3cc(F)ccc23)CC1] | ¹H NMR (500 MHz, DMSO- d₆): δ ppm 10.96 (s, 1H), 8.87 (d, J = 4.6 Hz, 1H), 8.46-8.43 (m, 1H), 7.79-7.69 (m, 3H), 7.62-7.58 (m, 1H), 7.43-7.41 (m, 3H), 4.54-4.52 (m, 1H), 3.96-3.93 (m, 1H), 3.86-3.81 (m, 1H), 3.49-3.44 (m, 1H), 3.09-3.04 (m, 1H), 2.01-1.93 (m, 2H), 1.83-1.69 |
| | | | (m, 2H). MS (ESI): m/z |
| | | | 412.2 [M + H]⁺. |
| 1/32 | Int 10/20 Int 20 | 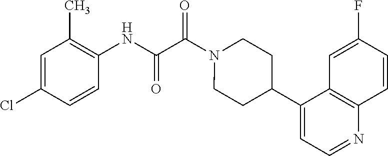 [SMILES: Cc1cc(Cl)ccc1NC(=O)C(=O)N1CCC(c2ccnc3ccc(F)cc23)CC1] | ¹H NMR (500 MHz, DMSO- d₆): δ ppm 10.32 (s, 1H), 8.84 (d, J = 4.5 Hz, 1H), 8.15-8.10 (m, 2H), 7.72-7.68 (m, 1H), 7.46-7.45 (m, 2H), 7.36 (s, 1H), 7.28-7.26 (m, 1H), 4.54-4.52 (m, 1H), 3.99-3.97 (m, 1H), 3.76-3.74 (m, 1H), 3.52-3.47 (m, 1H), 3.09-3.04 (m, 1H), 2.25 (s, |
| | | | 3H), 1.82-1.79 (m, 2H), |
| | | | 1.73-1.70 (m, 2H). MS (ESI): |
| | | | m/z 426.2 [M + H]⁺. |
| 1/33 | Int 10/3 Int 20/4 | 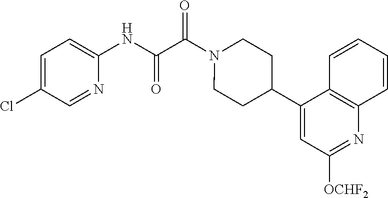 [SMILES: O=C(Nc1ccc(Cl)cn1)C(=O)N1CCC(c2cc(OC(F)F)nc3ccccc23)CC1] | ¹H NMR (500 MHz, DMSO- d₆): δ ppm 11.43 (s, 1H), 8.44-8.31 (m, 2H), 8.12-8.10 (m, 1H), 8.05-7.76 (m, 4H), 7.64-7.61 (m, 1H), 7.12 (s, 1H), 4.49-4.46 (m, 1H), 3.81-3.79 (m, 2H), 3.49- 3.44 (m, 1H), 3.05-3.00 (m, 1H), 1.97-1.76 (m, 4H), MS (ESI): m/z 461.0 [M + H]⁺. |
| 1/34 | Int 10/3 Int 20/3 | 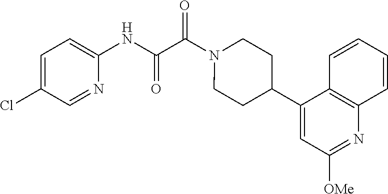 [SMILES: COc1cc(C2CCN(C(=O)C(=O)Nc3ccc(Cl)cn3)CC2)c2ccccc2n1] | ¹H NMR (500 MHz, DMSO- d₆): δ ppm 11.43 (s, 1H), 8.43 (s, 1H), 8.18 (d, J = 8.5 Hz, 1H), 8.13-8.09 (m, 1H), 7.97-7.95 (m, 1H), 7.80 (d, J = 8.0 Hz, 1H), 7.68-7.65 (m, 1H), 7.49-7.46 (m, 1H), 6.88 (s, 1H), 4.48-4.45 (m, 1H), 3.97 (s, 3H), 3.80-3.68 (m, 2H), 3.47-3.42 (m, 1H), 3.04-2.98 (m, 1H), 1.96-1.72 (m, 4H). MS (ESI): m/z |
| | | | 425.0 [M + H]⁺. |
| 1/35 | Int 10/3 Int 22 | 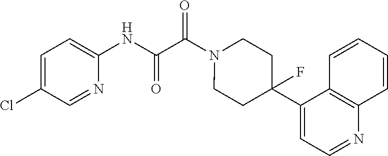 [SMILES: O=C(Nc1ccc(Cl)cn1)C(=O)N1CCC(F)(c2ccnc3ccccc23)CC1] | ¹H NMR (500 MHz, CD₃OD): δ ppm 9.12-9.10 (m, 1H), 8.75-8.73 (m, 1H), 8.36 (s, 1H), 8.24-8.20 (m, 2H), 8.08-7.86 (m, 4H), 4.72-4.68 (m, 1H), 4.34-4.32 (m, 1H), 3.85-3.78 (m, 1H), 3.46-3.40 (m, 1H), 2.72-2.46 (m, 4H). MS (ESI): m/z 413.1 [M + H]⁺. |
| 1/36 | Int 10/22 Int 20 | 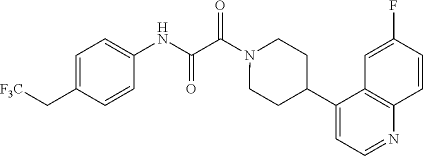 [SMILES: O=C(Nc1ccc(CC(F)(F)F)cc1)C(=O)N1CCC(c2ccnc3ccc(F)cc23)CC1] | ¹H NMR (500 MHz, DMSO- d₆): δ ppm 10.88 (s, 1H), 8.85-8.84 (m, 1H), 8.15-8.10 (m, 2H), 7.72-7.67 (m, 3H), 7.47-7.46 (m, 1H), 7.36-7.34 (m, 2H), 4.54-4.52 (m, 1H), 3.96-3.93 (m, 1H), 3.76-3.74 (m, 1H), 3.64-3.57 (m, 2H), 3.50-3.45 (m, 1H), 3.10-3-06 (m, 1H), 2.00-1.68 (m, 4H). |
| | | | MS (ESI): m/z 460.2 [M + H]⁺. |
| 1/37 | Int 10/23 Int 20 | 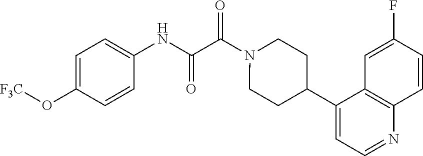 [SMILES: O=C(Nc1ccc(OC(F)(F)F)cc1)C(=O)N1CCC(c2ccnc3ccc(F)cc23)CC1] | ¹H NMR (500 MHz, DMSO- d₆): δ ppm 11.03 (s, 1H), 8.85-8.84 (m, 1H), 8.15-8.10 (m, 2H), 7.80-7.77 (m, 2H), 7.71-7.68 (m, 1H), 7.47-7.46 (m, 1H), 7.39-7.37 (m, 2H), 4.54-4.51 (m, 1H), 3.96-3.93 (m, 1H), 3.78-3.73 (m, 1H), 3.51-3.45 (m, 1H), 3.11-3-06 (m, 1H), 2.00-1.68 (m, 4H). |
| | | | MS (ESI): m/z 462.2 [M + H]⁺. |
| 1/38 | Int 10/24 Int 20 | 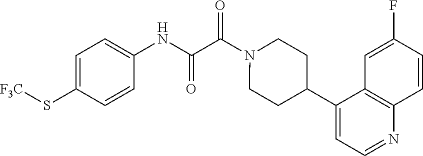 [SMILES: O=C(Nc1ccc(SC(F)(F)F)cc1)C(=O)N1CCC(c2ccnc3ccc(F)cc23)CC1] | ¹H NMR (500 MHz, DMSO- d₆): δ ppm 11.17 (s, 1H), 8.85-8.84 (m, 1H), 8.15-8.10 (m, 2H), 7.85-7.84 (m, 2H), 7.74-7.68 (m, 3H), 7.47-7.46 (m, 1H), 4.55-4.52 (m, 1H), 3.96-3.93 (m, 1H), 3.78-3.74 (m, 1H), 3.51-3.46 (m, 1H), 3.12-3-07 (m, 1H), 2.01-1.69 (m, 4H). MS (ESI): m/z |
| | | | 478.2 [M + H]⁺. |
| 1/39 | Int 10/27 Int 20 | 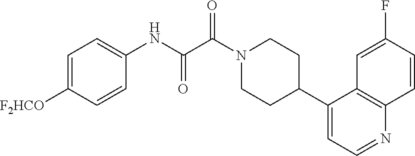 [SMILES: O=C(Nc1ccc(OC(F)F)cc1)C(=O)N1CCC(c2ccnc3ccc(F)cc23)CC1] | ¹H NMR (500 MHz, DMSO- d₆): δ ppm 10.91 (s, 1H), 8.85-8.84 (m, 1H), 8.15-8.10 (m, 2H), 7.72-7.68 (m, 3H), 7.47-7.46 (m, 1H), 7.28-7.27 (m, 1H), 7.20-7.19 (m, 2H), 7.18 (t, J = 74.0 Hz, 1H), 4.54-4.51 (m, 1H), 3.96-3.93 (m, 1H), 3.78-3.73 (m, 1H), 3.50-3.45 (m, 1H), 3.10-3-05 |
| | | | (m, 1H), 2.00-1.67 (m, 4H). |
| | | | MS (ESI): m/z 442.1 [M − H]⁻. |
| 1/40 | Int 10/28 Int 20 | 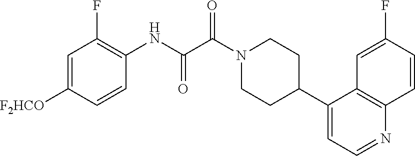 [SMILES: O=C(Nc1ccc(OC(F)F)cc1F)C(=O)N1CCC(c2ccnc3ccc(F)cc23)CC1] | ¹H NMR (500 MHz, DMSO- d₆): δ ppm 10.73 (s, 1H), 8.85-8.84 (m, 1H), 8.15-8.10 (m, 2H), 7.81-7.68 (m, 2H), 7.47-7.46 (m, 1H), 7.29 (t, J = 75.5 Hz, 1H), 7.28-7.27 (m, 1H), 7.09-7.07 (m, 1H), 4.53-4.51 (m, 1H), 3.96-3.93 (m, 1H), 3.78-3.73 (m, 1H), 3.52-3.46 (m, 1H), 3.10-3-05 |
| | | | (m, 1H), 1.99-1.69 (m, 4H). |
| | | | MS (ESI): m/z 460.0 [M − H]⁻. |
| 1/41 | Int 10/29 Int 20 | 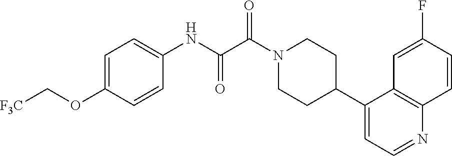 [SMILES: O=C(Nc1ccc(OCC(F)(F)F)cc1)C(=O)N1CCC(c2ccnc3ccc(F)cc23)CC1] | ¹H NMR (500 MHz, DMSO- d₆) δ: 10.77 (s, 1H), 8.85 (d, J = 4.5 Hz, 1H), 8.19-8.05 (m, 2H), 7.77-7.57 (m, 3H), 7.47 (d, J = 4.6 Hz, 1H), 7.12-6.97 (m, 2H), 4.74 (q, J = 8.9 Hz, 2H), 4.55-4.51 (m, 1H), 3.98-3.94 (m, 1H), 3.76 (t, J = 11.9 Hz, 1H), 3.61- 3.41 (m, 1H), 3.10-3.05 (m, |
| | | | 1H), 2.01-1.92 (m, 2H), |
| | | | 1.81-1.66 (m, 2H). MS (ESI): |
| | | | m/z 474.1 [M − H]⁻. |
| 1/42 | Int 10/1 Int 24 | 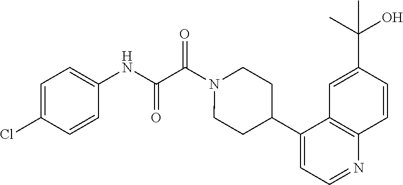 [SMILES: CC(C)(O)c1ccc2nccc(C3CCN(C(=O)C(=O)Nc4ccc(Cl)cc4)CC3)c2c1] | ¹H NMR (400 MHz, DMSO- d₆): δ ppm 10.99 (s, 1H), 8.80 (d, J = 4.5 Hz, 1H), 8.29 (s, 1H), 7.98 (d, J = 8.8 Hz, 1H), 7.87 (dd, J = 8.8, 1.6 Hz, 1H), 7.72 (d, J = 8.9 Hz, 2H), 7.44-7.39 (m, 3H), 5.32 (s, 1H), 4.57-4.52 (m, 1H), 3.99-3.95 (m, 1H), 3.80-3.77 (m, 1H), 3.52-3.46 (m, 1H), 3.12-3.06 (m, 1H), 2.05-1.96 (m, 2H), 1.89-1.62 (m, 2H), |
| | | | 1.55 (s, 6H). MS (ESI): m/z |
| | | | 452.2 [M + H]⁺. |
| 1/43 | Int 10/1 Int 25 | 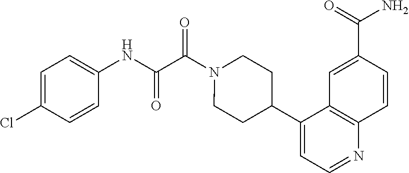 [SMILES: NC(=O)c1ccc2nccc(C3CCN(C(=O)C(=O)Nc4ccc(Cl)cc4)CC3)c2c1] | ¹H NMR (400 MHz, DMSO- d₆): δ ppm 11.00 (s, 1H), 9.04 (d, J = 4.7 Hz, 1H), 8.85 (s, 1H), 8.41 (s, 1H), 8.30 (d, J = 8.8 Hz, 1H), 8.16 (d, J = 8.8 Hz, 1H), 7.74-7.66 (m, 4H), 7.44-7.42 (m, 2H), 4.59-4.55 (m, 1H), 4.02-3.96 (m, 2H), 3.53-3.47 (m, 1H), 3.13-3.07 (m, 1H), 2.05-1.97 (m, 2H), 1.92-1.63 (m, 2H). |
| | | | MS (ESI): m/z 437.2 [M + H]⁺. |
| 1/44 | Int 10/1 Int 26 | 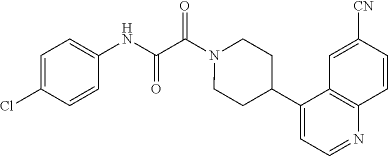 [SMILES: N#Cc1ccc2nccc(C3CCN(C(=O)C(=O)Nc4ccc(Cl)cc4)CC3)c2c1] | ¹H NMR (500 MHz, DMSO- d₆): δ ppm 10.98 (s, 1H), 9.05-9.01 (m, 2H), 8.19 (d, J = 8.7 Hz, 1H), 8.08 (dd, J = 8.7, 1.5 Hz, 1H), 7.71 (d, J = 8.9 Hz, 2H), 7.60 (d, J = 4.6 Hz, 1H), 7.43 (d, J = 8.9 Hz, 2H), 4.55-4.51 (m, 1H), 3.97-3.90 (m, 2H), 3.52-3.45 (m, 1H), 3.12-3.06 (m, 1H), |
| | | | 2.01-1.93 (m, 2H), 1.84-1.66 |
| | | | (m, 2H). MS (ESI): m/z |
| | | | 419.1 [M + H]⁺. |
| 1/45 | Int 10/1 Int 23/2 | 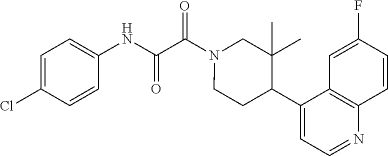 [SMILES: CC1(C)CN(C(=O)C(=O)Nc2ccc(Cl)cc2)CCC1c1ccnc2ccc(F)cc12] | ¹H NMR (500 MHz, DMSO- d₆): δ ppm 10.99 (s, 1H), 8.85 (t, J = 4.9 Hz, 1H), 8.30 (ddd, J = 11.5, 4.6, 2.9 Hz, 1H), 8.10 (dd, J = 9.1, 5.9 Hz, 1H), 7.83-7.57 (m, 3H), 7.51-7.23 (m, 3H), 4.58 (d, J = 13.1 Hz, 0.5H), 4.17 (d, J = 11.7 Hz, 0.5H), 4.02 (d, J = 13.2 Hz, 0.5H), 3.89-3.76 |
| | | | (m, 1H), 3.56-3.45 (m, 1H), |
| | | | 3.32 (d, J = 2.7 Hz, 0.5H), |
| | | | 3.04 (d, J = 12.6 Hz, 0.5H), |
| | | | 2.96 (dd, J = 12.8, 9.8 Hz, |
| | | | 0.5H), 2.35-2.11 (m, 1H), |
| | | | 1.66-1.58 (m, 1H), 0.92 (m, |
| | | | 1.5 H), 0.89 (m, 1.5 H), 0.60 |
| | | | (m, 1.5 H), 0.60 (m, 1.5 H). |
| | | | MS (ESI): m/z 440.2 [M + H]⁺. |
| 1/46 | Int 10/1 Int 28 | 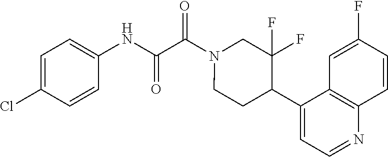 [SMILES: O=C(Nc1ccc(Cl)cc1)C(=O)N1CCC(c2ccnc3ccc(F)cc23)C(F)(F)C1] | ¹H NMR (500 MHz, DMSO- d₆): δ ppm 11.10-11-05 (m, 1H), 8.91 (d, J = 4.6 Hz, 1H), 8.27-8.04 (m, 2H), 7.82-7.66 (m, 3H), 7.64-7.53 (m, 1H), 7.49-7.30 (m, 2H), 4.80-4.50 (m, 2H), 4.43-4.35 (s, 0.5H), 4.19-3.97 (m, 1H), 3.72-3.44 (m, 1H), 3.16 (t, J = 11.5 Hz, 0.5H), 2.30-2.00 (m, 2H). |
| | | | MS (ESI): m/z 448.1 [M + H]⁺. |
| 1/47 | Int 10/1 Int 20/16 | 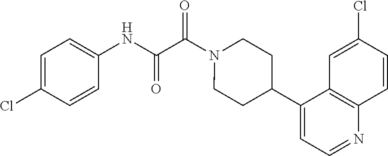 [SMILES: O=C(Nc1ccc(Cl)cc1)C(=O)N1CCC(c2ccnc3ccc(Cl)cc23)CC1] | ¹H NMR (500 MHz, DMSO- d₆): δ ppm 10.96 (s, 1H), 8.88 (d, J = 4.6 Hz, 1H), 8.43 (d, J = 2.1 Hz, 1H), 8.07 (d, J = 9.0 Hz, 1H), 7.79 (dd, J = 8.9, 2.2 Hz, 1H), 7.71 (d, J = 8.9 Hz, 2H), 7.49 (d, J = 4.6 Hz, 1H), 7.43 (d, J = 8.9 Hz, 2H), 4.56-4.49 (m, 1H), 3.97-3.90 (m, 1H), 3.88-3.80 |
| | | | (m, 1H), 3.49-3.44 (m, 1H), |
| | | | 3.16-3.03 (m, 1H), 2.00-1.89 |
| | | | (m, 2H), 1.82-1.65 (m, 2H). |
| | | | MS (ESI): m/z 428.1 [M + H]⁺. |
| 1/48 | Int 10/1 Int 20/17 | 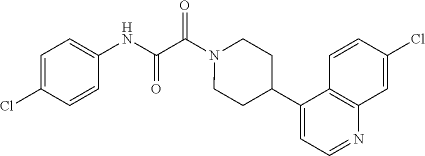 [SMILES: O=C(Nc1ccc(Cl)cc1)C(=O)N1CCC(c2ccnc3cc(Cl)ccc23)CC1] | ¹H NMR (400 MHz, DMSO- d₆): δ ppm 10.97 (s, 1H), 8.89 (d, J = 4.6 Hz, 1H), 8.39 (d, J = 9.2 Hz, 1H), 8.10 (d, J = 2.2 Hz, 1H), 7.76-7.63 (m, 3H), 7.48-7.41 (m, 3H), 4.56-7.49 (m, 1H), 3.98-3.91 (m, 1H), 3.85-3.78 (m, 1H), 3.51-3.41 (m, 1H), 3.11-3.01 (m, 1H), 2.05-1.88 (m, 2H), |
| | | | 1.87-1.58 (m, 2H). MS (ESI): |
| | | | m/z 428.1 [M + H]⁺. |

### Example 2: N-(5-Chloropyridin-2-yl)-2-(4-(6-fluoroquinolin-4-yl)piperidin-1-yl)-2-oxoacetamide (2)

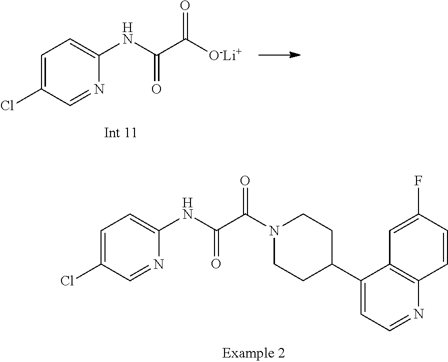

A mixture of lithium 2-((5-chloropyridin-2-yl)(methyl)amino)-2-oxoacetate (Int 11) (100 mg, 0.5 mmol), 6-fluoro-4-(piperidin-4-yl)quinoline (Int 20) (122 mg, 0.5 mmol), HATU (225 mg, 0.6 mmol) and DIPEA (194 mg, 1.5 mmol) in DMF (10 mL) was stirred at rt overnight. The mixture was concentrated to dryness and the residue was purified by preparative HPLC to afford the title compound as a white solid. ¹H NMR (DMSO-d₆, 400 MHz): δ ppm 10.44 (br s, 1H), 8.85 (d, J=4.4 Hz, 1H), 8.44 (s, 1H), 8.16-8.10 (m, 3H), 7.98-7.96 (m, 1H), 7.72-7.67 (m, 1H), 7.47 (d, J=4.4 Hz, 1H), 4.50-4.46 (m, 1H), 3.82-3.71 (m, 2H), 3.51-3.45 (m, 1H), 3.08-3.02 (m, 1H), 1.97-1.72 (m, 4H). MS (ESI): m/z 413.0 [M+H]⁺.

### Examples 2/1 to 2/6

The following Examples were prepared similar as described for Example 2 using the appropriate building blocks.

| | Building | | |
|---|---|---|---|
| # | blocks | Structure | Analytical data |
| 2/1 | Int 11/1 Int 20 | 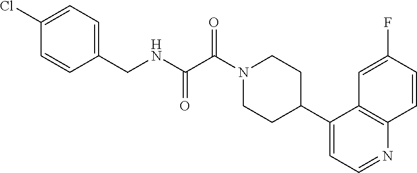 [SMILES: O=C(NCc1ccc(Cl)cc1)C(=O)N1CCC(c2ccnc3ccc(F)cc23)CC1] | ¹H NMR (DMSO-d₆, 400 MHz): δ ppm 9.28 (t, J = 6.0 Hz, 1H), 8.94 (d, J = 4.8 Hz, 1H), 8.23 (dd, J = 10.8, 2.8 Hz, 1H), 8.17 (dd, J = 9.2, 5.6 Hz, 1H), 7.79 (dd, J = 8.8, 2.8 Hz, 1H), 7.58 (d, J = 4.8 Hz, 1H), 7.42-7.40 (m, 2H), 7.33-7.31 (m, 2H), 4.50-4.47 (m, 1H), 4.35 (d, J = 6.0 Hz, 2H), 3.91-3.78 (m, 1H), 3.43-3.37 (m, 1H), 3.03-2.97 |
| | | | (m, 1H), 1.96-1.89 (m, 2H), |
| | | | 1.77-1.65 (m, 2H). MS (ESI): |
| | | | m/z 426.0 [M + H]⁺. |
| 2/2 | Int 11/2 Int 20 | 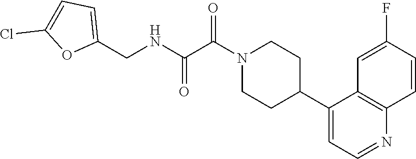 [SMILES: O=C(NCc1ccc(Cl)o1)C(=O)N1CCC(c2ccnc3ccc(F)cc23)CC1] | ¹H NMR (DMSO-d₆, 400 MHz): δ ppm 9.26 (t, J = 5.2 Hz, 1H), 8.84 (d, J = 4.4 Hz, 1H), 8.15- 8.09 (m, 2H), 7.72-7.68 (m, 1H), 7.44 (d, J = 4.4 Hz, 1H), 6.42-6.40 (m, 2H), 4.47-4.34 (m, 1H), 4.35 (d, J = 5.6 Hz, 2H), 3.85-3.82 (m, 1H), 3.75- 3.69 (m, 1H), 3.43-3.40 (m, 1H), 3.02-2.96 (m, 1H), 1.95- |
| | | | 1.87 (m, 2H), 1.80-1.62 (m, |
| | | | 2H). MS (ESI): m/z 416.0 |
| | | | [M + H]⁺. |
| 2/3 | Int 12 Int 20 | 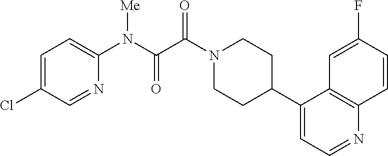 [SMILES: CN(C(=O)C(=O)N1CCC(c2ccnc3ccc(F)cc23)CC1)c1ccc(Cl)cn1] | ¹H NMR (DMSO-d₆, 400 MHz): δ ppm 8.85 (d, J = 4.4 Hz, 1H), 8.45 (s, 1H), 8.14-8.04 (m, 3H), 7.72-7.67 (m, 1H), 7.48-7.45 (m, 2H), 4.34-4.31 (m, 1H), 3.76-3.66 (m, 2H), 3.52-3.48 (m, 1H), 3.38 (s, 3H), 2.99- 2.94 (m, 1H), 1.94-1.86 (m, 2H), 1.66-1.58 (m, 2H). MS (ESI): m/z 427.0 [M + H]⁺. |
| 2/4 | Int 11 Int 20/6 | 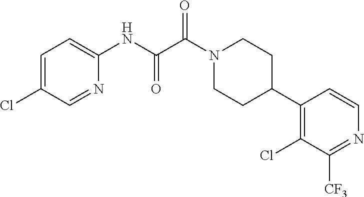 [SMILES: O=C(Nc1ccc(Cl)cn1)C(=O)N1CCC(c2ccnc(C(F)(F)F)c2Cl)CC1] | ¹H NMR (CDCl₃, 400 MHz): δ ppm 9.60 (s, 1H), 8.56-8.54 (m, , 1H), 8.32 (s, 1H), 8.20-8.17 (m, 1H), 7.73-7.71 (m, 1H), 7.39-7.38 (m, 1H), 5.29-5.26 (m, 1H), 4.85-4.81 (m, 1H), 3.53-3.47 (m, 1H), 3.32-3.26 (m, 1H), 2.97-2.90 (m, 1H), 2.08-2.02 (m, 2H), 1.83-1.68 (m, 2H). MS (ESI): m/z 446.9 [M + H]⁺. |
| 2/5 | Int 11, 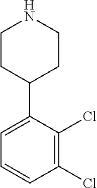 [SMILES: Clc1cccc(C2CCNCC2)c1Cl] | 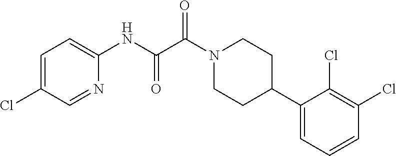 [SMILES: O=C(Nc1ccc(Cl)cn1)C(=O)N1CCC(c2cccc(Cl)c2Cl)CC1] | ¹H NMR (400 MHz, DMSO-d₆): δ ppm 11.43 (s, 1H), 8.43 (s, 1H), 8.12-8.09 (m, 1H), 7.98- 7.95 (m, 1H), 7.55-7.51 (m, 1H), 7.39-7.36 (m, 2H), 4.46- 4.43 (m, 1H), 3.79-3.75 (m, 1H), 3.40-3.29 (m, 2H), 2.91- 2.84 (m, 1H), 1.74-1.61 (m, 4H). MS (ESI): m/z 411.9 [M + H]⁺. |
| 2/6 | Int 11 Int 20/5 | 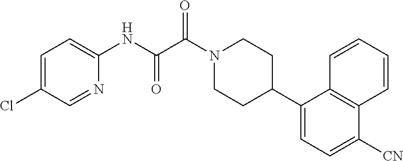 [SMILES: N#Cc1ccc(C2CCN(C(=O)C(=O)Nc3ccc(Cl)cn3)CC2)c2ccccc12] | ¹H NMR (400 MHz, DMSO-d₆): δ ppm 10.71 (s br, 1H), 8.50- 8.44 (m, 2H), 8.18-8.11 (m, 2H), 7.98-7.96 (m, 1H), 7.85- 7.77 (m, 2H), 7.62-7.60 (m, 1H), 4.51-4.47 (m, 1H), 3.91- 3.80 (2H), 3.52-3.39 (m, 1H), 3.09-3.03 (m, 1 H), 1.98-1.74 (m, 4H). MS (ESI): m/z 419.0 [M + H]⁺. |

### Example 3: N-(5-Chloropyridin-2-yl)-2-oxo-2-(4-(quinolin-4-yl)piperidin-1-yl)acetamide (3)

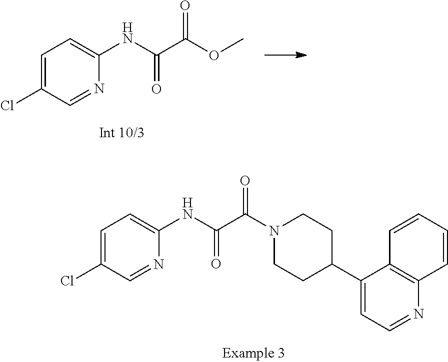 [SMILES: COC(=O)C(=O)Nc1ccc(Cl)cn1 ; O=C(Nc1ccc(Cl)cn1)C(=O)N1CCC(c2ccnc3ccccc23)CC1]

To a solution of methyl 2-((5-chloropyridin-2-yl)amino)-2-oxoacetate (Int 10/3) (200 mg, 0.93 mmol, 1.0 eq.) in THF/H₂O (5 mL, v/v=4:1) was added LiOH.H₂O (79 mg, 1.87 mmol, 2.0 eq.). The mixture was stirred at rt overnight and was evaporated to dryness to afford 300 mg of a white solid. The residue (150 mg, 0.75 mmol, 1.0 eq.) was dissolved in DMF (5 mL). 4-(Piperidin-4-yl)quinoline hydrochloride (129 mg, 0.75 mmol, 1.0 eq.), HATU (205 mg, 0.54 mmol, 1.2 eq.) and DIPEA (117 mg, 0.90 mmol, 2.0 eq.) were added and the mixture was stirred at rt for 2 h. The reaction mixture was concentrated in vacuo. The residue was purified by prep-HPLC to afford the title compound as a white solid. ¹H NMR (CD₃OD, 400 MHz): δ ppm 8.80-8.78 (m, 1H), 8.34-8.32 (m, 2H), 8.19-8.17 (m, 1H), 8.06-8.04 (m, 1H), 7.83-7.77 (m, 2H), 7.70-7.66 (m, 1H), 7.48-7.46 (m, 1H), 4.73-4.69 (m, 1H), 4.26-4.23 (m, 1H), 3.89-3.83 (m, 1H), 3.55-3.49 (m, 1H), 3.14-3.08 (m, 1H), 2.09-1.90 (m, 4H). MS (ESI): m/z 395.0 [M+H]⁺.

### Example 4: N-((1r,4r)-4-Methylcyclohexyl)-2-oxo-2-(4-(quinolin-4-yl)piperidin-1-yl)acetamide (4)

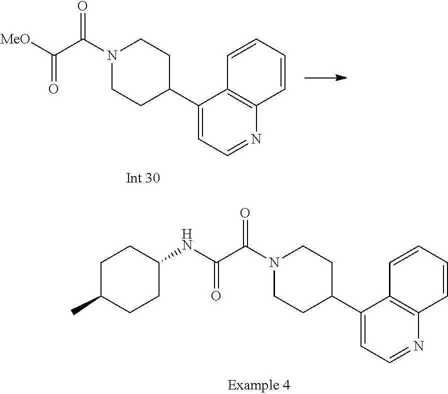 [SMILES: COC(=O)C(=O)N1CCC(c2ccnc3ccccc23)CC1 ; C[C@H]1CC[C@H](NC(=O)C(=O)N2CCC(c3ccnc4ccccc34)CC2)CC1]

To a solution of methyl 2-oxo-2-(4-(quinolin-4-yl)piperidin-1-yl)acetate (Int 30) (460 mg of crude, 0.52 mmol, 1.0 eq.) in THF/H₂O (5 mL, v/v=1:1) was added LiOH.H₂O (44 mg, 1.04 mmol, 2.0 eq.). The mixture was stirred at rt overnight and was concentrated to dryness to give the crude lithium salt as a white solid. The solid was dissolved in DMF (10 mL) and 4-(piperidin-4-yl)quinoline hydrochloride (252 mg, 1.68 mmol, 1.0 eq.), HATU (766 mg, 1.01 mmol, 1.2 eq.) and DIPEA (436 mg, 3.38 mmol, 2.0 eq.) were added and the mixture was stirred at rt for 2 h. The reaction mixture was concentrated in vacuo. The residue was purified by prep-HPLC to give the title compound as a white solid. ¹H NMR (CD₃OD, 400 MHz): δ ppm 8.78 (d, J=5.2 Hz, 1H), 8.31 (d, 1H, J=8.0 Hz, 1H), 8.04 (d, J=8.4 Hz, 1H), 7.80-7.76 (m, 1H), 7.69-7.65 (m, 1H), 7.44 (d, J=4.8 Hz, 1H), 4.66-4.63 (m, 1H), 4.07-4.04 (m, 1H), 3.85-3.81 (m, 1H), 3.69-3.65 (m, 1H), 3.47-3.44 (m, 1H), 3.05-3.00 (m, 1H), 2.06-1.74 (m, 8H), 1.36-1.29 (m, 3H), 1.08-1.04 (m, 2H), 0.93-0.90 (m, 3H). MS (ESI): m/z 380.1 [M+H]⁺.

### Example 5: (S)-2-(4-(6-Fluoroquinolin-4-yl)piperidin-1-yl)-2-oxo-N-(1,2,3,4-tetrahydro-naphthalen-2-yl)acetamide (5)

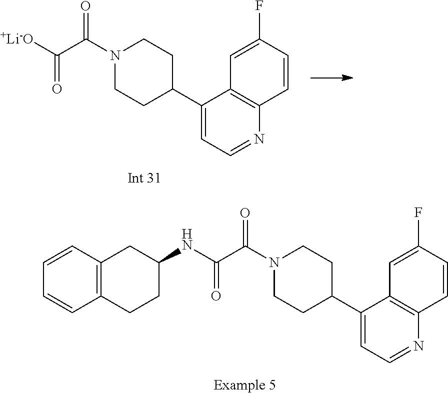

A solution of lithium 2-(4-(6-fluoroquinolin-4-yl)piperidin-1-yl)-2-oxoacetate (Int 31) (130.0 mg, 0.4 mmol, 1.0 eq.), (S)-1,2,3,4-tetrahydronaphthalen-2-amine (67.6 mg, 0.5 mmol, 1.1 eq.), HATU (197.9 mg, 0.5 mmol, 1.2 eq.) and DIPEA (162.5 mg, 1.2 mmol, 3.0 eq.) in DMF (11 mL) was stirred at rt under N₂ overnight. The reaction solution was concentrated in vacuo and purified by prep-HPLC to afford the title compound as a white solid. ¹H NMR (DMSO-d₆, 400 MHz): δ ppm 8.85-8.82 (m, 2H), 8.14-8.09 (m, 2H), 7.72-7.67 (m, 1H), 7.44-7.43 (m, 1H), 7.09 (s, 4H), 4.49-4.44 (m, 1H), 4.08-4.04 (m, 1H), 3.89-3.85 (m, 1H), 3.72 (t, J=10.8 Hz, 1H), 3.41 (t, J=12.0 Hz, 1H), 3.04-2.96 (m, 2H), 2.89-2.85 (m, 2H), 2.74-2.68 (m, 1H), 2.02-1.87 (m 3H), 1.77-1.60 (m 3H). MS (ESI): m/z 432.1 [M+H]⁺.

### Examples 5/1 to 5/10

The following Examples were prepared similar as described for Example 5 using the appropriate building blocks.

| | Building | | |
|---|---|---|---|
| # | blocks | Structure | Analytical data |
| 5/1 | Int 31, 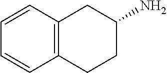 [SMILES: N[C@@H]1CCc2ccccc2C1] | 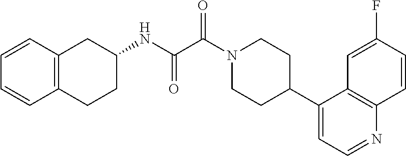 [SMILES: O=C(N[C@@H]1CCc2ccccc2C1)C(=O)N1CCC(c2ccnc3ccc(F)cc23)CC1] | ¹H NMR (DMSO-d₆, 400 MHz): δ ppm 8.85-8.82 (m, 2H), 8.14- 8.09 (m, 2H), 7.72-7.67 (m, 1H), 7.44-7.42 (m, 1H), 7.09 (s, 4H), 4.48-4.44 (m, 1H), 4.08- 4.04 (m, 1H), 3.89-3.85 (m, 1H), 3.72 (t, J = 10.8 Hz, 1H), 3.41 (t, J = 12.1 Hz, 1H), 3.04- 2.96 (m, 2H), 2.89-2.85 (m, 2H), 2.74-2.68 (m, 1H), 2.02- |
| | | | 1.87 (m, 3H), 1.78-1.60 (m, |
| | | | 3H). MS (ESI): m/z 432.1 |
| | | | [M + H]⁺. |
| 5/2 | Int 31, 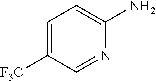 [SMILES: Nc1ccc(C(F)(F)F)cn1] | 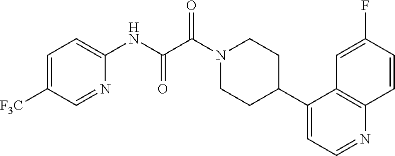 [SMILES: O=C(Nc1ccc(C(F)(F)F)cn1)C(=O)N1CCC(c2ccnc3ccc(F)cc23)CC1] | ¹H NMR (DMSO-d₆, 400 MHz): δ ppm 11.73 (s br, 1H), 8.86- 8.84 (m, 1H), 8.78 (s, 1H), 8.26 (s, 2H), 8.15-8.09 (m, 2H), 7.70 (t, J = 7.6 Hz, 1H), 7.49-7.47 (m, 1H), 4.50-4.46 (m, 1H), 3.81-3.71 (m, 2H), 3.49 (t, J = 12.4 Hz, 1H), 3.06 (t, J = 12.0 Hz, 1H), 1.99-1.73 (m, 4H). MS (ESI): m/z 447.0 [M + H]⁺. |
| 5/3 | Int 31, 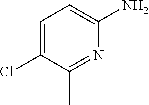 | 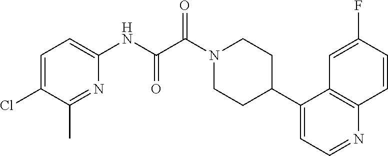 [SMILES: Cc1nc(NC(=O)C(=O)N2CCC(c3ccnc4ccc(F)cc34)CC2)ccc1Cl] | ¹H NMR (CDCl₃, 400 MHz): δ ppm 9.50 (s, 1H), 8.85-8.83 (m, 1H), 8.18-8.14 (m, 1H), 8.01 (d, J = 8.8 Hz, 1H), 7.70-7.65 (m, 2H), 7.54-7.49 (m, 1H), 7.29- 7.27 (m, 1H), 5.33-5.28 (m, 1H), 4.88-4.85 (m, 1H), 3.55- 3.50 (m, 1H), 3.42-3.35 (m, 1H), 3.06-2.98 (m, 1H), 2.55 (s, 3H), 2.16-2.09 (m, 2H), 2.00- |
| | | | 1.81 (m, 2H). MS (ESI): m/z |
| | | | 427.0 [M + H]⁺. |
| 5/4 | Int 31, 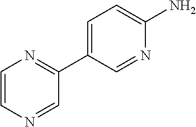 [SMILES: Nc1ccc(-c2cnccn2)cn1] | 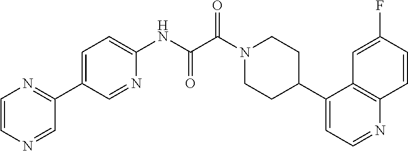 [SMILES: O=C(Nc1ccc(-c2cnccn2)cn1)C(=O)N1CCC(c2ccnc3ccc(F)cc23)CC1] | ¹H NMR (CDCl₃, 400 MHz): δ ppm 9.76 (s, 1H), 9.06 (s, 1H), 9.03 (s, 1H), 8.85 (d, J = 4.4 Hz, 1H), 8.67 (s, 1H), 8.57 (d, J = 2.4 Hz, 1H), 8.44-8.36 (m, 2H), 8.16 (dd, J = 9.6, 6.0 Hz, 1H), 7.70 (dd, J = 10.4, 2.4 Hz, 1H), 7.54-7.49 (m, 1H), 7.30 (d, J = 4.4 Hz, 1H), 5.36-5.31 (m, 1H), 4.91-4.86 (m, 1H), 3.57-3.51 |
| | | | (m, 1H), 3.45-3.38 (m, 1H), |
| | | | 3.08-3.01 (m, 1H), 2.18-2.13 |
| | | | (m, 2H), 2.05-1.86 (m, 2H). MS |
| | | | (ESI): m/z 457.0 [M + H]⁺. |
| 5/5 | Int 31, 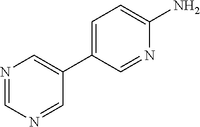 [SMILES: Nc1ccc(-c2cncnc2)cn1] | 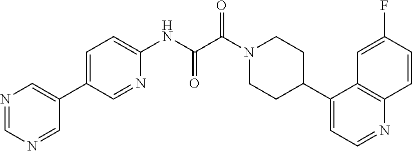 [SMILES: O=C(Nc1ccc(-c2cncnc2)cn1)C(=O)N1CCC(c2ccnc3ccc(F)cc23)CC1] | ¹H NMR (DMSO-d₆, 400 MHz): δ ppm 11.49 (s, 1H), 9.22 (s, 3H), 8.86 (s, 2H), 8.35-8.32 (m, 1H), 8.23-8.10 (m, 3H), 7.72- 7.67 (m, 1H), 7.49 (d, J = 4.4 Hz, 1H), 4.51-4.47 (m, 1H), 3.85-3.72 (m, 2H), 3.54-3.52 (m, 1H), 3.07 (t, J = 12.0 Hz, 1H), 1.98-1.75 (m, 4H). MS (ESI): m/z 457.0 [M + H]⁺. |
| 5/6 | Int 31, 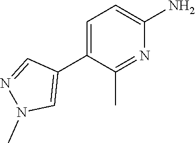 | 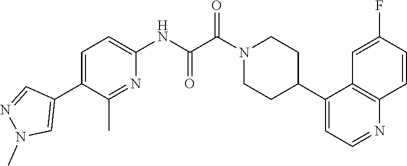 [SMILES: Cc1nc(NC(=O)C(=O)N2CCC(c3ccnc4ccc(F)cc34)CC2)ccc1-c1cnn(C)c1] | ¹H NMR (DMSO-d₆, 400 MHz): δ ppm 11.19 (s, 1H), 8.86 (d, J = 4.4 Hz, 1H), 8.16-8.10 (m, 2H), 8.01 (s, 1H), 7.93 (d, J = 8.0 Hz, 1H), 7.81 (d, J = 8.0 Hz, 1H), 7.72-7.68 (m, 2H), 7.49- 7.47 (m, 1H), 4.49-4.45 (m, 1H), 3.89 (s, 3H), 3.82-3.74 (m, 2H), 3.51-3.44 (m, 1H), 3.03 (t, J = 11.6 Hz, 1H), 2.53 (s, 3H), 1.95-1.74 (m, 4H). MS (ESI): m/z 473.0 [M + H]⁺. |
| 5/7 | Int 31, 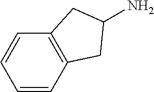 | 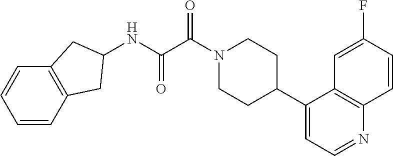 [SMILES: O=C(NC1Cc2ccccc2C1)C(=O)N1CCC(c2ccnc3ccc(F)cc23)CC1] | ¹H NMR (DMSO-d₆, 400 MHz): δ ppm 9.05 (d, J = 6.8 Hz, 1H), 8.83 (d, J = 4.4 Hz, 1H), 8.14- 8.09 (m, 2H), 7.76-7.67 (m, 1H), 7.43 (d, J = 4.4 Hz, 1H), 7.24-7.23 (m, 2H), 7.17-7.15 (m, 2H), 4.57-4.52 (m, 1H), 4.47-4.44 (m, 1H), 3.88-3.84 (m, 1H), 3.71 (t, J = 11.2 Hz, 1H), 3.43-3.40 (m, 1H), 3.25- |
| | | | 3.20 (m, 2H), 2.98 (t, J = 12.0 |
| | | | Hz, 1H), 2.89-2.81 (m, 2H), |
| | | | 1.96-1.86 (m, 2H), 1.79-1.57 |
| | | | (m, 2H). MS (ESI): m/z 418.0 |
| | | | [M + H]⁺. |
| 5/8 | Int 31, 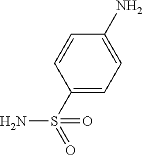 [SMILES: Nc1ccc(S(N)(=O)=O)cc1] | 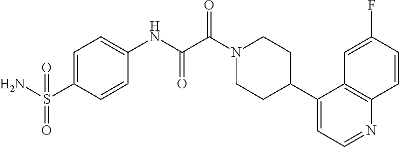 [SMILES: NS(=O)(=O)c1ccc(NC(=O)C(=O)N2CCC(c3ccnc4ccc(F)cc34)CC2)cc1] | ¹H NMR (DMSO-d₆, 400 MHz): δ ppm 11.17 (s, 1H), 8.85 (d, J = 8.0 Hz, 1H), 8.15-8.09 (m, 2H), 7.85-7.79 (m, 4H), 7.71- 7.67 (m, 1H), 7.47-7.46 (m, 1H), 7.31 (s, 2H), 4.54-4.51 (m, 1H), 3.94-3.91 (m, 1H), 3.76- 3.48 (m, 1H), 3.11-3.06 (m, 1H), 2.07-1.92 (m, 2H), 1.79- 1.71 (m, 2H). MS (ESI): m/z 456.9 [M + H]⁺. |
| 5/9 | Int 31, 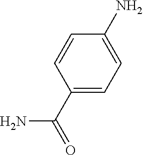 | 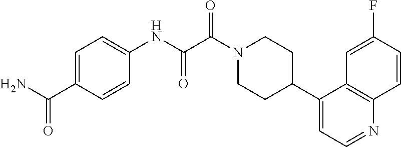 [SMILES: NC(=O)c1ccc(NC(=O)C(=O)N2CCC(c3ccnc4ccc(F)cc34)CC2)cc1] | ¹H NMR (DMSO-d₆, 400 MHz): δ ppm 11.04 (s, 1H), 8.85 (d, J = 4.4 Hz, 1H), 8.15-8.11 (m, 2H), 7.92-7.87 (m, 3H), 7.74- 7.67 (m, 3H), 7.47-7.46 (m, J = 4.4 Hz, 1H), 7.31 (s, 2H), 4.54-4.51 (m, 1H), 3.95-3.92 (m, 1H), 3.78-3.74 (m, 1H), 3.53-3.44 (m, 1H), 3.12-3.06 (m, 1H), 2.01-1.92 (m, 2H), 1.80-1.67 (m, 2H). MS (ESI): m/z 421.0 [M + H]⁺. |
| 5/10 | Int 31, 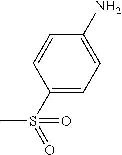 | 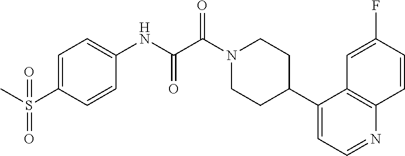 [SMILES: CS(=O)(=O)c1ccc(NC(=O)C(=O)N2CCC(c3ccnc4ccc(F)cc34)CC2)cc1] | ¹H NMR (DMSO-d₆, 400 MHz): δ ppm 11.29) (s, 1H), 8.85 (d, J = 4.4 Hz, 1H), 8.15-8.09 (m, 2H), 7.94-7.92 (m, 4H), 7.72- 7.67 (m, 1H), 7.47 (d, J = 4.4 Hz, 1H), 4.56-4.49 (m, 1H), 3.96-3.89 (m, 1H), 3.81-3.70 (m, 1H), 3.54-3.44 (m, 1H), 3.18 (s,3H), 3.18-3.09 (m, 1H), 2.01-1.92 (m, 2H), 1.81-1.71 (m, 2H). MS (ESI): m/z 455.9 [M + H]⁺. |

### Example 6: N-(5-Chloropyridin-2-yl)-2-(4-(naphthalen-1-yl)piperidin-1-yl)-2-oxoacetamide (6)

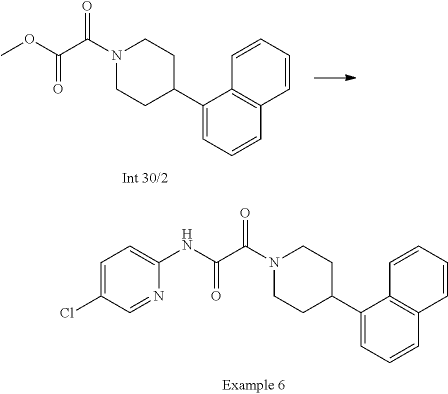 [SMILES: COC(=O)C(=O)N1CCC(c2cccc3ccccc23)CC1 ; O=C(Nc1ccc(Cl)cn1)C(=O)N1CCC(c2cccc3ccccc23)CC1]

To a solution of 5-chloropyridin-2-amine (180 mg, 1.4 mmol) in 1,4-dioxane (10 ml), AlMe₃ (1 M, 1.7 ml) was added, then stirred at rt for 2 h. Methyl 2-(4-(naphthalen-1-yl)piperidin-1-yl)-2-oxoacetate (Int 30/2) (200 mg, 0.67 mmol) was added and the mixture was stirred at 80° C. overnight. Water was added (10 mL) and the mixture was extracted with EtOAc (3×20 ml). The combined organic phases were washed with water (20 ml), dried over Na₂SO₄, filtered and concentrated under reduced pressure. The residue was purified on silica gel column (PE/EtOAc=5/1 (v/v)) to afford the title compound as a white solid. ¹H NMR (400 MHz, DMSO-d₆): δ ppm 11.43 (s, 1H), 8.43 (s, 1H), 8.27 (d, J=8.0 Hz, 1H), 8.12 (d, J=8.4 Hz, 1H), 7.97-7.93 (m, 2H), 7.81 (d, J=8.0 Hz, 1H), 7.59-7.42 (m, 4H), 4.51 (d, J=8.4 Hz, 1H), 3.83-3.71 (m, 2H), 3.49-3.45 (m, 1H), 3.07-3.01 (m, 1H), 1.99-1.74 (m, 4H). MS-ESI: m/z 394.1 [M+H]⁺.

### Examples 6/1 to 6/2

The following Examples were prepared similar as described for Example 6 using the appropriate building blocks.

| | Building | | |
|---|---|---|---|
| # | blocks | Structure | Analytical data |
| 6/1 | Int 30/3, 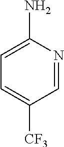 [SMILES: Nc1ccc(C(F)(F)F)cn1] | 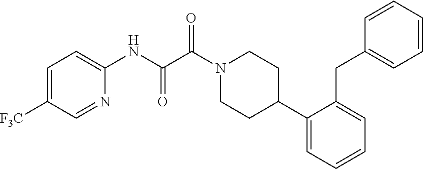 [SMILES: O=C(Nc1ccc(C(F)(F)F)cn1)C(=O)N1CCC(c2ccccc2Cc2ccccc2)CC1] | ¹H NMR (400 MHz, DMSO- d₆): δ ppm 11.67 (s br, 1H), 8.77 (s, 1H), 8.24 (s, 2H), 7.29-7.15 (m, 9H), 4.35-4.32 (m, 1H), 4.07 (s, 2H), 3.66- 3.63 (m, 1H), 3.19-3.09 (m, 2H), 2.76-.69 (m, 1H), 1.67- 1.39 (m, 4H). MS-ESI: m/z 468.3 [M + H]⁺. |
| 6/2 | Int 30/4, 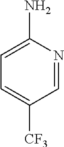 [SMILES: Nc1ccc(C(F)(F)F)cn1] | 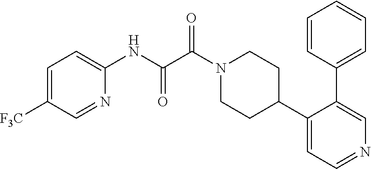 [SMILES: O=C(Nc1ccc(C(F)(F)F)cn1)C(=O)N1CCC(c2ccncc2-c2ccccc2)CC1] | ¹H NMR (400 MHz, DMSO- d₆): δ ppm 11.4 (s br, 1H), 8.77 (s, 1H), 8.57 (d, J = 5.2 Hz, 1H), 8.36 (s, 1H), 8.25 (s, 2H), 7.53-7.44 (m, 4H), 7.39- 7.37 (m, 2H), 4.37-4.33 (m, 1H), 3.69-3.65 (m, 1H), 3.07- 2.91 (m, 2H), 2.67-2.57 (m, 1H), 1.85-1.67 (m, 4H). MS- ESI: m/z 455.2 [M + H]⁺. |

### Example 7: N-(4-(2-Chloroethyl)phenyl)-2-(4-(6-fluoroquinolin-4-yl)piperidin-1-yl)-2-oxoacetamide (7)

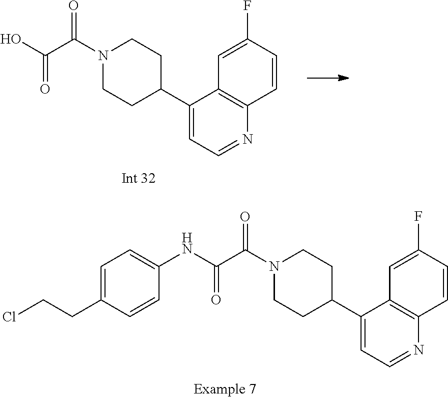 [SMILES: O=C(O)C(=O)N1CCC(c2ccnc3ccc(F)cc23)CC1 ; O=C(Nc1ccc(CCCl)cc1)C(=O)N1CCC(c2ccnc3ccc(F)cc23)CC1]

To a solution of 2-(4-(6-fluoroquinolin-4-yl)piperidin-1-yl)-2-oxoacetic acid (Int 32) (100 mg, 0.33 mmol), HATU (189 mg, 0.49 mmol) and TEA (67 mg, 0.66 mmol) in DCM (5 mL) was added 4-(2-chloroethyl)aniline (54 mg, 0.35 mmol) at rt. After stirring overnight, the mixture was concentrated and the residue was redissolved in DCM (50 mL) and washed with water (50 mL) twice. The organic layer was dried over Na₂SO₄, the combined organic layer was concentrated under vacuum, and then purified by silica gel column chromatography (EA:PE=1:2, v/v) to give the title compound as a white solid. ¹H NMR (500 MHz, DMSO-d₆): δ ppm 10.79 (s, 1H), 8.95 (d, J=4.0 Hz, 1H), 8.25-8.16 (m, 2H), 7.82-7.78 (m, 1H), 7.82-7.60 (m, 3H), 7.28-7.26 (m, 2H), 4.54-4.52 (m, 1H), 3.95-3.93 (m, 1H), 3.84-3.79 (m, 3H), 3.50-3.45 (m, 1H), 3.10-2.98 (m, 3H), 2.00-1.93 (m, 2H), 1.82-1.70 (m, 2H). MS-ESI: m/z=440.2 [M+1]⁺.

### Examples 7/1 to 7/2

The following Examples were prepared similar as described for Example 7 using the appropriate building blocks.

| | Building | | |
|---|---|---|---|
| # | blocks | Structure | Analytical data |
| 7/1 | Int 32 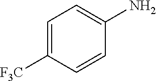 [SMILES: Nc1ccc(C(F)(F)F)cc1] | 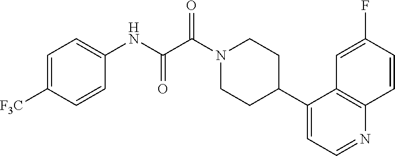 [SMILES: O=C(Nc1ccc(C(F)(F)F)cc1)C(=O)N1CCC(c2ccnc3ccc(F)cc23)CC1] | ¹H NMR (500 MHz, DMSO- d₆): δ ppm 11.21 (s, 1H), 8.96- 8.95 (m, 1H), 8.25-8.16 (m, 2H), 7.90-7.88 (m, 2H), 7.81- 7.74 (m, 3H), 7.61-7.60 (m, 1H), 4.55-4.52 (m, 1H), 3.96- 3.80 (m, 2H), 3.52-3.47 (m, 1H), 3.12-3.08 (m, 1H), 2.02- 1.94 (m, 2H), 1.83-1.71 (m, 2H). MS (ESI): m/z 446.1 [M + H]⁺. |
| 7/2 | Int 32 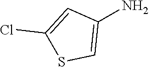 | 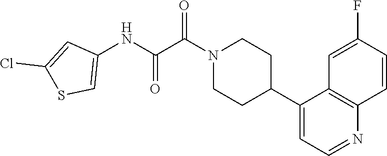 [SMILES: O=C(Nc1csc(Cl)c1)C(=O)N1CCC(c2ccnc3ccc(F)cc23)CC1] | ¹H NMR (500 MHz, DMSO- d₆): δ ppm 11.24 (s, 1H), 8.97- 8.96 (m, 1H), 8.27-8.17 (m, 2H), 7.83-7.79 (m, 1H), 7.63- 7.62 (m, 1H), 7.50 (s, 1H), 7.18 (s, 1H), 4.54-4.52 (m, 1H), 4.04-4.01 (m, 1H), 3.83- 3.81 (m, 1H), 3.49-3.43 (m, 1H), 3.11-3.05 (m, 1H), 2.01- 1.94 (m, 2H), 1.81-1.68 (m, |
| | | | 2H). MS (ESI): m/z 418.1 |
| | | | [M + H]⁺. |

### Example 8: 2-(4-(6-Fluoroquinolin-4-yl)piperidin-1-yl)-2-oxo-N-(1-phenylethyl)acetamide (8)

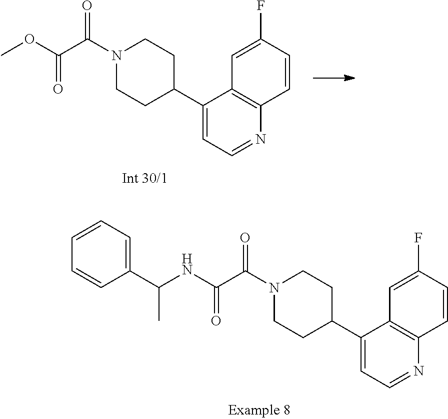 [SMILES: COC(=O)C(=O)N1CCC(c2ccnc3ccc(F)cc23)CC1 ; CC(NC(=O)C(=O)N1CCC(c2ccnc3ccc(F)cc23)CC1)c1ccccc1]

To a mixture of methyl 2-(4-(6-fluoroquinolin-4-yl)piperidin-1-yl)-2-oxoacetate (Int 30/1) (100 mg, 0.32 mmol) in MeOH (1 mL) was added compound 1-phenylethanamine (46 mg, 0.38 mmol) and the mixture was stirred at 70° C. for 5 h. The mixture was purified by preparative HPLC to give the title compound as a white solid. ¹H NMR (500 MHz, DMSO-d₆): δ ppm 9.26-9.21 (m, 1H), 8.97-8.94 (m, 1H), 8.25-8.16 (m, 2H), 7.82-7.79 (m, 1H), 7.60-7.58 (m, 1H), 7.37-7.22 (m, 4H), 5.04-4.98 (m, 1H), 4.49-4.46 (m, 1H), 3.78-3.60 (m, 2H), 3.39-3.35 (m, 1H), 3.01-2.95 (m, 1H), 1.95-1.70 (m, 4H), 1.42-1.39 (m, 3H). MS (ESI): m/z 406.2 [M+H]⁺.

### Examples 8/1 to 8/6

The following Examples were prepared similar as described for Example 8 using the appropriate building blocks.

| | Building | | |
|---|---|---|---|
| # | blocks | Structure | Analytical data |
| 8/1 | Int 30/1 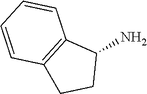 | 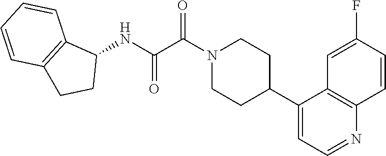 [SMILES: O=C(N[C@@H]1CCc2ccccc21)C(=O)N1CCC(c2ccnc3ccc(F)cc23)CC1] | ¹H NMR (500 MHz, CD₃OD): δ ppm 9.05-9.04 (sm 1H), 8.37- 8.35 (m, 1H), 8.31-8.28 (m, 1H), 8.00-7.88 (m, 2H), 7.35- 7.20 (m, 4H), 5.51-5.48 (m, 1H), 4.72-4.70 (m, 1H), 4.23- 4.20 (m, 1H), 3.99-3.94 (m, 1H), 3.59-3.53 (m, 1H), 3.11- 3.08 (m, 2H), 2.94-2.91 (m, 1H), 2.59-2.57 (m, 1H), 2.13- |
| | | | 1.92 (m, 5H). MS (ESI): m/z |
| | | | 418.2 [M + H]⁺. |
| 8/2 | Int 30/1 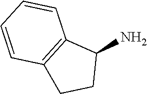 | 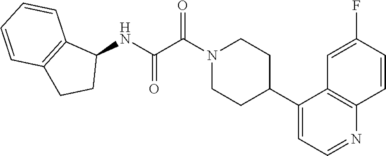 [SMILES: O=C(N[C@H]1CCc2ccccc21)C(=O)N1CCC(c2ccnc3ccc(F)cc23)CC1] | ¹H NMR (500 MHz, CD₃OD): δ ppm 9.04-9.03 (m, 1H), 8.36- 8.34 (m, 1H), 8.30-8.27 (m, 1H), 8.00-7.88 (m, 2H), 7.35- 7.21 (m, 4H), 5.51-5.49 (m, 1H), 4.72-4.70 (m, 1H), 4.23- 4.20 (m, 1H), 3.99-3.94 (m, 1H), 3.59-3.53 (m, 1H), 3.11- 3.08 (m, 2H), 2.94-2.91 (m, 1H), 2.59-2.57 (m, 1H), 2.13- |
| | | | 1.92 (m, 5H). MS (ESI): m/z |
| | | | 418.2 [M + H]⁺. |
| 8/3 | Int 30/1 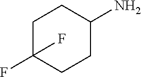 | 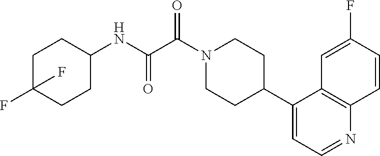 | ¹H NMR (500 MHz, DMSO-d₆): δ ppm 8.95 (s, 1H), 8.78-8.76 (m, 1H), 8.25-8.16 (m, 2H), 7.81-7.78 (m, 1H), 7.59-7.58 (m, 1H), 4.48-4.46 (m, 1H), 3.87-3.76 (m, 3H), 3.44-3.38 (m, 1H), 3.02-2.96 (m, 1H), 2.02-1.52 (m, 12H). MS (ESI): m/z 420.2 [M + H]⁺. |
| 8/4 | Int 30/1 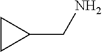 | 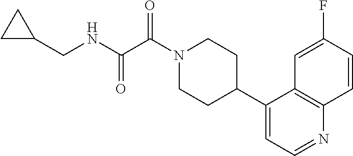 [SMILES: O=C(NCC1CC1)C(=O)N1CCC(c2ccnc3ccc(F)cc23)CC1] | ¹H NMR (500 MHz, DMSO-d₆): δ ppm 8.84-8.79 (m, 2H), 8.14- 8.10 (m, 2H), 7.72-7.68 (m, 1H), 7.45 (s, 1H), 4.49-4.46 (m, 1H), 3.91-3.89 (m, 1H), 3.72- 3.69 (m, 1H), 3.42-3.37 (m, 1H), 3.05-2.96 (m, 3H), 1.95- 1.89 (m, 2H), 1.77-1.63 (m, 2H), 0.97-0.94 (m, 1H), 0.44- 0.41 (m, 2H), 0.20-0.18 (m, |
| | | | 2H). MS (ESI): m/z 356.2 |
| | | | [M + H]⁺. |
| 8/5 | Int 30/1 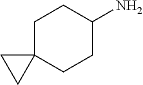 | 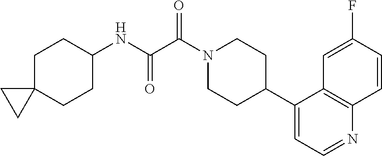 [SMILES: O=C(NC1CCC2(CC1)CC2)C(=O)N1CCC(c2ccnc3ccc(F)cc23)CC1] | ¹H NMR (500 MHz, DMSO-d₆): δ ppm 8.84 (s, 1H), 8.65-8.64 (m, 1H), 8.14-8.09 (m, 2H), 7.71-7.67 (m, 1H), 7.45-7.44 (m, 1H), 4.47-4.45 (m, 1H), 3.82-3.68 (m, 3H), 3.42-3.36 (m, 1H), 3.00-2.95 (m, 1H), 1.94-1.88 (m, 2H), 1.78-1.63 (m, 6H), 1.42-1.37 (m, 2H), 0.99-0.97 (m, 2H), 0.29-0.18 |
| | | | (m, 4H). MS (ESI): m/z 410.2 |
| | | | [M + H]⁺. |
| 8/6 | Int 30/1 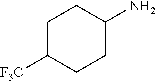 [SMILES: NC1CCC(C(F)(F)F)CC1] | 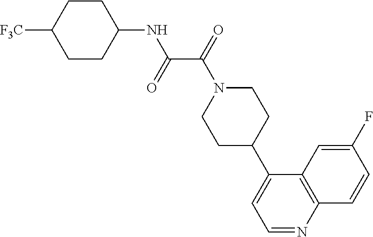 [SMILES: O=C(NC1CCC(C(F)(F)F)CC1)C(=O)N1CCC(c2ccnc3ccc(F)cc23)CC1] | ¹H NMR (500 MHz, DMSO-d₆): δ ppm 8.84-8.69 (m, 2H), 8.14- 8.09 (m, 2H), 7.71-7.67 (m, 1H), 7.45-7.43 (m, 1H), 4.48- 4.45 (m, 1H), 4.01-3.70 (m, 3H), 3.43-3.38 (m, 1H), 3.01- 2.96 (m, 1H), 2.29-2.28 (m, 1H), 1.94-1.62 (m, 11H), 1.36- 1.30 (m, 1H). ). MS (ESI): m/z 452.2 [M + H]⁺. |

### Example 9: 4-(1-(2-((5-Chloropyridin-2-yl)amino)-2-oxoacetyl)piperidin-4-yl)-1-naphthoic Acid (9)

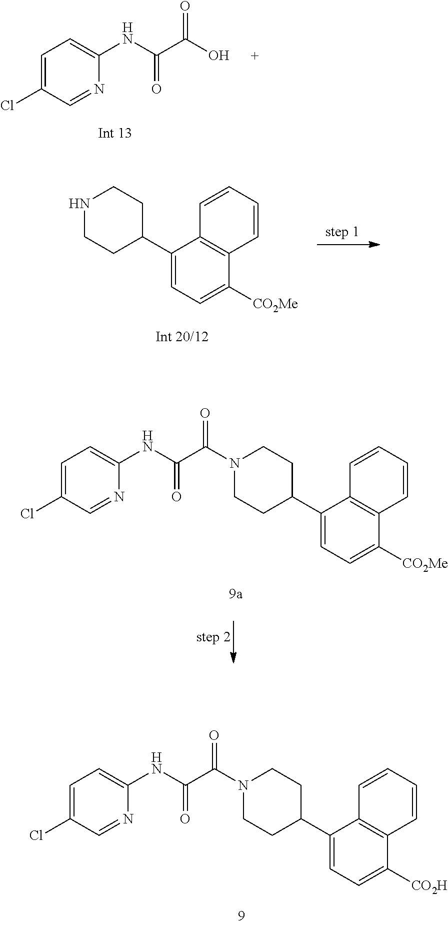 [SMILES: O=C(O)C(=O)Nc1ccc(Cl)cn1 ; COC(=O)c1ccc(C2CCNCC2)c2ccccc12 ; COC(=O)c1ccc(C2CCN(C(=O)C(=O)Nc3ccc(Cl)cn3)CC2)c2ccccc12 ; O=C(Nc1ccc(Cl)cn1)C(=O)N1CCC(c2ccc(C(=O)O)c3ccccc23)CC1]

### Step 1: Methyl 4-(1-(2-((5-chloropyridin-2-yl)amino)-2-oxoacetyl)piperidin-4-yl)-1-naphthoate (9a)

To a stirred solution of 2-((5-chloropyridin-2-yl)amino)-2-oxoacetic acid (Int 13) (160 mg, 0.80 mmol), HOBt (108 mg, 0.80 mmol), DIPEA (620 mg, 4.80 mmol), and EDCl (305 mg, 1.60 mmol) in DMF (6 mL) was added methyl 4-(piperidin-4-yl)-1-naphthoate (Int 20/12) (269 mg, 0.88 mmol) at rt. The mixture was stirred at rt for 16 h, diluted with H₂O (20 mL) and extracted with EtOAc (3×20 mL). The combined organic layers were washed with brine (30 mL), dried over Na₂SO₄, filtered and concentrated. The residue was purified by silica gel column chromatography, eluting with PE:EtOAc=1:1 (v/v) to give the title compound as a white solid.

### Step 2: 4-(1-(2-((5-Chloropyridin-2-yl)amino)-2-oxoacetyl)piperidin-4-yl)-1-naphthoic Acid (9)

To a solution of methyl 4-(1-(2-((5-chloropyridin-2-yl)amino)-2-oxoacetyl)piperidin-4-yl)-1-naphthoate (9a) (200 mg, 0.40 mmol) in THE (5 mL), MeOH (5 mL) and H₂O (5 mL) was added LiOH.H₂O (60 mg, 1.20 mmol). The mixture was stirred at rt for 12 h, concentrated and the residue was diluted with H₂O (10 mL). It was neutralized with 1 N HCl solution to pH=6-7 and extracted with EtOAc (3×30 mL). The combined organic layers were washed with brine (30 mL), dried over Na₂SO₄, filtered and concentrated. The residue was purified by prep-HPLC to give the title compound as a white solid. ¹H NMR (500 MHz, CD₃OD): δ ppm 8.96-8.95 (m, 1H), 8.40-8.30 (m, 2H), 8.22-8.20 (m, 1H), 8.15-8.13 (m, 1H), 7.88-7.85 (m, 1H), 7.66-7.57 (m, 2H), 7.51 (d, J=7.6 Hz, 1H), 4.74-4.71 (m, 1H), 4.25-4.22 (m, 1H), 3.86 (t, J=11.6 Hz, 1H), 3.55 (t, J=11.8 Hz, 1H), 3.20-3.08 (m, 1H), 2.15-1.91 (m 4H). MS-ESI: m/z=438.0 [M+1]⁺.

### Example 10a and 10b: trans-N-(4-Chlorophenyl)-2-(4-(6-fluoroquinolin-4-yl)-3-hydroxypiperidin-1-yl)-2-oxoacetamide (Separated Enantiomers) (10a, 10b)

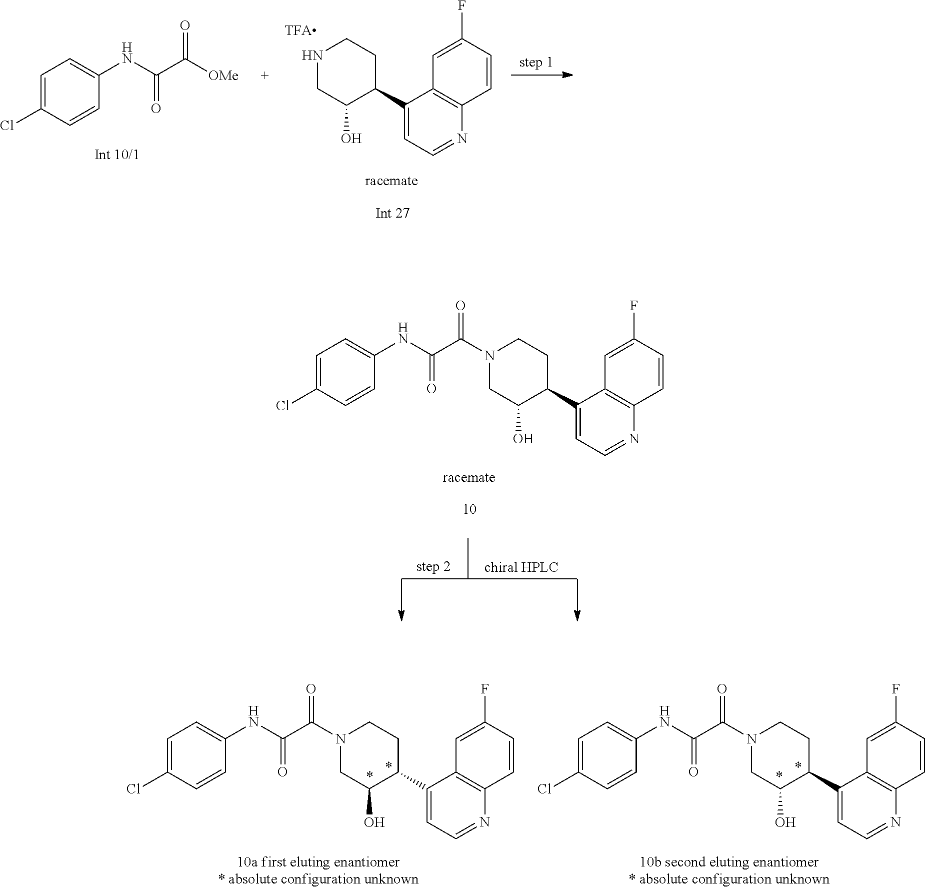

### Step 1: trans-N-(4-Chlorophenyl)-2-(4-(6-fluoroquinolin-4-yl)-3-hydroxypiperidin-1-yl)-2-oxoacetamide (racemate) (10)

To a solution of trans-4-(6-fluoroquinolin-4-yl)piperidin-3-ol trifluoroacetic acid salt (Int 27) (114 mg, 0.33 mmol) and methyl 2-((4-chlorophenyl)amino)-2-oxoacetate (Int 10/1) (86 mg, 0.40 mmol) in MeOH (5 mL) was added TEA (101 mg, 1.00 mmol) and the mixture was stirred at 80° C. overnight. The mixture was diluted with H₂O (50 mL) and extracted with EtOAc (3×50 mL). The combined organic layers were washed with brine (50 mL), dried over Na₂SO₄, filtered and concentrated to dryness to give the title compound as a yellow solid.

### Step 2: trans-N-(4-Chlorophenyl)-2-(4-(6-fluoroquinolin-4-yl)-3-hydroxypiperidin-1-yl)-2-oxoacetamide (Separated Enantioners) (10a, 10b)

Chiral HPLC separation (IE column, n-hexane/0.1% DEA:EtOH/0.1% DEA=50:50) of trans-N-(4-chlorophenyl)-2-(4-(6-fluoroquinolin-4-yl)-3-hydroxypiperidin-1-yl)-2-oxoacetamide (racemate) (10) (92 mg, 0.22 mmol) afforded trans-N-(4-chlorophenyl)-2-(4-(6-fluoroquinolin-4-yl)-3-hydroxypiperidin-1-yl)-2-oxoacetamide (10a, first eluting enantiomer with a retention time of 8.64 min) and trans-N-(4-chlorophenyl)-2-(4-(6-fluoroquinolin-4-yl)-3-hydroxypiperidin-1-yl)-2-oxoacetamide (10b, second eluting enantiomer with a retention time of 10.67 min).

Example 10a: ¹H NMR (500 MHz, DMSO-d₆): δ ppm 11.01 (s, 0.5H), 10.97 (s, 0.5H), 8.87-8.85 (m, 1H), 8.20-8.01 (m, 2H), 7.79-7.61 (m, 3H), 7.58-7.50 (m, 1H), 7.47-7.34 (m, 2H), 5.21-5.16 (1, 1H), 4.59-4.40 (m, 1H), 3.99-3.82 (m, 2H), 3.68-3.60 (m, 2H), 3.45-2.78 (m, 2H), 1.91-1.61 (m, 2H). MS-ESI: m/z=428.2 [M+1]⁺.

Example 10b: ¹H NMR (500 MHz, DMSO-d₆): δ ppm 11.01 (m, 0.5H), 10.97 (s, 0.5H), 8.87-8.85 (m, 1H), 8.23-7.98 (m, 2H), 7.79-7.60 (m, 3H),7.58-7.50 (m, 1H), 7.47-7.37 (m, 2H), 5.21-5.16 (m, 1H), 4.59-4.41 (m, 1H), 3.99-3.82 (m, 2H), 3.68-3.60 (m, 1H), 3.41-2.78 (m, 2H), 1.93-1.64 (m, 2H). MS-ESI: m/z=428.2 [M+1]⁺.

### Examples 10/1a, 10/1b

The following Examples were prepared similar as described for Example 10a and 10b using the appropriate building blocks.

| | Building | | |
|---|---|---|---|
| # | blocks | Structure | Analytical data |
| 10/1a | Int 10/1, Int 29 | 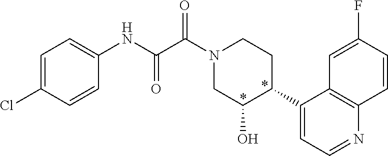 [SMILES: O=C(Nc1ccc(Cl)cc1)C(=O)N1CC[C@H](c2ccnc3ccc(F)cc23)[C@H](O)C1] first eluting enantiomer * absolute configuration unknown | ¹H NMR (500 MHz, DMSO-d₆): δ ppm 10.90 (s, 0.4H), 10.81 (s, 0.6H), 8.81 (d, J = 10.0 Hz, 1H), 8.18-8.01 (m, 2H), 7.85- 7.57 (m, 3H), 7.51-7.31 (m, 3H), 4.89-4.88 (m, 1H), 4.56- 4.41 (m, 1H), 4.02-3.77 (m, 4H), 3.40-3.37 (m, 0.4H), 3.02- 2.98 (m, 0.6H), 2.36-2.32 (m, 1H), 1.65-1.63 (m, 1H). MS- ESI: m/z = 428.2 [M + 1]⁺. Chiral HPLC separation (IE column, n-hexane/0.1% DEA: |
| | | | EtOH/0.1% DEA = 50:50): |
| | | | first eluting enantiomer, |
| | | | retention time 14.58 min) |
| 10/1b | Int 10/1, Int 29 | 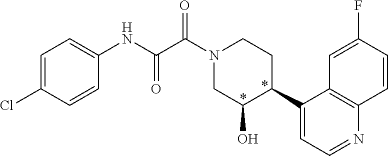 [SMILES: O=C(Nc1ccc(Cl)cc1)C(=O)N1CC[C@@H](c2ccnc3ccc(F)cc23)[C@@H](O)C1] second eluting enantiomer * absolute configuration unknown | ¹H NMR (500 MHz, DMSO-d₆) δ: 10.90 (s, 0.4H), 10.81 (m, 0.6H), 8.81 (d, J = 9.5 Hz, 1H), 8.22-8.02 (m, 2H), 7.82-7.58 (m, 3H), 7.49-7.31 (m, 3H), 4.89-4.88 (m, 1H), 4.56-4.41 (m, 1H), 4.00-3.77 (m, 1H), 3.40-3.37 (m, 0.4H), 3.00-2.98 (m, 0.6H), 2.36-2.32 (m, 1H), 1.65-1.63 (m, 1H). MS-ESI: m/z = 428.2 [M + 1]⁺. Chiral HPLC separation (IE column, n-hexane/0.1% DEA: |
| | | | EtOH/0.1% DEA = 50:50): first |
| | | | eluting enantiomer, retention |
| | | | time 27.26 min. |
| 10/2a | Int 10/1, Int 29/1 | 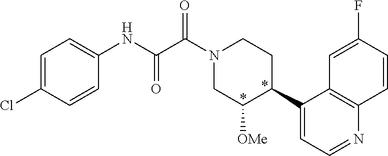 [SMILES: CO[C@@H]1CN(C(=O)C(=O)Nc2ccc(Cl)cc2)CC[C@H]1c1ccnc2ccc(F)cc12] first eluting enantiomer * absolute configuration unknown | ¹H NMR (400 MHz, DMSO-d₆) δ: 11.02 (s, 0.5H), 10.98 (s, 0.5H), 8.88-8.86 (m, 1H), 8.14- 8.08 (m, 2H), 7.76-7.66 (m, 3H), 7.61-7.58 (m, 1H), 7.46- 7.43 (m, 2H), 4.89-4.85 (m, 0.5H), 4.37-4.30 (m, 1H), 3.82- 3.77 (m, 2H), 3.71-3.56 (m, 0.5H), 3.49-3.44 (m, 0.5H), 3.21-2.95 (m, 4H), 2.78-2.71 (m, 0.5H), 2.02-1.82 (m, 1H), 1.73-1.65 (m, 1H). MS-ESI: m/z = 442.1 [M + 1]⁺. Chiral |
| | | | HPLC (SFC, cellulose-SC |
| | | | column, isopropanol/0.1% DEA: |
| | | | CO₂ = 30:70): first eluting |
| | | | enantiomer, retention time |
| | | | 1.29 min. |
| 10/2b | Int 10/1, Int 29/1 | 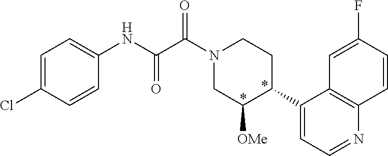 [SMILES: CO[C@H]1CN(C(=O)C(=O)Nc2ccc(Cl)cc2)CC[C@@H]1c1ccnc2ccc(F)cc12] second eluting enantiomer * absolute configuration unknown | ¹H NMR (400 MHz, DMSO-d₆) δ: 11.02 (s, 0.5H), 10.98 (s, 0.5H), 8.88-8.86 (m, 1H), 8.14- 8.11 (m, 2H), 7.75-7.68 (m, 3H), 7.60-7.58 (m, 1H), 7.46-7.41 (m, 2H), 4.89-4.85 (m, 0.5H), 4.40-4.28 (m, 1H), 3.80-3.75 (m, 2H), 3.72-3.55 (m, 0.5H), 3.50-3.45 (m, 0.5H), 3.22-2.93 (m, 4H), 2.78-2.71 (m, 0.5H), 2.02-1.86 (m, 1H), 1.75-1.65 (m, 1H). MS-ESI: m/z = 442.1 [M + 1]⁺. Chiral HPLC (SFC, |
| | | | cellulose-SC column, |
| | | | isopropanol/0.1% DEA:CO₂ = |
| | | | 30:70): second eluting |
| | | | enantiomer, retention time |
| | | | 1.63 min. |

### Examples 100 to 100/15

The following Examples were prepared similar as described for Example 1 using the appropriate building blocks.

| | Building | | |
|---|---|---|---|
| # | blocks | Structure | Analytical data |
| 100 | Int 10/3, 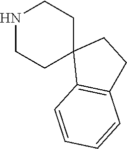 [SMILES: c1ccc2c(c1)CCC21CCNCC1] | 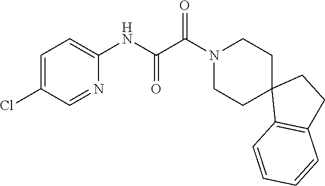 [SMILES: O=C(Nc1ccc(Cl)cn1)C(=O)N1CCC2(CCc3ccccc32)CC1] | ¹H NMR (500 MHz, DMSO-d₆): δ ppm 11.42 (s, 1H), 8.43 (s, 1H), 8.12-8.10 (m, 1H), 7.98- 7.95 (m, 1H), 7.22-7.14 (m, 4H), 4.32-4.29 (m, 1H), 3.66- 3.64 (m, 1H), 3.36-3.31 (m, 1H), 2.97-2.87 (m, 3H), 2.11- 2.06 (m, 2H), 1.87-1.79 (m, 2H), 1.56-1.49 (m, 2H). MS (ESI): m/z 370.1 [M + H]⁺. |
| 100/1 | Int 10/3, 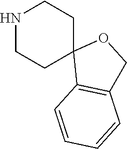 [SMILES: c1ccc2c(c1)COC21CCNCC1] | 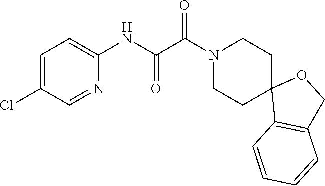 [SMILES: O=C(Nc1ccc(Cl)cn1)C(=O)N1CCC2(CC1)OCc1ccccc12] | 1H NMR (500 MHz, DMSO-d₆): δ ppm 11.40 (s, 1H), 8.43 (d, J = 2.4 Hz, 1H), 8.10 (s, 1H), 7.97 (dd, J = 8.9, 2.5 Hz, 1H), 7.32-7.26 (m, 4H), 5.12-4.94 (m, 2H), 4.33-4.31 (m, 1H), 3.67-3.65 (m, 1H), 3.45-3.42 (m, 1H), 3.08-3-02 (m, 1H), 2.05-1.86 (m, 2H), 1.73-1.99 (m, 1H). MS (ESI): m/z 372.0 [M + H]⁺. |
| 100/2 | Int 10/3, 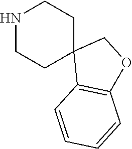 [SMILES: c1ccc2c(c1)OCC21CCNCC1] | 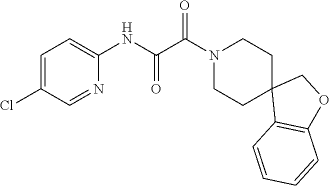 [SMILES: O=C(Nc1ccc(Cl)cn1)C(=O)N1CCC2(CC1)COc1ccccc12] | ¹H NMR (500 MHz, DMSO-d₆): δ ppm 11.43 (s, 1H), 8.44 (d, J = 2.0 Hz, 1H), 8.10 (d, J = 8.9 Hz, 1H), 7.97 (d, J = 7.1 Hz, 1H), 7.24 (d, J = 7.3 Hz, 1H), 7.14 (t, J = 7.7 Hz, 1H), 6.89 (t, J = 7.3 Hz, 1H), 6.79 (d, J = 7.9 Hz, 1H), 4.51-4.49 (m, 2H), 4.26-4.23 (m, 1H), 3.67-3.65 (m, 1H), 3.31-3.29 (m, 1H), 2.96-2.91 (m, 1H), 1.93-1.84 (m, 2H), 1.78-1.63 (m, 1H). MS (ESI): m/z 372.0 [M + H]⁺. |
| 100/3 | Int 10/3, 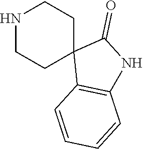 [SMILES: O=C1Nc2ccccc2C12CCNCC2] | 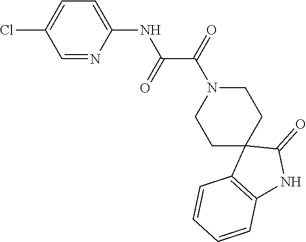 [SMILES: O=C(Nc1ccc(Cl)cn1)C(=O)N1CCC2(CC1)C(=O)Nc1ccccc12] | 1H NMR (500 MHz, DMSO-d₆): δ ppm 11.47 (s, 1H), 10.48 (s, 1H), 8.44 (s, 1H), 8.12-8.11 (m, 1H), 7.98-7.96 (m, 1H), 7.45- 7.43 (m, 1H), 7.23-7.20 (m, 1H), 7.01-6.98 (m, 1H), 6.88- 6.87 (m, 1H), 3.95-3.80 (m, 1H), 3.63-3.60 (m, 1H), 1.84- 1.78 (s, 4H). MS (ESI): m/z 372.0 [M + H]⁺. |
| 100/4 | Int 10/3, 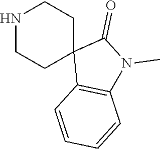 | 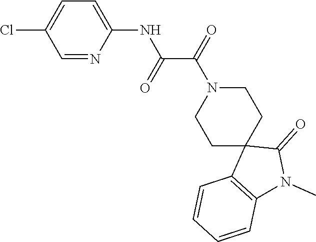 [SMILES: CN1C(=O)C2(CCN(C(=O)C(=O)Nc3ccc(Cl)cn3)CC2)c2ccccc21] | ¹H NMR (500 MHz, DMSO-d₆): δ ppm 11.22 (s, 1H), 8.45 (s, 1H), 8.12-8.11 (m, 1H), 7.98- 7.96 (m, 1H), 7.50-7.49 (m, 1H), 7.34-7.30 (m, 1H), 7.09- 7.04 (m, 2H), 3.98-3.79 (m, 3H), 3.65-3.62 (m, 1H), 3.15 (s, 1H), 1.87-1.78 (m, 1H). MS (ESI): m/z 399.0 [M + H]⁺. |
| 100/5 | Int 10/3, 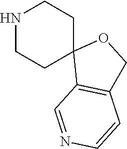 [SMILES: c1cc2c(cn1)C1(CCNCC1)OC2] | 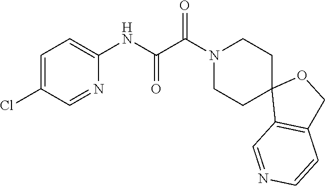 [SMILES: O=C(Nc1ccc(Cl)cn1)C(=O)N1CCC2(CC1)OCc1ccncc12] | ¹H NMR (500 MHz, DMSO-d₆): δ ppm 11.43 (s, 1H), 8.56-8.53 (m, 2H), 8.44 (d, J = 2.5 Hz, 1H), 8.15-8.13 (m, 1H), 8.01- 7.99 (m, 1H), 7.43-7.42 (m, 1H), 5.16-4.97 (m, 2H), 4.36- 4.34 (m, 1H), 3.74-3.71 (m, 1H), 3.51-3.46 (m, 1H), 3.13- 3.07 (m, 1H), 2.11-2.01 (m, 2H), 1.83-1.76 (m, 2H). MS (ESI): m/z 373.0 [M + H]⁺. |
| 100/6 | Int 10/3, 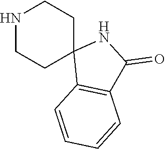 [SMILES: O=C1NC2(CCNCC2)c2ccccc21] | 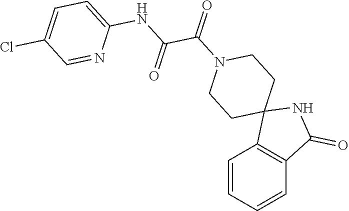 [SMILES: O=C(Nc1ccc(Cl)cn1)C(=O)N1CCC2(CC1)NC(=O)c1ccccc12] | ¹H NMR (500 MHz, DMSO-d₆): δ ppm 11.45 (s, 1H), 9.52 (s, 1H), 8.45 (s, 1H), 8.12-8.10 (m, 1H), 7.99-7.97 (m, 1H), 7.67- 7.61 (m, 3H), 7.52-7.49 (m, 1H), 4.40-4.37 (m, 1H), 3.76- 3.74 (m, 1H), 3.58-3.53 (m, 1H), 3.20-3.14 (m, 1H), 2.26- 2.15 (m, 2H), 1.48-1.42 (m, 2H). MS (ESI): m/z 385.0 [M + H]⁺. |
| 100/7 | Int 10/3, 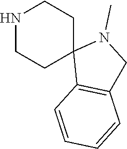 | 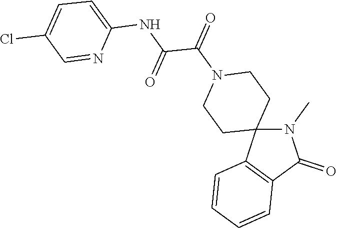 [SMILES: CN1C(=O)c2ccccc2C12CCN(C(=O)C(=O)Nc1ccc(Cl)cn1)CC2] | ¹H NMR (500 MHz, DMSO-d₆): δ ppm 11.49 (s, 1H), 8.45 (d, J = 2.5 Hz, 1H), 8.14-8.12 (m, 2H), 7.99-7.97 (m, 1H), 7.75- 7.74 (m, 1H), 7.64-7.54 (m, 2H), 4.44-4.42 (m, 1H), 3.83- 3.81 (m, 2H), 3.46-3.41 (m, 1H), 2.98 (s, 1H), 2.37-2.16 (m, 2H), 1.48-1.42 (m, 2H). MS (ESI): m/z 399.0 [M + H]⁺. |
| 100/8 | Int 10/3, 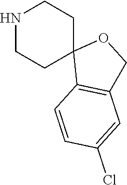 [SMILES: Clc1ccc2c(c1)COC21CCNCC1] | 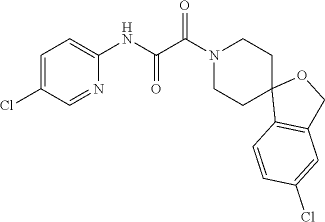 [SMILES: O=C(Nc1ccc(Cl)cn1)C(=O)N1CCC2(CC1)OCc1cc(Cl)ccc12] | ¹H NMR (500 MHz, DMSO-d₆): δ ppm 11.36 (s, 1H), 8.43 (d, J = 2.5 Hz, 1H), 8.09 (br s, 1H), 7.97-7.95 (m, 1H), 7.40-7.36 (m, 2H), 7.31-7.29 (m, 1H), 5.05-4.99 (m, 2H), 4.33-4.30 (m, 1H), 3.67-3.65 (m, 1H), 3.45-3.40 (m, 1H), 3.06-3.00 (m, 1H), 1.97-1.90 (m, 1H), 1.73-1.66 (m, 1H). MS (ESI): m/z 406.1 [M + H]⁺. |
| 100/9 | Int 10/1, 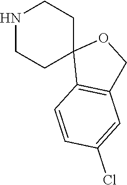 [SMILES: Clc1ccc2c(c1)COC21CCNCC1] | 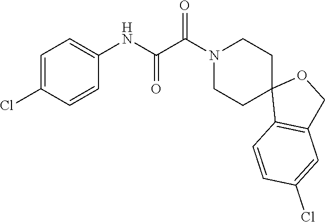 [SMILES: O=C(Nc1ccc(Cl)cc1)C(=O)N1CCC2(CC1)OCc1cc(Cl)ccc12] | ¹H NMR (500 MHz, DMSO-d₆): δ ppm 10.93 (s, 1H), 7.70 (d, J = 8.8 Hz, 2H), 7.42-7.31 (m, 5H), 5.03-5.01 (m, 2H), 4.37- 4.35 (m, 1H), 3.83-3.80 (m, 1H), 3.45-3.40 (m, 1H), 3.08- 3.03 (m, 1H), 1.95-1.86 (m, 2H), 1.76-1.70 (m, 2H). MS (ESI): m/z 405.0 [M + H]⁺. |
| 100/10 | Int 10/21, 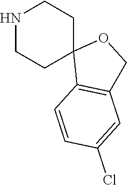 [SMILES: Clc1ccc2c(c1)COC21CCNCC1] | 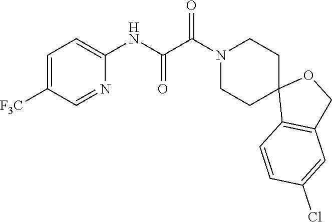 [SMILES: O=C(Nc1ccc(C(F)(F)F)cn1)C(=O)N1CCC2(CC1)OCc1cc(Cl)ccc12] | ¹H NMR (500 MHz, DMSO-d₆): δ ppm 11.18 (s, 1H), 7.88 (d, J = 8.3 Hz, 2H), 7.73 (d, J = 8.6 Hz, 2H), 7.40-7.32 (m, 3H), 5.05-5.00 (m, 2H), 4.39-4.36 (m, 1H), 3.83-3.80 (m, 1H), 3.47-3.41 (m, 1H), 3.10-3.04 (m, 1H), 1.99-1.87 (m, 2H), 1.77-1.67 (m, 2H). MS (ESI): m/z 439.1 [M + H]⁺. |
| 100/11 | Int 10/3, 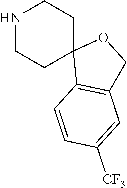 [SMILES: FC(F)(F)c1ccc2c(c1)COC21CCNCC1] | 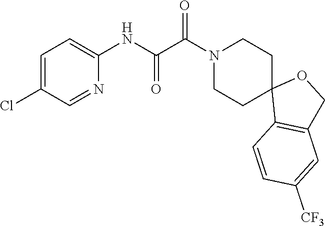 [SMILES: O=C(Nc1ccc(Cl)cn1)C(=O)N1CCC2(CC1)OCc1cc(C(F)(F)F)ccc12] | ¹H NMR (500 MHz, DMSO-d₆): δ ppm 11.38 (s, 1H), 8.44-8.43 (m, 1H), 8.18-7.86 (m, 1H), 7.47-7.46 (m, 1H), 7.27-7.26 (m, 1H), 7.14 (s, 1H), 4.64-4.60 (m, 2H), 4.28-4.25 (m, 1H), 3.69-3.66 (m, 1H), 3.33-3.28 (m, 1H), 2.97-2.92 (m, 1H), 1.94-1.88 (m, 2H), 1.80-1.73 (m, 2H). MS (ESI): m/z 440.1 [M + H]⁺. |
| 100/12 | Int 10/1, 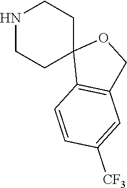 [SMILES: FC(F)(F)c1ccc2c(c1)COC21CCNCC1] | 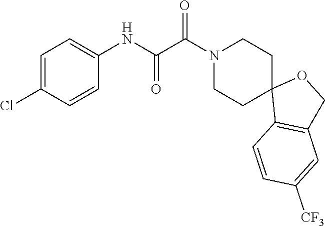 [SMILES: O=C(Nc1ccc(Cl)cc1)C(=O)N1CCC2(CC1)OCc1cc(C(F)(F)F)ccc12] | ¹H NMR (500 MHz, DMSO-d₆): δ ppm 10.97 (s, 1H), 7.72-7.69 (m, 2H), 7.49-7.47 (m, 1H), 7.44-7.41 (m, 2H), 7.26-7.25 (m, 1H), 7.15 (s, 1H), 4.65-4.60 (m, 2H), 4.33-4.30 (m, 1H), 3.84-3.81 (m, 1H), 3.33-3.29 (m, 1H), 3.01-2.95 (m, 1H), 1.93-1.77 (m, 4H). MS (ESI): m/z 437.0 [M − H]⁻. |
| 100/13 | Int 10/1 Int 40 | 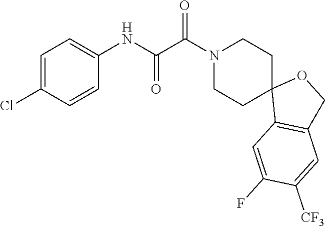 [SMILES: O=C(Nc1ccc(Cl)cc1)C(=O)N1CCC2(CC1)OCc1cc(C(F)(F)F)c(F)cc12] | ¹H NMR (400 MHz, DMSO-d₆): δ ppm 10.94 (s, 1H), 7.76-7.65 (m, 2H), 7.52 (d, J = 10.2 Hz, 1H), 7.45-7.38 (m, 2H), 7.17 (d, J = 5.5 Hz, 1H), 4.74-4.51 (m, 2H), 4.34-4.26 (m, 1H), 3.89- 3.81 (m, 1H), 3.32-3.34 (m, 1H), 3.04-2.79 (m, 1H), 2.00- 1.72 (m, 4H). MS (ESI): m/z 457.1 [M + H]⁺. |
| 100/14 | Int 10/1 Int 40/1 | 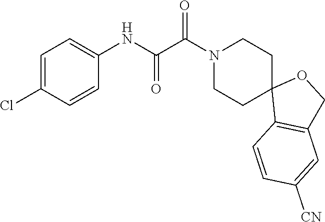 [SMILES: N#Cc1ccc2c(c1)COC21CCN(C(=O)C(=O)Nc2ccc(Cl)cc2)CC1] | ¹H NMR (400 MHz, DMSO-d₆): δ ppm 10.94 (s, 1H), 7.71-7.61 (m, 2H), 7.45 (d, J = 7.7 Hz, 1H), 7.42-7.37 (m, 2H), 7.35 (dd, J = 7.7, 1.4 Hz, 1H), 7.29 (d, J = 1.1 Hz, 1H), 4.72-4.45 (m, 2H), 4.32-4.25 (m, 1H), 3.83-3.75 (m, 1H), 3.30-3.22 (m, 1H), 2.98-2.88 (m, 1H), 1.96-1.62 (m, 4H). MS (ESI): m/z 396.2 [M + H]⁺. |
| 100/15 | Int 10/3, 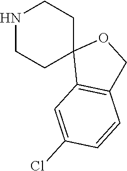 [SMILES: Clc1ccc2c(c1)C1(CCNCC1)OC2] | 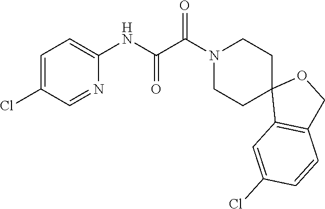 [SMILES: O=C(Nc1ccc(Cl)cn1)C(=O)N1CCC2(CC1)OCc1ccc(Cl)cc12] | ¹H NMR (500 MHz, DMSO-d₆): δ ppm 11.36 (s, 1H), 8.44 (d, J = 2.4 Hz, 1H), 8.11 (d, J = 8.8 Hz, 1H), 7.98 (dd, J = 8.9, 2.1 Hz, 1H), 7.39-7.33 (m, 3H), 5.09-4.92 (m, 1H), 4.34-4.28 (m, 1H), 3.69-3.65 (m, 1H), 3.46-3.41 (m, 1H), 3.05-3.00 (m, 1H), 2.03-1.94 (m, 2H), 1.74-1.68 (m, 2H). MS (ESI): m/z 406.1 [M + H]⁺. |

### Example 101: N-(4-Chlorophenyl)-2-(6-(difluoromethyl)-2H-spiro[benzofuran-3,4′-piperidin]-1′-yl)-2-oxoacetamide (101)

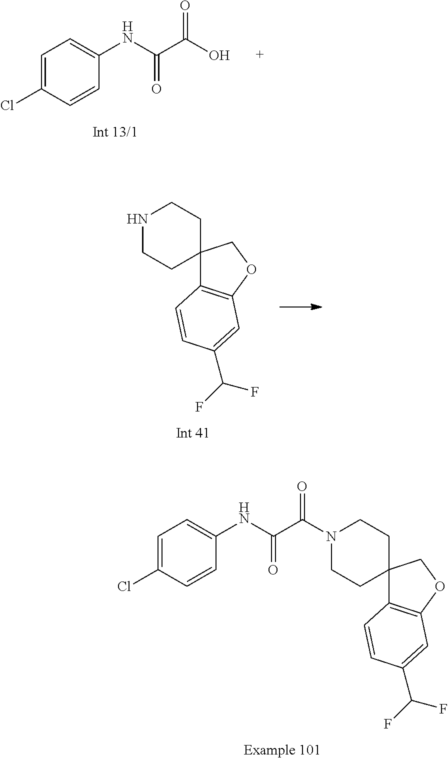 [SMILES: O=C(O)C(=O)Nc1ccc(Cl)cc1 ; FC(F)c1ccc2c(c1)OCC21CCNCC1 ; O=C(Nc1ccc(Cl)cc1)C(=O)N1CCC2(CC1)COc1cc(C(F)F)ccc12]

A mixture of 6-(difluoromethyl)-2H-spiro[benzofuran-3,4′-piperidine] (Int 41) (69 mg, 0.29 mmol), 2-(4-chlorophenylamino)-2-oxoacetic acid (Int 13/1) (58 mg, 0.29 mmol), BOPCl (74 mg, 0.29 mmol) and DIEA (75 mg, 0.58 mmol) in DCM (10 mL) was stirred at rt overnight. Water (20 mL) was added and the mixture was extracted with DCM (3×10 mL). The combined organic layers were washed with brine (20 mL), dried over Na₂SO₄, filtered and concentrated to dryness. The residue was purified by preparative HPLC to give the title compound as a white solid. ¹H NMR (400 MHz, CDCl₃): δ ppm 9.23 (s, 1H), 7.63-7.51 (m, 2H), 7.39-7.30 (m, 2H), 7.18 (d, J=7.6 Hz, 1H), 7.05 (d, J=7.6 Hz, 1H), 7.05 (s, 1H), 6.58 (t, J=56.4 Hz, 1H), 5.22-5.14 (m, 1H), 4.65-4.42 (m, 3H), 3.47-3.28 (m, 1H), 3.08-2.88 (m, 1H), 2.19-1.80 (m, 4H). MS (ESI): m/z 421.1 [M+H]⁺.

### Example 102: N-(4-Chlorophenyl)-2-oxo-2-(6′-(trifluoromethyl)-2′H-spiro[azepane-4,3′-benzofuran]-1-yl)acetamide (102)

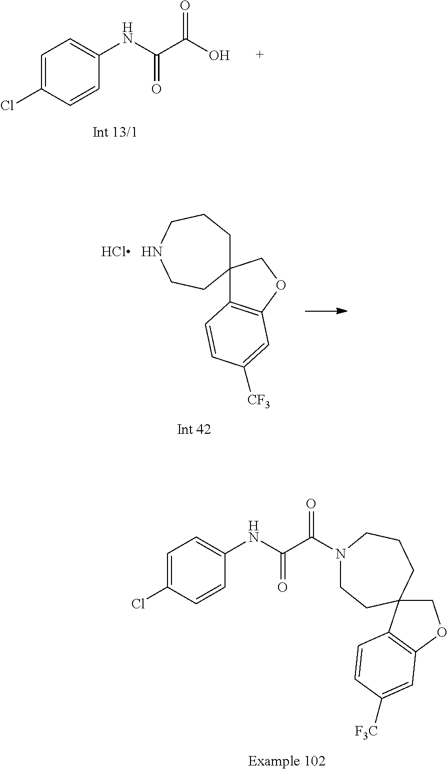 [SMILES: O=C(O)C(=O)Nc1ccc(Cl)cc1 ; FC(F)(F)c1ccc2c(c1)OCC21CCCNCC1 ; O=C(Nc1ccc(Cl)cc1)C(=O)N1CCCC2(CC1)COc1cc(C(F)(F)F)ccc12]

To a solution of 2-((4-chlorophenyl)amino)-2-oxoacetic acid (Int 13/1) (40 mg, 0.20 mmol), EDCl.HCl (58 mg, 0.30 mmol) and HOBt (40 mg, 0.30 mmol) in CH₂Cl₂ (10 mL) was added N-methylmorpholine (0.5 mL) and the mixture was stirred at rt for 30 min. 6′-(Trifluoromethyl)-2′H-spiro[azepane-4,3′-benzofuran] hydrochloride (Int 42) (60 mg, 0.2 mmol) was added and the mixture was stirred at rt overnight. The mixture was poured into water (20 mL) and extracted with EtOAc (2×20 mL). The combined organic layers were concentrated to dryness and the residue was purified by preparative HPLC to give the title compound as a white solid. ¹H NMR (400 MHz, DMSO-d₆): δ ppm 10.96 (s, 1H), 7.78-7.61 (m, 2H), 7.50-7.36 (m, 3H), 7.28-7.23 (m, 1H), 7.13-7.11 (m, 1H), 4.59-4.38 (m, 2H), 3.85-3.38 (m, 4H), 2.12-1.69 (m, 6H). MS (ESI): m/z 453.1 [M+H]⁺.

### Example 103: N-(4-Fluorobicyclo[4.2.0]octa-1(6),2,4-trien-7-yl)-2-oxo-2-(6-(trifluoromethyl)-2H-spiro[benzofuran-3,4′-piperidin]-1′-yl)acetamide (103)

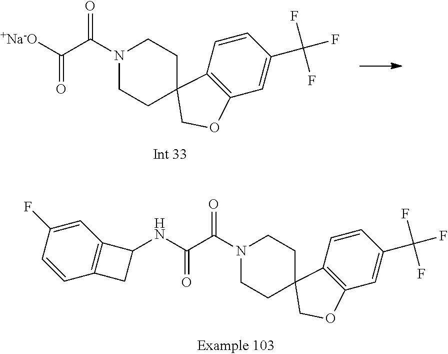

To a mixture of sodium 2-oxo-2-(6-(trifluoromethyl)-2H-spiro[benzofuran-3,4′-piperidin]-1′-yl)acetate (Int 33) (30 mg, 0.085 mmol), HATU (48.7 mg, 0.128 mmol) and TEA (0.030 mL, 0.214 mmol) in DCM (1.0 mL) 4-fluorobicyclo[4.2.0]octa-1(6),2,4-trien-7-amine hydrochloride (Int 3) (14.8 mg, 0.085 mmol) was added and the mixture was stirred at rt overnight. The mixture was concentrated to dryness and the residue was purified by preparative HPLC to afford the title compound as a white solid. ¹H NMR (DMSO-d₆, 300 MHz): δ ppm 9.46 (d, J=6.7 Hz, 1H), 7.47-7.42 (m, 1H), 7.25-7.03 (m, 5H), 5.34-5.26 (m, 1H), 4.62-4.60 (m, 2H), 4.28-4.23 (m, 1H), 3.84-3.74 (m, 1H), 3.59-3.52 (m, 1H), 3.30-3.20 (m, 1H), 3.11-3.00 (m, 1H), 2.97-2.84 (m, 1H), 1.88-1.74 (m, 4H). MS (ESI): m/z 449.3 [M+H]⁺.

### Example 103/1: N-(bicyclo[4.2.0]octa-1(6),2,4-trien-7-yl)-2-oxo-2-(6-(trifluoromethyl)-2H-spiro[benzofuran-3,4′-piperidin]-1′-yl)acetamide (103/1)

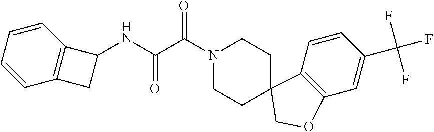 [SMILES: O=C(NC1Cc2ccccc21)C(=O)N1CCC2(CC1)COc1cc(C(F)(F)F)ccc12]

The title compound was prepared similar as described for Example 103 using bicyclo[4.2.0]octa-1(6),2,4-trien-7-amine hydrogen chloride in place of 4-fluorobicyclo[4.2.0]octa-1(6),2,4-trien-7-amine hydrochloride (Int 3). ¹H NMR (DMSO-d₆, 300 MHz): δ ppm 9.43 (d, J=7.1 Hz, 1H), 7.44 (d, J=7.5 Hz, 1H), 7.34-7.20 (m, 6H), 5.35-5.31 (m, 1H), 4.62-4.60 (m, 2H), 4.28-4.20 (m, 1H), 3.84-3.73 (m, 1H), 3.61 (dd, J=14.2, 5.3 Hz, 1H), 3.27-3.23 (m, 1H), 3.15-3.07 (m, 1H), 2.95-2.85 (m, 1H), 1.88-1.73 (m, 4H). MS (ESI): m/z 431.3 [M+H]⁺.

Biological Assays

SKOV-3 Cellular Indoleamine 2,3-Dioxygenase Assay

SKOV-3 cells were obtained from the American Type Culture Collection (ATCC® HTB-77™) and maintained in McCoy's medium (Pan Biotech) supplemented with 10% fetal bovine serum and 1% Penicilin/Streptomycin. Cells were kept at 37° C. in a humidified incubator with 5% CO₂. For assay preparation, cells were seeded at a density of 2*10⁵/m into black clear bottom 96 well plates in 100 μl medium/well supplemented with 50 ng/ml Interferon gamma (eBioscience, Thermo Fisher Scientific). After cells fully adhered to the plate, dilution series of compounds were added in medium containing additional L-Tryptophan to a final L-Tryptophan concentration of 100 μM. The cells were incubated for 24 hours. Detection of produced N-Formylkynurenin was performed by addition of 3-Methylpiperidine to a final concentration of 200 mM. The plates were sealed and heated to 65° C. for 20 minutes in a water bath. After cooling the fluorescence of each well was recorded with a Victor™ X4 (PerkinElmer) plate reader at an emission wavelength of 535 nm and excitation at 405 nm (Tomek et al.; Anal Bioanal Chem (2013) 405:2515-2524., Tomek et al.; Biochim Biophys Acta. 2015 September; 1850(9):1772-80).

The IC₅₀ values of the example compounds are shown in Table 1 below (A=IC₅₀<100 nM, B=100 nM≤IC₅₀≤1 μM, C=IC₅₀>1 μM).

TABLE 1

| Example # | cell based activity |
|---|---|
| 1 | A |
| 1/1 | A |
| 1/2 | A |
| 1/3 | A |
| 1/4 | B |
| 1/5 | B |
| 1/6 | A |
| 1/7 | C |
| 1/8 | A |
| 1/9 | C |
| 1/10 | B |
| 1/11 | A |
| 1/12 | A |
| 1/13 | A |
| 1/14 | A |
| 1/15 | B |
| 1/16 | C |
| 1/17 | A |
| 1/18 | A |
| 1/19 | A |
| 1/20 | C |
| 1/21 | B |
| 1/22 | A |
| 1/23 | A |
| 1/24 | A |
| 1/25 | A |
| 1/26 | A |
| 1/27 | A |
| 1/28 | A |
| 1/29 | A |
| 1/30 | A |
| 1/31 | A |
| 1/32 | B |
| 1/33 | C |
| 1/34 | C |
| 1/35 | B |
| 1/36 | B |
| 1/37 | A |
| 1/38 | A |
| 1/39 | A |
| 1/40 | A |
| 1/41 | B |
| 1/42 | C |
| 1/43 | C |
| 1/44 | B |
| 1/45 | A |
| 1/46 | A |
| 1/47 | A |
| 1/48 | A |
| 2 | A |
| 2/1 | C |
| 2/2 | C |
| 2/3 | C |
| 2/4 | B |
| 2/5 | B |
| 2/6 | B |
| 3 | A |
| 4 | B |
| 5 | B |
| 5/1 | C |
| 5/2 | B |
| 5/3 | B |
| 5/4 | B |
| 5/5 | C |
| 5/6 | C |
| 5/7 | B |
| 5/8 | C |
| 5/9 | C |
| 5/10 | C |
| 6 | B |
| 6/1 | C |
| 6/2 | C |
| 7 | A |
| 7/1 | A |
| 7/2 | A |
| 8 | C |
| 8/1 | C |
| 8/2 | C |
| 8/3 | C |
| 8/4 | C |
| 8/5 | C |
| 8/6 | C |
| 9 | C |
| 10a | A |
| 10b | A |
| 10/1a | B |
| 10/1b | C |
| 10/2a | A |
| 10/2b | A |
| 100 | A |
| 100/1 | B |
| 100/2 | B |
| 100/3 | C |
| 100/4 | C |
| 100/5 | C |
| 100/6 | C |
| 100/7 | C |
| 100/8 | B |
| 100/9 | A |
| 100/10 | B |
| 100/11 | A |
| 100/12 | A |
| 100/13 | A |
| 100/14 | B |
| 100/15 | C |
| 101 | A |
| 102 | B |
| 103 | C |

1. A compound according to Formula (1) or (2) 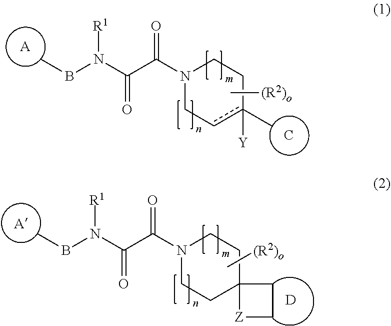
an enantiomer, diastereomer, tautomer or pharmaceutically acceptable salt thereof
wherein
A and A′ represent C₃₋₁₀ cycloalkyl, which may be optionally fused with a phenyl ring being unsubstituted or substituted with 1 to 3 R^{a}, 3- to 10-membered heterocycloalkyl containing 1 to 4 heteroatoms independently selected from O, N and S, 6- to 10-membered mono or bicyclic aryl or 5- to 10-membered mono or bicyclic heteroaryl containing 1 to 4 heteroatoms independently selected from O, N and S,
wherein cycloalkyl, heterocycloalkyl, aryl and heteroaryl are unsubstituted or substituted with 1 to 5 substituents independently selected from the group consisting of halogen, OH, R, O—R^{x}, OC(O)—R^{x}, S—R^{x}, S(O)₂—R^{x}, S(O)₂N(R)₂, N(R¹)₂, NR¹C(O)R^{x}, C(O)N(R¹)₂, C(O)O—R^{x}, C(O)—R^{x}, CN, COOH, 5- or 6-membered heteroaryl containing 1 to 4 heteroatoms independently selected from O, N and S and 6-membered aryl,
wherein heteroaryl and aryl are unsubstituted or substituted with 1 to 5 substituents independently selected from the group consisting of halogen, CN, OR¹, C₁₋₆-alkyl, C₃₋₆-cycloalkyl and halo-C₁₋₆-alkyl,
or
two substituents on the same carbon atom or on two different carbon atoms form together with the carbon atom to which they are attached a C₃₋₁₀ cycloalkyl group, wherein optionally one carbon atom in the cycloalkyl ring may be replaced by a heteroatom selected from O, N and S and wherein the (hetero)cyclic ring may be unsubstituted or substituted by 1 to 3 substituents independently selected from the group consisting of halogen, C₁₋₆-alkyl, halo-C₁₋₆-alkyl or oxo;
R^{a} represents halogen, CN, C₁₋₆-alkyl, halo-C₁₋₆-alkyl, hydroxy-C₁₋₆-alkyl, C₃₋₆-cycloalkyl or halo-C₃₋₆-cycloalkyl;
R^{x} represents C₁₋₆-alkyl, C₃₋₆-cycloalkyl or 3- to 6 membered heterocyclyl containing 1 to 2 heteroatoms independently selected from O, N and S,
wherein alkyl, cycloalkyl and heterocycloalkyl are unsubstituted or substituted with 1 or 6 substituents independently selected from the group consisting of halogen, C₁₋₆-alkyl, halo C₁₋₆-alkyl, OR¹ and CN;
B represents a bond or C₁₋₂-alkylene,
wherein alkylene is unsubstituted or substituted with one or two C₁₋₄-alkyl;
C represents 6- to 10-membered mono- or bicyclic aryl or 5- to 14-membered mono-, bi- or tricyclic heteroaryl containing 1 to 4 heteroatoms independently selected from O, N and S,
wherein aryl and heteroaryl are unsubstituted or substituted with 1 to 5 substituents independently selected from the group consisting of halogen, OH, R^{y}, O—R^{y}, OC(O)—R^{y}, S—R^{y}, S(O)₂—R^{y}, S(O)₂N(R¹)₂, N(R¹)₂, NR¹C(O)R^{y}, C(O)N(R)₂, C(O)O—R^{y}, C(O)—R^{y}, CN, COOH, 5- or 6-membered heteroaryl containing 1 to 4 heteroatoms independently selected from O, N and S and 6-membered aryl,
wherein aryl and heteroaryl are unsubstituted or substituted with 1 to 5 substituents independently selected from the group consisting of halogen, CN, OR¹, C₁₋₆-alkyl, C₃₋₆-cycloalkyl, and halo-C₁₋₆-alkyl,
or
two substituents on the aryl or heteroaryl ring systems together with the carbon atoms to which they are attached form a 5- or 6-membered heterocyclic ring containing 1 or 2 heteroatoms independently selected from O, N and S,
wherein the heterocylic ring is unsubstituted or substituted with 1 or 2 substituents independently selected from the group consisting of halogen, C₁₋₆-alkyl, halo-C₁₋₆-alkyl, CN and oxo;
R^{y} represents C₁₋₆-alkyl, C₃₋₆-cycloalkyl or 3- to 6 membered heterocyclyl containing 1 to 2 heteroatoms independently selected from O, N and S,
wherein alkyl, cycloalkyl and heterocycloalkyl are unsubstituted or substituted with 1 to 6 substituents independently selected from the group consisting of halogen, C₁₋₆-alkyl, halo-C₁₋₆-alkyl, OR¹, CN and phenyl;
D represents 6- to 10-membered mono- or bicyclic aryl or 5- to 10-membered mono- or bicyclic heteroaryl containing 1 to 4 heteroatoms independently selected from O, N and S,
wherein aryl and heteroaryl are unsubstituted or substituted with 1 to 5 substituents independently selected from the group consisting of halogen, OH, R^{z}, O—R^{z}, OC(O)—R^{z}, S—R^{z}, S(O)₂—R^{z}, S(O)₂N(R¹)₂, N(R¹)₂, NR¹C(O)R^{z}, C(O)N(R)₂, C(O)O—R^{z}, C(O)—R^{z}, CN, COOH, 5- or 6-membered heteroaryl containing 1 to 4 heteroatoms independently selected from O, N and S and 6-membered aryl,
wherein aryl and heteroaryl are unsubstituted or substituted with 1 to 5 substituents independently selected from the group consisting of halogen, CN, OR¹, C₁₋₆-alkyl, C₃₋₆-cycloalkyl and halo C₁₋₆-alkyl,
or
two substituents on the aryl or heteroaryl ring systems together with the carbon atom to which they are attached form a 5- or 6-membered heterocyclic ring containing 1 or 2 heteroatoms independently selected from O, N and S,
wherein the heterocylic ring is unsubstituted or substituted with 1 or 2 substituents independently selected from the group consisting of halogen, C₁₋₆-alkyl, halo-C₁₋₆-alkyl, CN and oxo;
R^{z} represents C₁₋₆-alkyl, C₃₋₆-cycloalkyl or 3- to 6 membered heterocyclyl containing 1 to 2 heteroatoms independently selected from O, N and S,
wherein alkyl, cycloalkyl and heterocycloalkyl are unsubstituted or substituted with 1 to 6 substituents independently selected from the group consisting of halogen, C₁₋₆-alkyl, halo-C₁₋₆-alkyl, OR¹ and CN;
Y is absent or represents hydrogen, OR⁴, halogen, C₁₋₆-alkyl or halo-C₁₋₆-alkyl;
Z represents —C₂₋₃-alkylene-, —O—C₁₋₂-alkylene-, —C₁₋₂-alkylene-O—, —NR³C(O)—C₀₋₁-alkylene-, —C(O)NR³—C₀₋₁-alkylene-, —C₀₋₁-alkylene-NR³C(O)—, —C₀₋₁-alkylene C(O)NR³—, —S(O)ₜ—C₁₋₂-alkylene-, —C₁₋₂-alkylene-S(O)ₜ—, —NR⁹—C₁₋₂-alkylene- or —C₁₋₂-alkylene-NR⁹—,
wherein alkylene is unsubstituted or substituted with 1 or 2 substituents independently selected from the group consisting of OR⁴, C₁₋₆-alkyl, halogen and halo-C₁₋₆-alkyl;
R⁹ is hydrogen, C₁₋₆-alkyl, halo-C₁₋₆-alkyl, C(O)—C₁₋₆-alkyl, or C(O)-halo-C₁₋₆-alkyl;
R¹ is hydrogen or C₁₋₆-alkyl;
R² is halogen, C₁₋₆-alkyl, C₃₋₆-cycloalkyl, halo-C₁₋₆-alkyl, OR⁴, S(O)₂N(R¹)₂, S(O)₂—C₁₋₆-alkyl, S(O)₂—C₃₋₆-cycloalkyl, S(O)₂-halo-C₁₋₆-alkyl, C(O)N(R¹)₂, CN, C(O)OR⁴ or oxo,
or
two R² on the same carbon atom form together with the carbon atom to which they are attached a C₃₋₁₀ cycloalkyl group,
or
two R² at different carbon atoms form together a —CH₂—, —CH(CH₃)—, —C(CH₃)₂—, CH₂—CH(CH₃)—, —CH₂—CH₂— or —CH₂—CH₂—CH₂— group;
R³ is hydrogen or C₁₋₆-alkyl;
R⁴ is hydrogen or C₁₋₆-alkyl;
m is 0-2;
n is 0-2;
o is 0-4; and
t is 0, 1 or 2;
with the proviso that C is not 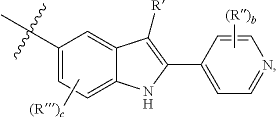
wherein R′ is H, Cl, CN, C₁₋₄-alkyl, C₁₋₃-fluoroalkyl, C₁₋₃-hydroxy-fluoroalkyl, C₃₋₆-cycloalkyl, —C(O)O(C₁₋₃-alkyl) or tetrahydropyranyl, R″ is halogen, CN, OH, C₁₋₃-alkyl, C₁₋₂-fluoroalkyl, O—C₁₋₃-alkyl or C₃₋₆-cycloalkyl, R′″ is F, Cl, CN, C₁₋₂-alkyl, C₁₋₂-fluoro-alkyl or OCH₃, b is 0 to 4 and c is 0, 1 or 2.

2. A compound according to claim 1, wherein
A and A′ represent C₃₋₁₀ cycloalkyl, which may be optionally fused with a phenyl ring, 3- to 10-membered heterocycloalkyl containing 1 to 4 heteroatoms independently selected from O, N and S, 6- to 10-membered mono or bicyclic aryl or 5- to 10-membered mono or bicyclic heteroaryl containing 1 to 4 heteroatoms independently selected from O, N and S,
wherein cycloalkyl, heterocycloalkyl, aryl and heteroaryl are unsubstituted or substituted with 1 to 5 substituents independently selected from the group consisting of halogen, OH, R, O—R^{x}, OC(O)—R^{x}, S—R^{x}, S(O)₂—R^{x}, S(O)₂N(R¹)₂, N(R¹)₂, NR¹C(O)R^{x}, C(O)N(R¹)₂, C(O)O—R^{x}, C(O)—R^{x}, CN, COOH, 5- or 6-membered heteroaryl containing 1 to 4 heteroatoms independently selected from O, N and S and 6-membered aryl,
wherein heteroaryl and aryl are unsubstituted or substituted with 1 to 5 substituents independently selected from the group consisting of halogen, CN, OR¹, C₁₋₆-alkyl, C₃₋₆-cycloalkyl and halo-C₁₋₆-alkyl,
or
two substituents on the same carbon atom or on two different carbon atoms form together with the carbon atom to which they are attached a C₃₋₁₀ cycloalkyl group, wherein optionally one carbon atom in the cycloalkyl ring may be replaced by a heteroatom selected from O, N and S and wherein the (hetero)cyclic ring may be unsubstituted or substituted by 1 to 3 substituents independently selected from the group consisting of halogen, C₁₋₆-alkyl, halo-C₁₋₆-alkyl or oxo;
R^{x} represents C₁₋₆-alkyl, C₃₋₆-cycloalkyl or 3- to 6 membered heterocyclyl containing 1 to 2 heteroatoms independently selected from O, N and S,
wherein alkyl, cycloalkyl and heterocycloalkyl are unsubstituted or substituted with 1 or 6 substituents independently selected from the group consisting of halogen, C₁₋₆-alkyl, halo C₁₋₆-alkyl, OR¹ and CN;
B represents a bond or C₁₋₂-alkylene,
wherein alkylene is unsubstituted or substituted with one or two C₁₋₄-alkyl;
C represents 6- to 10-membered mono- or bicyclic aryl or 5- to 14-membered mono-, bi- or tricyclic heteroaryl containing 1 to 4 heteroatoms independently selected from O, N and S,
wherein aryl and heteroaryl are unsubstituted or substituted with 1 to 5 substituents independently selected from the group consisting of halogen, OH, R^{y}, O—R^{y}, OC(O)—R^{y}, S—R^{y}, S(O)₂—R^{y}, S(O)₂N(R)₂, N(R)₂, NR¹C(O)R^{y}, C(O)N(R)₂, C(O)O—R^{y}, C(O)—R^{y}, CN, COOH, 5- or 6-membered heteroaryl containing 1 to 4 heteroatoms independently selected from O, N and S and 6-membered aryl,
wherein aryl and heteroaryl are unsubstituted or substituted with 1 to 5 substituents independently selected from the group consisting of halogen, CN, OR¹, C₁₋₆-alkyl, C₃₋₆-cycloalkyl, and halo-C₁₋₆-alkyl,
or
two substituents on the aryl or heteroaryl ring systems together with the carbon atoms to which they are attached form a 5- or 6-membered heterocyclic ring containing 1 or 2 heteroatoms independently selected from O, N and S,
wherein the heterocylic ring is unsubstituted or substituted with 1 or 2 substituents independently selected from the group consisting of halogen, C₁₋₆-alkyl, halo-C₁₋₆-alkyl, CN and oxo;
R^{y} represents C₁₋₆-alkyl, C₃₋₆-cycloalkyl or 3- to 6 membered heterocyclyl containing 1 to 2 heteroatoms independently selected from O, N and S,
wherein alkyl, cycloalkyl and heterocycloalkyl are unsubstituted or substituted with 1 to 6 substituents independently selected from the group consisting of halogen, C₁₋₆-alkyl, halo-C₁₋₆-alkyl, OR¹, CN and phenyl;
D represents 6- to 10-membered mono- or bicyclic aryl or 5- to 10-membered mono- or bicyclic heteroaryl containing 1 to 4 heteroatoms independently selected from O, N and S,
wherein aryl and heteroaryl are unsubstituted or substituted with 1 to 5 substituents independently selected from the group consisting of halogen, OH, R^{z}, O—R^{z}, OC(O)—R^{z}, S—R^{z}, S(O)₂—R^{z}, S(O)₂N(R¹)₂, N(R¹)₂, NR¹C(O)R^{z}, C(O)N(R)₂, C(O)O—R^{z}, C(O)—R^{z}, CN, COOH, 5- or 6-membered heteroaryl containing 1 to 4 heteroatoms independently selected from O, N and S and 6-membered aryl,
wherein aryl and heteroaryl are unsubstituted or substituted with 1 to 5 substituents independently selected from the group consisting of halogen, CN, OR¹, C₁₋₆-alkyl, C₃₋₆-cycloalkyl and halo C₁₋₆-alkyl,
or
two substituents on the aryl or heteroaryl ring systems together with the carbon atom to which they are attached form a 5- or 6-membered heterocyclic ring containing 1 or 2 heteroatoms independently selected from O, N and S,
wherein the heterocylic ring is unsubstituted or substituted with 1 or 2 substituents independently selected from the group consisting of halogen, C₁₋₆-alkyl, halo-C₁₋₆-alkyl, CN and oxo;
R^{z} represents C₁₋₆-alkyl, C₃₋₆-cycloalkyl or 3- to 6 membered heterocyclyl containing 1 to 2 heteroatoms independently selected from O, N and S,
wherein alkyl, cycloalkyl and heterocycloalkyl are unsubstituted or substituted with 1 to 6 substituents independently selected from the group consisting of halogen, C₁₋₆-alkyl, halo-C₁₋₆-alkyl, OR¹ and CN;
Y is absent or represents hydrogen, OR⁴, halogen, C₁₋₆-alkyl or halo-C₁₋₆-alkyl;
Z represents —C₂₋₃-alkylene-, —O—C₁₋₂-alkylene-, —C₁₋₂-alkylene-O—, —NR³C(O)—C₀₋₁-alkylene-, —C(O)NR³—C₀₋₁-alkylene-, —C₀₋₁-alkylene-NR³C(O)—, —C₀₋₁-alkylene C(O)NR³—, —S(O)ₜ—C₁₋₂-alkylene-, —C₁₋₂-alkylene-S(O)ₜ—, —NR⁹—C₁₋₂-alkylene- or —C₁₋₂-alkylene-NR⁹—,
wherein alkylene is unsubstituted or substituted with 1 or 2 substituents independently selected from the group consisting of OR⁴, C₁₋₆-alkyl, halogen and halo-C₁₋₆-alkyl;
R⁹ is hydrogen, C₁₋₆-alkyl, halo-C₁₋₆-alkyl, C(O)—C₁₋₆-alkyl, or C(O)-halo-C₁₋₆-alkyl;
R¹ is hydrogen or C₁₋₆-alkyl;
R² is C₁₋₆-alkyl, C₃₋₆-cycloalkyl, halo-C₁₋₆-alkyl, OR⁴, S(O)₂N(R)₂, S(O)₂—C₁₋₆-alkyl, S(O)₂—C₃₋₆-cycloalkyl, S(O)₂-halo-C₁₋₆-alkyl, C(O)N(R¹)₂, CN, C(O)OR⁴ or oxo,
or
two R² on the same carbon atom form together with the carbon atom to which they are attached a C₃₋₁₀ cycloalkyl group,
or
two R² at different carbon atoms form together a —CH₂—, —CH(CH₃)—, —C(CH₃)₂—, CH₂—CH(CH₃)—, —CH₂—CH₂— or —CH₂—CH₂—CH₂— group;
R³ is hydrogen or C₁₋₆-alkyl;
R⁴ is hydrogen or C₁₋₆-alkyl;
m is 0-2;
n is 0-2;
o is 0-4; and
t is 0, 1 or 2.

3. The compound according to claim 1 which is represented by the following formulae (2-1) and (2-2) 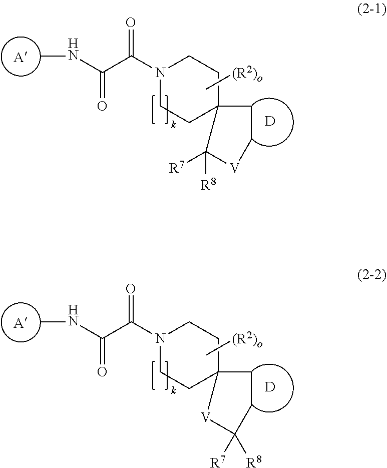
wherein
A′ represents C₃₋₁₀ cycloalkyl, which may be optionally fused with a phenyl ring being unsubstituted or substituted with 1 to 3 R^{a}, 3- to 10-membered heterocycloalkyl containing 1 to 4 heteroatoms independently selected from O, N and S, 6- to 10-membered mono or bicyclic aryl or 5- to 10-membered mono or bicyclic heteroaryl containing 1 to 4 heteroatoms independently selected from O, N and S,
wherein cycloalkyl, heterocycloalkyl, aryl and heteroaryl are unsubstituted or substituted with 1 to 5 substituents independently selected from the group consisting of halogen, OH, R, O—R^{x}, OC(O)—R^{x}, S—R^{x}, S(O)₂—R^{x}, S(O)₂N(R)₂, N(R¹)₂, NR¹C(O)R^{x}, C(O)N(R¹)₂, C(O)O—R^{x}, C(O)—R^{x}, CN, COOH, 5- or 6-membered heteroaryl containing 1 to 4 heteroatoms independently selected from O, N and S and 6-membered aryl,
wherein heteroaryl and aryl are unsubstituted or substituted with 1 to 5 substituents independently selected from the group consisting of halogen, CN, OR¹, C₁₋₆-alkyl, C₃₋₆-cycloalkyl and halo-C₁₋₆-alkyl,
or
two substituents on the same carbon atom or on two different carbon atoms form together with the carbon atom to which they are attached a C₃₋₁₀ cycloalkyl group, wherein optionally one carbon atom in the cycloalkyl ring may be replaced by a heteroatom selected from O, N and S;
R^{a} represents halogen, CN, C₁₋₆-alkyl, halo-C₁₋₆-alkyl, hydroxy-C₁₋₆-alkyl, C₃₋₆-cycloalkyl or halo-C₃₋₆-cycloalkyl;
R^{x} represents C₁₋₆-alkyl, C₃₋₆-cycloalkyl or 3- to 6 membered heterocyclyl containing 1 to 2 heteroatoms independently selected from O, N and S,
wherein alkyl, cycloalkyl and heterocycloalkyl are unsubstituted or substituted with 1 to 6 substituents independently selected from the group consisting of halogen, C₁₋₆-alkyl, halo-C₁₋₆-alkyl, OR¹ and CN;
D represents 6- to 10-membered mono- or bicyclic aryl or 5- to 10-membered mono- or bicyclic heteroaryl containing 1 to 4 heteroatoms independently selected from O, N and S,
wherein aryl and heteroaryl are unsubstituted or substituted with 1 to 5 substituents independently selected from halogen, OH, R^{z}, O—R^{z}, OC(O)—R^{z}, S—R^{z}, S(O)₂—R^{z}, S(O)₂N(R¹)₂, N(R¹)₂, NR¹C(O)R^{z}, C(O)N(R¹)₂, C(O)O—R^{z}, C(O)—R^{z}, CN, COOH, 5- or 6-membered heteroaryl containing 1 to 4 heteroatoms independently selected from O, N and S and 6-membered aryl,
wherein aryl and heteroaryl are unsubstituted or substituted with 1 to 5 substituents independently selected from the group consisting of halogen, CN, OR¹, C₁₋₆-alkyl; C₃₋₆-cycloalkyl, and halo-C₁₋₆-alkyl,
or
two substituents on the aryl or heteroaryl ring systems together with the carbon atom to which they are attached form a 5- or 6-membered heterocyclic ring containing 1 or 2 heteroatoms independently selected from O, N and S,
wherein the heterocylic ring is unsubstituted or substituted with 1 or 2 substituents independently selected from the group consisting of halogen, C₁₋₆-alkyl, halo-C₁₋₆-alkyl, CN and oxo;
R^{z} represents C₁₋₆-alkyl, C₃₋₆-cycloalkyl or 3- to 6 membered heterocyclyl containing 1 to 2 heteroatoms independently selected from O, N and S,
wherein alkyl, cycloalkyl and heterocycloalkyl are unsubstituted or substituted with 1 to 6 substituents independently selected from the group consisting of halogen, C₁₋₆-alkyl, halo-C₁₋₆-alkyl, OR¹ and CN;
V is O or CR⁷R⁸;
R² is halogen, C₁₋₆ alkyl, C₃₋₆ cycloalkyl, halo-C₁₋₆-alkyl, OR⁴, S(O)₂N(R¹)₂, S(O)₂—C₁₋₆-alkyl, S(O)₂—C₃₋₆-cycloalkyl, S(O)₂-halo-C₁₋₆-alkyl, S(O)₂N(R¹)₂, C(O)N(R¹)₂, CN, C(O)OR⁴ or oxo,
or
two R² on the same carbon atom form together with the carbon atom to which they are attached a C₃₋₁₀ cycloalkyl group,
or
two R² at different carbon atoms form together a —CH₂—, —CH(CH₃)—, —C(CH₃)₂—, CH₂—CH(CH₃)—, —CH₂—CH₂— or —CH₂—CH₂—CH₂— group;
R⁷ and R⁸ are independently selected from the group consisting of hydrogen, halogen, C₁₋₆ alkyl, halo-C₁₋₆-alkyl and OR⁴;
o is 0-4; and
k is 1 or 2.

4. The compound according to claim 1 or 3 which is represented by the following formulae (2-3) and (2-4) 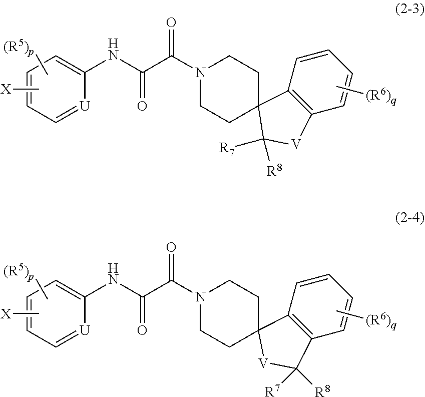
wherein
X is hydrogen, halogen, C₁₋₆-alkyl, C₃₋₆-cycloalkyl, O—C₁₋₆-alkyl, S—C₁₋₆-alkyl, CN or 5- or 6-membered heteroaryl containing 1 to 4 heteroatoms independently selected from O, N and S,
wherein alkyl and cycloalkyl are unsubstituted or substituted with halogen, C₁₋₆-alkyl, halo-C₁₋₆-alkyl, OR¹ or CN;
R⁵ is independently selected from hydrogen, halogen and C₁₋₆-alkyl;
R⁶ is independently selected from halogen, OH, R^{z}, O—R^{z}, OC(O)—R^{z}, S—R^{z}, S(O)₂—R^{z}, S(O)₂N(R)₂, N(R)₂, NR¹C(O)R^{z}, C(O)N(R)₂, C(O)O—R^{z}, C(O)—R^{z}, CN, COOH, 5- or 6-membered heteroaryl containing 1 to 4 heteroatoms independently selected from O, N and S and 6-membered aryl,
wherein aryl and heteroaryl are unsubstituted or substituted with 1 to 5 substituents independently selected from the group consisting of halogen, CN, OR¹, C₁₋₆-alkyl; C₃₋₆-cycloalkyl, and halo-C₁₋₆-alkyl,
R^{z} represents C₁₋₆-alkyl, C₃₋₆-cycloalkyl or 3- to 6 membered heterocyclyl containing 1 to 2 heteroatoms independently selected from O, N and S,
wherein alkyl, cycloalkyl and heterocycloalkyl are unsubstituted or substituted with 1 to 6 substituents independently selected from the group consisting of halogen, C₁₋₆-alkyl, halo-C₁₋₆-alkyl, OR¹ and CN;
R⁷ and R⁸ are independently selected from the group consisting of hydrogen, halogen, C₁₋₆ alkyl, halo-C₁₋₆-alkyl and OR⁴;
U is N or CR⁵;
V is O or CR⁷R⁸;
p is 0, 1, 2 or 3; and
q is 0, 1, 2, 3 or 4.

5. The compound according to claim 1, which is represented by the following formula (1-1) 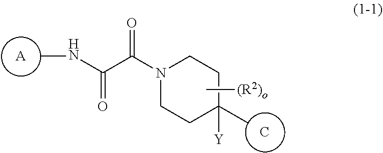
wherein
A represents C₃₋₁₀ cycloalkyl, which may be optionally fused with a phenyl ring being unsubstituted or substituted with 1 to 3 R^{a}, 3- to 10-membered heterocycloalkyl containing 1 to 4 heteroatoms independently selected from O, N and S, 6- to 10-membered mono or bicyclic aryl or 5- to 10-membered mono or bicyclic heteroaryl containing 1 to 4 heteroatoms independently selected from O, N and S,
wherein cycloalkyl, heterocycloalkyl, aryl and heteroaryl are unsubstituted or substituted with 1 to 5 substituents independently selected from the group consisting of halogen, OH, R, O—R^{x}, OC(O)—R^{x}, S—R^{x}, S(O)₂—R^{x}, S(O)₂N(R)₂, N(R¹)₂, NR¹C(O)R, C(O)N(R¹)₂, C(O)O—R^{x}, C(O)—R^{x}, CN, COOH, 5- or 6-membered heteroaryl containing 1 to 4 heteroatoms independently selected from O, N and S and 6-membered aryl,
wherein heteroaryl and aryl are unsubstituted or substituted with 1 to 5 substituents independently selected from the group consisting of halogen, CN, OR¹, C₁₋₆-alkyl, C₃₋₆-cycloalkyl and halo-C₁₋₆-alkyl,
or
two substituents on the same carbon atom or on two different carbon atoms form together with the carbon atom to which they are attached a C₃₋₁₀-cycloalkyl group, wherein optionally one carbon atom in the cycloalkyl ring may be replaced by a heteroatom selected from O, N and S;
R^{a} represents halogen, CN, C₁₋₆-alkyl, halo-C₁₋₆-alkyl, hydroxy-C₁₋₆-alkyl, C₃₋₆-cycloalkyl or halo-C₃₋₆-cycloalkyl;
R^{x} represents C₁₋₆-alkyl, C₃₋₆-cycloalkyl or 3- to 6 membered heterocyclyl containing containing 1 to 2 heteroatoms independently selected from O, N and S,
wherein alkyl, cycloalkyl and heterocycloalkyl are unsubstituted or substituted with 1 to 6 substituents independently selected from the group consisting of halogen, C₁₋₆-alkyl, halo-C₁₋₆-alkyl, OR¹ and CN;
C represents 6- to 10-membered mono- or bicyclic aryl or 5- to 14-membered mono-, bi- or tricyclic heteroaryl containing 1 to 4 heteroatoms independently selected from O, N and S,
wherein aryl and heteroaryl are unsubstituted or substituted with 1 to 5 substituents independently selected from the group consisting of halogen, OH, R^{y}, O—R^{y}, OC(O)—R^{y}, S—R^{y}, S(O)₂—R^{y}, S(O)₂N(R)₂, N(R)₂, NR¹C(O)R^{y}, C(O)N(R)₂, C(O)O—R^{y}, C(O)—R^{y}, CN, COOH, 5- or 6-membered heteroaryl containing 1 to 4 heteroatoms independently selected from O, N and S and 6-membered aryl,
wherein aryl and heteroaryl are unsubstituted or substituted with 1 to 5 substituents independently selected from the group consisting of halogen, CN, OR¹, C₁₋₆-alkyl; C₃₋₆-cycloalkyl, and halo C₁₋₆-alkyl,
or
two substituents on the aryl or heteroaryl ring systems together with the carbon atoms to which they are attached form a 5- or 6-membered heterocyclic ring containing 1 or 2 heteroatoms independently selected from O, N and S,
wherein the heterocylic ring is unsubstituted or substituted with 1 or 2 substituents independently selected from the group consisting of halogen, C₁₋₆-alkyl, halo-C₁₋₆-alkyl, CN and oxo;
R^{y} represents C₁₋₆-alkyl, C₃₋₆-cycloalkyl or 3- to 6 membered heterocyclyl containing 1 to 2 heteroatoms independently selected from O, N and S,
wherein alkyl, cycloalkyl and heterocycloalkyl are unsubstituted or substituted with 1 or 2 substituents independently selected from the group consisting of halogen, C₁₋₆-alkyl, halo-C₁₋₆-alkyl, OR¹ and CN;
Y is hydrogen, OR⁴, halogen, C₁₋₆-alkyl or halo-C₁₋₆-alkyl;
R² is halogen, C₁₋₆ alkyl, C₃₋₆-cycloalkyl, halo-C₁₋₆-alkyl, OR⁴, S(O)₂N(R¹)₂, S(O)₂—C₁₋₆-alkyl, S(O)₂—C₃₋₆-cycloalkyl, S(O)₂-halo-C₁₋₆-alkyl, S(O)₂N(R¹)₂, C(O)N(R¹)₂, CN, C(O)OR⁴ or oxo,
or
two R² on the same carbon atom form together with the carbon atom to which they are attached a C₃₋₁₀-cycloalkyl group,
or
two R² at different carbon atoms form together a —CH₂—, —CH(CH₃)—, —C(CH₃)₂—, CH₂—CH(CH₃)—, —CH₂—CH₂— or —CH₂—CH₂—CH₂— group; and
o is 0-4.

6. The compound according to claim 1 or 5, which is represented by the following formula (1-2) 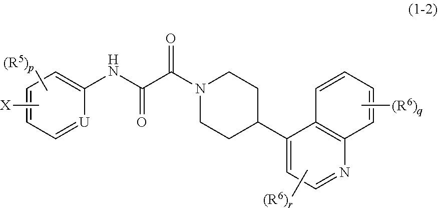
wherein
R⁵ is independently selected from hydrogen, halogen and C₁₋₆-alkyl;
R⁶ is independently selected from the group consisting of halogen, OH, R, O—R^{x}, OC(O)—R^{x}, S—R^{x}, S(O)₂—R^{x}, S(O)₂N(R¹)₂, N(R¹)₂, NR¹C(O)R, C(O)N(R¹)₂, C(O)O—R^{x}, C(O)—R^{x}, CN, COOH, 5- or 6-membered heteroaryl containing 1 to 4 heteroatoms independently selected from O, N and S and 6-membered aryl,
wherein heteroaryl and aryl are unsubstituted or substituted with 1 to 5 substituents independently selected from the group consisting of halogen, CN, OR¹, C₁₋₆-alkyl, C₃₋₆-cycloalkyl and halo-C₁₋₆-alkyl;
R^{x} represents C₁₋₆-alkyl, C₃₋₆-cycloalkyl or 3- to 6 membered heterocyclyl containing 1 to 2 heteroatoms independently selected from O, N and S,
wherein alkyl, cycloalkyl and heterocycloalkyl are unsubstituted or substituted with 1 or 6 substituents independently selected from the group consisting of halogen, C₁₋₆-alkyl, halo C₁₋₆-alkyl, OR¹ and CN;
X is hydrogen, halogen, C₁₋₆-alkyl, C₃₋₆-cycloalkyl, O—C₁₋₆-alkyl, S—C₁₋₆-alkyl, CN or 5- or 6-membered heteroaryl containing 1 to 4 heteroatoms independently selected from O, N and S,
wherein alkyl and cycloalkyl are unsubstituted or substituted with halogen, C₁₋₆-alkyl, halo-C₁₋₆-alkyl, OR¹ or CN;
U is N or CR⁵;
p is 0, 1, 2 or 3;
q is 0, 1, 2, 3 or 4; and
r is 0, 1 or 2.

7. The compound according to claim 4 or 6, wherein U is CR⁵.

8. The compound according to any of claims 1, 2 and 5 to 7, which is selected from the group consisting of

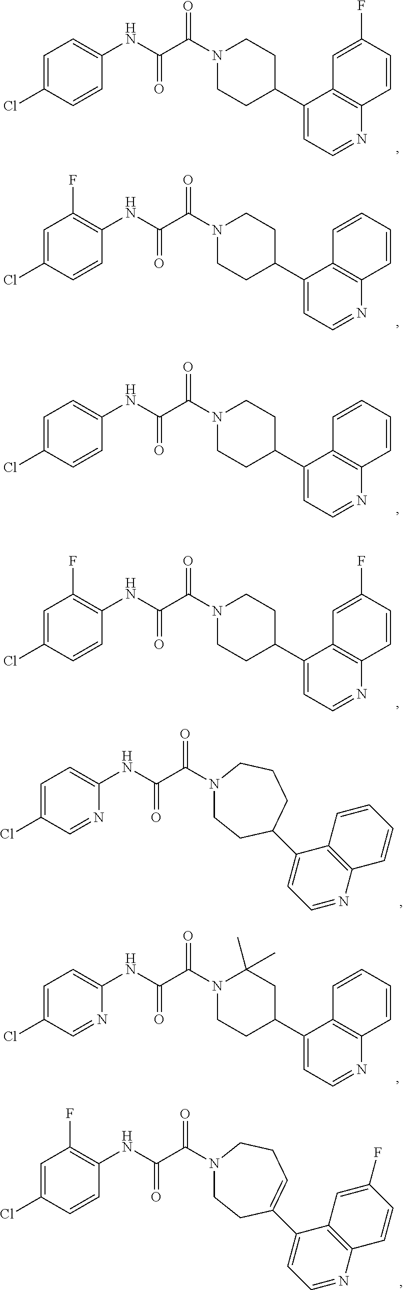

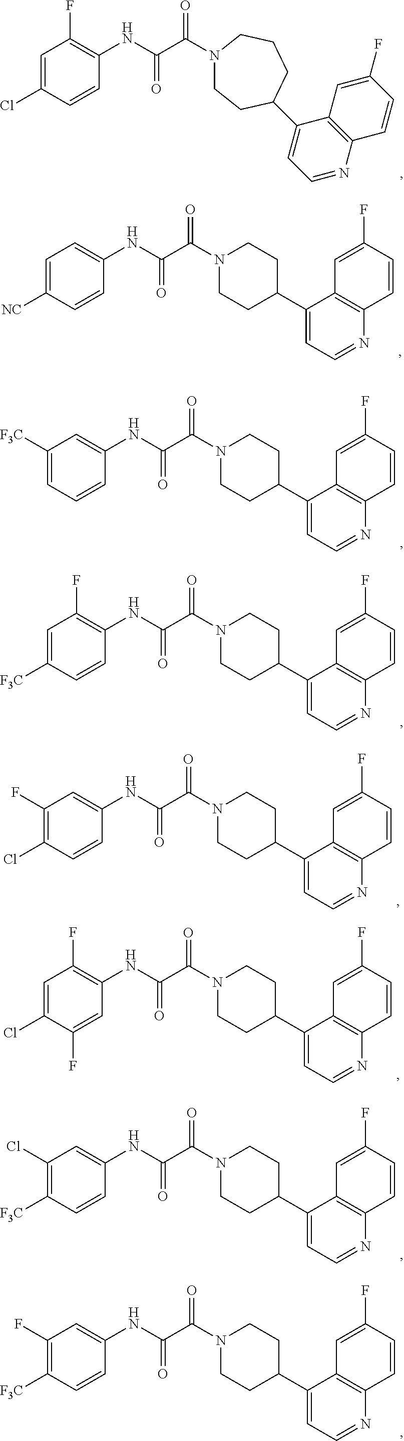

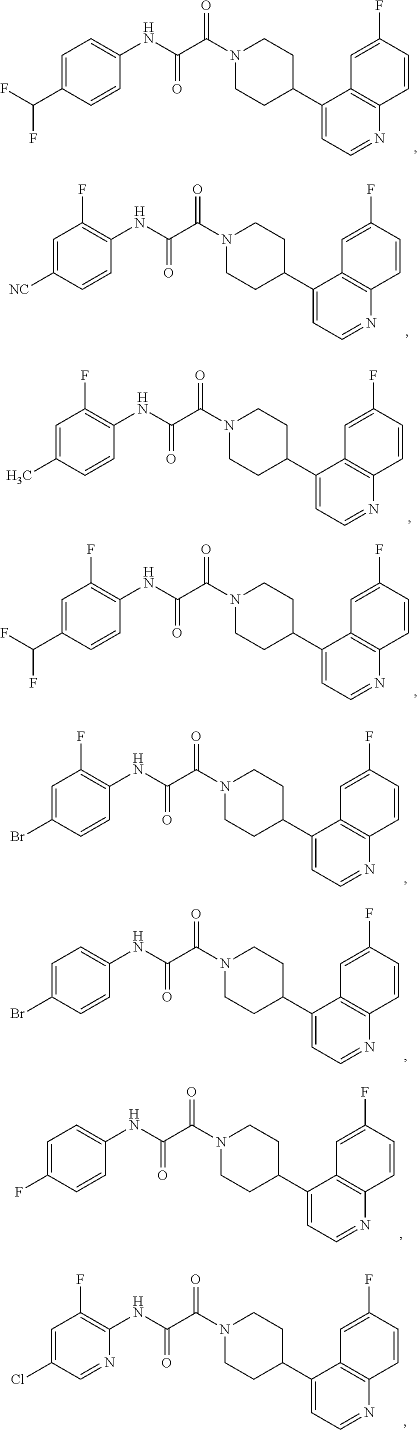

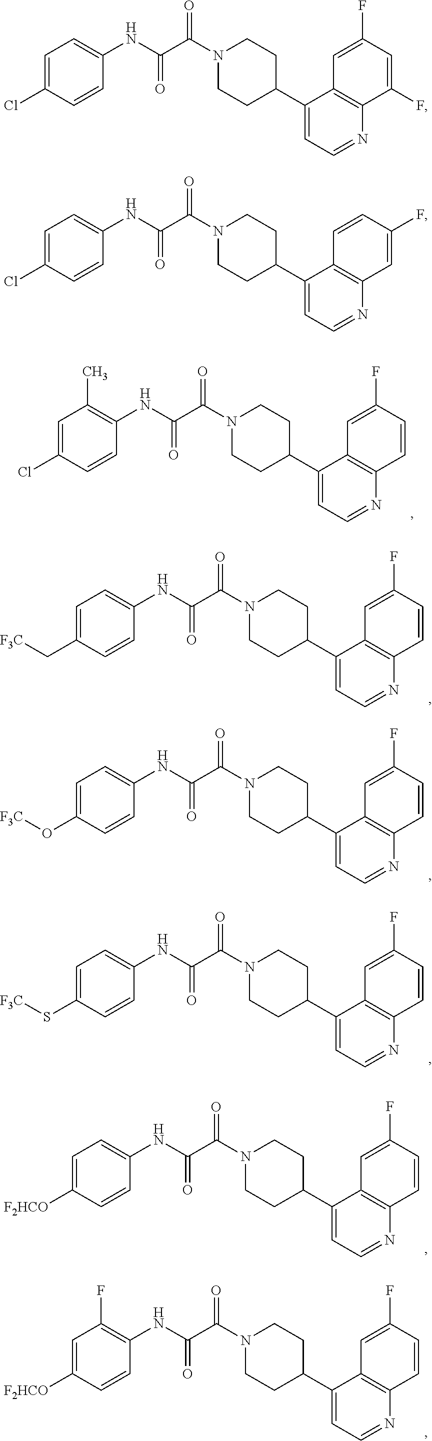

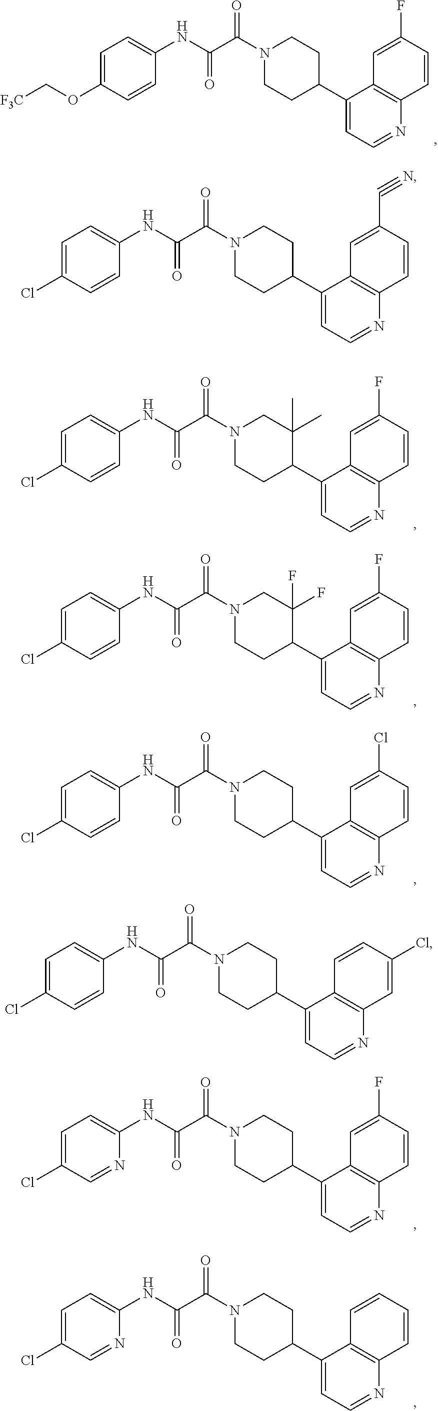

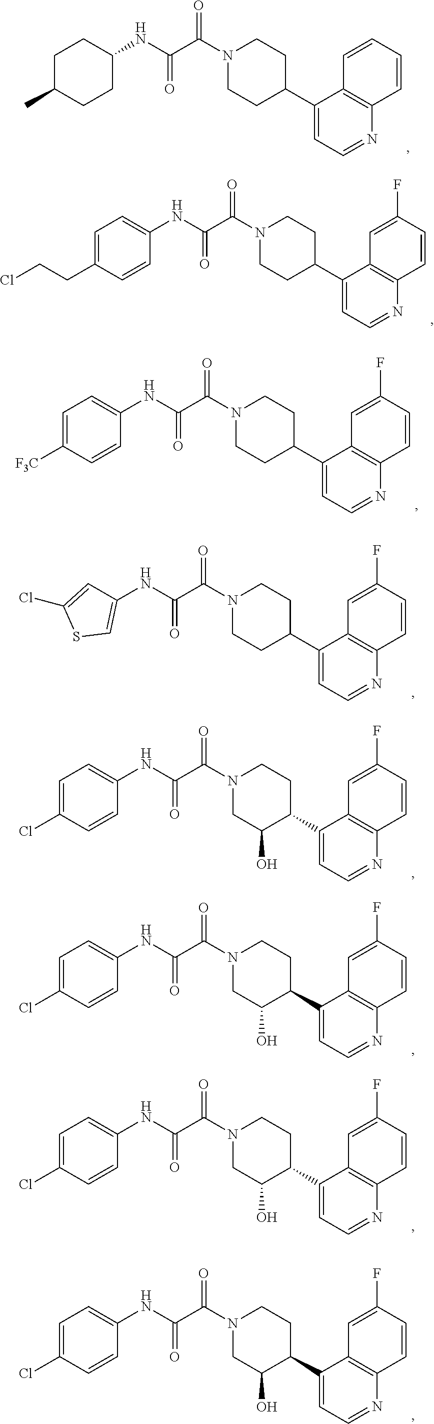

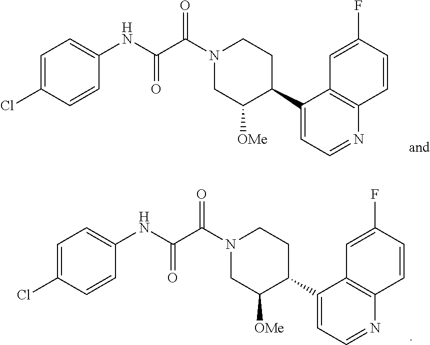 [SMILES: CO[C@@H]1CN(C(=O)C(=O)Nc2ccc(Cl)cc2)CC[C@H]1c1ccnc2ccc(F)cc12 ; CO[C@H]1CN(C(=O)C(=O)Nc2ccc(Cl)cc2)CC[C@@H]1c1ccnc2ccc(F)cc12]

9. The compound according to any of claims 1 to 4 and 7, wherein the compound is selected from the group consisting of

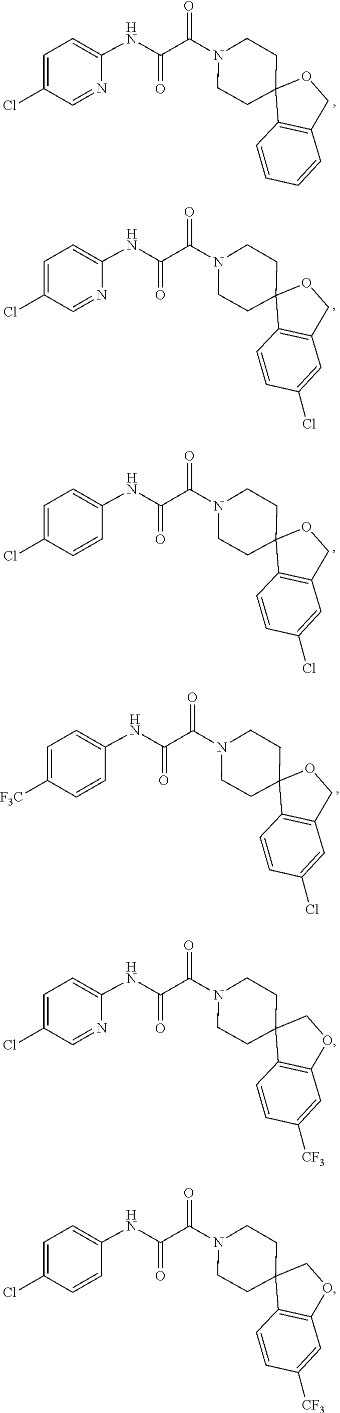 [SMILES: O=C(Nc1ccc(Cl)cn1)C(=O)N1CCC2(CC1)OCc1ccccc12 ; O=C(Nc1ccc(Cl)cn1)C(=O)N1CCC2(CC1)OCc1cc(Cl)ccc12 ; O=C(Nc1ccc(Cl)cc1)C(=O)N1CCC2(CC1)OCc1cc(Cl)ccc12 ; O=C(Nc1ccc(C(F)(F)F)cc1)C(=O)N1CCC2(CC1)OCc1cc(Cl)ccc12 ; O=C(Nc1ccc(Cl)cn1)C(=O)N1CCC2(CC1)COc1cc(C(F)(F)F)ccc12 ; O=C(Nc1ccc(Cl)cc1)C(=O)N1CCC2(CC1)COc1cc(C(F)(F)F)ccc12]

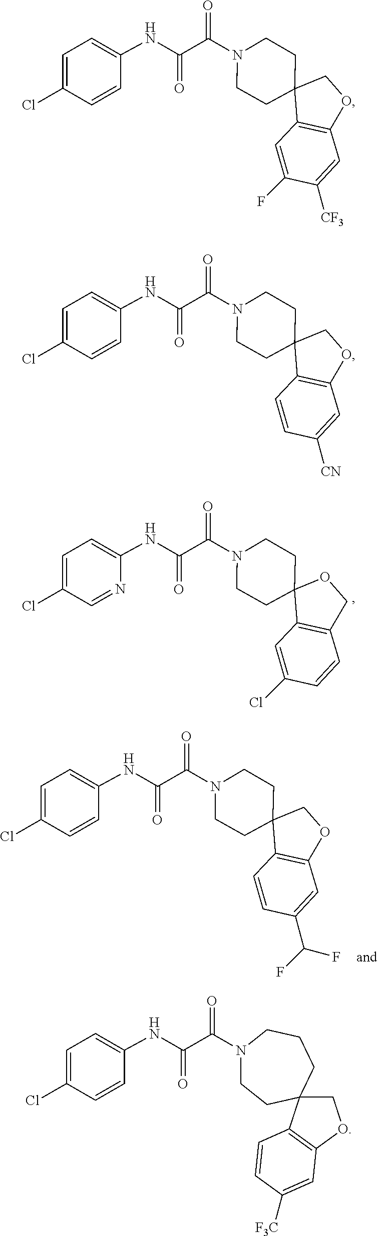 [SMILES: O=C(Nc1ccc(Cl)cc1)C(=O)N1CCC2(CC1)COc1cc(C(F)(F)F)c(F)cc12 ; N#Cc1ccc2c(c1)OCC21CCN(C(=O)C(=O)Nc2ccc(Cl)cc2)CC1 ; O=C(Nc1ccc(Cl)cn1)C(=O)N1CCC2(CC1)OCc1ccc(Cl)cc12 ; O=C(Nc1ccc(Cl)cc1)C(=O)N1CCC2(CC1)COc1cc(C(F)F)ccc12 ; O=C(Nc1ccc(Cl)cc1)C(=O)N1CCCC2(CC1)COc1cc(C(F)(F)F)ccc12]

10. The compound according to any of claims 1 to 9 for use as a medicament.

11. The compound according to any of claims 1 to 9 for use in the prophylaxis and/or treatment of a disease or condition mediated by indoleamine 2,3-dioxygenase.

12. The compound for use according to claim 11 wherein the disease or condition is selected from the group consisting of cancer, viral and bacterial infections such as HIV infection, hanta virus infection, tuberculosis, leprae, depression, epilepsy, schizophrenia, neurodegenerative diseases such as Alzheimer's disease and Huntington's disease, trauma, age-related cataracts, organ transplantation, cardiovascular disease, endometriosis, type 2 diabetic nephropathy, chronic obstructive pulmonary disease (COPD), osteoporosis, asthma, rheumatoid arthritis, multiple sclerosis, inflammatory bowel disease, psoriasis, and systemic lupus erythematosus.

13. The compound for use according to claim 12 wherein the disease or condition is cancer.

14. The compound for use according to claim 13 wherein the compound is administered with one or more therapeutic agents for cancer selected from the group consisting of PD-1 agent, PD-L1 agent, CTLA-4 agent, chemotherapeutic agent, anticancer vaccine, oncolytic viruses, cytokine therapy, TLR agonists, STING agonists, as well as other immuno oncology therapeutics, or wherein the compound is administered under irradiation therapy.

15. A pharmaceutical composition comprising a compound according to any of claims 1 to 9 and pharmaceutically acceptable excipients.
